(19) United States
(12) Patent Application Publication
(10) Pub. No.: US 2026/0027096 A1
(43) Pub. Date: Jan. 29, 2026

(54) PHD INHIBITORS

(71) Applicants: Oxford University Innovation Limited, Oxford (GB); Queen Mary University of London, London (GB)

(72) Inventors: Christopher Joseph Schofield, Oxford (GB); James Preston Holt-Martyn, Oxford (GB); Kamil Kranc, Sutton London (GB); Hannah Lawson, Sutton London (BG); Jozef Durko, Trnava (SK)

(21) Appl. No.: 19/150,008

(22) PCT Filed: Jan. 25, 2024
(86) PCT No.: PCT/GB2024/050203

§ 371 Date: Jul. 22, 2025

(30) Foreign Application Priority Data
Jan. 25, 2023 (GB) 2301059.8

(51) Int. Cl. A61K 31/4439; A61K 31/222; A61K 31/444; A61K 31/501; A61K 31/506; A61K 31/513; A61K 31/5377; A61K 31/635; A61K 45/06; A61P 35/02; C07D 401/04; C07D 401/14; C07D 403/04; C07D 413/14; C07D 417/14; C07D 471/14

(52) U.S. Cl. CPC A61K 31/4439; A61K 31/222; A61K 31/444; A61K 31/501; A61K 31/506; A61K 31/513; A61K 31/5377; A61K 31/635; A61K 45/06; A61P 35/02; C07D 401/04; C07D 401/14; C07D 403/04; C07D 413/14; C07D 417/14; C07D 471/14

(57) ABSTRACT

A hypoxia inducible factor prolyl hydroxy lase inhibitor (PHD inhibitor) for use in the treatment of blood cancer.

### FIELD OF THE INVENTION

The invention relates to the treatment of blood cancers, including acute myeloid leukaemia (AML), chronic myeloid leukaemia (CML) and multiple myeloma (MM), by increasing hypoxia inducible factor alpha (HIF-α), typically by using a hypoxia inducible factor prolyl hydroxylase inhibitor (a PHD inhibitor). Thus, in one aspect the invention relates to PHD inhibitors for use in the treatment of blood cancers.

### BACKGROUND TO THE INVENTION

Blood cancer is the fifth most common cancer and has a very high mortality rate (https://bloodcancer.org.uk/news/blood-cancer-facts/). There are 3 main groups of blood cancer; leukaemia, lymphoma, and myeloma. Acute myeloid leukaemia (AML) is an aggressive clonal disorder of haematopoietic stem cells (HSCs) and progenitors, in which they acquire driver mutations, resulting in generation of treatment-resistant leukaemic stem cells (LSCs) (Horton, S. J., et al., (2012), Haematologica 97, 966-974; Shlush, L. I., et al. (2014) Nature 506, 328-333). LSCs are characterised by an uncontrolled self-renewal capacity and impaired differentiation potential, and have the ability to initiate and propagate leukaemia. AML is manifested by over-proliferation of primitive myeloid progenitors, referred to as blasts, which infiltrate bone marrow (BM), thus inhibiting normal multilineage haematopoiesis and causing a range of severe clinical symptoms. Current conventional therapies, which have remained largely unchanged for the past 4 decades, effectively target the bulk AML population, but often fail to fully eradicate LSCs (Dohner, H., et al. (2015) N Engl J Med 373, 1136-1152). The surviving LSCs drive minimal residual disease, ultimately causing often incurable and fatal disease relapses. Recent advances in AML treatment, including the introduction of the B-cell lymphoma 2 (BCL2) inhibitor Venetoclax in combination with hypomethylating agents⁹⁹, has set the stage for the new era on AML treatment as the field awaits the long-term outcomes of these promising therapies (DiNardo, C. D., et al. (2020) N Engl J Med 383, 617-629). However, for the time being, effective AML treatment remains an unmet clinical need with an overall 5-year survival rate of only approximately 30%, highlighting the immense importance of searching for novel therapeutic targets to combat this disease.

Chronic myeloid leukaemia (CML) results from acquisition of the BCR-ABL1 translocation in HSCs, transforming them into LSCs, which give rise to and sustain a myeloproliferative disease (Chronic myeloid leukaemia; Cortes, Jorge et al.; The Lancet, Volume 398, Issue 10314, 1914-1926). Despite developments in the field using tyrosine kinase inhibitors (TKIs), only ˜10% of CML patients maintain a therapy-free remission (Holyoake T L, Vetrie D; The chronic myeloid leukemia stem cell: stemming the tide of persistence; Blood. 2017 Mar. 23;129 (12): 1595-1606).

Multiple myeloma (MM) is a cancer of blood plasma cells (Roman-Trufero M, Auner H W and Edwards C M (2022); Multiple myeloma metabolism—a treasure trove of therapeutic targets? Front. Immunol. 13:897862). MM is the second most common blood cancer in high-income countries, with limited effective therapies, resulting in the majority of patients dying from MM (Multiple myeloma; van de Donk, Niels W C J et al, The Lancet, Volume 397, Issue 10272, 410-427).

Given the hypoxic nature of the bone marrow, therapeutic manipulation of the hypoxia signalling pathways offers an opportunity to improve blood cancer outcomes. Cellular responses to hypoxia are mediated by HIF-1 and HIF-2, which are dimers of oxygen-regulated a subunits (collectively called HIF-α) and stable HIF-β subunits (Gezer, D., et al. (2014). Stem Cells 32, 1390-1397; Kaelin, W. G., Jr et al (2008) Mol Cell 30, 393-402; Schofield, C. J., et al (2004) Nat Rev Mol Cell Biol 5, 343-354).

Under normoxia, HIF-α are hydroxylated by oxygen-dependent HIF prolyl hydroxylases (PHDs, namely PHD1, PHD2 and PHD3, with PHD2 being the predominant HIF-α regulator), resulting in VHL-mediated HIF-α degradation (Semenza, G. L. (1998). Curr Opin Genet Dev 8, 588-594; Semenza, G. L. (2013) J Clin Invest 123, 3664-3671; Semenza, G. L. (2014) Annu Rev Pathol 9, 47-71; Yamamoto, A., et al. (2019) J Clin Invest 130, 3640-3656; Yang, M., et al. (2014) Hypoxia 2, 127-142).

In hypoxia, when PHD activity is decreased, HIF-α isoforms are stabilised and translocate to the nucleus where they bind HIF-β and promote transcription of hypoxia-inducible genes (Mole, D. R., et al. (2009) J Biol Chem 284, 16767-16775; Schodel, J.,et al. (2013) Biol Chem 394, 507-517; Schodel, J., et al (2011) Blood 117, e207-217). Despite the interest in the role of the HIF pathway in blood cancer, its therapeutic significance has remained unresolved. While earlier findings indicated that knockdown of HIF1α or HIF2α may compromise human AML (Rouault-Pierre, K., et al. (2013) Cell Stem Cell 13, 549-563; Wang, Y., et al. (2011) Cell Stem Cell 8, 399-411), genetic studies in mice imply that deletion of Hif-1α, Hif-2α or both Hif-1α and Hif-2α accelerated leukaemogenesis (Vukovic et al., J Exp Med. 2015 Dec. 14; 212 (13): 2223-34).

Acute myeloid leukaemia (AML) is an aggressive clonal disease of haematopoietic stem cells (HSCs) and their primitive progenitors, which acquire diverse mutations to drive disease initiation and progression^{1,2}. Despite recent advances³⁻⁸, most AML cases remain highly aggressive, resulting in an overall 5-year survival rate of ˜30% ^{9,10}. Hence, there is a clear unmet clinical need to identify new non-toxic therapeutic strategies for improved AML treatment.

HIF-α, but not HIF-β, levels are regulated by HIF prolyl hydroxylase (PHD1-3) catalysis, with PHD2 considered to be the key contributor to setting the steady-state levels of HIF-1α under normoxic conditions¹⁷⁻²⁵. Under normal physiological conditions, when O₂ levels are not limiting, the PHDs efficiently catalyse C-4 hydroxylation of proline-residues in the a subunits of HIF-1 and HIF-2 (collectively HIF-α). This post-translational modification strengthens binding of HIF-α to the von Hippel Lindau (VHL) protein, a targeting component of a ubiquitin ligase complex, resulting in HIF-α ubiquitination and subsequent degradation²⁶⁻²⁹. In hypoxia, PHD activity decreases and thus HIF-α isoforms are stabilised and translocate to the nucleus where they bind HIF-β and promote transcription of HIF target genes, which function to ameliorate the effects of hypoxia³⁰⁻³². Activation of HIF-mediated gene expression in the absence of hypoxia can be achieved by pharmacological inhibition of the PHDs³³⁻³⁶. Small-molecule PHD inhibitors have shown non-toxic therapeutic utility; Roxadustat and Daprodustat (Dap), inter alia, stimulate erythropoietin production in a HIF-α dependent manner to enhance erythropoiesis for anaemia treatment in patients with chronic renal failure^{37,38}. Roxadustat has also been shown experimentally in mouse model studies to suppress M2 macrophage polarisation to protect from renal fibrosis³⁹ and activate phagocytosis in a subset of tumour-infiltrating macrophages to promote their antitumour potential and inhibit tumour growth⁴⁰. Furthermore, pharmacological PHD inhibition enhances the antibacterial activity of skin phagocytes and keratinocytes⁴¹ and boosts mucosal protection during colitis⁴²⁻⁴⁴. Notably, dimethyloxalylglycine (DMOG), a prodrug precursor of N-oxalylglycine, which inhibits multiple 2-oxoglutarate (2OG)-dependent oxygenases⁴⁵, including the PHDs, and which stabilises HIF-α in cells, decreases survival of human THP-1 AML cells with MLL-AF9 translocation⁴⁶. However, the therapeutic significance of selective pharmacological PHD inhibition with consequent HIF-α upregulation in many diseases and malignancies, including AML remains unknown.

### SUMMARY OF THE INVENTION

The present inventors have addressed the hypothesis that inhibition of prolyl hydroxylase domain enzymes (PHDs) and therefore activation of HIF-dependent pathways is beneficial for blood cancer treatment. The present inventors found that while PHD2 is required for AML initiation, and its ablation in established AML compromises the disease progression, loss of PHD2 has no substantial impact on multilineage haematopoiesis. Furthermore, the present inventors have found that inactivation of PHD1 and PHD2 can compromise both AML initiation and disease progression and hinder LSC (leukaemic stem cell) maintenance. Notably, pharmacological PHD inhibtion using a currently available PHD targeting drug (e.g. Daprodustat) or a novel more selective PHD inhibitor (e.g. a compound of formula I or IV herein) or other known method of PHD inhibition leads to AML, CML and MM cell apoptosis. It is a finding of the invention that prolyl hydroxylase domain enzyme catalysis is required for the initiation of AML driven by Meis1 and Hoxa9. The inventors have also found that PHD catalysis is required for MLL-AF9-driven leukaemogenesis. PHD2 knockdown in established leukaemia cells can compromise leukaemia progression. Taken together, the data show that PHD activity is important for AML cell survival and efficient AML disease progression, highlighting its value as a therapeutic target in leukemia. In addition, the inventors have found that, in in vivo models, PHD inhibition has no major impact on survival and normal haematopoiesis. The findings show that inhibition of PHDs is a very promising clinical pathway to the treatment of blood cancer. The inventors have validated this by testing both currently-available and newly-generated PHD inhibitors on a range of genetically diverse AML, CML and MM cells, and have demonstrated that a range of PHD inhibitors, with distinct modes of action, do indeed compromise blood cancer cells. Thus, the inventors have provided both genetic and pharmacological evidence that PHD inhibition is a very promising therapeutic strategy for blood cancer. Given that the primary function of the PHDs are to promote hypoxia inducible factor-α (HIF-α) degradation, the inventors measured HIF-1α levels following the treatment of AML cells with PHD inhibitors, and found increased HIF-1α levels in the treated cells. They went on to find that a PHD inhibitor did not compromise the proliferation of AML cells lacking both HIF-1α and HIF-2α and but did compromise the proliferation of control AML cells, implying that PHD inhibition exhibits its anti-leukaemic effect in a HIF-dependent manner. These and other findings herein show that the increase of hypoxia inducible factor (HIF), and in particular HIF-α, is a promising clinical pathway to the treatment of leukaemia and other blood cancers. Furthermore, while PHD inhibition strongly activates the expression of pro-apoptotic BNIP3 expression which activates BAX/BAX-mediated mitochondrial outer membrane destbilisation, additional inactivation of BCL-2 (by Venetoclax), an inhibitor of BAX/BAX-dependent apoptosis, potentiates this effect. Thus, the inventors' results also set the stage for a novel therapy employing combined PHD and BCL-2 inhibition in AML.

In a broad aspect, therefore, the present invention provides a method of treating blood cancer by increasing HIF. One method of increasing HIF in order to treat blood cancer is to employ a hypoxia inducible factor prolyl hydroxylase inhibitor (which is also referred to herein as a PHD inhibitor). However, any other method of increasing HIF will also be applicable and other such methods are discussed further herein.

The present invention also therefore provides a hypoxia inducible factor prolyl hydroxylase inhibitor (i.e. a PHD inbibitor) for use in the treatment of blood cancer.

The PHD inhibitor for use in the treatment of blood cancer may be an inhibitor of PHD2, i.e. it may be a PHD2 inhibitor. Alternatively, the PHD inhibitor may be a PHD1 inhibitor, or a PHD3 inhibitor, or it may be a PHD inhibitor which inhibits any combination of PHDs. For instance, the PHD inhibitor may be an inhibitor of two or more of PHD1, PHD2 and PHD3, for example it may be an inhibitor of PHD2 and PHD1, an inhibitor of PHD2 and PHD3, an inhibitor of PHD1 and PHD3, or an inhibitor of PHD1, PHD2 and PHD3. A variety of PHD inhibitors are known in the art and any of these, or derivatives of them optimised for blood cancer treatment, may be employed as the PHD inhibitor in the present invention. The PHDs use active site bound Fe(II) as a cofactor and O₂ and 2-oxoglutarate as cosubstrates. One mechanism of PHD inhibition involves small molecule that chelates the active site Fe(II) chelation and which competes with 2-oxoglutarate for binding at the active site. Such inhibitors may or may not compete with the HIF-alpha substrate for binding at the active site.

Accordingly, the invention provides a PHD inhibitor for use in the treatment of blood cancer (which may for instance be AML, CML or MM). The PHD inhibitor may be any one of the following types of PHD inhibitor, or indeed any other PHD inhibitor: a cobalt compound, for instance a cobalt salt (e.g. cobalt dichloride); a copper compound, for instance a copper salt; a nickel compound, for instance a nickel salt; an iron chelator, for instance deferoxamine, 3,4-dihydroxybenzoic acid, a 1,10-phenanthroline or quercetin; a 2-oxoglutarate (2-OG) derivative, mimic, or competitor (with respect to PHD binding), for instance dimethyloxalylglycine (DMOG) which is a prodrug form of N-oxalylglycine (NOG); FG-2216; roxadustat; a quinolone, for instance JNJ-42905343; a quinoxaline; a benzamidazole derivative, for instance JNJ-42041935; an isoquinolone derivative; a 5-hydroxy-1,7 naphthyridine derivative, for instance ISM5411; a monocyclic pyridine compound, for instance vadadustat or AKB6899; a pyrazolopyrimidine derivative; a pyrimidine-trione, for instance daprodustat; an N-alkoxyquinolone, for instance desidustat; a tetrahydropyran derivative; a dihydrothienopyridone derivative; a dihydrofuropyridoene derivative; a quinazoline-2,4-dione; a 4-oxo-2-thioxo-7-quinasoline; a 5-aminocarbonyl-4-hydroxypyrimidine derivative, for instance MK8617; a spiroindolone; a 2,8-diazaspori[4,5]-decan-lone; a pyrazolone derivative, for instance molidustat; a triazole substituted heteroaryl amide; a phenolic compound, for instance (2S)-({[2-(5-cyano-3-hydroxypyridin-2-yl)-1,3-thiazol-4-yl]acetyl}amino) (phenyl) ethanoic acid); a bicyclic heteroaryl derivative, for instance 1,2,4-triazolo-[1,5-a]pyridine); a diacylhydrazine; or pyrathione Zn or (5-(3-(4-chlorophenoxyl) prop-1-yn-1-yl)-3-hydroxypicolinoyl)glycine.

The PHD inhibitor may for instance be daprodustat or a structurally-related PHD inhibitor. Accordingly, the present invention also provides a PHD inhibitor for use in the treatment of blood cancer wherein said PHD inhibitor is a compound of formula (II) or a pharmaceutically acceptable salt thereof

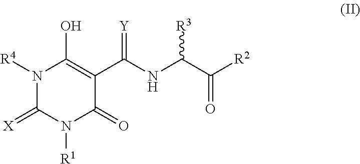

wherein
R¹ and R⁴ are each independently selected from the group consisting of H, —NR⁵R⁶, unsubstituted or substituted C₁₋₁₀ alkyl, unsubstituted or substituted C₂₋₁₀ alkenyl, unsubstituted or substituted C₂₋₁₀ alkynyl, unsubstituted or substituted C₃₋₈ cycloalkyl, unsubstituted or substituted —C₃₋₈ cycloalkylene-C₁₋₁₀ alkyl, unsubstituted or substituted C₅₋₈ cycloalkenyl, unsubstituted or substituted —C₅₋₈ cycloalkenylene-C₁₋₁₀ alkyl, unsubstituted or substituted C₃₋₈ heterocyclyl, unsubstituted or substituted —C₃₋₈ heterocyclylene-C₁₋₁₀ alkyl, unsubstituted or substituted aryl, unsubstituted or substituted-arylene-C₁₋₁₀ alkyl, unsubstituted or substituted-heteroaryl and unsubstituted or substituted-heteroarylene-C₁₋₁₀ alkyl; R² is —NR⁷R⁸ or —OR⁹;
R³ is H or unsubstituted or substituted C₁₋₄ alkyl;
where R⁵ and R⁶ are each independently selected from the group consisting of H, unsubstituted or substituted C₁₋₁₀ alkyl, unsubstituted or substituted —C₃₋₈ cycloalkyl, unsubstituted or substituted —C₃₋₈ cycloalkylene-C₁₋₁₀ alkyl, —C₃₋₈ heterocyclyl, unsubstituted or substituted —C₃₋₈ heterocyclylene-C₁₋₁₀ alkyl, unsubstituted or substituted aryl, unsubstituted or substituted-arylene-C₁₋₁₀ alkyl, unsubstituted or substituted heteroaryl, unsubstituted or substituted-heteroarylene-C₁₋₁₀ alkyl, unsubstituted or substituted —C(O)C₁₋₄ alkyl, unsubstituted or substituted —C(O)C₃₋₆ cycloalkyl, —C(O)C₃₋₆ heterocyclyl, unsubstituted or substituted —C(O) aryl, unsubstituted or substituted-C(O) heteroaryl and unsubstituted or substituted —S(O)₂C₁₋₄ alkyl, or, when R⁵ and R⁶ are attached to the same nitrogen, R⁵ and R⁶ taken together with the nitrogen to which they are attached form a 5-or 6-or 7-membered saturated heterocyclic ring which is unsubstituted or substituted and which optionally contains one other heteroatom selected from oxygen, nitrogen and sulphur,
R⁷ and R⁸ are each independently selected from the group consisting of H, unsubstituted or substituted C₁₋₁₀ alkyl, unsubstituted or substituted C₂₋₁₀ alkenyl, unsubstituted or substituted C₂₋₁₀ alkynyl, unsubstituted or substituted C₃₋₈ cycloalkyl, unsubstituted or substituted C₃₋₈ heterocyclyl, unsubstituted or substituted aryl, and unsubstituted or substituted heteroaryl, and
R⁹ is H or C₁₋₁₀ alkyl which is unsubstituted or substituted with one or more substituents independently selected from the group consisting of unsubstituted or substituted C₃₋₆ cycloalkyl, unsubstituted or substituted C₃₋₈ heterocyclyl, unsubstituted or substituted aryl, and unsubstituted or substituted heteroaryl;
X is O or S; and
Y is O or S.

The compound of formula (II) may for instance be daprodustat.

Alternatively, for instance, the PHD inhibitor may be molidustat or a structurally-related PHD inhibitor. Accordingly, the present invention also provides a PHD inhibitor for use in the treatment of blood cancer wherein said PHD inhibitor is a compound of formula (III) or a pharmaceutically acceptable salt thereof

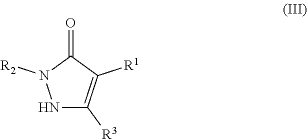

in which
R¹ represents a heteroaryl group of the formula

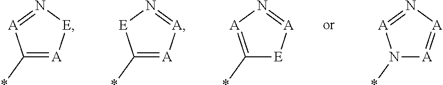

wherein * denotes the linkage point with the dihydropyrazolone ring,
A in each individual occurrence denotes C—R⁴ or N, wherein at most two ring members A represent N at the same time, and
E denotes O, S or N—R⁵,
R² represents a heteroaryl group of the formula

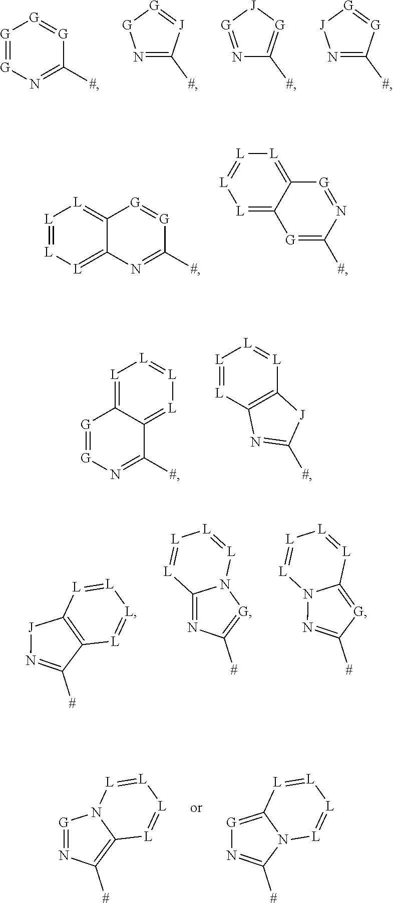

wherein #denotes the linkage point with the dihydropyrazolone ring, G in each individual occurrence denotes C—R⁶ or N, wherein at most two ring members G represent N at the same time,
   J denotes O, S or N—R⁷, and
   L in each individual occurrence denotes C—R⁸ or N, wherein at most two ring members L represent N at the same time,
   wherein R⁴, R⁶ and R⁸ are the same or different and are each independently selected from H or a substituent chosen from the series consisting of halogen, —CN, nitro, C₁₋₆ alkyl, —C₃₋₇-cycloalkyl, 4-to 10-membered heterocyclyl, phenyl, 5 or 6-membered heteroaryl, —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹, wherein
   (i) C₁₋₆ alkyl is unsubstituted or substituted one to three times by the same or different groups independently selected from halogen, CN, oxo, —C₃₋₇-cycloalkyl, 4-to 10-membered heterocyclyl, phenyl, 5-or 6-membered heteroaryl —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹, wherein the cycloalkyl, heterocyclyl, phenyl and heteroaryl groups may unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxy-carbonyl,
   (ii) C₃₋₇ cycloalkyl, 4-to 10-membered heterocyclyl, phenyl and 5-or 6-membered heteroaryl may be unsubstituted or substituted one to three times by the same or different groups independently selected from halogen, —CN, nitro, C₁₋₆ alkyl, —C₃₋₇-cycloalkyl, 4-to 10 membered heterocyclyl, phenyl, 5 or 6-membered heteroaryl, —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹,
   wherein the alkyl group is unsubstituted or substituted one to three times by the same or different groups independently selected from halogen, CN, hydroxyl, trifluoromethoxy, (C₁₋₄)-alkoxy, amino, mono-(C₁₋₄)-alkylamino, di-(C₁₋₄)-alkylamino, hydroxycarbonyl, (C₁₋₄)-alkoxycarbonyl, (C₃₋₇)-cycloalkyl, 4-to 7-membered heterocyclyl, phenyl and/or 5-or 6-membered heteroaryl,
   (iii) R⁹, R¹⁰, R¹¹, R¹³, R¹⁴, R¹⁷, R¹⁹, R²¹, R²⁴, R²⁵, R²⁶, R²⁸, R²⁹ and R³⁰ independently of one another for each individual occurrence represent groups selected from H, C₁₋₆ alkyl, C₃₋₇ cycloalkyl, 4-to 10-membered heterocyclyl, phenyl and 5-or 6-membered heteroaryl, wherein
   C₃₋₇ cycloalkyl, 4-to 10-membered heterocyclyl, phenyl and 5-or 6-membered heteroaryl may be unsubstituted or substituted one to three times by the same or different groups independently selected from halogen, CN, C₁₋₄-alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl and
   C₁₋₆ alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl, (C₁₋₄)-alkoxycarbonyl, C₃₋₇ cycloalkyl, C₄₋₇ heterocycloalkyl, phenyl and/or 5-or 6-membered heteroaryl,
   (iv) R¹², R¹⁵, R¹⁶, R¹⁸, R²⁰, R²², R²³, R²⁷ and R³¹ independently of one another for each individual occurrence represent groups selected from H and C₁₋₆ alkyl,
   wherein C₁₋₆ alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl and/or (C₁₋₄)-alkoxycarbonyl, and/or wherein
   (v) R¹¹ and R¹², R¹⁴ and R¹⁵, R¹⁶ and R¹⁷, R¹⁸ and R¹⁹, R²⁰ and R²¹, R²¹ and R²², R²³ and R²⁴, R²⁶ and R²⁷ and R³⁰ and R³¹ in each case paired together with the atoms to which they are bonded can form a 5-or 6-membered heterocyclyl ring, which may be unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl,
and
   R⁵ and R⁷ are the same or different and independently and are each selected from H, C₁₋₆ alkyl, C₃₋₇ cycloalkyl, C₄₋₇ heterocycloalkyl, phenyl and 5-or 6-membered heteroaryl, wherein
   (i) C₁₋₆ alkyl is unsubstituted or substituted one to three times by the same or different groups independently selected from halogen, —CN, nitro, —C₃₋₇-cycloalkyl, 4-to 10-membered heterocyclyl, phenyl, 5 or 6-membered heteroaryl, —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹,
   wherein the cycloalkyl, heterocyclyl, phenyl and heteroaryl groups may be unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl, and
   (ii) C₃₋₇ cycloalkyl, 4 to 7 membered heterocyclyl, phenyl and 5-or 6-membered heteroaryl are unsubstituted or substituted one to three times by the same or different groups independently selected from C₁₋₆ alkyl, halogen, —CN, nitro, —C₃₋₇-cycloalkyl, 4-to 10-membered heterocyclyl, phenyl, 5 or 6-membered heteroaryl, —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹,
   wherein the alkyl group is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl, C₁₋₄ alkoxycarbonyl, C₃₋₇ cycloalkyl, 4-to 7-membered heterocyclyl, phenyl and/or 5-or 6-membered heteroaryl,
   wherein
   (a) R⁹, R¹⁰, R¹¹, R¹³, R¹⁴, R¹⁷, R¹⁹, R²¹, R²⁴, R²⁵, R²⁶, R²⁸, R²⁹ and R³⁰ independently of one another for each individual occurrence represent a group selected from H, C₁₋₆ alkyl, C₃₋₇ cycloalkyl, 4-to 7-membered heterocyclyl, phenyl and 5-or 6-membered heteroaryl, wherein
   C₃₋₇ cycloalkyl, 4-to 7-membered heterocycloalkyl, phenyl and 5-or 6-membered heteroaryl are unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl, and
   (C₁₋₆)-alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl, C₁₋₄ alkoxycarbonyl, C₃₋₇ cycloalkyl, 4-to 7-membered heterocyclyl, phenyl and/or 5-or 6-membered heteroaryl
   (b) R¹², R¹⁵, R¹⁶, R¹⁸, R²⁰, R²², R²³, R²⁷ and R³¹ independently of one another for each individual occurrence represent a group selected from H and C₁₋₆ alkyl, wherein C₁₋₆ alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl and/or (C₁₋₄)-alkoxycarbonyl, and/or
   (c) R¹¹ and R¹², R¹⁴ and R¹⁵, R¹⁶ and R¹⁷, R¹⁸ and R¹⁹, R²⁰ and R²¹, R²¹ and R²², R²³ and R²⁴, R²⁶ and R²⁷ and R³⁰ and R³¹ in each case paired together with the atoms to which they are bonded can form a 5-or 6-membered heterocyclyl ring, which can be unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl, and
   R³ represents H, C₁₋₆ alkyl or C₃₋₇ cycloalkyl.

The compound of formula (III) may for instance be molidustat.

The PHD inhibitor may be a 5-hydroxy-1,7 naphthyridine derivative. The 5-hydroxy-1,7 naphthyridine derivative may be a compound as defined in US2023/0192688A1 (which is incorporated herein in its entirety by reference). The 5-hydroxy-1,7 naphthyridine derivative may be a compound as defined in Xu et al., J. Med. Chem. 2024 67 (2), 1393-1405 (DOI: 10.1021/acs.jmedchem.3c01932) (which is incorporated herein in its entirety by reference).

In a preferred embodiment the 5-hydroxy-1,7 naphthyridine derivative is ISM5411 or a pharmaceutically acceptable salt thereof. Alternatively, the 5-hydroxy-1,7 naphthyridine derivative may be a compound of Formula (I) or a pharmaceutically acceptable salt, solvate or stereomer thereof as defined in paragraphs [0063]-[0084] of US2023/0192688A1. Alternatively, the 5-hydroxy-1,7 naphthyridine derivative may be a compound of Formula (I) or a pharmaceutically acceptable salt, solvate or stereomer thereof as defined in paragraphs [0085]-[0111] of US2023/0192688A1. Alternatively, the 5-hydroxy-1,7 naphthyridine derivative may be any one of the compounds of Table 1 or a pharmaceutically acceptable salt, solvate or stereomer thereof as shown on pages 21 to 38 of US2023/0192688A1. Alternatively, the 5-hydroxy-1,7 naphthyridine derivative may be any one of the compounds of claims 1 to 30 or a pharmaceutically acceptable salt or stereomer thereof as shown on pages 58 to 64 of US2023/0192688A1. The 5-hydroxy-1,7 naphthyridine derivative may for instance be a compound of the following formula (T) or a pharmaceutically acceptable salt, solvate or stereomer thereof (such compounds are disclosed in US2023/0192688A1):

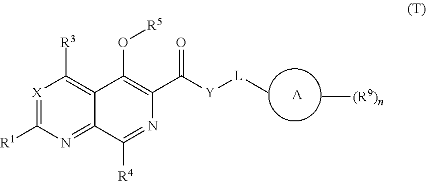

wherein:
R¹ is monocyclic heterocycloalkyl which is optionally and independently substituted;
X is N or CR²;
R² is hydrogen, fluoro, chloro, bromo, —CN, —NO₂, —OH, —OR^{a}, —C(═O)R^{a}, —C(═O)OR^{b}, —C(═O)NR^{c}R^{d}, C₁-C₆ alkyl, C₁-C₆ haloalkyl, C₁-C₆ hydroxyalkyl, C₁-C₆ aminoalkyl, or C₁-C₆ heteroalkyl;
R³ is hydrogen, halogen, —CN, —NO₂, —OH, —OR^{a}, —C(═O)R^{a}, —C(═O)OR^{b}, C(═O)NR^{c}R^{d}, C₁-C₆ alkyl, C₁-C₆ haloalkyl, C₁-C₆ hydroxyalkyl, C₁-C₆ aminoalkyl, or C₁-C₆ heteroalkyl;
R⁴ is hydrogen, halogen, —CN, —NO₂, —OH, —OR^{a}, —C(═O)R^{a}, —C(═O)OR^{b}, —C(═O)NR^{c}R^{d}, C₁-C₆ alkyl, C₁-C₆ haloalkyl, C₁-C₆ hydroxyalkyl, C₁-C₆ aminoalkyl, or C₁-C₆ heteroalkyl;
R⁵ is hydrogen, C₁-C₆ alkyl, C₁-C₆ haloalkyl, C₁-C₆ hydroxyalkyl, C₁-C₆ aminoalkyl, or C₁-C₆ heteroalkyl;
Y is —O—, —S—, or —NR⁶;
R⁶ is hydrogen, C₁-C₆ alkyl, C₁-C₆ haloalkyl, C₁-C₆ hydroxyalkyl, C₁-C₆ aminoalkyl, or C₁-C₆ heteroalkyl;
L is —(CR⁷R⁸)ₚ;
each R⁷ and R⁸ are independently hydrogen, C₁-C₆ alkyl, C₁-C₆ haloalkyl, C₁-C₆ hydroxyalkyl, C₁-C₆ aminoalkyl, or C₁-C₆ heteroalkyl;
or R⁷ and R⁸ on the same carbon are taken together to form a cycloalkyl or heterocycloalkyl; each optionally substituted with one or more R^{7a},
each R^{7a} is independently halogen, —CN, —NO₂, —OH, —OR^{a}, —NR^{c}R^{d},
C(═O)R^{a}, —C(═O)OR^{b}, —C(═O)NR^{c}R^{d}, C₁-C₆ alkyl, C₁-C₆ haloalkyl, C₁-C₆ hydroxyalkyl, C₁-C₆ aminoalkyl, or C₁-C₆ heteroalkyl;
p is 0-4;
Ring A is cycloalkyl, heterocycloalkyl, aryl, or heteroaryl;
each R⁹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{d}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted;
n is 0-4;
each R^{a} is independently C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene (cycloalkyl), C₁-C₆ alkylene (heterocycloalkyl), C₁-C₆ alkylene (aryl), or C₁-C₆ alkylene (heteroaryl); wherein each alkyl, alkenyl, alkynyl, cycloalkyl, heterocycloalkyl, aryl, and heteroaryl is independently optionally substituted;
each R^{b} is independently hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene (cycloalkyl), C₁-C₆ alkylene (heterocycloalkyl), C₁-C₆ alkylene (aryl), or C₁-C₆ alkylene (heteroaryl); wherein each alkyl, alkenyl, alkynyl, cycloalkyl, heterocycloalkyl, aryl, and heteroaryl is independently optionally substituted;
and
each R^{c} and R^{d} are independently hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene (heterocycloalkyl), C₁-C₆ alkylene (aryl), or C₁-C₆ alkylene (heteroaryl); wherein each alkyl, alkenyl, alkynyl, cycloalkyl, heterocycloalkyl, aryl, and heteroaryl is independently optionally substituted; or R^{c} and R^{d} are taken together with the atom to which they are attached to form an optionally substituted heterocycloalkyl.

The compound of formula (T) may be as further defined anywhere in US 2023/0192688 A1, the contents of which are incorporated herein by reference in their entirety. The present invention also provides a series of novel compounds that have been shown have high efficacy and specificity as human PHD inhibitors, and which are also therefore be useful for treating blood cancer in accordance with the invention. Novel compounds of the invention have been shown to have an IC₅₀ for PHD2 of less than 200 nM, which is a substantial improvement compared to known clinically applied inhibitors (e.g. roxadustat has an IC₅₀ of 2.7 μM in the PHD2 liquid chromatography based hydroxylation assay).

As well as their potency, compounds of the invention have been found to be highly selective for the PHDs compared to related enzymes, for example with greater than 100-fold selectivity compared to other tested 2OG oxygenases.

As well as these desirable biochemical properties, compounds of the invention have been shown to have desirable physical properties including good solubility and permeability in cells. These physical properties mean that compounds of the invention of have been found to increase cellular HIF-1α at concentrations in the nM range. The compounds of the invention may be for use in treating leukemia.

Accordingly the invention additionally provides a compound which is a substituted azine of formula (I) or a pharmaceutically acceptable salt thereof

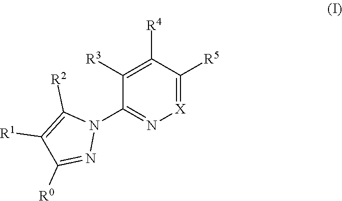

wherein
X is CR⁶ or N;
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —N(R¹)C(O)N(R^{u})(R^{v}), —CN, —C(O)OR^{w} or —C(O)N(R^{x})R⁷;
R² is H, —OR⁴ or unsubstituted or substituted C₁₋₆ alkyl; and R³ is H, —OR⁸ or unsubstituted or substituted C₁₋₆ alkyl; or R² is —N═ and R³ is ═C(R^{y})— and R² and R³ together form a group of formula —N═C(R^{y})—;
R⁴ is H, unsubstituted or substituted C₁₋₆ alkyl, —OR⁹ or —C(O)OR¹⁰;
R⁵ is H, unsubstituted or substituted C₁₋₆ alkyl, unsubstituted or substituted aryl, unsubstituted or substituted heteroaryl, —N(R^{t})C(O)N(R^{u})(R^{v}), —CN, —C(O)OR^{w}, or —C(O)N(R^{x})R⁷;
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)—Cyc, —CH₂C≡CCH₃, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR² or unsubstituted or substituted C₁₋₄ alkyl;
R⁸, R⁹ and R¹⁰ are each independently selected from H and unsubstituted or substituted C₁₋₆ alkyl; and
R^{t}, R^{u}, R^{v}, R^{w}, R^{x}, R^{y}, and R^{z} are each independently selected from H, unsubstituted or substituted C₁₋₆ alkyl, and unsubstituted or substituted phenyl; R^{q} is H, unsubstituted or substituted C₁₋₆ alkyl, or unsubstituted or substituted phenyl;
provided that one of R¹ and R⁵ is —C(O)N(R^{x})R⁷ and the other of R¹ and R⁵ is H, unsubstituted or substituted C₁₋₆ alkyl, unsubstituted or substituted aryl, unsubstituted or substituted heteroaryl, —N(R^{t})C(O)N(R^{u})(R^{v}), —CN or —C(O)OR^{w}.

Preferred embodiments of the compound which is a substituted azine of formula (I) or a pharmaceutically acceptable salt thereof are described hereinbelow, including the substituted azines of formulae (Ia), (Ib), (Ic) and (Id) as defined hereinbelow and pharmaceutically acceptable salts thereof.

The invention also provides a compound which is a substituted pyrimidine of formula (IV) or a pharmaceutically acceptable salt thereof

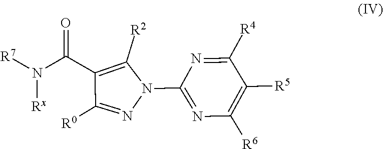

wherein
R⁰ is H or unsubstituted or substituted C₁₋₆ alkyl;
R² is H, —OR^{q} or unsubstituted or substituted C₁₋₆ alkyl;
R⁴ is —OR⁹;
R⁵ is H, unsubstituted or substituted C₁₋₆ alkyl, unsubstituted or substituted aryl, unsubstituted or substituted heteroaryl, —CN or —C(O)OR^{w};
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)—Cyc, —Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R⁹ is selected from H and unsubstituted or substituted C₁₋₆ alkyl;
R is H, unsubstituted or substituted C₁₋₄ alkyl, or unsubstituted or substituted phenyl;
R^{w} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.

The invention also provides a pharmaceutical composition comprising a compound of the invention as defined above and a pharmaceutically acceptable carrier or diluent. The pharmaceutical composition may further comprise one or more additional active agents, for instance as mentioned below.

In another aspect the invention provides a novel compound of the invention as defined above, or the pharmaceutical composition of the invention, for use in the treatment of the human or animal body by therapy.

The invention also provides a compound of the invention as defined above, or a pharmaceutical composition of the invention, for use as a modulator of PHD activity.

Typically the compound or composition is for use as an inhibitor of PHD activity. Thus, the invention also provides a compound of the invention as defined above for use as a PHD inhibitor.

The invention also provides a compound of the invention as defined above, or a pharmaceutical composition of the invention, for use in the treatment of blood cancer.

The invention also provides a method of treating a subject suffering from or susceptible to blood cancer, which method comprises administering to said subject an effective amount of a PHD inhibitor. The PHD inhibitor may be as further defined anywhere herein.

Similary the invention provides the use of a PHD inhibitor in the manufacture of a medicament for use in the treatment of blood cancer. The PHD inhibitor may be as further defined anywhere herein.

The invention also provides a PHD inhibitor as defined anywhere herein for use in a method of treating blood cancer, wherein said method comprises administering said compound and subsequently, sequentially, or simultaneously administering one or more biologically active agents for use in the treatment of blood cancer.

The invention also provides a method of treating a subject suffering from or susceptible to blood cancer, which method comprises increasing HIF in said subject. HIF may be increased in the subject by any suitable method, as discussed further herein. In one embodiment, HIF is increased by administering to the subject an effective amount of a HIF increasing agent. The HIF increasing agent may for instance be a PHD inhibitor, which may be a PHD inhibitor as further defined anywhere herein, or a compound of the invention as defined anywhere herein. Alternatively, HIF may be increased by other suitable methods which may for instance comprise increasing HIF by administering a double stranded RNA, a small interfering RNA, or by using CRISPR and guide RNA, a zinc finger protein, a transcription activator-like effector nuclease, a designer receptor exclusively activated by designer drugs, an amino acid, a signalling molecule, a peptide, a protein, an antibody, a nucleic acid, an oligonucleotide, or a cell.

The invention also therefore provides a HIF increasing agent for use in treating blood cancer.

The invention further provides the use of a HIF increasing agent in the manufacture of a medicament for use in the treatment of blood cancer.

### BRIEF DESCRIPTION OF THE FIGURES

FIG. 1 shows immunoblots of Hep3B cells treated with 117, 119, 122 and 123 (Examples 93, 95, 98 and 99) at 100 μM (A) and 20 μM (B) PHD inhibitors for 3 hours. The blots show protein levels of HIF1-α and β-actin at the 3^{rd} hour after treatment.

FIG. 2 shows immunoblots of HEK293 T cells treated with compound 68 (Example 46) at 0.5, 1, 5, 10, 20, 50 and 100 UM for 18 hours. The blots show protein levels of HIF1-α and GAPDH, at the 18^{th} hour after treatment.

FIG. 3 shows immunoblots of HEK293 T cells treated with clinically used HIF PHD inhibitors Roxadustat (1), Daprodustat (2) and Molidustat (5) at 1, 5, 10 and 20 μM for 18 hours. The western blots show protein levels of HIF1-α and GAPDH results, at the 18^{th} hour after treatment.

FIG. 4 shows loss of Phd2 significantly impairs leukemogenesis in murine AML models (A) Phd2^{fl/fl} (Control) and Phd2^{fl/fl}; Vav-iCre (Phd2^{cKO}) foetal liver (FL) c-Kit⁺ cells were co-transduced with Meis1 and Hoxa9 retroviruses, serially replated in CFC assays, and transplanted into recipient mice. (B) CFC counts of Control and Phd2^{cKO} cells after each re-plating (n=3). (C) Proliferation analyses with Control and Phd2^{cKO} cells (n=9). (D) Percentage of leukaemic cells in the peripheral blood (PB) of recipient mice (n=15). (E) Survival curve of mice transplanted with Control and Phd2^{cKO} leukaemic cells (n=15). (F) rtTA (Control) and shPhd2/rtTA (shPhd2) FL c-Kit⁺ cells were co-transduced with Meis1 and Hoxa9 retroviruses. Control and shPhd2 leukemic cells were serially replated in colony forming assays. (G) Control and shPhd2 leukaemic cells were serially replated in CFC assays +/− doxycycline (Dox) (n=4-6). (H) Lin⁻Sca-1⁺ c-Kit⁺ (LSK) cells from iMLL-AF9;Control and iMLL-AF9;shPhd2 mice were sorted for in vitro or in vivo assays. (I) CFC counts of iMLL-AF9;Control and iMLL-AF9;shPhd2 leukaemic cells +Dox (n=8). (J) Percentage of leukaemic cells in PB of recipient mice 6 weeks following transplantation of iMLL-AF9;Control and iMLL-AF9;shPhd2 cells. Mice were continuously treated with Dox (n=9). (K) Survival curve of mice transplanted with iMLL-AF9;Control and iMLL-AF9;shPhd2 leukaemic cells (n=9) (L) rtTA (Control) and shPhd2/rtTA (shPhd2) FL c-Kit⁺ cells were co-transduced with Meis1 and Hoxa9 retroviruses. Control and shPhd2 leukaemic cells were serially replated and transplanted into recipient mice. 8 weeks post-transplant, recipient mice were treated with Dox. (M) Percentage of apoptotic cells in Control and shPhd2 leukaemic cells +Dox (n=4). (N) Percentage of leukaemic cells in PB of recipient mice (n=12). (O) Survival curve of mice transplanted with Control and shPhd2 leukaemic cells (n=12). Data represent mean±SEM; * p<0.05; ** p<0.01; *** p<0.001; **** p<0.0001.

FIG. 5 shows model validation of iMLL-AF9;shPhd2 mice and Meis1 Hoxa9 transduced shPhd2 AML cells. (A) iMLL-AF9 mice were crossed to rtTA and shPhd2 rtTA mice, generating iMLL-AF9; ctl and iMLL-AF9;shPhd2 mice, respectively. Upon Dox administration, iMLL-AF9;shPhd2 cells will induce MIL-AF9 expression, a short-hairpin targeting Phd2 and GFP. Cells from iMLL-AF9; ctl and iMLL-AF9;shPhd2 mice were cultured in Dox-treated media and GFP expression was measured by flow cytometry (n=3). (B) Meis1 Hoxa9 transduced shPhd2 AML cells were cultured in Dox-treated media and GFP expression was measured by flow cytometry (n=3). (C) Proliferation curve of iMLL-AF9; ctl and iMLL-AF9;shPhd2 cells cultured in Dox-treated media (n=4). Data represent mean±SEM; * p<0.05; *** p<0.001.

FIG. 6 shows that loss of Phd2 has no significant impact on steady state or transplantation haematopoiesis. (A) Steady state analyses of 8-to 10-week-old Control and Phd2^{cKO} mice. (B) Total numbers of LSKCD48⁺CD150⁺ hematopoietic stem cells (HSCs); LSKCD48⁻CD150⁻ multipotent progenitors (MPPs); primitive hematopoietic progenitor cells (i.e., LSKCD48⁺CD150⁻ HPC-1 and LSKCD48⁺CD150⁺ HPC-2 populations) (n=4-8). (C) Total BM cellularity (n=4-8). (D) Total number of B Cells (CD19⁺ B220⁺), granulocytes (Cd11b⁺ Gr-1+) and monocytes (Cd11b⁺) in BM (n=4-8). (E) PB counts of white blood cells (WBCs), red blood cells (RBCs), haemoglobin (HGB), haematocrit (HCT), platelets (PLTs), T cells (CD4⁺CD8⁺), B Cells, granulocytes and monocytes (n=4-8) (F) 5×10⁶ CD45.2⁺ BM cells from 8-to 10-week-old Control and Phd2^{cKO} mice were transplanted into lethally irradiated syngeneic CD45.1⁺/CD45.2⁺ recipient mice together with 5×10⁶ competitor CD45.1⁺ BM cells. (G) The percentage of CD45.2⁺ cells in the overall PB compartment in recipient mice (n=4-5). (H) The percentage of CD45.2⁺ cells in the B cell, T cell and myeloid (Cd11b⁺, Cd11b Gr1⁺) compartments in recipient mice (n=4-5). (I) Steady state analyses of 13-to 14-week-old Control and shPhd2 mice treated with Dox for 8 weeks. (J) Total numbers of HSC, MPP, HPC-1 and HPC-2 populations (n=10-11). (K) PB counts of WBCs, RBCs, HGB, HCT, PLTs, T cells, B Cells, granulocytes and monocytes (n=5-6). (L) 200 HSCs cells from 8-to 10-week-old Control and shPhd2 mice were transplanted into lethally irradiated syngeneic CD45.1⁺/CD45.2⁺ recipient mice together with 5×10⁶ competitor CD45.1⁺ BM cells. Recipient mice were treated with Dox 6 weeks post-transplantation (n=10). (M) The percentage of CD45.2⁺ cells in the overall PB, B cell, T cell and myeloid compartments in recipient mice (n=10). (N) The percentage of CD45.2⁺ cells within the HSC, MPP, HPC-1 and HPC-2 compartments of recipient mice 16 weeks after transplantation (n=10). Data represent mean±SEM; * p<0.05; ** p<0.01.

FIG. 7 shows that loss of Phd2 has no impact on haematopoietic progenitors or the ability of HSCs to repopulate the haematopoietic system following transplantation. (A) Steady state analyses of 8-to 10-week-old Control and Phd2^{cKO} mice. Total numbers of EryA, EryB, EryC and ProE erythrocyte progenitors (n=4-8). (B) Total numbers of Pre-GM (Granulocyte Monocyte progenitor) and GMP (Granulocyte Monocyte Progenitor) (n=4-8). (C) Total numbers of Pre-MegE (Pre-Megakaryocyte-Erythrocyte progenitor), Pre CFU-E (Colony Forming Unit-Erythroid), CFU-E (n=4-8). (D) Total number of LKs and LSKs (n=4-8). (E) Steady state analyses of 13-to 14-week-old Control and shPhd2 mice treated with Dox for 8 weeks. GFP+ cells in the BM (n=10-11). (F) Total number of LKs and LSKs (n=10-11). (G) Total BM cellularity (n=10-11). (H) Total numbers of B cells, granulocytes and monocytes (n=10-11). (I) 200 HSCs cells from 8-to 10-week-old Control and shPhd2 mice were transplanted into lethally irradiated syngeneic CD45.1⁺/CD45.2⁺ recipient mice together with 5×10⁶ competitor CD45.1⁺ BM cells. Recipient mice were treated with Dox 6 weeks post-transplantation. Percentage of GFP⁺ cells in the CD45.2⁺ compartment of recipient mice (n=10). (J) Percentage of CD45.2⁺ cells within the B cell, granulocyte and monocyte compartments of recipient mice 16 weeks after transplantation (n=10). (K) Percentage of CD45.2⁺ cells within the LK and LSK compartments of recipient mice 16 weeks after transplantation (n=10). Data represent mean±SEM; * p<0.05; **** p<0.0001.

FIG. 8 shows the anti-leukaemic effect of PHD inhibitors in multiple AML cell lines. (A) Immunoblot of MOLM13, OCI-AML3, MV411 and THP-1 treated with vehicle control, 50 μM Daprodustat, 50 μM IOX5 (68, example 46) for 48 hrs. (B-E) Proliferation and apoptosis of murine AML cell lines Meis1Hoxa9 and iMLL-AF9 treated with vehicle control, 50 μM Daprodustat, 50 μM IOX5 (68, example 46). (F-G) Proliferation and apoptosis of human AML cell line THP-1 treated with vehicle control, 50 μM Daprodustat, 50 μM IOX5 (68, example 46). (H-I) Proliferation and apoptosis of human leukemic cell lines K562 (BM cells from blast crisis CML), OCI-AML3 (PB cells from AML with NPM1 and DNM3TA mutations), KASUMI 1 (PB cells from AML with AML-ETO), MV411 (PB cells with FLT3-ITD) and MOLM13 (PB cells with FLT3-ITD and MLL-AF9) treated with vehicle control, 50 μM Daprodustat (2), 50 μM IOX5 (68, example 46). (J) Apoptosis of human AML patient samples treated with vehicle control, 50 μM Daprodustat, 50 μM IOX5 (68, example 46). Data represent mean±SEM; * p<0.05; ** p<0.01, *** p<0.001, **** p<0.0001.

FIG. 9 shows the anti-leukaemic effect of Molidustat. (A-B) Proliferation and apoptosis of human AML cell lines THP-1, MOLM13, OCI-AML3 and MV411 treated with vehicle control and 50 μM Molidustat (5). Data represent mean±SEM; **** p<0.0001.

FIG. 10 shows that the transcriptional analysis of IOX5 (68, example 46) treated AML cells (A) Expression scatter plot of iMLL-AF9 cells treated with vehicle control or 50 μM IOX5 (68, example 46). (B) Gene set enrichment analysis (GSEA) data of up-and down-regulated pathways in iMLL-AF9 cells treated with 50 μM IOX5 (68, example 46) (left) and Hif½α^{DKO} Meis1 Hoxa9 cells exposed to hypoxia (right). Data represent mean±SEM; *** p<0.001, **** p<0.0001.

FIG. 11 shows that the anti-leukaemic activity of IOX5 (68, example 46) is dependent on upregulation of HIF-dependent transcripts. (A) Proliferation of Hif½α CTL and Hif½α^{DKO} Meis1 Hoxa9-transformed leukemic cells treated with 50 μM IOX5 (68, example 46). (B) Expression of up-regulated HIF target genes BNIP3, PDK1, EGLN3 and ALDOA upon IOX5 (68, example 46) treatment. (C) qPCR analysis of BNIP3, BAX and BAK transcripts following treatment of vehicle control, 50 μM Daprodustat (2), 50 μM IOX5 (68, example 46) in MOLM-13 human AML cells.

FIG. 12 shows the anti-leukaemic potential of PHD inhibition and BCL-2 inhibition combination therapy. (A-B) Proliferation and apoptosis of human AML cell lines MOLM-13, OCI-AML3, MV411 and THP-1 treated with vehicle control, 50 μM Daprodustat (2), 50 μM IOX5 (68, example 46), 0.1 μM Venetoclax (MOLM-13 and MV411), 0.01 μM Venetoclax (OCI-AML3 and THP-1) and combination therapy. (C-D) Early-(PI⁺Annexin⁻) and late stage (PI⁺ Annexin⁺) apoptosis of human AML patient samples treated with vehicle control, 50 μM Daprodustat (2), 50 μM IOX5 (68, example 46), 0.01 μM Venetoclax and combination therapy. Data represent mean±SEM; * p<0.05; ** p<0.01, *** p<0.001, **** p<0.0001.

FIG. 13 shows the pro-apoptotic potential of PHD inhibitor Daprodustat in multiple myeloma (MM) cell lines. Apoptosis of multiple human MM cell lines derived from different patients (AWO^{WT}, H292, KMS12, RPMI, U266 and MM.1S) treated with vehicle control, 50 μM Daprodustat (2). Data represent mean±SEM; * p<0.05; ** p<0.01, *** p<0.001.

FIG. 14 shows IC₅₀ of compound 68 (Example 46) of the invention when tested with off-target sites commonly inhibited by existing PHD inhibitors.

FIG. 15 shows that loss of Phd2 significantly impairs leukaemogenesis in murine AML models. (A) shows PHD2 (EGLN1) and PHD1 (EGLN2) expression in human CD34+ cells, bone marrow mononuclear cells (BM MNCs) and AML patient samples. (B) shows PHD2 (EGLN1) and PHD1 (EGLN2) expression in control (CTL), cytologically normal with intermediate prognosis (CNI), cytologically abnormal not otherwise specified (CAO), and different cytogenetic subgroups of human AML bone marrow samples.

FIG. 16 shows PHD2 (EGLN1) expression in AML patient samples with adverse, intermediate and favourable prognosis, respectively. Data from Beat AML 1.0 and Beat AML 2.0 data sets, respectivelyl^{28,129}.

FIG. 17 shows Percentage of GFP+ cells after 3 rounds of re-plating+DOX (n=8).

FIG. 18 shows percentage of Annexin-V⁺ToPro⁺ cells after 3 rounds of re-plating+DOX (n=6).

FIG. 19 shows CFC counts of iMLL-AF9; Control and iMLL-AF9;shPhd2 cells +DOX (n=8).

FIG. 20 shows proliferation of iMLL-AF9; Control and iMLL-AF9;shPhd2 cells in the presence of DOX (n=9-12).

FIG. 21 shows Lin-Sca-1⁺c-Kit⁺ (LSK) cells from iMLL-AF9;Control and iMLL-AF9;shPhd2 mice were sorted for in vitro and in vivo assays.

FIG. 22 shows (K-L) Percentage of leukaemic and GFP+ cells, respectively, in PB of recipient mice 6 weeks following transplantation. Mice were continuously treated with DOX (n=5-6). (M) Survival curve of mice transplanted with iMLL-AF9;Control and iMLL-AF9;shPhd2 LSK cells (n=6-7). (N) Limiting dilution assay (LDA) in secondary recipients transplanted with indicated doses of CD45.2+BM cells from primary recipients (n=6-7 per dose). (O) Plot showing Poisson statistical analysis. circles represent the percentages of negative mice for each cell dose, triangles represent any data values with zero negative responses. Solid lines indicate the best-fit linear model, and dotted lines represent 95% CIs. LSC frequencies were calculated using the ELDA software¹²¹.

FIG. 23 shows LDA in secondary recipients transplanted with cells from primary recipients (n=6-7 per dose). Lower, estimate and upper LSC frequencies were calculated using the ELDA software¹²¹. Data represent mean±SEM; * p<0.05; *** p<0.001.

FIG. 24 shows that 200 HSCs from 8-to 10-week-old Control and shPhd2 mice were transplanted into lethally irradiated syngeneic CD45.1+/CD45.2+ recipient mice together with 5×10⁶ competitor CD45.1+ BM cells. Recipient mice were treated with DOX 6 weeks post-transplantation (n=10-11).

FIG. 25 shows (A) percentage of GFP+ cells in CD45.2+ in overall PB compartment (n=8-10). (B) Relative levels of Phd2 mRNA (normalised to β-actin) in total BM and LSK CD45.2+ sorted cells 16-weeks post-transplantation (n=3-4).

FIG. 26 shows western blot of HIF-2α in MOLM13, OCI-AML3, MV411 and THP-1 cells treated with IOX5 (68, example 46) or vehicle control for 48 hrs. α-Histone 3 (H3) used as a loading control.

FIG. 27 (A-B) shows proliferation and Annexin-V+DAPI+ analyses, respectively, of FLT3-ITD-, PML-RARα-and AML1-ETO-transformed murine cells¹²⁷ treated with Dap, IOX5 (68, example 46) or vehicle control (n=3-6).

FIG. 28 shows Proliferation and Annexin-V″DAPI″ analyses of murine NPM1/Flt3-ITD cells treated with Daprodustat (Dap), IOX5 (68, example 46) or vehicle control (n=5).

FIG. 29 shows a description of karyotype and mutational status of responder patient samples used in pharmacological studies. For FLT3, NPM1 and TP53 mutational status; red (i.e. the shading in the boxes for AML 1-5, 7 and 10-12)=mutation, grey (i.e. the shading in the boxes for AML 9)=unknown, white=WT.

FIG. 30 shows percentage of Annexin-V⁺PI⁺ cells (matched to vehicle control) of individual patient samples treated with Dap or IOX5 (68, example 46) (n=12 independent samples).

FIG. 31 shows proliferation analyses of THP-1 cells treated with Roxadustat or vehicle control (n=2-3).

FIG. 32 shows Western blot of HIF-1α in THP-1 cells treated with IOX5 (68, example 46) for described timepoints. For vehicle control and positive control, cells were treated with DMSO and Dap, respectively, for either 48 or 96 hrs. β-actin used as a loading control. Data represent mean±SEM; ** p<0.01; *** p<0.001; **** p<0.0001.

FIG. 33 shows loss of Phd1 compromises AML propagation and maintenance. (A) Phd1^{fl/fl} (Control) and Phd1^{fl/fl}, Vav-iCre (Phd1^{cKO}) foetal liver (FL) c-Kit⁺ cells were co-transduced with Meis1 and Hoxa9 retroviruses, serially replated in FC assays, and transplanted into lethally irradiated recipient mice. Leukaemic cells harvested from primary recipient mice were then transplanted in lethally irradiated secondary recipients. (B) CFC counts of Control and Phd1^{cKO} cells after each re-plating (n=5-10). (C) Proliferation analyses with Control and Phd1^{cKO} cells (n=3-6). (D) Percentage of leukaemic cells in the peripheral blood (PB) of recipient mice in primary transplant (n=15). (E) Survival curve of mice transplanted with Control and Phd1^{cKO} leukaemic cells in primary transplant (n=18). (F) Percentage of CD45.2+leukaemic cells in BM of primary recipient mice at the end of the experiment. (n=3). (G) Percentage of c-Kit⁺ cells in BM of primary recipient mice at the end of the experiment. (n=3). (H) Proliferation analyses with Control and Phd1^{KO} cells collected from primary recipients (n=6). (I) Percentage of leukaemic cells in the peripheral blood (PB) of recipient mice in secondary transplant (n=22). (J) Survival curve of mice transplanted with Control and Phd1^{cKO} leukaemic cells in secondary transplant (n=12-13). (K) Steady-state analyses of 8-to 10-week-old Control and Phd1cKO mice. (L) Total BM cellularity (n=8-10). (M) Total numbers of haematopoietic stem cells (HSCs); multipotent progenitors (MPPs); primitive haematopoietic progenitor cells (HPC-1 and HPC-2) (n=6-7). Data represent mean±SEM; * p<0.05; ** p<0.01; *** p<0.001; ****p<0.0001.

FIG. 34 shows that PHD inhibition decreases AML engraftment and increases survival in vivo, but does not affect normal haematopoiesis. (A) 100,000 THP-1 cells were transplanted into NBSGW recipient mice. 14 days following transplantation, recipient mice were treated with Dap, IOX5 (68, example 46) or vehicle control 2× daily via i.p. injection for 21 days (n=4-5). (B-C) Percentage of human CD45+CD33+ and human CD45+CD14+ cells in the BM and spleen, respectively (n=4-5). (D) 100,000 OCI-AML3 cells were transplanted into NBSGW recipient mice. 14 days following transplantation, recipient mice were treated with IOX5 (68, example 46) or vehicle control 2× daily via i.p. injection for 21 days (n=4-7). (E) Percentage of human CD45+CD33+CD14+ cells in the liver, BM and PB, respectively (n=3-7). (F) 2,000 LSK cells from iMLL-AF9;Control mice were sorted and transplanted into irradiated recipient mice. 40 days following transplantation, recipient mice were treated with IOX5 (68, example 46) or vehicle control 2× daily via i.p. injection for 14 days (n=7). (G) Survival curve of mice transplanted with iMLL-AF9;Control LSK cells treated with IOX5 (68, example 46) or a vehicle control (n=7). (H) LDA in secondary recipients transplanted with indicated doses of CD45.2+BM cells from primary recipients (n=9-10 per dose). (I) Plot showing Poisson statistical analysis. Circles represent the percentages of negative mice for each cell dose, triangles represent any data values with zero negative responses. Solid lines indicate the best-fit linear model, and dotted lines represent 95% CIs. LSC frequencies were calculated using the ELDA software 121. (J) Steady-state analyses of 8-to 10-week-old C₅₇B16 mice treated with IOX5 or vehicle control 2× daily via i.p. injection for 14 days (n=5-7). (K) PB counts (n=6). (L) Total BM cellularity (n=5-9). (M) Total numbers of HSC, MPP, HPC-1 and HPC-2 populations (n=5-7). Data represent mean±SEM; * p<0.05; ** p<0.01; *** p<0.001; **** p<0.0001.

FIG. 35 shows anti-leukemic potential of PHD inhibition in vivo (A) Weight of animals pre- and post-treatment with Dap, IOX5 (68, example 46) or vehicle control 2× daily via i.p. injection for 21 days (n=3-5). (B) Percentage of leukaemic CD45.2+ cells and myeloid cells in the peripheral blood (PB) of recipient mice pre-treatment with IOX5 (68, example 46) or vehicle control (n=7-9). (C) LDA analyses in secondary recipients transplanted with cells from primary recipients (n=9-10 per dose). Lower, estimate and upper LSC frequencies were calculated using the ELDA software 121. (D-K) Analyses of 8-to 10-week-old C₅₇B16 mice treated with IOX5 (68, example 46) or vehicle control 2× daily via i.p. injection for 14 days (n=6-9). (D) Total spleen cellularity (n=5-9). (E) Total numbers of B cells, Granulocytes and Monocytes in the BM (n=6-9). (F) Total numbers of B cells, Granulocytes, Monocytes and T cells in the spleen (n=5-9). (G) Fold change of RBC, HCT and HGB in PB post-/pre-treatment (n=6-11). (H) Total numbers of erythrocyte progentiors in the BM (n=6-9). (I) Total numbers of erythrocyte progenitors in the spleen (n=5-9). (J) Total numbers of LSK and LK cells in the BM (n=5-9). (K) Total numbers of Pre-GM, GMP, Pre-MegE, Pre-CFU-E and CFU-E progenitors in the BM (n=6). Data represent mean±SEM; * p<0.05; ** p<0.01; *** p<0.001; **** p<0.0001.

FIG. 36 shows dysregulated transcripts and pathways following PHD inhibition (A) Scatterplot of transcripts in control and IOX5 (68, example 46)-treated iMLL-AF9 cells (n=4) (identical to 10A). Transcripts significantly up-(red) and down-regulated (blue) in IOX5 (68, example 46)-treated iMLL-AF9 cells are highlighted. (B) Volcano plot of differentially expressed genes in IOX5 (68, example 46)-treated iMLL-AF9 cells. Genes positively-and negatively-regulated by HIF-1α 31 (C) Ingenuity canonical pathway analysis in IOX5 (68, example 46)-treated iMLL-AF9 and Hif½aDKO Meis1/Hoxa9 cells (n=4). (D) Expression of Hspa8, Nup98, Kpnb1 and Rbm15 in IOX5 (68, example 46)-treated iMLL-AF9 cells. Violin plots showing distribution of log 2 expression values (transcripts per million). (E) Gene expression of Kdm5b, Bnip3, and Fam 162a in IOX5 (68, example 46)-treated iMLL-AF9 cells. Violin plots show the distribution of log 2 expression values (transcripts per million). (F) Relative levels of BNIP3 mRNA (normalised to β-ACTIN) in MOLM13 cells transduced with lentiviruses expressing scrambled short hairpin RNA (shCTL) and a shRNA targeting BNIP3 (shBNIP3). (G) Proliferation analyses of shCTL-and shBNIP3-transduced MOLM13 cells treated with IOX5 (68, example 46) or vehicle control (n=3). Data represent mean±SEM; ** p<0.001; *** p<0.001; **** p<0.0001.

FIG. 37 shows that targeting PHD2 upregulates HIF-target genes, including the pro-apoptotic BNIP3 (C) Venn-diagram of overlapping genes between up-and down-regulated genes in IOX5 (68, example 46)-treated iMLL-AF9 and Hif½aDKO Meis1/Hoxa9 cells. Fisher's exact test statistical analyses shown. Odds ratio 4.04 and 2.85, respectively. (D) Scatter plot showing the inverse correlation between up-and down-regulated transcripts as shown in (C). Statistical significance was evaluated using Spearman's correlation test. Bnip3 is annotated. (E) Relative levels of BNIP3 mRNA (normalised to ACTB) in MOLM13, OCI-AML3, MV411 and THP-1 cells treated with Daprodustat, IOX5 (68, example 46) or vehicle control (n=3-4). (F) Western blot of BNIP3 in THP-1 cells treated with IOX5 (68, example 46) or vehicle control. β-actin used as a loading control. * Indicates a nonspecific band. (G) MOLM13 cells were transduced with lentiviruses expressing scrambled short hairpin RNA (shCTL) and a shRNA targeting BNIP3 (shBNIP3). Annexin-V+DAPI+analyses of shCTL and shBNIP3 MOLM13 cells treated with IOX5 (68, example 46) or vehicle control (n=3). Data represent mean±SEM; ** p<0.01; *** p<0.001; **** p<0.0001.

FIG. 38 shows that PHD inhibition combined with Venetoclax ablates AML growth in vitro and in vivo (D) 100,000 MV411 cells were transplanted into NBSGW recipient mice. 14 days following transplantation, recipient mice were treated with IOX5 (68, example 46), Venetoclax or vehicle control. Dosing regimen consisted of 2× daily via i.p. injection (IOX5 (68, example 46) or vehicle) and/or 1× daily via o.g. (Venetoclax or vehicle). After 14 days treatment, half of the cohort were analysed for human AML cell engraftment, while the other half were observed for survival analyses. (E-G) Percentage of human CD45+CD33+CD14+ cells in the BM, spleen and liver, respectively (n=5). (H) Survival curve of mice treated with IOX5 (68, example 46), Venetoclax, IOX5 (68, example 46)+Venetoclax or vehicle control (n=6-7). (I-J) Proliferation and Annexin-V+DAPI+analyses, respectively, of THP-1 cells treated with Ven, IOX5, FIH inhibitor (FIHi; DM-NOFD), IOX5 (68, example 46)+FIHi, IOX5 (68, example 46)+Ven, FIHi+Ven, IOX5 (68, example 46)+FIHi+Ven or vehicle control (n=6). Statistical significance represented as in (A-B). Data represent mean±SEM; *p<0.05; ** p<0.01; *** p<0.001; **** p<0.0001.

### DETAILED DESCRIPTION OF THE INVENTION

### Definitions

The term “alkyl”, as used herein, refers to a linear or branched chain saturated hydrocarbon radical. A “Cₙ₋ₘ alkyl” refers to an alkyl having from n to m carbon atoms. Thus, an alkyl group may be a C₁₋₂₀ alkyl group, a C₁₋₁₈ alkyl group, a C₁₋₁₄ alkyl group, a C₁₋₁₀ alkyl group, a C₁₋₆ alkyl group or a C₁₋₄ alkyl group. Examples of a C₁₋₁₀ alkyl group are methyl, ethyl, propyl, butyl, pentyl, hexyl, heptyl, octyl, nonyl or decyl. Examples of C₁₋₆ alkyl groups are methyl, ethyl, propyl, butyl, pentyl or hexyl. Examples of C₁₋₄ alkyl groups are methyl, ethyl, i-propyl, n-propyl, t-butyl, s-butyl or n-butyl. If the term “alkyl” is used without a prefix specifying the number of carbons anywhere herein, it has from 1 to 6 carbons. For the avoidance of doubt, where two alkyl moieties are present in a group, the alkyl moieties may be the same or different.

The term “cycloalkyl”, as used herein, refers to a saturated cyclic hydrocarbon radical. A “Cₙ₋ₘ cycloalkyl” refers to a cycloalkyl having from n to m carbon atoms. Thus, a cycloalkyl group may be a C₃₋₂₀ cycloalkyl group, a C₃₋₁₀ cycloalkyl group, a C₃₋₈ cycloalkyl group or a C₃₋₆ cycloalkyl group. Examples of a C₃₋₈ cycloalkyl group include cyclopropyl, cyclobutyl, cyclopentyl, cyclohexyl, cycloheptyl and cyclooctyl. Examples of a C₃₋₆ cycloalkyl group include cyclopropyl, cyclobutyl, cyclopentyl, and cyclohexyl.

The term “alkenyl”, as used herein, refers to a linear or branched chain hydrocarbon radical containing one or more double bonds. A “Cₙ₋ₘ alkenyl” refers to an alkenyl having from n to m carbon atoms. Thus, an alkenyl group may be a C₂₋₁₈ alkenyl group, a C₂₋₁₄ alkenyl group, a C₂₋₁₀ alkenyl group, a C₂₋₆ alkenyl group or a C₂₋₄ alkenyl group. Examples of a C₂₋₁₀ alkenyl group are ethenyl (vinyl), propenyl, butenyl, pentenyl, hexenyl, heptenyl, octenyl, nonenyl or decenyl. Examples of C₂₋₆ alkenyl groups are ethenyl, propenyl, butenyl, pentenyl or hexenyl. Examples of C₂₋₄ alkenyl groups are ethenyl, i-propenyl, n-propenyl, s-butenyl or n-butenyl. Alkenyl groups typically comprise one or two double bonds.

The term “cycloalkenyl” as used herein, refers to a partially unsaturated cyclic hydrocarbon radical. A “Cₙ₋ₘ cycloalkenyl” refers to a cycloalkenyl having from n to m carbon atoms. Thus, a cycloalkenyl group may be a C₃₋₂₀ cycloalkenyl group, a C₃₋₁₀ cycloalkenyl group, a C₃₋₈ cycloalkenyl group or a C₅₋₈ cycloalkenyl group. Examples of a C₅₋₈ cycloalkenyl group include, cyclohex-1,3-dienyl.

The term “alkynyl”, as used herein, refers to a linear or branched chain hydrocarbon radical containing one or more triple bonds. A “Cₙ₋ₘ alkynyl” refers to an alkynyl having from n to m carbon atoms. Thus, an alkynyl group may be a C₂₋₁₈ alkynyl group, a C₂₋₁₄ alkynyl group, a C₂₋₁₀ alkynyl group, a C₂₋₆ alkynyl group or a C₂₋₄ alkynyl group. Examples of a C₂₋₁₀ alkynyl group are ethynyl, propynyl, butynyl, pentynyl, hexynyl, heptynyl, octynyl, nonynyl or decynyl. Examples of C₁₋₆ alkynyl groups are ethynyl, propynyl, butynyl, pentynyl or hexynyl. Alkynyl groups typically comprise one or two triple bonds.

A C₃₋₂₀ heterocyclyl group is a monovalent moiety obtained by removing a hydrogen atom from a ring atom of a heterocyclic compound, which moiety has from 3 to 20 ring atoms (unless otherwise specified), of which from 1 to 10 are ring heteroatoms. A “Cₙ₋ₘ heterocyclyl” refers to a heterocyclyl having from n to m ring atoms. Preferably, the ring has from 3 to 7 ring atoms (i.e. it is a C₃₋₇ heterocyclyl), of which from 1 to 4 are ring heteroatoms.

Examples of 5-and 6-membered saturated heterocyclyl groups include piperazine, piperidine, morpholine, 1,3-oxazinane, pyrrolidine, imidazolidine, and oxazolidine, including quaternised derivatives thereof, as defined herein. Examples of 5-and 6-membered partially saturated heterocyclyl groups include tetrahydropyrazine, tetrahydropyridine, dihydro-1,4-oxazine, tetrahydropyrimidine, dihydro-1,3-oxazine, dihydropyrrole, dihydroimidazole and dihydrooxazole, including quaternised derivatives thereof, as defined herein. Thus, heterocyclyl groups include pyrazolidinyl, piperidyl, piperazinyl, thiomorpholinyl, S-oxo-thiomorpholinyl, S,S-dioxo-thiomorpholinyl, morpholinyl, pyrrolidinyl, pyrrolinyl, imidazolidinyl, imidazolinyl, 1,3-dioxolanyl, 1,4-dioxolyl and pyrazolinyl groups and moieties. Pyrazolidinyl, piperidyl, piperazinyl, pyrazolidinyl morpholinyl and imidazolidinyl groups and moieties are typical examples.

Examples of 9-and 10-membered fused heterobicyclyl groups include 9-membered fused heterobicyclic groups such as indoline, 2,3-dihydrobenzofuran, 2,3-dihydrobenzo[b]thiophene, 2,3-dihydro-1H-benzo[d]imidazole, 2,3-dihydrobenzo[d]oxazole, 2,3-dihydrobenzo[d]thiazole, benzo[d][1,3]dioxole, 4,5,6,7-tetrahydrothiazolo[5,4-c]pyridine and 4,5,6,7-tetrahydrothiazolo[4,5-c]pyridine, including quaternised derivatives thereof, as defined herein; and 10-membered heterobicyclyl groups such as 1,2,3,4-tetrahydroquinoline, 1,2,3,4-tetrahydroisoquinoline, chromane, isochromane, thiochromane, isothiochromane, 1,2,3,4-tetrahydroquinoxaline, 1,2,3,4-tetrahydroquinazoline, 1,4-dihydro-2H-benzo[d][1,3]oxazine, 3,4-dihydro-2H-benzo[b][1,4]oxazine, 3,4-dihydro-2H-benzo[b][1,4]thiazine, 1,4-dihydro-2H-benzo[d][1,3]thiazine, 4H-benzo[d][1,3]dioxine and 2,3-dihydrobenzo[b][1,4]dioxine, including quaternised derivatives thereof. Preferably, the fused heterobicyclyl group comprises 1, 2 or 3, preferably 1 or 2 nitrogen atoms.

For the avoidance of doubt, references to a heterocyclyl group also include fused polycyclic ring systems, including for instance fused bicyclic systems in which a heterocyclic group is fused to an aryl group. When the heterocyclyl group is such a fused heterocyclyl group, preferred examples are fused ring systems wherein a 5-to 6-membered heterocyclyl group is fused to a phenyl group. References to a heterocyclyl group also include spiro ring systems, for example 7-membered heterocyclic groups e.g. 2,6-diazaspiro[3.3]heptane.

The term “aryl”, as used herein, refers to a monocyclic, bicyclic or polycyclic aromatic ring which contains up to 14 carbon atoms, typically from 6 to 10 carbon atoms, in the ring portion. Examples include phenyl, naphthyl, indenyl and indanyl groups. Phenyl is preferred.

The term “heteroaryl”, as used herein, refers to monocyclic or bicyclic heteroaromatic rings which typically contains from five to ten, for instance from six to ten, atoms in the ring portion including one or more heteroatoms. A heteroaryl group is generally a 5-or 6-membered ring, containing at least one heteroatom selected from O, S, N, P, Se and Si, more typically selected from O, S and N. It may contain, for example, one, two or three heteroatoms. Examples of heteroaryl groups include pyridyl, pyrazinyl, pyrimidinyl, pyridazinyl, furanyl, thienyl, pyrazolidinyl, pyrrolyl, oxazolyl, oxadiazolyl, isoxazolyl, thiadiazolyl, thiazolyl, isothiazolyl, imidazolyl, pyrazolyl, pyridazolyl, quinolyl and isoquinolyl. Furanyl, thienyl, pyridazolyl, pyrazolyl, pyrimidinyl and thiazolyl groups are typical examples.

The terms “alkylene”, “cycloalkylene”, “heterocyclylene”, “alkenylene”, “cycloalkenylene” “alkynylene”, “arylene” and “heteroarylene”, as used herein, refer to bivalent groups obtained by removing a hydrogen atom from an alkyl, cycloalkyl, heterocyclyl, alkenyl, cycloalkenyl, alkynyl, aryl or heteroaryl group, respectively. Such bidentate groups may be substituted or unsubstituted. An alkylene group may be a C₁₋₂₀ alkylene group, a C₁₋₁₈ alkylene group, a C₁₋₁₄ alkylene group, a C₁₋₁₀ alkylene group, a C₁₋₆ alkylene group or a C₁₋₄ alkylene group. Examples of C₁₋₆ alkylene groups are methylene, ethylene, propylene, butylene, pentylene and hexylene. A cycloalkylene group may be a C₃₋₁₀ cycloalkylene group, a C₃₋₈ cycloalkylene group or a C₃₋₆ cycloalkylene group. Examples of C₃₋₆ cycloalkylene groups include cyclopentylene and cyclohexylene. An alkenylene group may be a C₂₋₁₈ alkenylene group, a C₂₋₁₄ alkenylene group, a C₂₋₁₀ alkenylene group, a C₂₋₆ alkenylene group or a C₂₋₄ alkenylene group. Examples of a C₂₋₄ alkenylene group include ethenylene (vinylene), propenylene and butenylene. A cycloalkenylene group may be a C₅₋₈ cycloalkenylene group.

Examples of a C₅₋₈ cycloalkenylene group include cyclohex-1,3-dienylene. An alkynylene group may be a C₂₋₁₈ alkynylene group, a C₂₋₁₄ alkynylene group, a C₂₋₁₀ alkynylene group, a C₂₋₆ alkynylene group or a C₂₋₄ alkynylene group. Examples of a C₂₋₄ alkynylene group include ethynylene and propynylene. Examples of arylene groups include phenylene, and examples of heteroarylene groups include, for instance, a diradical derived from pyridine, a diradical derived from thiophene, a diradical derived from chromane, and a diradical derived from chromanol. For alkylene, cycloalkylene, alkenylene, alkynylene, arylene and heteroarylene, these groups may be bonded to other groups at any two positions on the group (which positions are typically carbon atoms in the case of heteroarylene and heterocyclylene). Thus, propylene includes —CH₂CH₂CH₂— and —CH₂CH(CH₃)—, and phenylene includes ortho-, meta-and para-phenylene.

The term “substituted”, as used herein, in the context of substituted organic compounds and groups, refers to an organic compound or group (e.g. an alkyl group, an alkylene group, a cycloalkyl group, a heterocyclyl group, an aryl group, an arylene group, a heteroaryl group, or a heteroarylene group) which bears one or more substituents selected from C₁₋₁₀ alkyl, C₃₋₁₀ cycloalkyl, C₃₋₇ heterocyclyl, aryl, heteroaryl, cyano, amino, nitro, C₂₋₁₀ alkenyl, C₂₋₁₀ alkynyl, C₁₋₁₀ alkylamino, di(C₁₋₁₀)alkylamino, arylamino, diarylamino, aryl(C₁₋₁₀)alkylamino, amido, acylamido, hydroxy, oxo, halo, carboxy, ester, acyl, acyloxy, C₁₋₁₀ alkoxy, aryloxy, halo(C₁₋₁₀)alkyl, sulfonic acid, thiol, C₁₋₁₀ alkylthio, arylthio, sulfonyl, phosphoric acid, phosphate ester, phosphonic acid, phosphonate ester and SO₃⁻. Typically, the one or more substituents are selected from cyano, amino, nitro, amido, acylamido, hydroxy, oxo, halo, carboxy, ester, acyl, acyloxy, sulfonic acid, thiol, sulfonyl, phosphoric acid, phosphate ester, phosphonic acid, phosphonate ester and SO₃⁻. When a compound or group is substituted, it typically bears 1, 2, 3 or 4 substituents. For instance, a substituted compound or group may have 1, 2 or 3 substituents, or for example 1 or 2 substituents.

However, when a group is halo-substituted, for instance fluoro-substituted, the group may bear 1, 2, 3 or 4 halo substituents, or it may bear more than four halo substituents. In fact, the group may be perhalo-substituted, i.e. all hydrogen atoms of the group may be replaced by halogen atoms. The group may for instance be perfluoro-substituted, i.e. perfluorinated, i.e. all hydrogen atoms of the group may be replaced by fluorine atoms. Accordingly, the term “substituted”, as used herein, in the context of substituted organic groups, for instance in the context of substituted hydrocarbyl groups, substituted alkyl groups, substituted cycloalkyl groups, substituted alkenyl groups, substituted alkynyl groups, substituted aryl groups, substituted hydrocarbylene groups, substituted alkylene groups, substituted cycloalkylene groups, substituted alkenylene groups, substituted alkynylene groups, and substituted arylene (including substituted heteroarylene) groups, encompasses the perhalo-substituted groups, in particular the perfluoro-substituted groups. Thus, for example, the term “substituted Cₙ₋ₘ alkyl” as used herein encompasses Cₙ₋ₘ perfluoroalkyl, the term “substituted Cₙ₋ₘ alkylene” as used herein encompasses Cₙ₋ₘ perfluoroalkylene, the term “substituted Cₙ₋ₘ hydrocarbyl” as used herein encompasses Cₙ₋ₘ perfluorohydrocarbyl and the term “substituted Cₙ₋ₘ hydrocarbylene” as used herein encompasses Cₙ₋ₘ perfluorohydrocarbylene, the term “substituted Cₙ₋ₘ alkoxy” as used herein encompasses Cₙ₋ₘ perfluoroalkoxy, and so-on.

As used herein the term oxo represents a group of formula: ═O As used herein the term acyl represents a group of formula: —C(═O)R, wherein R is an acyl substituent, for example, a substituted or unsubstituted C₁₋₂₀ alkyl group, a substituted or unsubstituted C₃₋₂₀ heterocyclyl group, or a substituted or unsubstituted aryl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

As used herein the term ester (or carboxylate, carboxylic acid ester or oxycarbonyl) represents a group of formula: —C(═O)OR, wherein R is an ester substituent, for example, a substituted or unsubstituted C₁₋₂₀ alkyl group, a substituted or unsubstituted C₃₋₂₀ heterocyclyl group, or a substituted or unsubstituted aryl group (typically a phenyl group). Examples of ester groups include, but are not limited to, —C(═O)OCH₃, —C(═O)OCH₂CH₃, —C(═O)OC(CH₃)₃, and —C(═O)OPh.

As used herein the term “hydroxycarbonyl” represents a group of formula: —C(═O)OH. The term carboxylic acid group, can also be used.

As used herein the term acyloxy (or reverse ester) represents a group of formula: —OC(═O)R, wherein R is an acyloxy substituent, for example, substituted or unsubstituted C₁₋₂₀ alkyl group, a substituted or unsubstituted C₃₋₂₀ heterocyclyl group, or a substituted or unsubstituted aryl group, typically a C₁₋₆ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

As used herein the term phosphonic acid represents a group of the formula: —P(═O)(OH)₂. As would be understood by the skilled person, a phosphonic acid group can exist in protonated and deprotonated forms (i.e. —P(═O)(OH)₂, —P(═O)(O⁻)₂ and —P(═O)(OH)(O⁻)) all of which are within the scope of the term “phosphonic acid”.

As used herein the term phosphonic acid salt represents a group which is a salt of a phosphonic acid group. For example a phosphonic acid salt may be a group of the formula —P(═O)(OH)(O⁻X⁺) wherein X is a monovalent cation. X⁺ may be an alkali metal cation. X⁺ may be Na⁺ or K⁺, for example.

As used herein the term phosphonate ester represents a group of one of the formulae:
—P(═O)(OR)₂ and —P(═O)(OR)O⁻ wherein each R is independently a phosphonate ester substituent, for example, —H, substituted or unsubstituted C₁₋₂₀ alkyl, substituted or unsubstituted C₃₋₂₀ heterocyclyl, C₃₋₂₀ heterocyclyl substituted with a further C₃₋₂₀ heterocyclyl, substituted or unsubstituted C₁₋₂₀ alkylene-C₃₋₂₀ heterocyclyl, substituted or unsubstituted C₃₋₂₅ cycloalkyl, substituted or unsubstituted C₁₋₂₀ alkylene-C₃₋₂₅ cycloalkyl, aryl, substituted or unsubstituted C₁₋₂₀ alkylene-aryl. Examples of phosphonate ester groups include, but are not limited to, —P(═O)(OCH₃)₂, —P(═O)(OCH₂CH₃)₂, —P(═O)(O-t-Bu)₂, and —P(═O)(OPh)₂.

As used herein the term phosphoric acid represents a group of the formula: —OP(═O)(OH)₂.

As used herein the term phosphate ester represents a group of one of the formulae:
—OP(═O)(OR)₂ and —OP(═O)(OR)O⁻ wherein each R is independently a phosphate ester substituent, for example, —H, substituted or unsubstituted C₁₋₂₀ alkyl, substituted or unsubstituted C₃₋₂₀ heterocyclyl, C₃₋₂₀ heterocyclyl substituted with a further C₃₋₂₀ heterocyclyl, substituted or unsubstituted C₁₋₂₀ alkylene-C₃₋₂₀ heterocyclyl, substituted or unsubstituted C₃₋₂₅ cycloalkyl, substituted or unsubstituted C₁₋₂₀ alkylene-C₃₋₂₅ cycloalkyl, aryl, substituted or unsubstituted C₁₋₂₀ alkylene-aryl. Examples of phosphate ester groups include, but are not limited to, —OP(═O)(OCH₃)₂, —OP(═O)(OCH₂CH₃)₂, —OP(═O)(O-t-Bu)₂, and —OP(═O)(OPh)₂.

As used herein the term amino represents a group of formula —NH₂. The term C₁-C₁₀ alkylamino represents a group of formula —NHR′ wherein R′ is a C₁₋₁₀ alkyl group, preferably a C₁₋₆ alkyl group, as defined previously. The term di(C₁₋₁₀)alkylamino represents a group of formula —NR′R″ wherein R′ and R″ are the same or different and represent C₁₋₁₀ alkyl groups, preferably C₁₋₆ alkyl groups, as defined previously. The term arylamino represents a group of formula —NHR′ wherein R′ is an aryl group, preferably a phenyl group, as defined previously. The term diarylamino represents a group of formula —NR′R″ wherein R′ and R″ are the same or different and represent aryl groups, preferably phenyl groups, as defined previously. The term arylalkylamino represents a group of formula —NR′R′ wherein R′ is a C₁₋₁₀ alkyl group, preferably a C₁₋₆ alkyl group, and R″ is an aryl group, preferably a phenyl group.

As used herein the term amido represents a group of formula: —C(═O)NR′R″, wherein R′ and R″ are independently amino substituents, as defined for di(C₁₋₁₀)alkylamino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R′ and R″, together with the nitrogen atom to which they are attached, form a heterocyclic structure as in, for example, piperidinocarbonyl, morpholinocarbonyl, thiomorpholinocarbonyl, and piperazinocarbonyl.

As used herein the term acylamido represents a group of formula: —NR¹C(═O)R², wherein R¹ is an amide substituent, for example, hydrogen, a C₁₋₂₀alkyl group, a C₃₋₂₀ heterocyclyl group, an aryl group, preferably hydrogen or a C₁₋₂₀ alkyl group, and R² is an acyl substituent, for example, a C₁₋₂₀ alkyl group, a C₃₋₂₀ heterocyclyl group, or an aryl group, preferably hydrogen or a C₁₋₂₀ alkyl group. Examples of acylamide groups include, but are not limited to, —NHC(═O)CH₃, —NHC(═O)CH₂CH₃, —NHC(═O)Ph, —NHC(═O)C₁₅H₃₁ and —NHC(═O)C₉H₁₉. Thus, a substituted C₁₋₂₀ alkyl group may comprise an acylamido substituent defined by the formula —NHC(═O)—C₁₋₂₀ alkyl, such as —NHC(═O)C₁₅H₃₁ or —NHC(═O)C₉H₁₉. R¹ and R² may together form a cyclic structure, as in, for example, succinimidyl, maleimidyl, and phthalimidyl:

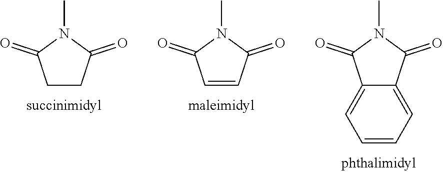

A C₁₋₁₀ alkylthio group is a said C₁₋₁₀ alkyl group, preferably a C₁₋₆ alkyl group, attached to a thio group. An arylthio group is an aryl group, preferably a phenyl group, attached to a thio group.

A C₁₋₂₀ alkoxy group is a said substituted or unsubstituted C₁₋₂₀ alkyl group attached to an oxygen atom. A C₁₋₆ alkoxy group is a said substituted or unsubstituted C₁₋₆ alkyl group attached to an oxygen atom. A C₁₋₄ alkoxy group is a substituted or unsubstituted C₁₋₄ alkyl group attached to an oxygen atom. A substituted C₁₋₂₀ alkoxy group includes a C₁₋₂₀ perfluoroalkoxy group. A C₁₋₂₀ perfluoroalkoxy group is a C₁₋₂₀ perfluoroalkyl group attached to an oxygen atom. An example of a C₁₋₂₀ perfluoroalkoxy group is a tert-nonafluorobutyloxy group, —OC(CF₃)₃.

An aryloxy group is a substituted or unsubstituted aryl group, as defined herein, attached to an oxygen atom. It may for instance be unsubstituted or substituted phenoxy. An example of an aryloxy group is —OPh (phenoxy).

The term “amino acid”, as used herein, in connection with any of the compounds described herein, means an amino acid residue. The amino acid residue is typically bonded via its C-terminus or via its N-terminus to the atom in the compound described herein to which it is said to be bonded. For example, as would be understood by the skilled person, when an amino acid is said to be bonded to a carbon atom of a carbonyl group in a compound described herein, the nitrogen atom at the N-terminus of the amino acid is typically bonded to that carbon atom. Similarly, where an amino acid is said to be bonded to a nitrogen atom of an amine group in a compound described herein, the carbon atom of the C-terminus of the amino acid would generally be bonded to that nitrogen atom. The carbon atom of the C-terminus of an amino acid may alternatively be bonded to an oxygen atom in a compound as described herein. An amino acid in any of the compounds described herein may for instance be an amino acid residue selected from arginine (Arg), histidine (His), lysine (Lys), aspartic acid (Asp), glutamic acid (Glu), serine (Ser), threonine (Thr), asparagine (Asn), glutamine (Gln), cysteine (Cys), selenocysteine (Sec), glycine (Gly), proline (Pro), alanine (Ala), valine (Val), isoleucine (Ile), leucine (Leu), methionine (Met), phenylalanine (Phe), tyrosine (Tyr) and tryptophan (Trp).

The term azine, as used herein, means a heterocyclic compound containing a 6-membered aromatic ring, in which one or more of the ring carbon atoms has been replaced by a nitrogen atom. For example, pyridine is an azine, as is pyridazine.

Unless otherwise specified, included in the above are the well known ionic, salt, solvate, and protected forms of these substituents. For example, a reference to carboxylic acid or carboxyl group (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms.

The compounds of the invention can exist in various tautomeric forms and it is to be understood that the invention encompasses all such tautomeric forms.

In certain of the compounds of the invention, dependant on the nature of the substituent, there may be chiral carbon atoms and therefore the compounds may exist as stereoisomers. The invention extends to all optical isomers such as stereoisomeric forms of the compounds of the invention, including enantiomers, diastereomers and mixtures thereof, such as racemates. The different stereoisomeric forms may be separated or resolved one from the other by conventional methods or any given isomer may be obtained by conventional stereoselective or sterospecific syntheses.

It is also to be understood that any atom present in a compound of the invention may be present in any available naturally-occuring isotopic form. For instance, a carbon atom may be ¹²C or ¹³C. A hydrogen atom may be ¹H or ²H (deuterium). As used herein, the terms “treat”, “treating” and “treatment” refer to both therapeutic treatment and prophylactic or preventative measures, wherein the object is to prevent or slow down (lessen) an undesired physiological change or disorder, such as the development or spread of disease. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “pharmaceutically acceptable” indicates that the substance or composition must be compatible chemically and/or toxicologically with the other ingredients comprising a formulation, and/or the patient being treated therewith.

### Compounds for Use in the Treatment of Blood Cancer

The present invention provides a hypoxia inducible factor (HIF) prolyl hydroxylase (PHD) inhibitor for use in the treatment of blood cancer. That a PHD inhibitor may be useful in the treatment of blood cancer has been shown by the present inventors.

In particular, as described in Example 163, it is a finding of the invention that PHD2 is required for the initiation of acute myeloid leukaemia driven by Meis1 and Hoxa9. As described in Example 163, the present inventors have also found that PHD2 is required for MII-AF9-driven leukamogenesis.

In addition, the present inventors established that acute PHD2 knockdown in established AML cells compromises leukaemia progression. Taken together, the data show that PHD2 is required for AML cell survival and efficient disease progression, highlighting its value as a therapeutic target in leukemia.

The inventors have validated this by testing both currently-available and newly-generated PHD inhibitors on a range of genetically diverse blood cancer cells, and have demonstrated that a range of PHD inhibitors, with distinct modes of action, do indeed compromise AML, CML and MM cells (Example 167, 171). Thus, the inventors have provided both genetic and pharmacological evidence that PHD inhibition is a very promising therapeutic strategy for blood cancer.

Accordingly, the present invention relates to a PHD inhibitor for use in the treatment of blood cancer, for instance acute myeloid leukaemia, chronic myeloid leukaemia or multiple myeloma.

The PHD inhibitor for use according to the invention may be any compound, including any small molecule, salt or complex, or any biologic, capable of inhibiting HIF prolyl hydroxylase. A biologic as described herein includes an amino acid, a signalling molecule, a peptide, a protein, an antibody, a nucleic acid, an oligonucleotide, or a cell. The PHD inhibitor for use in the treatment of blood cancer may be an inhibitor of PHD2, i.e. it may be a PHD2 inhibitor. Alternatively, the PHD inhibitor may be a PHD1 inhibitor, or a PHD3 inhibitor, or it may be a PHD inhibitor which inhibits any combination of PHDs. For instance, the PHD inhibitor may be an inhibitor of two or more of PHD1, PHD2 and PHD3, for example it may be an inhibitor of PHD2 and PHD1, an inhibitor of PHD2 and PHD3, an inhibitor of PHD1 and PHD3, or an inhibitor of PHD1, PHD2 and PHD3.

A wide range of PHD inhibitors are known to the skilled person. The PHD inhibitor for use in the present invention may be any one of those known PHD inhibitors, or it may be a derivative of a known PHD inhibitor (for instance, a derivative optimised for blood cancer), or indeed any other PHD inhibitor may be employed. The PHD inhibitor for use according to the invention may for instance be any one of the following types of PHD inhibitor: a cobalt salt, such as cobalt dichloride, or a copper salt, or a nickel salt, or any other metal salt that is known to be capable of inhibiting HIF prolyl hydroxylase. Alternatively, any other known PHD inhibitor may be employed, especially for instance an iron chelator, such as deferoxamine, 3,4-dihydroxybenzoic acid, 1,10-phenanthrolines, or quercetin; a 2-OG derivative mimic, or competitor (with respect to PHD binding), such as dimethyloxalylglycine (DMOG) which is a prodrug form of N-oxalylglycine (NOG); FG-2216; roxadustat; a quinolone, such as JNJ-42905343; a quinoxaline; a benzamidazole derivative, such as JNJ-42041935; an isoquinolone derivative; a 5-hydroxy-1,7 naphthyridine derivative, such as ISM5411; a monocyclic pyridine compound, such as vadadustat, and AKB6899; a pyrazolopyrimidine derivative; a pyrimidine-trione, such as daprodustat; an N-alkoxyquinolone, such as desidustat; a tetrahydropyran derivative; a dihydrothienopyridone derivativel; a dihydrofuropyridoene derivative; a quinazoline-2,4-dione; a 4-oxo-2-thioxo-7-quinasoline; a 5-aminocarbonyl-4-hydroxypyrimidine derivative, such as MK8617; a spiroindolone; a 2,8-diazaspori[4,5]-decan-lone; a pyrazolone derivative, such as molidustat; a triazole substituted heteroaryl amide; a phenolic compound, such as ((S)-{2[2-(5-cyano-3-hydroxy-pyridin-2-yl)-thiazol-4-yl]-acetylamino}-phenyl-acetic acid); a bicyclic heteroaryl derivative, such as (1,2,4-triazolo-[1,5-a]pyridine); a diacylhydrazine; pyrathione Zn, or (5-(3-(4-chlorophenoxyl) prop-1-yn-1-yl)-3-hydroxypicolinoyl)glycine.

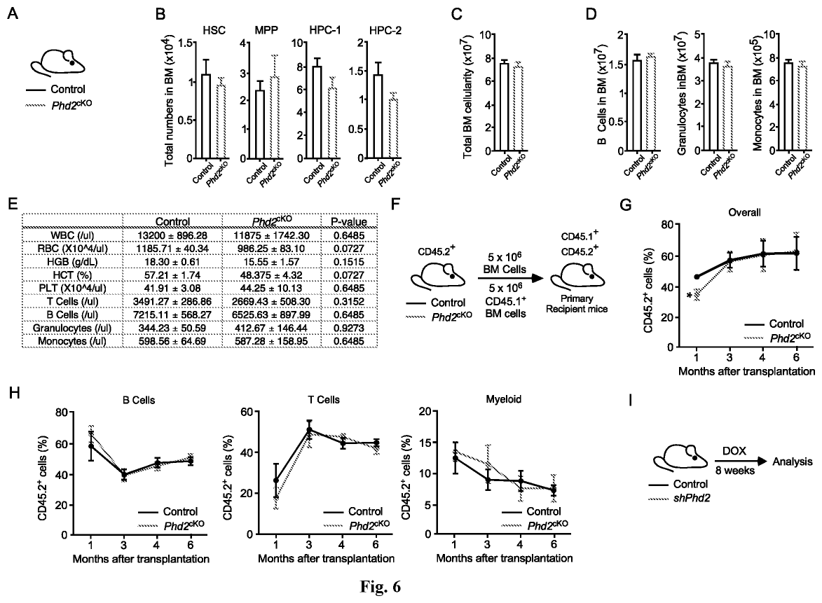

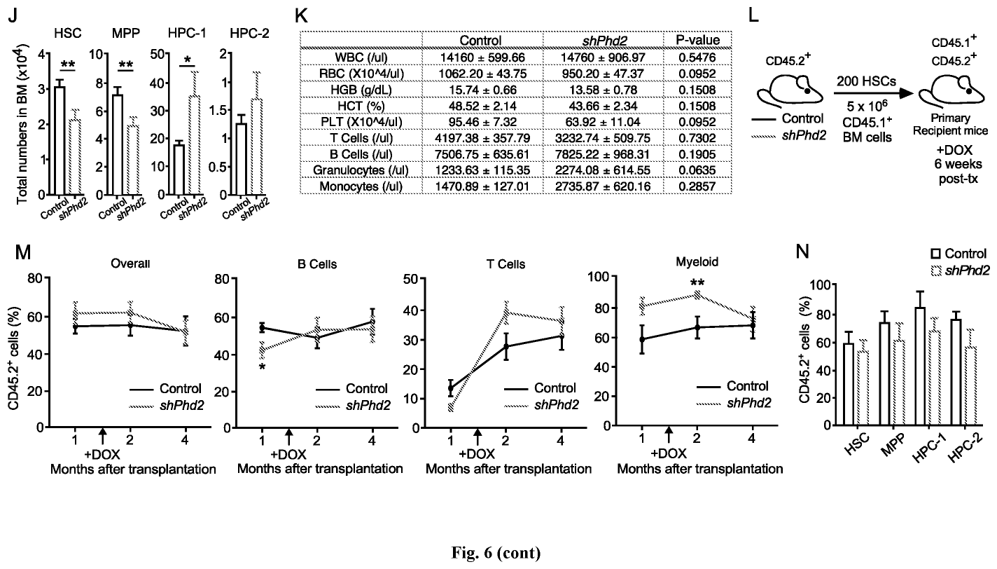

The PHD inhibitor for use according to the invention may be a compound of any one of the following structures or a pharmaceutically acceptable salt thereof. The following compounds are known PHD inhibitors, as described in the academic and patent literature such as described in Joharapurkar, A. A. et al., J. Med. Chem. 2018, 61, 6964-6982 (see FIGS. 6 to 10 thereof) and in Sabnis, R. W., ACS Med. Chem. Lett. 2021, 12, 1868-1869:

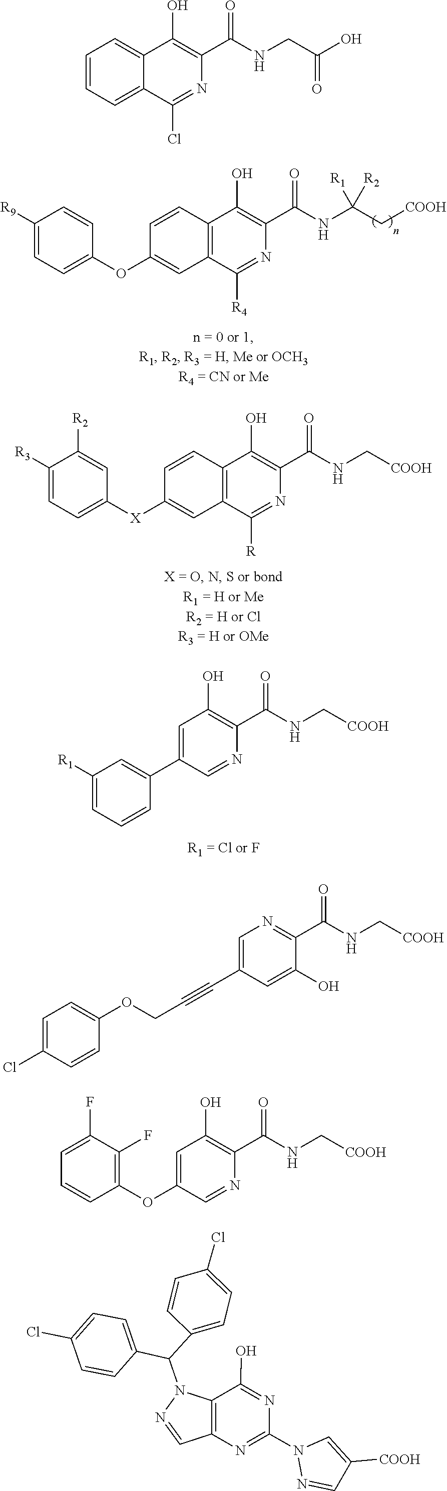

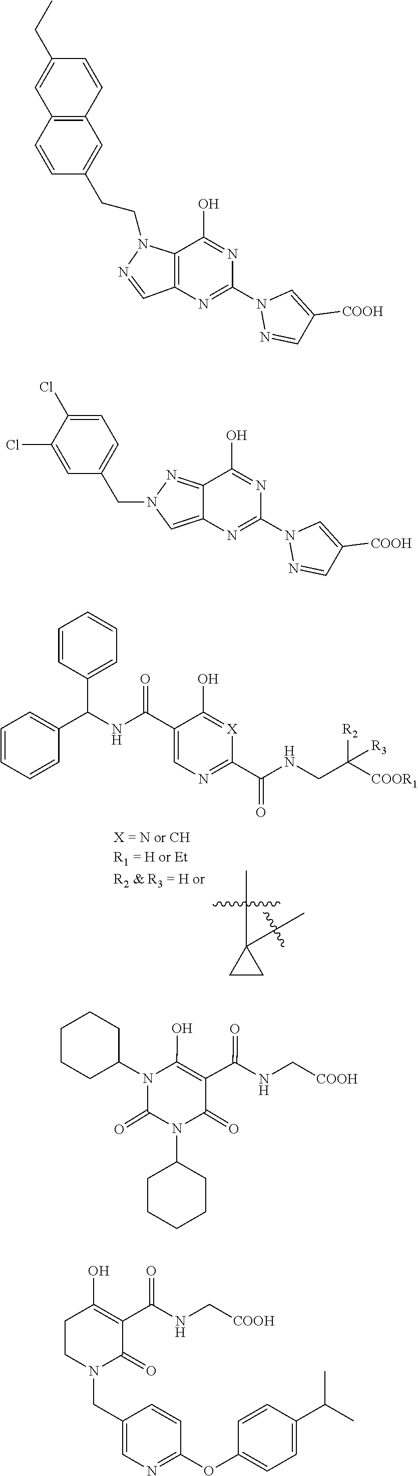

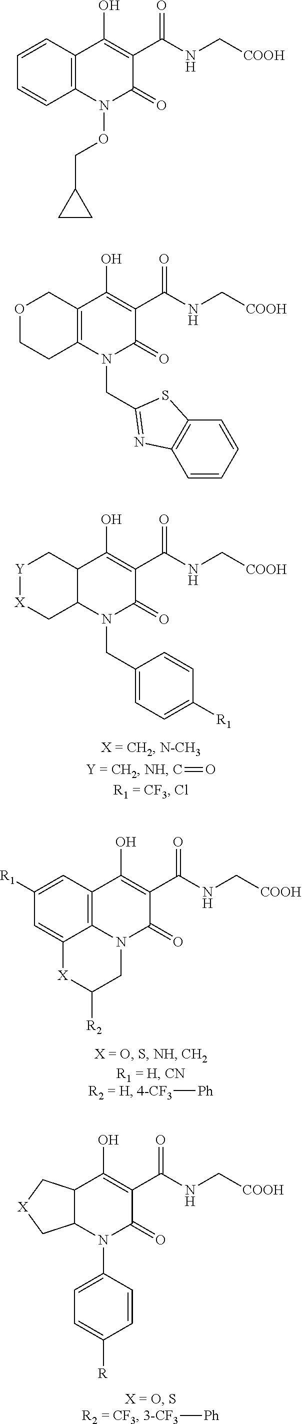

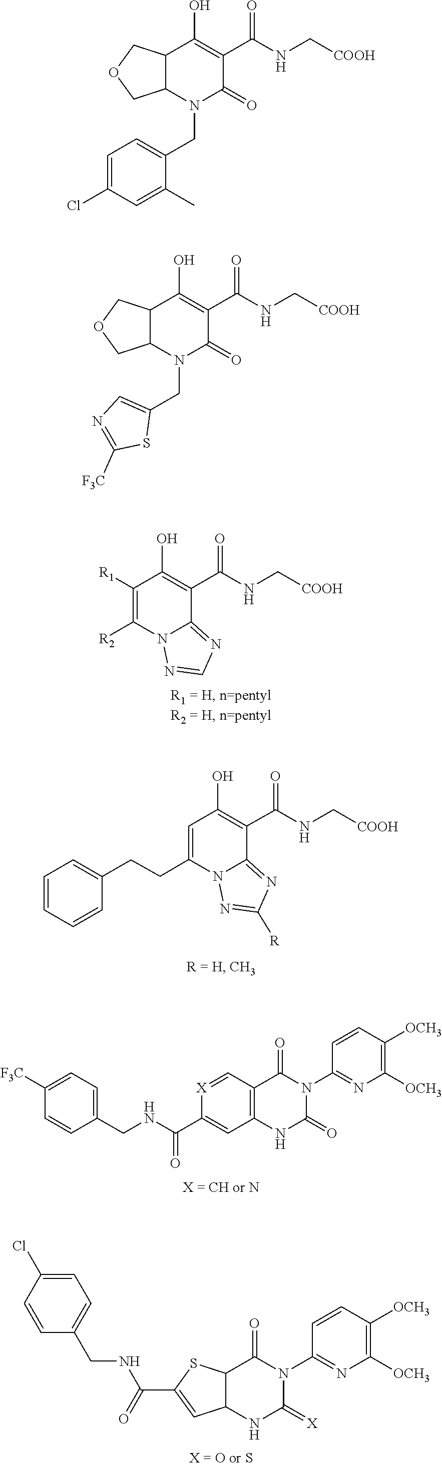

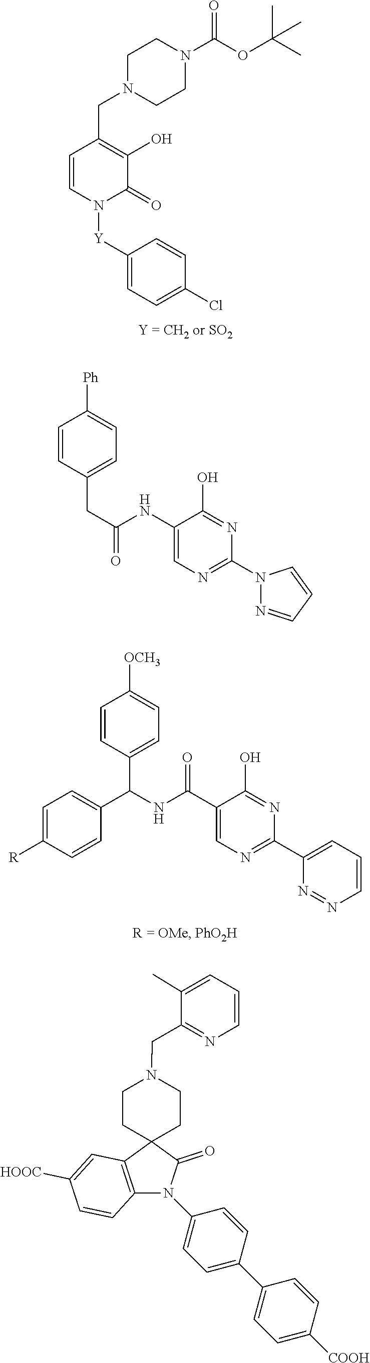

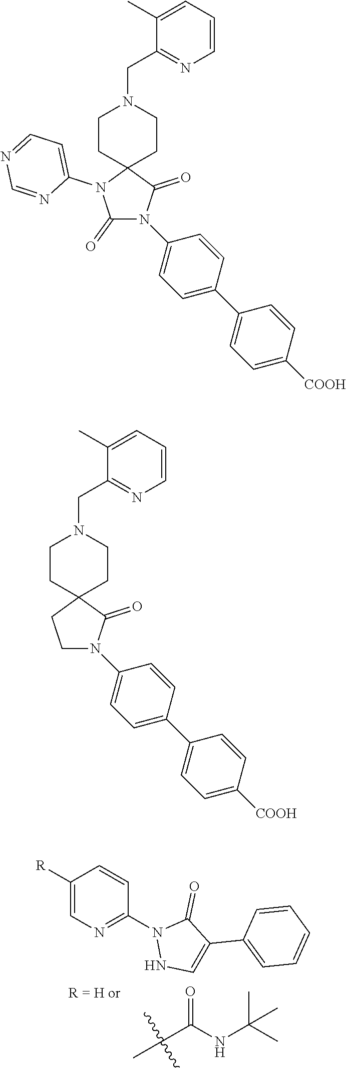

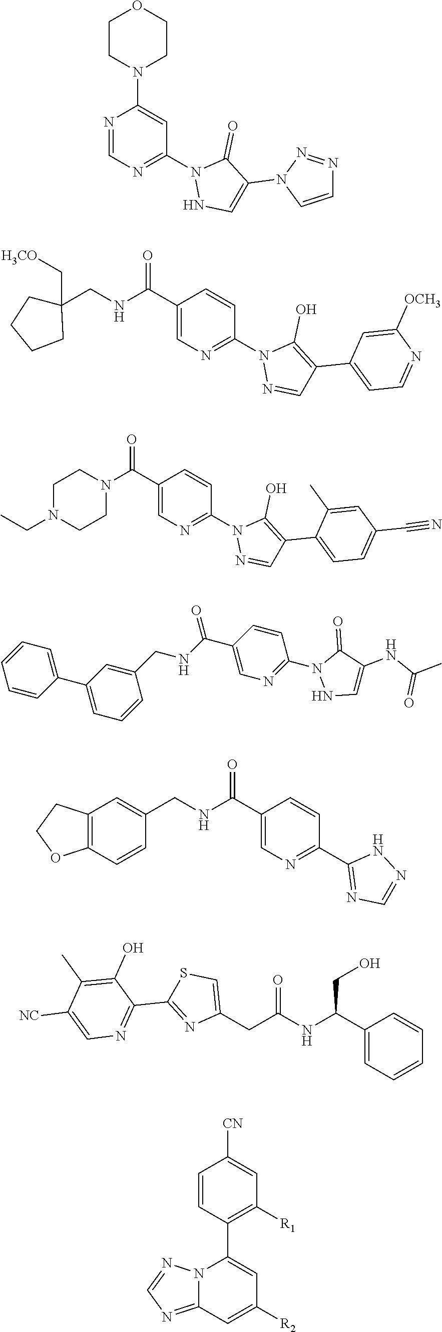

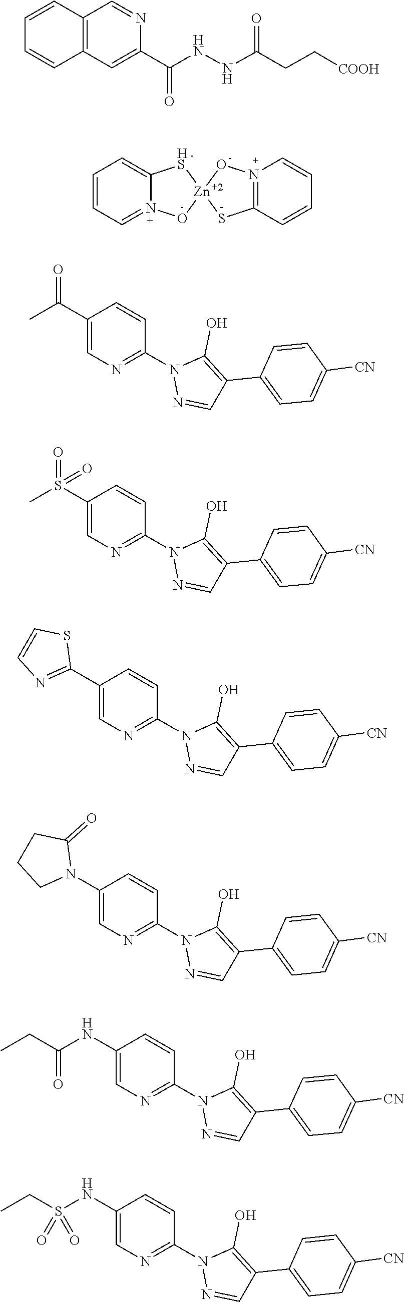

The PHD inhibitor for use in the treatment of blood cancer may for instance be FG-2216, molidustat, daprodustat, vadadustat, DS1093a, SSS 17, enarodustat, desidustat, BGE-175, JNJ42905343, MK-8617, IOX2, IOX3, IOX4, or AKB-6899.

The PHD inhibitor may for instance be daprodustat or a structurally-related PHD inhibitor. Accordingly, the present invention also provides a PHD inhibitor for use in the treatment of blood cancer (including AML, CML and MM) wherein said PHD inhibitor is a compound of formula (II) or a pharmaceutically acceptable salt thereof

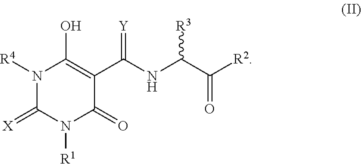

In the above formula (II), typically R¹ and R⁴ are each independently selected from the group consisting of H, —NR⁵R⁶, unsubstituted or substituted C₁₋₁₀ alkyl, unsubstituted or substituted C₂₋₁₀ alkenyl, unsubstituted or substituted C₂₋₁₀ alkynyl, unsubstituted or substituted C₃₋₈ cycloalkyl, unsubstituted or substituted —C₃₋₈ cycloalkylene-C₁₋₁₀ alkyl, unsubstituted or substituted C₅₋₈ cycloalkenyl, unsubstituted or substituted —C₅₋₈ cycloalkenylene-C₁₋₁₀ alkyl, unsubstituted or substituted C₃₋₈ heterocyclyl, unsubstituted or substituted —C₃₋₈ heterocyclylene-C₁₋₁₀ alkyl, unsubstituted or substituted aryl, unsubstituted or substituted-arylene-C₁₋₁₀ alkyl, unsubstituted or substituted-heteroaryl and unsubstituted or substituted-heteroarylene-C₁₋₁₀ alkyl.

Usually, in the above formula (II) R² is-NR⁷R⁸ or —OR⁹.

R³, in the above formula (II) may be H or unsubstituted or substituted C₁₋₄ alkyl. Typically in the above formula (II), R⁵ and R⁶ are each independently selected from the group consisting of H, unsubstituted or substituted C₁₋₁₀ alkyl, unsubstituted or substituted —C₃₋₈ cycloalkyl, unsubstituted or substituted —C₃₋₈ cycloalkylene-C₁₋₁₀ alkyl, —C₃₋₈ heterocyclyl, unsubstituted or substituted —C₃₋₈ heterocyclylene-C₁₋₁₀ alkyl, unsubstituted or substituted aryl, unsubstituted or substituted-arylene-C₁₋₁₀ alkyl, unsubstituted or substituted heteroaryl, unsubstituted or substituted-heteroarylene-C₁₋₁₀ alkyl, unsubstituted or substituted —C(O)C₁₋₄ alkyl, unsubstituted or substituted —C(O)C₃₋₆ cycloalkyl, —C(O)C₃₋₆ heterocyclyl, unsubstituted or substituted —C(O)aryl, unsubstituted or substituted —C(O) heteroaryl and unsubstituted or substituted —S(O)₂C₁₋₄ alkyl, or, when R⁵ and R⁶ are attached to the same nitrogen, R⁵ and R⁶ taken together with the nitrogen to which they are attached form a 5-or 6-or 7-membered saturated heterocyclic ring which is unsubstituted or substituted and which optionally contains one other heteroatom selected from oxygen, nitrogen and sulphur.

In the above formula (II), R⁷ and R⁸ are usually each independently selected from the group consisting of H, unsubstituted or substituted C₁₋₁₀ alkyl, unsubstituted or substituted C₂₋₁₀ alkenyl, unsubstituted or substituted C₂₋₁₀ alkynyl, unsubstituted or substituted C₃₋₈ cycloalkyl, unsubstituted or substituted C₃₋₈ heterocyclyl, unsubstituted or substituted aryl, and unsubstituted or substituted heteroaryl.

R⁹ in the above formula (II) is typically H or C₁₋₁₀ alkyl which is unsubstituted or substituted with one or more substituents independently selected from the group consisting of unsubstituted or substituted C₃₋₆ cycloalkyl, unsubstituted or substituted C₃₋₈ heterocyclyl, unsubstituted or substituted aryl, and unsubstituted or substituted heteroaryl.

Usually, X is O or S; and Y is O or S.

In the above formula (II), R¹ is usually unsubstituted or substituted C₃₋₈ cycloalkyl. Typically, R² is OH. R³ may be H. Usually, in the above formula (II) R⁴ is unsubstituted or substituted C₃₋₈ cycloalkyl. X is typically O. Y may also typically be O.

The compound of formula (II) may for instance be daprodustat. Thus, the PHD inhibitor may be a compound of formula (IIa), or a pharmaceutically acceptable salt thereof

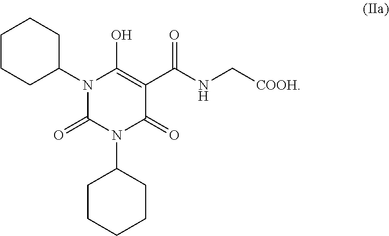 [SMILES: O=C(O)CNC(=O)c1c(O)n(C2CCCCC2)c(=O)n(C2CCCCC2)c1=O]

The compounds of formulae (II) and (Ila) shown above, their preparation, and their utility as PHD inhibitors, are described in WO 2007/150011, the entire contents of which are incorporated herein by reference.

Alternatively, for instance, the PHD inhibitor may be molidustat or a structurally-related PHD inhibitor. Accordingly, the present invention also provides a PHD inhibitor for use in the treatment of blood cancer wherein said PHD inhibitor is a compound of formula (III) or a pharmaceutically acceptable salt thereof

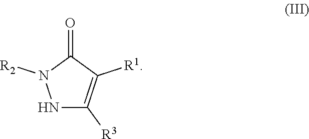

In the above formula (III), R¹ typically represents a heteroaryl group of the formula

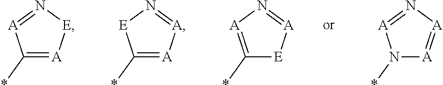

wherein * denotes a linkage point with the dihydropyrazolone ring. A, typically, in each individual occurrence denotes C—R⁴ or N, wherein at most two ring members A represent N at the same time. E usually denotes O, S or N—R⁵.

In the above formula (III) R² typically represents a heteroaryl group of the formula

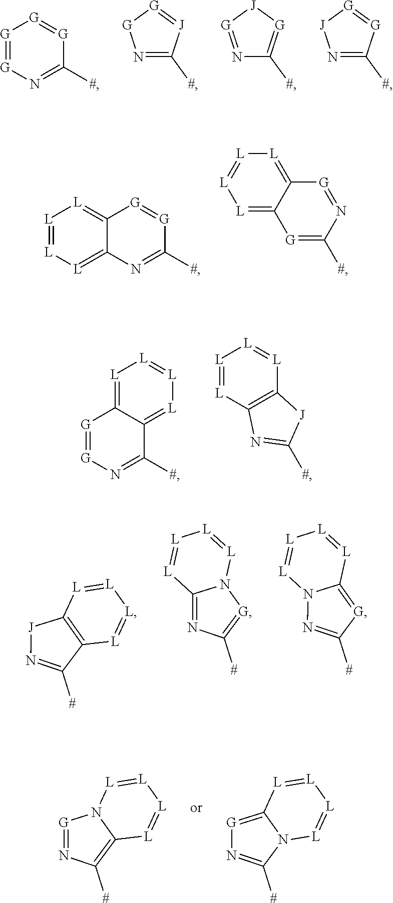

where #denotes the linkage point with the dihydropyrazolone ring, G in each individual occurrence denotes C—R⁶ or N, wherein at most two ring members G represent N at the same time.

Usually J denotes O, S, or N—R⁷.

L may in each individual occurrence denotes C—R⁸ or N, wherein at most two ring members L represent N at the same time.

R⁴, R⁶ and R⁸ in the above formula (III), are typically the same or different and are each independently selected from H or a substituent chosen from the series consisting of halogen, —CN, nitro, C₁₋₆ alkyl, —C₃₋₇-cycloalkyl, 4-to 10-membered heterocyclyl, phenyl, 5 or 6-membered heteroaryl, —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹.

When an alkyl group is referred to for the above formula (III), it may be unsubstituted or substituted one to three times by the same or different groups independently selected from halogen, CN, oxo, —C₃₋₇-cycloalkyl, 4-to 10-membered heterocyclyl, phenyl, 5-or 6-membered heteroaryl-C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹ wherein the cycloalkyl, heterocyclyl, phenyl and heteroaryl groups may unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxy-carbonyl.

When a C₃₋₇ cycloalkyl, 4-to 10-membered heterocyclyl, phenyl and 5-or 6-membered heteroaryl are referred to for formula (III), they may be unsubstituted or substituted one to three times by the same or different groups independently selected from halogen, —CN, nitro, C₁₋₆ alkyl, —C₃₋₇-cycloalkyl, 4-to 10 membered heterocyclyl, phenyl, 5 or 6-membered heteroaryl, —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹,

wherein the alkyl group is unsubstituted or substituted one to three times by the same or different groups independently selected from halogen, CN, hydroxyl, trifluoromethoxy, (C₁₋₄)-alkoxy, amino, mono-(C₁₋₄)-alkylamino, di-(C₁₋₄)-alkylamino, hydroxycarbonyl, (C₁₋₄)-alkoxycarbonyl, (C₃₋₇)-cycloalkyl, 4-to 7-membered heterocyclyl, phenyl and/or 5-or 6-membered heteroaryl.

R⁹, R¹⁰, R¹¹, R¹³, R¹⁴, R¹⁷, R¹⁹, R²¹, R²⁴, R²⁵, R²⁶, R²⁸, R²⁹ and R³⁰ as described in the above formula (III), may independently of one another for each individual occurrence represent groups selected from H, C₁₋₆ alkyl, C₃₋₇ cycloalkyl, 4-to 10-membered heterocyclyl, phenyl and 5-or 6-membered heteroaryl, wherein C₃₋₇ cycloalkyl, 4-to 10-membered heterocyclyl, phenyl and 5-or 6-membered heteroaryl may be unsubstituted or substituted one to three times by the same or different groups independently selected from halogen, CN, C₁₋₄-alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl and C₁₋₆ alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl, (C₁₋₄)-alkoxycarbonyl, C₃₋₇ cycloalkyl, C₄₋₇ heterocycloalkyl, phenyl and/or 5-or 6-membered heteroaryl.

In the above formula (III), R¹², R¹⁵, R¹⁶, R¹⁸, R²⁰, R²², R²³, R²⁷ and R³¹ independently of one another for each individual occurrence may represent groups selected from H and C₁₋₆ alkyl,

wherein C₁₋₆ alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl and/or (C₁₋₄)-alkoxycarbonyl.

Typically, in the above formula (III), R¹¹ and R¹², R¹⁴ and R¹⁵, R¹⁶ and R¹⁷, R¹⁸ and R¹⁹, R²⁰ and R²¹, R²¹ and R²², R²³ and R²⁴, R²⁶ and R²⁷ and R³⁰ and R³¹ in each case paired together with the atoms to which they are bonded can form a 5-or 6-membered heterocyclyl ring, which may be unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl.

Usually, R⁵ and R⁷ are the same or different and independently and are each selected from H, C₁₋₆ alkyl, C₃₋₇ cycloalkyl, C₄₋₇ heterocycloalkyl, phenyl and 5-or 6-membered heteroaryl.

When R⁵ and/or R⁷ of formula (III) are C₁₋₆ alkyl, they may be unsubstituted or substituted one to three times by the same or different groups independently selected from halogen, —CN, nitro, —C₃₋₇-cycloalkyl, 4-to 10-membered heterocyclyl, phenyl, 5 or 6-membered heteroaryl, —C(O)R⁹, —C(O)OR¹⁰, —C(O)NR¹¹R¹², —OC(O)R¹³, OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹

wherein the cycloalkyl, heterocyclyl, phenyl and heteroaryl groups may be unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl,

When R⁵ and/or R⁷ of formula (III) are C₃₋₇ cycloalkyl, 4 to 7 membered heterocyclyl, phenyl and 5-or 6-membered heteroaryl they may be unsubstituted or substituted one to three times by the same or different groups independently selected from C₁₋₆ alkyl, halogen, —CN, nitro, —C₃₋₇-cycloalkyl, 4-to 10-membered heterocyclyl, phenyl, 5 or 6-membered heteroaryl, —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹,

wherein the alkyl group is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl, C₁₋₄ alkoxycarbonyl, C₃₋₇ cycloalkyl, 4-to 7-membered heterocyclyl, phenyl and/or 5-or 6-membered heteroaryl.

Usually for formula (III), R⁹, R¹⁰, R¹¹, R¹³, R¹⁴, R¹⁷, R¹⁹, R²¹, R²⁴, R²⁵, R²⁶, R²⁸, R²⁹ and R³⁰ independently of one another for each individual occurrence represent a group selected from H, C₁₋₆ alkyl, C₃₋₇ cycloalkyl, 4-to 7-membered heterocyclyl, phenyl and 5-or 6-membered heteroaryl, wherein

C₃₋₇ cycloalkyl, 4-to 7-membered heterocycloalkyl, phenyl and 5-or 6-membered heteroaryl are unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl, and
(C₁₋₆)-alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl, C₁₋₄ alkoxycarbonyl, C₃₋₇ cycloalkyl, 4-to 7-membered heterocyclyl, phenyl and/or 5-or 6-membered heteroaryl.

Typically for formula (III), R¹², R¹⁵, R¹⁶, R¹⁸, R²⁰, R²², R²³, R²⁷ and R³¹ independently of one another for each individual occurrence represent a group selected from H and C₁₋₆ alkyl,

wherein C₁₋₆ alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl and/or (C₁₋₄)-alkoxycarbonyl.

For formula (III), R¹¹ and R¹², R¹⁴ and R¹⁵, R¹⁶ and R¹⁷, R¹⁸ and R¹⁹, R²⁰ and R²¹, R²¹ and R²², R²³ and R²⁴, R²⁶ and R²⁷ and R³⁰ and R³¹ may in each case be paired together with the atoms to which they are bonded can form a 5-or 6-membered heterocyclyl ring, which can be unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl.

For formula (III), R³ is typically H, C₁₋₆ alkyl, or C₃₋₇ cycloalkyl.

For preferred HIF PHD inhibitors of formula (III) or pharmaceutically acceptable salts thereof, R¹ denotes a group of the formula

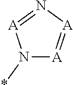

wherein * denotes the linkage point with the dihydropyrazolone ring. For such compounds A in each individual occurrence typically denotes CH or N, wherein at most two ring members A represent N at the same time.

Preferably, R² of formula (III), may represent a heteroaryl group of the formula

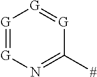

wherein #denotes the linkage point with the dihydropyrazolone ring. G in each individual occurrence usually denotes C—R⁶ or N, wherein at most two ring members G represent N at the same time. R⁶ in formula (III) usually represents H or 5 or 6-membered hetercyclyl. R³ is usually H.

The compound of formula (III) may for instance be molidustat. Thus the HIF PHD inhibitor for use according to the invention may be a compound of formula (IIIa) or a pharmaceutically acceptable salt thereof

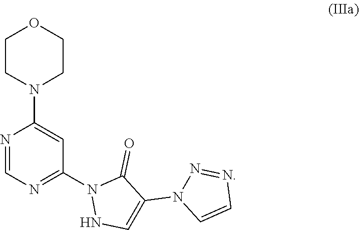 [SMILES: O=c1c(-n2ccnn2)c[nH]n1-c1cc(N2CCOCC2)ncn1]

The compounds of formulae (III) and (IIIa) shown above, their preparation, and their utility as PHD inhibitors, are described in WO 2008/067871, the entire contents of which are incorporated herein by reference.

### Compounds of the Invention

The invention also relates to a series of novel compounds and their use as hypoxia inducible factor prolyl hydroxylase domain inhibitors (PHD inhibitors). The compounds therefore have potential utility in treating conditions for which HIF-PHD is a therapeutic target. Thus, the PHD inhibitors are useful in the treatment of blood cancers. The inhibitors are for instance useful in the treatment of leukaemia, for instance acute myeloid leukaemia (AML), or chronic myeloid leukaemia (CML). The inhibitors are also useful in the treatment of multiple myeloma (MM).

Accordingly, the present invention provides a compound which is a substituted azine of formula (I) or a pharmaceutically acceptable salt thereof

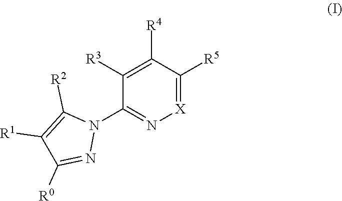

In the above formula (I), X is C(R⁶) or N. Preferably X is C(R⁶), i.e. preferably X is a ring-carbon atom bonded to R⁶ (in which case, the substituted azine is a substituted pyridine). Often, however, X is N (in which case, the substituted azine is a substituted pyridazine).

R⁰ is H or unsubstituted or substituted C₁₋₆ alkyl. Typically, R⁰ is H or unsubstituted C₁₋₆ alkyl. Usually, R⁰ is selected from H, methyl and ethyl. Often, R⁰ is H or methyl.

R¹ is H, unsubstituted or substituted C₁₋₆ alkyl, unsubstituted or substituted aryl, unsubstituted or substituted heteroaryl, —CN, —N(R^{t})C(O)N(R^{u})R^{v}, —C(O)OR^{w} or —C(O)N(R^{x})R⁷.

Typically R¹ is —C(O)N(R^{x})R⁷. This is especially typical when R⁵ is other than —C(O)N(R^{x})R⁷. Thus, R¹ being —C(O)N(R^{x})R⁷ is especially typical when R⁵ is H, unsubstituted or substituted C₁₋₆ alkyl, unsubstituted or substituted aryl, unsubstituted or substituted heteroaryl, —CN, —N(R^{t})C(O)N(R^{u})R^{v} or —C(O)OR^{w}.

R¹ may also typically be H, unsubstituted or substituted C₁₋₆ alkyl, unsubstituted or substituted aryl, unsubstituted or substituted heteroaryl, —CN, —N(R^{t})C(O)N(R^{u})R^{v}, or —C(O)OR^{w}. This is especially typical when R⁵ is —C(O)N(R^{x})R⁷. For instance, R¹ may be H, unsubstituted or substituted C₁₋₆ alkyl, —CN or —C(O)OR^{w}, preferably H, —CN, or —C(O)OR^{w}. These are often the case when R⁵ is —C(O)N(R^{x})R⁷.

Thus, R¹ may be selected from H, unsubstituted or substituted C₁₋₆ alkyl, —CN, —C(O)OR^{w} and —C(O)N(R^{x})R⁷. R¹ may for instance be selected from H, —CN, —C(O)OR^{w} and —C(O)N(R^{x})R⁷. R¹ may for instance be selected from H, unsubstituted or substituted C₁₋₆ alkyl, CN and —C(O)OR^{w}, or, for instance, from H, —CN and —C(O)OR^{w}.

R² is H, —OR^{q} or unsubstituted or substituted C₁₋₆ alkyl. Alternatively, R² is —N═, in which case R³ is ═C(R^{y})— and R² and R³ together form a group of formula —N═C(R^{y})—.

R² may for instance be H, —OR^{q} or unsubstituted or substituted C₁₋₆ alkyl. Typically, in that case, R² is H or unsubstituted or substituted C₁₋₆ alkyl. For instance, R² may be H or unsubstituted C₁₋₆ alkyl, for instance R² may be H, ethyl or methyl. R² may for example be H or methyl.

R³ is H, —OR⁸ or unsubstituted or substituted C₁₋₆ alkyl. Alternatively, R³ is ═C(R^{y})—, in which case R² is —N═ and and R² and R³ together form a group of formula —N═C(R^{y})—.

R³ may for instance be H, —OR⁸ or unsubstituted or substituted C₁₋₆ alkyl. Typically, in that case, R³ is H or —OR⁸. R³ may also usually be selected from H or unsubstituted or substituted C₁₋₆ alkyl. R³ is often, however, —OR⁸.

In some embodiments, R² is —N═ and R³ is ═C(R^{y})— and R² and R³ together form a group of formula —N═C(R^{y})—.

R⁴ is H, unsubstituted or substituted C₁₋₆ alkyl, —OR⁹ or —C(O)OR¹⁰. R⁴ may for instance be H, unsubstituted C₁₋₆ alkyl, —OR⁹ or —C(O)OR¹⁰. Often, however, R⁴ is H, —OR⁹ or —C(O)OR¹⁰. Typically R⁴ is selected from —OR⁹ and —C(O)OR¹⁰, or R⁴ is —OR⁹. Alternatively, R⁴ may be selected from H or unsubstituted or substituted C₁₋₆ alkyl, for instance R⁴ may be H. Often, though, R⁴ is C(O)OH or OH. R⁴ is often, for instance, OH.

R⁵ is H, unsubstituted or substituted C₁₋₆ alkyl, unsubstituted or substituted aryl, unsubstituted or substituted heteroaryl, —CN, —N(R^{t})C(O)N(R^{u})R^{v}, —C(O)OR^{w}, or —C(O)N(R^{x})R⁷.

R⁵ is typically H, unsubstituted or substituted C₁₋₆ alkyl, unsubstituted or substituted aryl, unsubstituted or substituted heteroaryl, —CN, —N(R^{t})C(O)N(R^{u})R^{v} or —C(O)OR^{w}. This is especially typical when R¹ is —C(O)N(R^{x})R⁷. For instance, R⁵ may be H, unsubstituted or substituted C₁₋₆ alkyl, —CN or —C(O)OR^{w}, preferably H, —CN, or —C(O)OR^{w}. These are often the case when R¹ is —C(O)N(R^{x})R⁷.

R⁵ may also typically be —C(O)N(R^{x})R⁷. This is especially typical when R¹ is other than —C(O)N(R^{x})R⁷. Thus, R⁵ being —C(O)N(R^{x})R⁷ is especially typical when R¹ is H, unsubstituted or substituted C₁₋₆ alkyl, unsubstituted or substituted aryl, unsubstituted or substituted heteroaryl, —CN, —N(R^{t})C(O)N(R^{u})R^{v} or —C(O)OR^{w}.

Typically, R⁵ is H, unsubstituted or substituted C₁₋₆ alkyl, unsubstituted or substituted aryl, unsubstituted or substituted heteroaryl, —CN, —C(O)OR^{w}, or —C(O)N(R^{x})R⁷. Often, R⁵ is selected from H, unsubstituted or substituted C₁₋₆ alkyl, —CN, —C(O)OR^{w} and —C(O)N(R^{x})R⁷. R⁵ may for instance be selected from H, —CN, —C(O)OR^{w} and —C(O)N(R^{x})R⁷. R⁵ may for instance be selected from H, unsubstituted or substituted C₁₋₆ alkyl, —CN and —C(O)OR^{w}, or, for instance, from H, —CN and —C(O)OR^{w}. R⁵ may for instance be —C(O)OR^{w}. Often R⁵ is —C(O)OH.

R⁶ is H or unsubstituted or substituted C₁₋₆ alkyl. Usually R⁶ is H or unsubstituted C₁₋₆ alkyl. Typically R⁶ is H.

R⁷ is —CH(R¹¹)—Ar, —CH(R¹¹)-Ary-Ar, -Ary-Ar, —CH(R¹¹)-Cyc, —CH₂C≡CCH₃, -Cyc or —Ar. R⁷ may for instance be —CH(R¹¹)—Ar, —CH(R¹¹)-Ary-Ar, -Ary—Ar or —CH(R¹¹)-Cyc. Ar is unsubstituted or substituted aryl or unsubstituted or substituted heteroaryl. For instance, Ar may be unsubstituted or substituted aryl or unsubstituted heteroaryl. For instance, Ar may be unsubstituted or substituted phenyl, or unsubstituted heteroaryl. Ar may for instance be selected from unsubstituted phenyl, unsubstituted pyrimidyl, unsubstituted benzothiazole, and phenyl substituted with —C(O)OH, —C(O)OMe, —C(O)OEt, —C(O)NH₂, —C(O)N(H)Me, —OMe or N-morpholino.

Ary is unsubstituted or substituted arylene, or unsubstituted or substituted heteroarylene. Ary may for instance be unsubstituted arylene, or unsubstituted heteroarylene. Typically Ary is unsubstituted phenylene or unsubstituted pyridylene.

Cyc is unsubstituted or substituted C₃₋₁₀ cycloalkyl. Typically, Cyc is unsubstituted or substituted cyclohexyl. For instance Cyc may be unsubstituted cyclohexyl or cyclohexyl substituted with —CF₃ or —OCF₃.

R¹¹ is H, —C(O)OR² or unsubstituted or substituted C₁₋₄ alkyl. R¹¹ may for instance be H, —C(O)OR² or unsubstituted C₁₋₄ alkyl. Usually, R¹¹ is H, —C(O)OR^{z} or methyl.

R⁸, R⁰ and R¹⁰ are each independently selected from H and unsubstituted or substituted C₁₋₆ alkyl.

Therefore R⁸ may be H or unsubstituted or substituted C₁₋₆ alkyl. Typically R⁸ is H.

R⁹ may also be selected from H or unsubstituted or substituted C₁₋₆ alkyl. Typically R⁹ is H.

R¹⁰ may also be selected from H or unsubstituted or substituted C₁₋₆ alkyl. Typically R¹⁰ is H.

Usually R⁸, R⁹ and R¹⁰, which may be the same or different, are each independently selected from H, unsubstituted C₁₋₆ alkyl, and C₁₋₆ alkyl which is substituted with phenyl or —OC(O)R⁹⁹. R⁹⁹ is phenyl, unsubstituted C₁₋₆ alkyl, —N(R^{a})(R^{b}), —C(O)R^{c}, —OR^{d} or an amino acid. R^{a}, R^{b}, R^{c} and R^{d} are each independently selected from H, unsubstituted or substituted C₁₋₆ alkyl and an amino acid.

Thus, often, R⁸ is selected from H, unsubstituted C₁₋₆ alkyl, and C₁₋₆ alkyl which is substituted with phenyl or —OC(O)R⁹⁹, wherein R⁹⁹ is as defined above.

Typically, R⁹ is selected from H, unsubstituted C₁₋₆ alkyl, and C₁₋₆ alkyl which is substituted with phenyl or —OC(O)R⁹⁹, wherein R⁹⁹ is as defined above.

Often, R¹⁰ is selected from H, unsubstituted C₁₋₆ alkyl, and C₁₋₆ alkyl which is substituted with phenyl or —OC(O)R⁹⁹, wherein R⁹⁹ is as defined above.

Structures of formula (I) in which R⁸, R⁹ or R¹⁰ is other than H include prodrug compounds. In particular, the substituted azines of formula (I) in which R⁸, R⁹ or R¹⁰ is unsubstituted or substituted C₁₋₆ alkyl, and particularly substituted C₁₋₆ alkyl, include prodrug compounds. For instance, substituted azines of formula (I) in which R⁸, R⁹ or R¹⁰ is C₁₋₆ alkyl which is substituted with phenyl or —OC(O)R⁹⁹, wherein R⁹⁹ is as defined above, include prodrug compounds. R⁸, R⁹ or R¹⁰ may for instance be substituted C₁₋₆ alkyl, wherein the, or one of the, substituents on the C₁₋₆ alkyl is a group of formula —OC(O)R⁹⁹, wherein R⁹⁹ is phenyl, unsubstituted C₁₋₆ alkyl, —N(R^{a})(R^{b}), —C(O)R^{c}, —OR^{d} or an amino acid, and wherein R^{a}, R^{b}, R^{c} and R^{d} are each independently selected from H, unsubstituted or substituted C₁₋₆ alkyl and an amino acid. Such compounds include prodrugs. Thus, often, R⁸ is C₁₋₆ alkyl which is substituted with —OC(O)R⁹⁹, wherein R⁹⁹ is as defined above. Similarly, R⁹ may be C₁₋₆ alkyl which is substituted with —OC(O)R⁹⁹, wherein R⁹⁹ is as defined above. R¹⁰ may be C₁₋₆ alkyl which is substituted with —OC(O)R⁹⁹, wherein R⁹⁹ is as defined above.

Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl.

R^{t}, R^{u}, R^{v}, R^{w}, R^{x}, R^{y}, and R^{z} are each independently selected from H, unsubstituted or substituted C₁₋₆ alkyl, and unsubstituted or substituted phenyl.

Usually R^{t} is unsubstituted or substituted C₁₋₄ alkyl, or H. Typically, R^{t} is unsubstituted C₁₋₄ alkyl or H. Usually, R^{t} is H.

Usually R^{u} is unsubstituted or substituted C₁₋₄ alkyl, or H. Typically, R^{u} is unsubstituted C₁₋₄ alkyl or H. Usually, R^{u} is H.

Usually R^{v} is unsubstituted or substituted C₁₋₄ alkyl, or H. Typically, R^{v} is unsubstituted C₁₋₄ alkyl or H. Usually, R^{v} is H.

Thus, often, R^{t}, R^{u} and R^{v} are all H.

Usually R is H.

Typically R^{y} is H or unsubstituted C₁₋₆ alkyl, for example R^{y} may be H or methyl.

Typically R^{z} is H.

R^{w} is H, unsubstituted or substituted C₁₋₆ alkyl, or unsubstituted or substituted phenyl. R^{w} may be H, or unsubstituted or substituted C₁₋₆ alkyl. Typically, for instance, R^{w} is H, unsubstituted C₁₋₆ alkyl, or C₁₋₆ alkyl which is substituted with phenyl or —OC(O)R^{ww}, wherein R^{ww} is phenyl, unsubstituted C₁₋₆ alkyl, —N(R^{a})(R^{b}), —C(O)R^{c}, —OR^{d} or an amino acid, wherein R^{a}, R^{b}, R^{c} and R^{d} are each independently selected from H, unsubstituted or substituted C₁₋₆ alkyl and an amino acid.

Substituted azines of formula (I) in which R^{w} is other than H include prodrug compounds. In particular, compounds of formula (I) in which R^{w} is unsubstituted or substituted C₁₋₆ alkyl, or unsubstituted or substituted phenyl, include prodrug compounds. For instance, compounds of formula (I) in which R^{w} is C₁₋₆ alkyl which is substituted with phenyl or —OC(O)R^{ww}, wherein R^{ww} is is as defined above, include prodrug compounds. R^{w} may for instance be substituted C₁₋₆ alkyl, wherein the, or one of the, substituents on the C₁₋₆ alkyl is a group of formula —OC(O)R^{ww}, wherein R^{ww} is phenyl, unsubstituted C₁₋₆ alkyl, —N(R^{a})(R^{b}), —C(O)R^{c}, OR^{d} or an amino acid, wherein R^{a}, R^{b}, R^{c} and R^{d} are each independently selected from H, unsubstituted or substituted C₁₋₆ alkyl and an amino acid. Such compounds include prodrugs. Thus, often, R^{w} is C₁₋₆ alkyl which is substituted with —OC(O)R^{ww}, wherein R^{ww} is as defined above. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl. R^{w} may be H, unsubstituted C₁₋₆ alkyl, or C₁₋₆ alkyl which is substituted with phenyl or —OC(O)R^{ww} wherein R^{ww} is phenyl or unsubstituted C₁₋₆ alkyl.

R^{w} may be H.

R^{q} is H, unsubstituted or substituted C₁₋₆ alkyl, or unsubstituted or substituted phenyl. R^{q} is typically H.

Typically, in formula (I), one of R¹ and R⁵ is —C(O)N(R^{x})R⁷ and the other of R¹ and R⁵ is other than —C(O)N(R^{x})R⁷. When one of R¹ and R⁵ is other than —C(O)N(R^{x})R⁷, it may be any of the other definitions for R¹ or R⁵ specified herein. Thus, it may be any of H, unsubstituted or substituted C₁₋₆ alkyl, unsubstituted or substituted aryl, unsubstituted or substituted heteroaryl, —CN, —N(R^{t})C(O)N(R^{u})R^{v} or —C(O)OR^{w}.

Often, in formula (I), one of R¹ and R⁵ is —C(O)N(R^{x})R⁷ and the other of R¹ and R⁵ is H, unsubstituted or substituted C₁₋₆ alkyl, unsubstituted or substituted aryl, unsubstituted or substituted heteroaryl, —CN, —N(R^{t})C(O)N(R^{u})R^{v} or —C(O)OR^{w}. Typically, one of R¹ and R⁵ is —C(O)N(R^{x})R⁷ and the other of R¹ and R⁵ is H, unsubstituted or substituted C₁₋₆ alkyl, —CN or —C(O)OR^{w}. For instance, one of R¹ and R⁵ may be —C(O)N(R^{x})R⁷ and the other of R¹ and R⁵ may be H, —CN or —C(O)OR^{w}.

In some embodiments of the substituted azine of formula (I):
R⁰ is H or unsubstituted C₁₋₆ alkyl;
R¹ is H, —CN, —C(O)OR^{w} or —C(O)N(R^{x})R⁷;
R² is H or unsubstituted C₁₋₆ alkyl; and R³ is H or —OR⁸; or R² is —N═ and R³ is ═C(R^{y})— and R² and R³ together form a group of formula —N═C(R^{y})—. In such embodiments, R⁴ may be H, —OR⁹ or —C(O)OR¹⁰; R⁵ may be H, —CN, —C(O)OR^{w}, or —C(O)N(R^{x})R⁷; and R⁶ is H. Furthermore, R⁷ is typically —CH(R¹¹)—Ar, —CH(R¹¹)-Ary-Ar, -Ary-Ar or CH(R¹¹)-Cyc, wherein Ar is unsubstituted or substituted aryl or unsubstituted or substituted heteroaryl, Ary is unsubstituted arylene or unsubstituted heteroarylene, Cyc is unsubstituted or substituted C₃₋₁₀ cycloalkyl, and R¹¹ is H, —C(O)OR² or unsubstituted C₁₋₄ alkyl.

Usually in such embodiments R⁸, R⁹ and R¹⁰ are each independently selected from H and unsubstituted or substituted C₁₋₆ alkyl. Typically, R^{x} is H, R^{z} is H, R^{y} is H or unsubstituted C₁₋₆ alkyl, and R^{w} is H, unsubstituted C₁₋₆ alkyl, or C₁₋₆ alkyl which is substituted with phenyl or —OC(O)R^{ww} wherein R^{ww} is phenyl, unsubstituted C₁₋₆ alkyl, —N(R^{a})(R^{b}), —C(O)R^{c}, —OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl.

Typically such compounds are provided wherein one of R¹ and R⁵ is —C(O)N(R^{x})R⁷ and the other of R¹ and R⁵ is H, —CN or —C(O)OR^{w}.

Thus, typically, R⁰ is H or unsubstituted C₁₋₆ alkyl; R¹ is H, —CN, —C(O)OR^{w} or —C(O)N(R^{x})R⁷; R² is H or unsubstituted C₁₋₆ alkyl; and R³ is H or —OR⁸; or R² is —N═ and R³ is ═C(R^{y})— and R² and R³ together form a group of formula —N═C(R^{w})—; R⁴ is H, OR⁹ or —C(O)OR¹⁰; R⁵ is H, —CN, —C(O)OR^{w}, or —C(O)N(R^{x})R⁷; R⁶ is H; R⁷ is —CH(R¹¹)—Ar, —CH(R¹¹)-Ary-Ar, -Ary-Ar or —CH(R¹¹)-Cyc, wherein Ar is unsubstituted or substituted aryl or unsubstituted or substituted heteroaryl, Ary is unsubstituted arylene or unsubstituted heteroarylene, Cyc is unsubstituted or substituted C₃₋₁₀ cycloalkyl, and R¹¹ is H, —C(O)OR² or unsubstituted C₁₋₄ alkyl; R⁸, R⁹ and R¹⁰ are each independently selected from H and unsubstituted or substituted C₁₋₆ alkyl; and R^{x} is H, R^{z} is H, R^{y} is H or unsubstituted C₁₋₆ alkyl, and R^{w} is H, unsubstituted C₁₋₆ alkyl, or C₁₋₆ alkyl which is substituted with phenyl or —OC(O)R^{ww} wherein R^{ww} is phenyl, unsubstituted C₁₋₆ alkyl, —N(R^{a})(R^{b}), —C(O)R^{c}, OR^{d} or an amino acid, wherein R^{a}, R^{b}, R^{c} and R^{d} are each independently selected from H, unsubstituted or substituted C₁₋₆ alkyl and an amino acid; provided that one of R¹ and R⁵ is —C(O)N(R^{x})R⁷ and the other of R¹ and R⁵ is H, —CN or —C(O)OR^{w}.

In some embodiments of the substituted azine of formula (I), R⁰ is H or methyl; R¹ is H, —CN, —C(O)OR^{w}, or —C(O)N(R^{x})R⁷; R² is H or methyl; and R³ is H or —OR⁸; or R² is —N═ and R³ is ═C(R^{y})— and R² and R³ together form a group of formula —N═C(R^{y})—.

Typically, in such compounds R⁴ is H, —OR′ or —C(O)OR¹⁰; R⁵ is H, —CN, —C(O)OR^{w}, or —C(O)N(R^{x})R⁷; R⁶ is H; and R⁷ is —CH(R¹¹)—Ar, —CH(R¹¹)-Ary-Ar, -Ary-Ar or —CH(R¹¹)-Cyc; wherein Ar is unsubstituted phenyl, unsubstituted pyrimidyl, unsubstituted benzothiazole or phenyl substituted with —C(O)OH, —C(O)OMe, —C(O)OEt, —C(O)NH₂, —C(O)N(H)Me, —OMe or N-morpholino; Ary is unsubstituted phenylene or unsubstituted pyridylene; Cyc is unsubstituted cyclohexyl or cyclohexyl substituted with —CF₃ or —OCF₃; and R¹¹ is H, —C(O)OR^{z} or methyl.

Usually, R⁸, R⁹ and R¹⁰ are each independently selected from H, unsubstituted C₁₋₆ alkyl, and C₁₋₆ alkyl which is substituted with phenyl or —OC(O)R⁹⁹ wherein R⁹⁹ is phenyl, unsubstituted C₁₋₆ alkyl, —N(R^{a})(R^{b}), —C(O)R^{c}, —OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, and unsubstituted or substituted C₁₋₆ alkyl. Usually, R^{a}, R^{b}, R^{c} and R^{d} are each independently selected from H, methyl or ethyl.

Usually, R^{x} is H; R^{z} is H; R^{w} is H, unsubstituted C₁₋₆ alkyl, or C₁₋₆ alkyl which is substituted with phenyl or —OC(O)R^{ww} wherein R^{ww} is phenyl or unsubstituted C₁₋₆ alkyl; and R^{y} is H or methyl.

For such compounds of the invention, it is usually the case that one of R¹ and R⁵ is —C(O)N(R^{x})R⁷ and the other of R¹ and R⁵ is H, —CN or —C(O)OR^{w}.

Thus, typically, R⁰ is H or methyl; R¹ is H, —CN, —C(O)OR^{w}, or —C(O)N(R^{x})R⁷; R² is H or methyl; and R³ is H or —OR⁸; or R² is —N═ and R³ is ═C(R^{y})— and R² and R³ together form a group of formula —N═C(R′); R⁴ is H, —OR⁹ or —C(O)OR¹⁰; R⁵ is H, —CN, —C(O)OR^{w}, or —C(O)N(R^{x})R⁷; R⁶ is H; R⁷ is —CH(R¹¹)—Ar, —CH(R¹¹)-Ary-Ar, -Ary—Ar or —CH(R¹¹) Cyc; wherein Ar is unsubstituted phenyl, unsubstituted pyrimidyl, unsubstituted benzothiazole or phenyl substituted with —C(O)OH, —C(O)OMe, —C(O)OEt, —C(O)NH₂, —C(O)N(H)Me, —OMe or N-morpholino; Ary is unsubstituted phenylene or unsubstituted pyridylene; Cyc is unsubstituted cyclohexyl or cyclohexyl substituted with —CF₃ or —OCF₃; and R¹¹ is H, —C(O)OR^{z} or methyl; R⁸, R⁹ and R¹⁰ are each independently selected from H, unsubstituted C₁₋₆ alkyl, and C₁₋₆ alkyl which is substituted with phenyl or —OC(O)R⁹⁹ wherein R⁹⁹ is phenyl, unsubstituted C₁₋₆ alkyl, —N(R^{a})(R^{b}), —C(O)R^{c}, —OR^{d} or an amino acid, wherein R^{a}, R^{b}, R^{c} and R^{d} are each independently selected from H, unsubstituted or substituted C₁₋₆ alkyl and an amino acid; and R^{x} is H; R^{z} is H; R^{w} is H, unsubstituted C₁₋₆ alkyl, or C₁₋₆ alkyl which is substituted with phenyl or —OC(O)R^{ww} wherein R^{ww} is phenyl or unsubstituted C₁₋₆ alkyl; and R^{y} is H or methyl; provided that one of R¹ and R⁵ is —C(O)N(R^{x})R⁷ and the other of R¹ and R⁵ is H, CN or —C(O)OR^{w}.

Often, in the substituted azine of formula (I), it is the case that (a) R⁵ is —C(O)N(R^{x})R⁷ and (b) R³ is —OR⁸ or R⁴ is —OR⁹, R^{x}, R⁷, R⁸ and R⁹, in these embodiments, may be as defined anywhere herein for the compounds of the invention.

In another typical case, in the substituted azine of formula (I), (a) R¹ is —C(O)N(R^{x})R⁷, and (b) R⁴ is —OR⁹ or —C(O)OR¹⁰, or R⁵ is —C(O)OR^{w}. R^{x}, R⁷, R⁸ and R⁹, in these embodiments, may be as defined anywhere herein for the compounds of the invention.

Thus, typically, in the substituted azine of formula (I), either:
(1) (a) R⁵ is —C(O)N(R^{x})R⁷ and (b) R³ is —OR⁸ or R⁴ is —OR⁹; or
(2) (a) R¹ is —C(O)N(R^{x})R⁷ and (b) R⁴ is —OR⁹ or —C(O)OR¹⁰, or R⁵ is —C(O)OR^{w}.
R^{x}, R⁷, R⁸ and R⁹ may be as further defined herein. In some embodiments, for instance, R^{x} is H, R⁸ is H, R⁹ is H, and R⁷ is as defined anywhere herein. For instance R⁷ may be —CH(R¹¹)—Ar, —CH(R¹¹)-Ary-Ar, -Ary-Ar or —CH(R¹¹) Cyc, wherein Ar is unsubstituted or substituted aryl or unsubstituted or substituted heteroaryl, Ary is unsubstituted arylene or unsubstituted heteroarylene, Cyc is unsubstituted or substituted C₃₋₁₀ cycloalkyl, and R¹¹ is H, —C(O)OR² or unsubstituted C₁₋₄ alkyl. R² may be as defined anywhere herein but is often H. R⁷ may for instance be —CH(R¹¹)—Ar, —CH(R¹¹)-Ary-Ar, -Ary-Ar or —CH(R¹¹) Cyc; wherein Ar is unsubstituted phenyl, unsubstituted pyrimidyl, unsubstituted benzothiazole or phenyl substituted with —C(O)OH, —C(O)OMe, —C(O)OEt, —C(O)NH₂, —C(O)N(H)Me, —OMe or N-morpholino; Ary is unsubstituted phenylene or unsubstituted pyridylene; Cyc is unsubstituted cyclohexyl or cyclohexyl substituted with —CF₃ or —OCF₃; and R¹¹ is H, —C(O)OR² or methyl. R² may be as defined anywhere herein but is often H.

In the compounds of the invention, the substituted azine may have the formula (Ia) shown below. Accordingly, in some embodiments the invention relates to a compound which is a substituted azine of formula (Ia) or a pharmaceutically acceptable salt thereof:

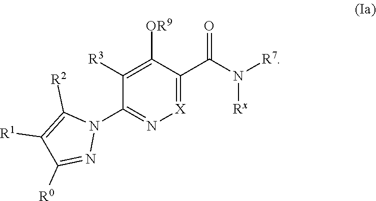

Each of R⁰, R⁹, X, R^{x} and R⁷ in formula (Ia) may be as defined anywhere herein for formula (I). R¹ in formula (Ia) may be H, unsubstituted or substituted C₁₋₆ alkyl, unsubstituted or substituted aryl, unsubstituted or substituted heteroaryl, —CN, —N(R¹)C(O)N(R^{u})R^{v} or —C(O)OR^{w}, wherein R^{t}, R^{u}, R^{v} and R^{w} may be as defined anywhere herein for formula (I). R² in formula (Ia) is H, —OR^{q} or unsubstituted or substituted C₁₋₆ alkyl, wherein R^{q} is H, unsubstituted or substituted C₁₋₆ alkyl, or unsubstituted or substituted phenyl. R³ in formula (Ia) is H or unsubstituted or substituted C₁₋₆ alkyl.

Thus, when the substituted azine of the compound of the invention has formula (Ia), typically X is C(R⁶) or N. Preferably X is C(R⁶). Alternatively, however, X may be N.

Typically R⁰ is H or unsubstituted or substituted C₁₋₆ alkyl. However, R⁰ in formula (Ia) may be as further defined anywhere herein for formula (I).

R¹ is usually H, unsubstituted or substituted C₁₋₆ alkyl, unsubstituted or substituted aryl, unsubstituted or substituted heteroaryl, —CN or —C(O)OR^{w}. However, R¹ in formula (Ia) may be as further defined anywhere herein for formula (I).

R² may be H, —OR^{q} or unsubstituted or substituted C₁₋₆ alkyl, and R³ may be H or unsubstituted or substituted C₁₋₆ alkyl. However, R² and R³ in formula (Ia) may be as further defined anywhere herein for formula (I).

Typically R⁶ is H or unsubstituted or substituted C₁₋₆ alkyl. However, R⁶ in formula (Ia) may be as further defined anywhere herein for formula (I).

R⁷ is usually-CH(R¹¹)—Ar, —CH(R¹¹)-Ary-Ar, -Ary-Ar, —CH(R¹¹)-Cyc, —CH₂C≡CCH₃, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is typically H, —C(O)OR² or unsubstituted or substituted C₁₋₄ alkyl. However, R⁷ and R¹¹ in formula (Ia) may be as further defined anywhere herein for formula (I).

In embodiments wherein the substituted azine has the formula (Ia), R⁹ is typically H or unsubstituted or substituted C₁₋₆ alkyl. R^{w}, R^{x} and R^{z} may be each independently selected from H, unsubstituted or substituted C₁₋₄ alkyl, and unsubstituted or substituted phenyl. R^{q} is typically H, unsubstituted or substituted C₁₋₆ alkyl, or unsubstituted or substituted phenyl. However, R⁹, R^{w}, R^{x}, R^{z} and R^{q} in formula (Ia) may be as further defined anywhere herein for formula (I).

In some preferred embodiments wherein the substituted azine has the formula (Ia), R⁹ is H.

The compound of the invention may be a substituted azine of formula (Ia) selected from any one of the following structures, or a pharmaceutically acceptable salt thereof:

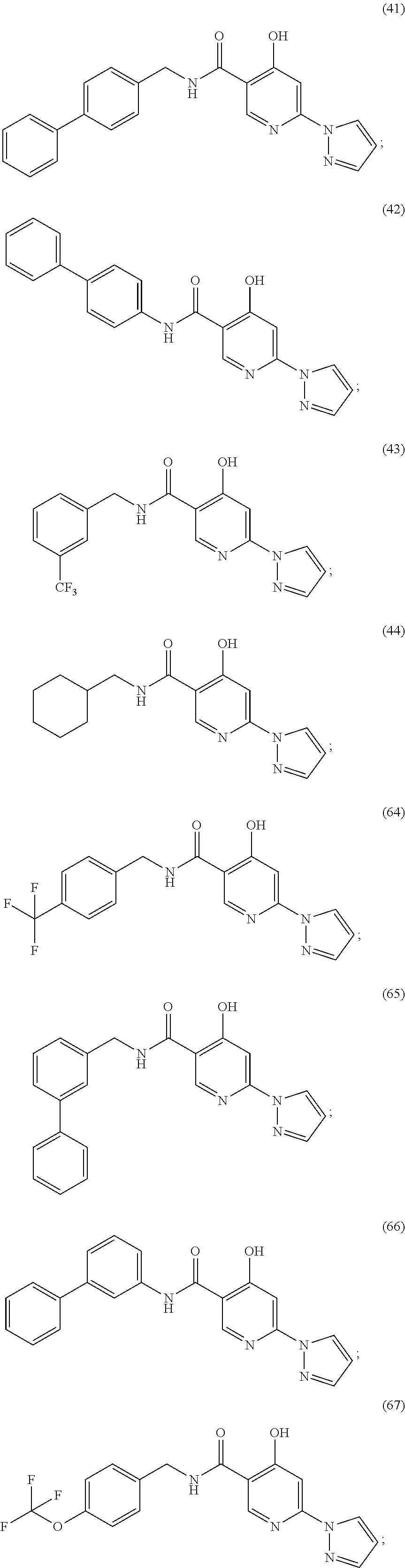

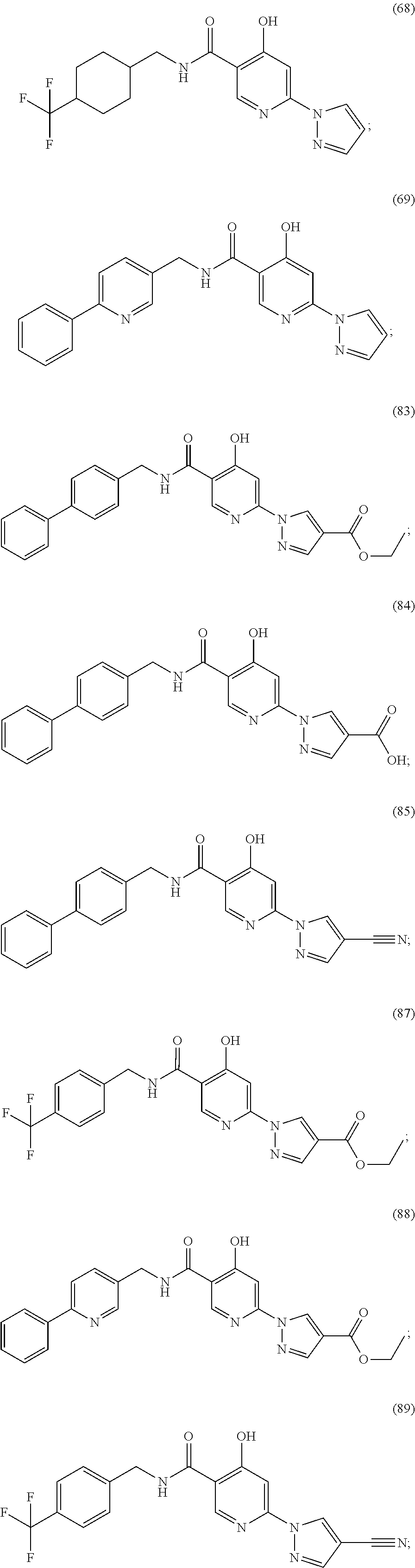

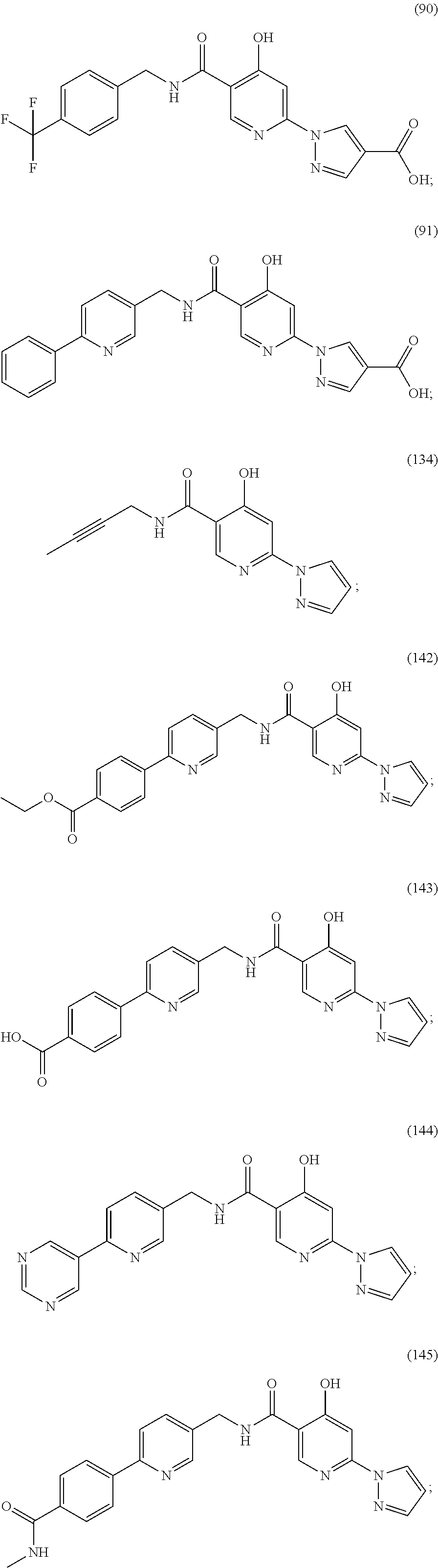

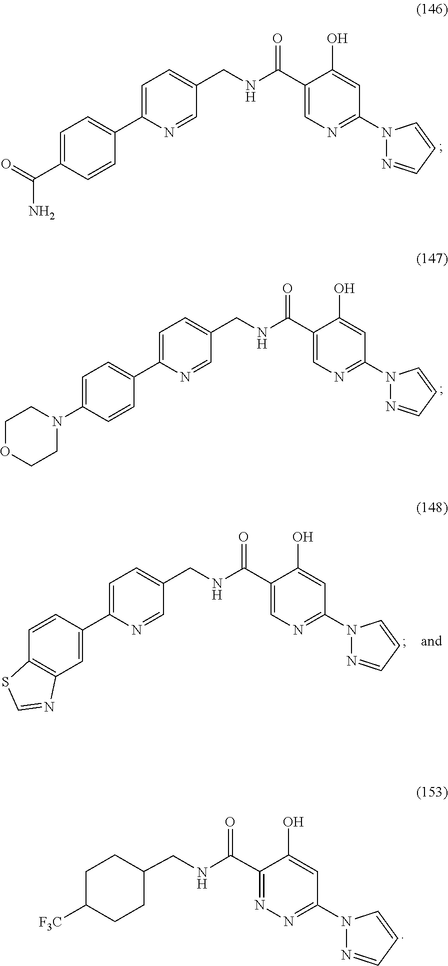 [SMILES: NC(=O)c1ccc(-c2ccc(CNC(=O)c3cnc(-n4cccn4)cc3O)cn2)cc1 ; O=C(NCc1ccc(-c2ccc(N3CCOCC3)cc2)nc1)c1cnc(-n2cccn2)cc1O ; O=C(NCc1ccc(-c2ccc3scnc3c2)nc1)c1cnc(-n2cccn2)cc1O ; O=C(NCC1CCC(C(F)(F)F)CC1)c1nnc(-n2cccn2)cc1O]

In some preferred embodiments wherein the substituted azine has the formula (Ia), R⁰, R¹, R², R³, R^{x} and R⁹ are H or unsubstituted C₁₋₄ alkyl. Typically, R⁰, R¹, R², R³, R^{x} and R⁹ are H.

Typically, for such embodiments, X is N.

R⁷ may preferably be —CH(R¹¹) Cyc, wherein Cyc is unsubstituted or substituted C₃₋₁₀ cycloalkyl, and wherein R¹¹ is unsubstituted C₁₋₄ alkyl or H, typically H. Typically, Cyc is substituted C₄₋₈ cycloalkyl, for example a substituted C₆ cycloalkyl (i.e. cyclohexyl). When Cyc is a substituted C₆ cycloalkyl it typically has only one substituent, and may be substituted with C₁₋₁₀ alkyl or halo(C₁₋₁₀)alkyl. Typically it is substituted with halo(C₁₋₄)alkyl, and is often a halo-substituted methyl group. A halo-substituted methyl group is often preferably trifluoromethyl.

Therefore, in one embodiment, the substituted azine has the formula (Ia), wherein R⁰, R¹, R², R³, R^{x} and R⁹ are H; X is N; and R⁷ is —CH(R¹¹)-Cyc, wherein R¹¹ is H, and Cyc is a trifluoromethyl-substituted cyclohexyl group (e.g. a para-trifluoromethyl-substituted cyclohexyl group).

The compound of the invention may therefore be a substituted azine of formula (Ia) with the following structure, or a pharmaceutically acceptable salt thereof:

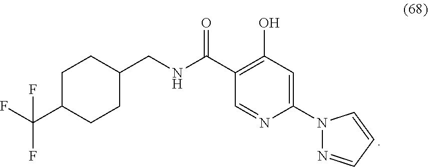 [SMILES: O=C(NCC1CCC(C(F)(F)F)CC1)c1cnc(-n2cccn2)cc1O]

The numbers in parentheses next to the structures above match the compound numbers given in the Examples section hereinbelow.

In the compounds of the invention, the substituted azine may have the formula (Ib) shown below. Accordingly, in some embodiments the invention relates to a compound which is a substituted azine of formula (Ib) or a pharmaceutically acceptable salt thereof:

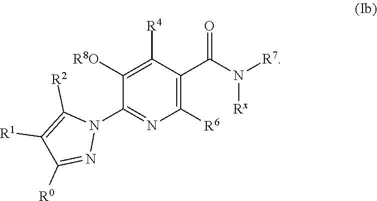

Each of R⁰, R⁴, R⁶, R⁷, R⁸ and R^{x} in formula (Ib) may be as defined anywhere herein for formula (I). R¹ in formula (Ib) may be H, unsubstituted or substituted C₁₋₆ alkyl, unsubstituted or substituted aryl, unsubstituted or substituted heteroaryl, —CN, —N(R^{t})C(O)N(R^{u})R^{v} or —C(O)OR^{w}, wherein R^{t}, R^{u}, R^{v} and R^{w} may be as defined anywhere herein for formula (I). R² in formula (Ib) is H, —OR^{q} or unsubstituted or substituted C₁₋₆ alkyl, wherein R^{q} is H, unsubstituted or substituted C₁₋₆ alkyl, or unsubstituted or substituted phenyl.

Thus, when the substituted azine of the compound of the invention has formula (Ib), typically R⁰ is H or unsubstituted or substituted C₁₋₆ alkyl. However, R⁰ in formula (Ib) may be as further defined anywhere herein for formula (I).

R¹ may be H, unsubstituted or substituted C₁₋₆ alkyl, unsubstituted or substituted aryl, unsubstituted or substituted heteroaryl, —CN or —C(O)OR^{w}. However, R¹ in formula (Ib) may be as further defined anywhere herein for formula (I).

Usually, R² is H, —OR^{q} or unsubstituted or substituted C₁₋₆ alkyl. However, R² in formula (Ib) may be as further defined anywhere herein for formula (I).

Typically, R⁴ in formula (Ib) is H or unsubstituted or substituted C₁₋₆ alkyl. R⁶ may be H or unsubstituted or substituted C₁₋₆ alkyl. For instance R⁴ may be H, or unsubstituted C₁₋₆ alkyl, and is often H. R⁴ in formula (Ib) may be as further defined anywhere herein for formula (I).

Typically R⁶ is H or unsubstituted or substituted C₁₋₆ alkyl. For instance R⁶ may be H, or unsubstituted C₁₋₆ alkyl, and is often H. R⁶ in formula (Ib) may be as further defined anywhere herein for formula (I).

Typically, R⁷ is —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR² or unsubstituted or substituted C₁₋₄ alkyl. However, R⁷ and R¹¹ in formula (Ib) may be as further defined anywhere herein for formula (I).

Typically, for compounds of formula (Ib), or pharmaceutically acceptable salts thereof, R⁸ is H or unsubstituted or substituted C₁₋₆ alkyl. However, R⁸ in formula (Ib) may be as further defined anywhere herein for formula (I).

Usually, R^{w}, R^{x} and R^{z} are each independently selected from H, unsubstituted or substituted C₁₋₄ alkyl, and unsubstituted or substituted phenyl. R^{q} is typically H, unsubstituted or substituted C₁₋₆ alkyl, or unsubstituted or substituted phenyl. R^{q} is often for instance H. However, R^{w}, R^{x}, R^{z} and R^{q} in formula (Ib) may be as further defined anywhere herein for formula (I).

In some preferred substituted azines of formula (Ib), R⁸ is H.

The compound of the invention may be a substituted azine of formula (Ib) selected from any one of the following structures, or a pharmaceutically acceptable salt thereof:

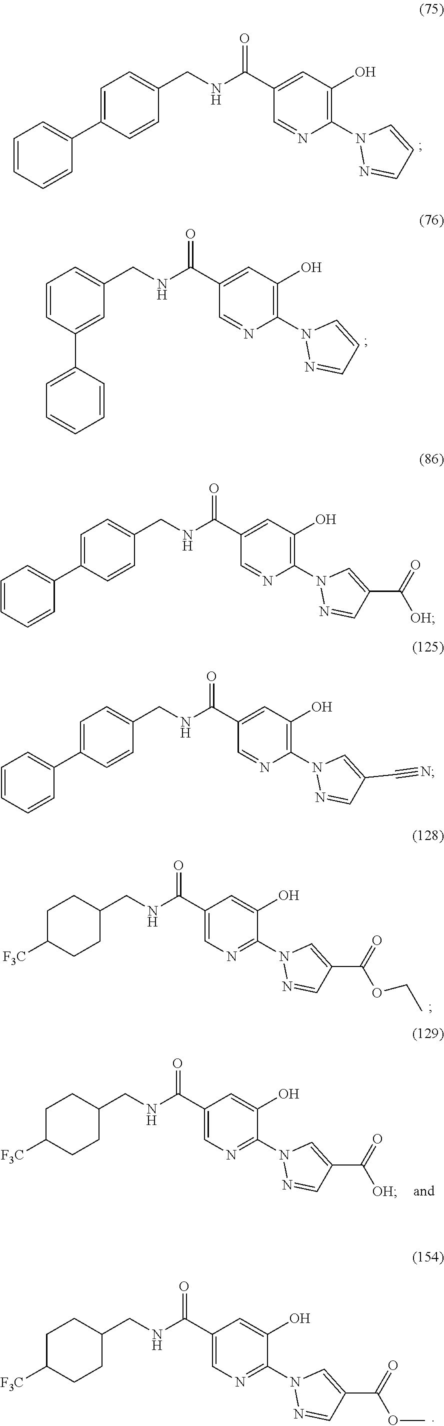

The numbers in parentheses next to the structures above match the compound numbers given in the Examples section hereinbelow.

In the compounds of the invention, the substituted azine may have the formula (Ic) shown below. Accordingly, in some embodiments the invention relates to a compound which is a substituted azine of formula (Ic) or a pharmaceutically acceptable salt thereof:

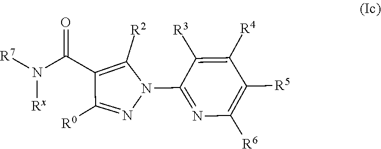

Each of R⁰, R⁴, R⁶, R^{x} and R⁷ in formula (Ic) may be as defined anywhere herein for formula (I). R⁵ in formula (Ic) may be H, unsubstituted or substituted C₁₋₆ alkyl, unsubstituted or substituted aryl, unsubstituted or substituted heteroaryl, —CN, —N(R^{t})C(O)N(R^{u})R^{v} or —C(O)OR^{w}, wherein R^{t}, R^{u}, R^{v} and R^{w} may be as defined anywhere herein for formula (I). R² in formula (Ic) is H, —OR^{q} or unsubstituted or substituted C₁₋₆ alkyl, wherein R^{q} is H, unsubstituted or substituted C₁₋₆ alkyl, or unsubstituted or substituted phenyl. R³ in formula (Ic) is H, —OR⁸ or unsubstituted or substituted C₁₋₆ alkyl, wherein R⁸ is selected from H and unsubstituted or substituted C₁. 6 alkyl.

Thus, when the substituted azine of the compound of the invention has formula (Ic), typically R⁰ is H or unsubstituted or substituted C₁₋₆ alkyl. However, R⁰ in formula (Ic) may be as further defined anywhere herein for formula (I). Usually, R² is H, —OR^{q} or unsubstituted or substituted C₁₋₆ alkyl. However, R² in formula (Ic) may be as further defined anywhere herein for formula (I). R³ may be H, —OR⁸ or unsubstituted or substituted C₁₋₆ alkyl. However, R³ in formula (Ic) may be as further defined anywhere herein for formula (I), as may R⁸. R³ in formula (Ic) is often H, or —OR⁸. R⁸ may for instance be unsubstituted C₁₋₆ alkyl.

For the substituted azine of formula (Ic), usually R⁴ is H, unsubstituted or substituted C₁₋₆ alkyl, —OR⁹ or —C(O)OR¹⁰, wherein R⁹ and R¹⁰ are as defined anywhere herein for formula (I). However, R⁴ in formula (Ic) may be as further defined anywhere herein for formula (I). R⁴ may for instance be selected from H, —OR⁹ or —C(O)OR¹⁰. R⁴ may for example be selected from H, —OH and —C(O)OH.

R⁵ in formula (Ic) may be H, unsubstituted or substituted C₁₋₆ alkyl, unsubstituted or substituted aryl, unsubstituted or substituted heteroaryl, —CN or —C(O)OR^{w}. However, R⁵ in formula (Ic) may be as further defined anywhere herein for formula (I), as may R^{w}.

Typically, R⁶ is H or unsubstituted or substituted C₁₋₆ alkyl. However, R⁶ in formula (Ic) may be as further defined anywhere herein for formula (I).

R⁷ may be —CH(R¹¹)—Ar, —CH(R¹¹)-Ary-Ar, -Ary-Ar, —CH(R¹¹) Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR² or unsubstituted or substituted C₁₋₄ alkyl. However, R⁷ and R¹¹ in formula (Ic) may be as further defined anywhere herein for formula (I).

In embodiments wherein the substituted azine has the formula (Ic), R⁸, R⁹ and R¹⁰ are each independently selected from H and unsubstituted or substituted C₁₋₆ alkyl. However, each of R⁸, R⁹ and R¹⁰ in formula (Ic) may be as further defined anywhere herein for formula (I).

R^{w}, R^{x} and R² are usually each independently selected from H, unsubstituted or substituted C₁₋₄ alkyl, and unsubstituted or substituted phenyl. However, each of R^{w}, R^{x} and R^{z} in formula (Ic) may be as further defined anywhere herein for formula (I).

R^{q} may be H, unsubstituted or substituted C₁₋₆ alkyl, or unsubstituted or substituted phenyl. However, R^{q} in formula (Ic) may be as further defined anywhere herein for formula (I).

Often, in the substituted azine of formula (Ic), R⁴ is OH or C(O)OH. For instance, in the substituted azine of formula (Ic), it is often the case that R⁴ is OH or C(O)OH and R⁵ is H. For instance R⁴ may be OH and R⁵ may be H. Also, typically, R⁴ is C(O)OH and R⁵ is H. Also, typically, R⁴ is OH or C(O)OH and R⁵ is CN. For instance R⁴ may be OH and R⁵ may be CN.

It is also often the case, in the substituted azine of formula (Ic), that R⁵ is C(O)OH. For instance, in the substituted azine of formula (Ic), it is often the case that R⁵ is C(O)OH and R⁴ is H. Also, typically, R⁵ is C(O)OH and R⁴ is OH.

Thus, in some preferred embodiments of the substituted azine of formula (Ic): (i) R⁴ is OH or C(O)OH, and/or (ii) R⁵ is C(O)OH.

Indeed, preferably, in the substituted azine of formula (Ic): (a) R⁴ is OH; or (b) R⁴ is C(O)OH; or (c) R⁵ is C(O)OH; or (d) R⁴ is OH and R⁵ is C(O)OH.

The compound of the invention may be a substituted azine of formula (Ic) selected from any one of the following structures, or a pharmaceutically acceptable salt thereof:

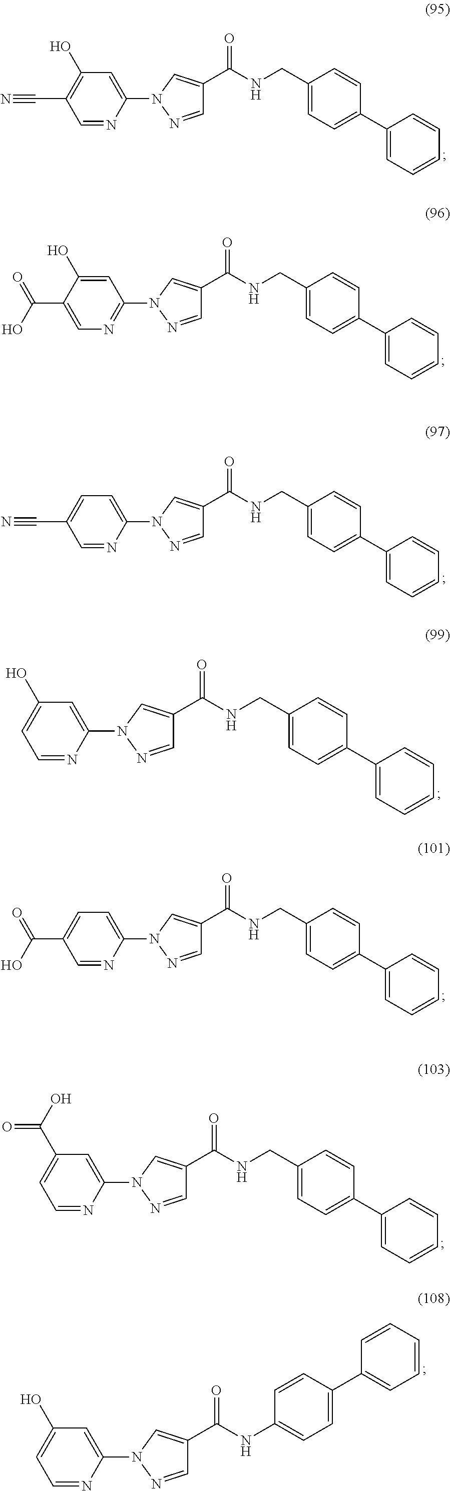

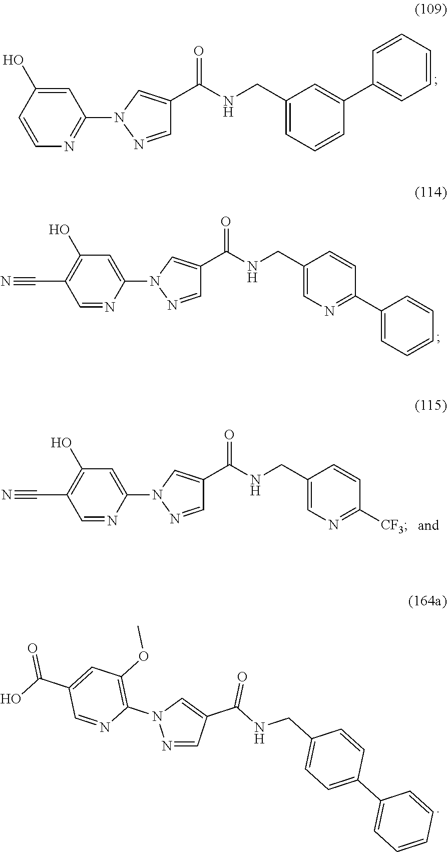 [SMILES: O=C(NCc1cccc(-c2ccccc2)c1)c1cnn(-c2cc(O)ccn2)c1 ; N#Cc1cnc(-n2cc(C(=O)NCc3ccc(-c4ccccc4)nc3)cn2)cc1O ; N#Cc1cnc(-n2cc(C(=O)NCc3ccc(C(F)(F)F)nc3)cn2)cc1O ; COc1cc(C(=O)O)cnc1-n1cc(C(=O)NCc2ccc(-c3ccccc3)cc2)cn1]

The numbers in parentheses next to the structures above match the compound numbers given in the Examples section hereinbelow.

In the compounds of the invention, the substituted azine may have the formula (Id) shown below. Accordingly, in some embodiments the invention relates to a compound which is a substituted azine of formula (Id) or a pharmaceutically acceptable salt thereof:

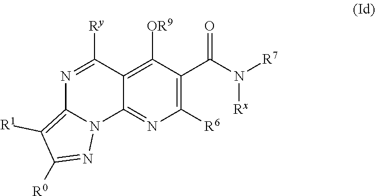

Each of R⁰, R¹, R⁶, R⁹, R^{y}, R^{x} and R⁷ in formula (Id) may be as defined anywhere herein for formula (I). However, R¹ in formula (Id) is usually H, unsubstituted or substituted C₁₋₆ alkyl, unsubstituted or substituted aryl, unsubstituted or substituted heteroaryl, —CN, —N(R^{t})C(O)N(R^{u})R^{v} or —C(O)OR^{w}, wherein R^{t}, R^{u}, R^{v} and R^{w} may be as defined anywhere herein for formula (I).

Thus, when the substituted azine of the compound of the invention has formula (Id), typically R⁰ is H or unsubstituted or substituted C₁₋₆ alkyl. However, R⁰ in formula (Id) may be as further defined anywhere herein for formula (I). R⁰ in formula (Id) is often H or methyl. Typically, it is methyl.

R¹ in formula (Id) is typically H, unsubstituted or substituted C₁₋₆ alkyl, unsubstituted or substituted aryl, unsubstituted or substituted heteroaryl, —CN or —C(O)OR^{w}. However, R¹ in formula (Id) may be as further defined anywhere herein for formula (I). R¹ in formula (Id) is preferably H.

R^{w} in formula (Id) is selected from H, unsubstituted or substituted C₁₋₄ alkyl, and unsubstituted or substituted phenyl. However, R^{w} in formula (Id) may be as further defined anywhere herein for formula (I).

R^{y} is typically selected from H, unsubstituted or substituted C₁₋₄ alkyl, and unsubstituted or substituted phenyl. R^{y} in formula (Id) may be as further defined anywhere herein for formula (I). Often, however, R^{y} in formula (Id) is H or methyl.

Often, in formula (Id), R^{y} and R⁰ are both methyl. R^{y} and R⁰ may both be H.

Typically R⁶ is H or unsubstituted or substituted C₁₋₆ alkyl. R⁶ in formula (Id) may be as further defined anywhere herein for formula (I). Often, however, R⁶ in formula (Id) is H.

For compounds of formula (Id) or pharmaceutically acceptable salts thereof, R⁷ is usually-CH(R¹¹)—Ar, —CH(R¹¹)-Ary-Ar, -Ary-Ar, —CH(R¹¹) Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is typically H, C(O)OR^{z} or unsubstituted or substituted C₁₋₄ alkyl. However, R⁷ and R¹¹ in formula (Id) may be as further defined anywhere herein for formula (I). R^{z} is selected from H, unsubstituted or substituted C₁₋₄ alkyl, and unsubstituted or substituted phenyl. However, R^{z} in formula (Id) may also be as further defined anywhere herein for formula (I).

R⁹ is typically H or unsubstituted or substituted C₁₋₆ alkyl.

R^{x} in formula (Id) is selected from H, unsubstituted or substituted C₁₋₄ alkyl, and unsubstituted or substituted phenyl. However, R^{x} in formula (Id) may be as further defined anywhere herein for formula (I). Usually, R^{x} in formula (Id) is H.

In some preferred embodiments wherein the substituted azine has the formula (Id), R⁹ is H.

The compound of the invention may be a substituted azine of formula (Id) selected from any one of the following structures, or a pharmaceutically acceptable salt thereof:

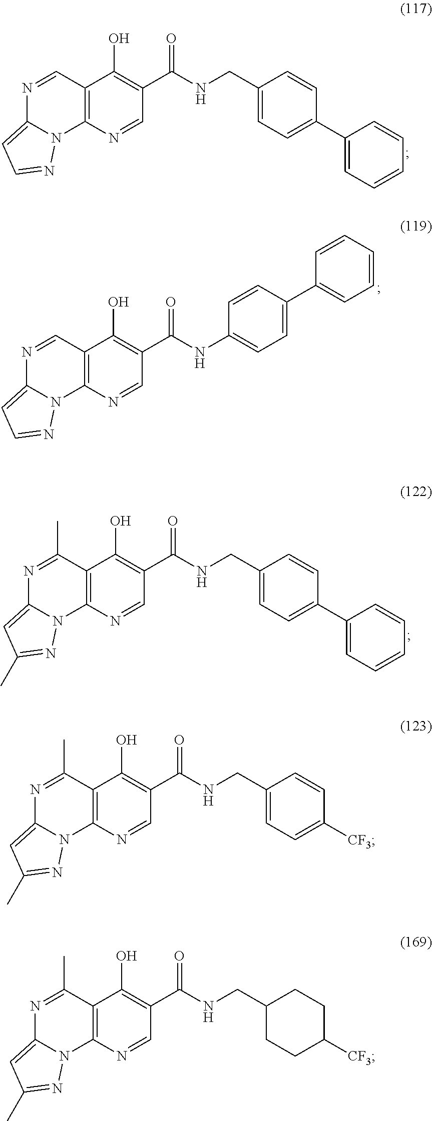 [SMILES: O=C(NCc1ccc(-c2ccccc2)cc1)c1cnc2c(cnc3ccnn32)c1O ; O=C(Nc1ccc(-c2ccccc2)cc1)c1cnc2c(cnc3ccnn32)c1O ; Cc1cc2nc(C)c3c(O)c(C(=O)NCc4ccc(-c5ccccc5)cc4)cnc3n2n1 ; Cc1cc2nc(C)c3c(O)c(C(=O)NCc4ccc(C(F)(F)F)cc4)cnc3n2n1 ; Cc1cc2nc(C)c3c(O)c(C(=O)NCC4CCC(C(F)(F)F)CC4)cnc3n2n1]

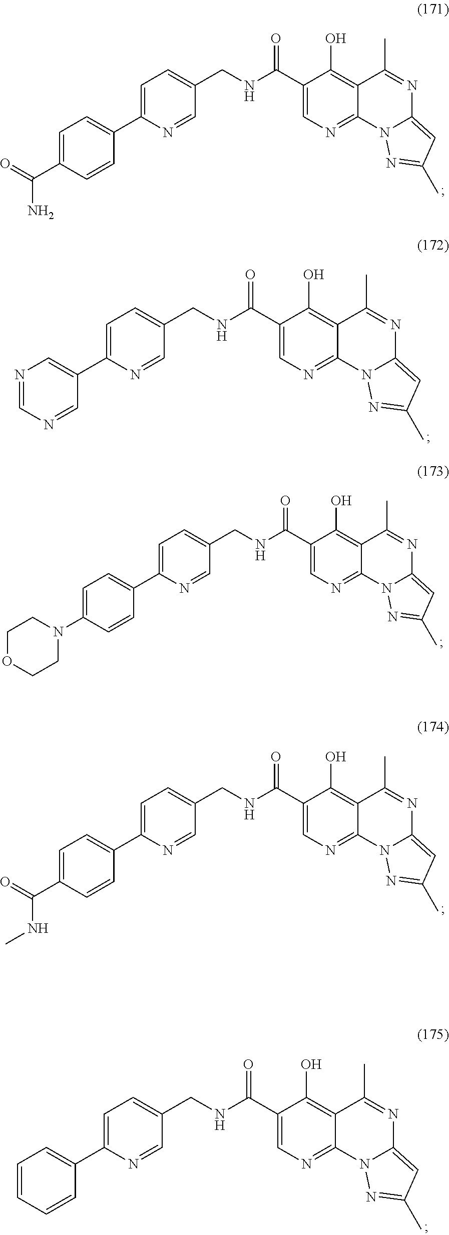 [SMILES: Cc1cc2nc(C)c3c(O)c(C(=O)NCc4ccc(-c5ccc(C(N)=O)cc5)nc4)cnc3n2n1 ; Cc1cc2nc(C)c3c(O)c(C(=O)NCc4ccc(-c5cncnc5)nc4)cnc3n2n1 ; Cc1cc2nc(C)c3c(O)c(C(=O)NCc4ccc(-c5ccc(N6CCOCC6)cc5)nc4)cnc3n2n1 ; CNC(=O)c1ccc(-c2ccc(CNC(=O)c3cnc4c(c(C)nc5cc(C)nn54)c3O)cn2)cc1 ; Cc1cc2nc(C)c3c(O)c(C(=O)NCc4ccc(-c5ccccc5)nc4)cnc3n2n1]

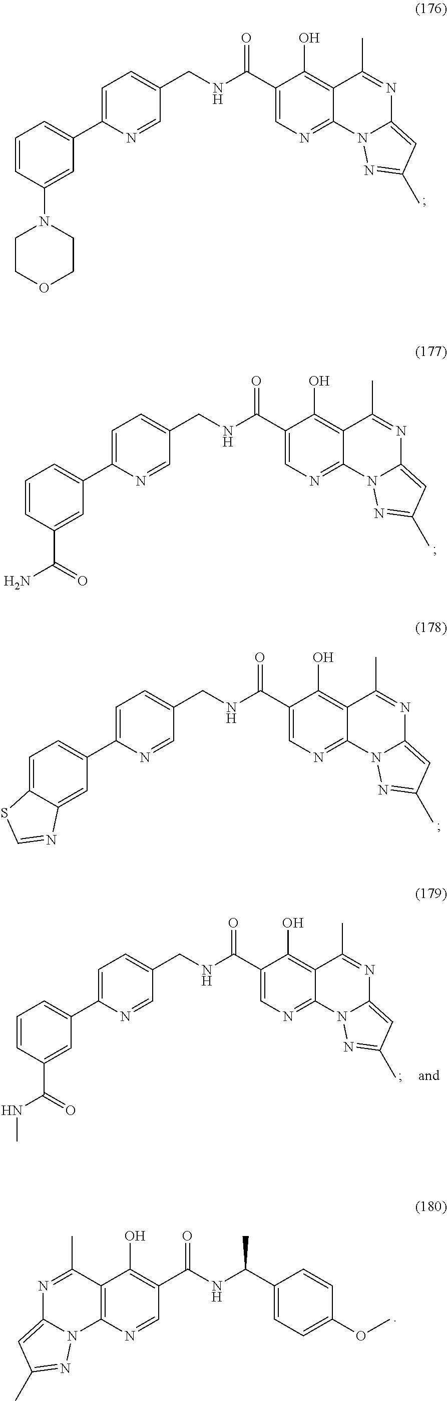

The numbers in parentheses next to the structures listed above correspond to the compound numbers given in the Examples section hereinbelow.

In another embodiment, the substituted azine of formula (I) has any one of the following structures. Accordingly, the invention provides a compound which is a substituted azine having any one of the following structures or a pharmaceutically acceptable salt thereof:

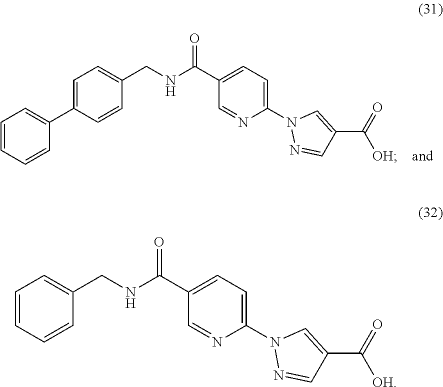 [SMILES: O=C(O)c1cnn(-c2ccc(C(=O)NCc3ccc(-c4ccccc4)cc3)cn2)c1 ; "and" ; O=C(O)c1cnn(-c2ccc(C(=O)NCc3ccccc3)cn2)c1]

The numbers in parentheses next to the structures listed above correspond to the compound numbers given in the Examples section hereinbelow.

The invention also provides a compound which is a substituted azine of formula (Ia) as defined herein, or a pharmaceutically acceptable salt thereof, wherein R⁹ in said formula (Ia) is other than H (and wherein X, R⁰, R¹, R², R³, R⁷ and R^{x} are as defined herein for formula Ia). Such a compound embraces prodrugs. Typically, R⁹ in this embodiment is unsubstituted or substituted C₁₋₆ alkyl. In one aspect of this embodiment, R⁹ is substituted C₁₋₆ alkyl. R⁹ may for instance be C₁₋₆ alkyl which is substituted with phenyl or —OC(O)R⁹⁹, wherein R⁹⁹ is as defined above. Thus R⁹⁹ may be phenyl, unsubstituted C₁₋₆ alkyl, —N(R^{a})(R^{b}), —C(O)R^{c}, OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl. R⁹ may for instance be C₁₋₆ alkyl which is substituted with —OC(O)R⁹⁹, wherein R⁹⁹ is as defined above. In another aspect of this embodiment, R⁹ is unsubstituted C₁₋₆ alkyl. R⁹ may for instance be methyl.

The substituted azine of formula (Ia) may for instance be selected from any one of the following structures

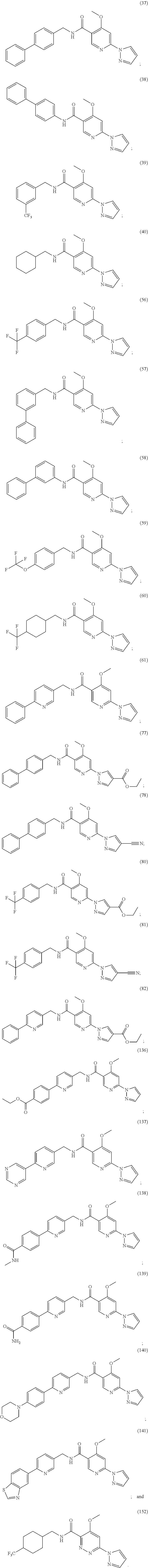

The numbers in parentheses next to the structures listed above correspond to the compound numbers given in the Examples section hereinbelow.

The invention also provides a compound which is a substituted azine of formula (Ib) as defined herein, or a pharmaceutically acceptable salt thereof, wherein R⁸ in said formula (Ib) is other than H (and wherein R⁰, R¹, R², R⁴, R⁶, R⁷ and R^{x} are as defined herein for formula Ib). Such a compound embraces prodrugs. Typically, R⁸ in this embodiment is unsubstituted or substituted C₁₋₆ alkyl. In one aspect of this embodiment, R⁸ is substituted C₁₋₆ alkyl. R⁸ may for instance be C₁₋₆ alkyl which is substituted with phenyl or —OC(O)R⁹⁹, wherein R⁹⁹ is as defined above. Thus R⁹⁹ may be phenyl, unsubstituted C₁₋₆ alkyl, —N(R^{a})(R^{b}), —C(O)R^{c}, —OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl. R⁸ may for instance be C₁₋₆ alkyl which is substituted with —OC(O)R⁹⁹, wherein R⁹⁹ is as defined above. In another aspect of this embodiment, R⁸ is unsubstituted C₁₋₆ alkyl. R⁸ may for instance be methyl. The substituted azine of formula (Ib) may for instance be selected from any one of the following structures

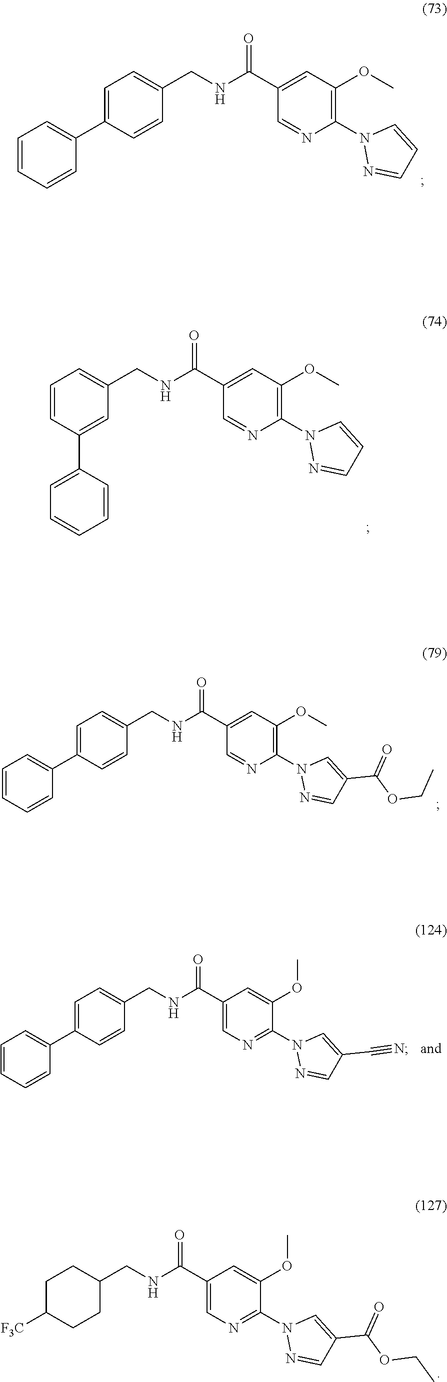 [SMILES: COc1cc(C(=O)NCc2ccc(-c3ccccc3)cc2)cnc1-n1cccn1 ; COc1cc(C(=O)NCc2cccc(-c3ccccc3)c2)cnc1-n1cccn1 ; CCOC(=O)c1cnn(-c2ncc(C(=O)NCc3ccc(-c4ccccc4)cc3)cc2OC)c1 ; COc1cc(C(=O)NCc2ccc(-c3ccccc3)cc2)cnc1-n1cc(C#N)cn1 ; CCOC(=O)c1cnn(-c2ncc(C(=O)NCC3CCC(C(F)(F)F)CC3)cc2OC)c1]

The numbers in parentheses next to the structures listed above correspond to the compound numbers given in the Examples section hereinbelow.

The invention also provides a compound which is a substituted azine of formula (Ic) as defined herein, or a pharmaceutically acceptable salt thereof, wherein R⁴ is —OR⁹ or —C(O)OR¹⁰, and/or R⁵ is —C(O)OR^{w}, in which R⁹, R¹⁰ and R^{w} are other than H (and wherein R⁰, R², R³, R⁴, R⁵, R⁶, R⁷ and R^{x} are otherwise as defined herein for formula Ic). Such a compound embraces prodrugs. Typically, in the substituted azine of formula (Ic): (a) R⁴ is —OR⁹; or (b) R⁴ is —C(O)OR¹⁰; or (c) R⁵ is —C(O)OR^{w}; or (d) R⁴ is —OR⁹ and R⁵ is —C(O)OR^{w}. R⁹, R¹⁰ and R^{w}, which are the same or different, are unsubstituted or substituted C₁₋₆ alkyl groups.

In one aspect of this embodiment, R⁹, R¹⁰ and R^{w} are substituted C₁₋₆ alkyl groups. R⁹ and R¹⁰ may for instance be C₁₋₆ alkyl which is substituted with phenyl or —OC(O)R⁹⁹, wherein R⁹⁹ is as defined above. Thus R⁹⁹ may be phenyl, unsubstituted C₁₋₆ alkyl, —N(R^{a})(R^{b}), —C(O)R^{c}, —OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl. R⁹ and R¹⁰ may for instance be C₁₋₆ alkyl which is substituted with —OC(O)R⁹⁹, wherein R⁹⁹ is as defined above. Similarly, R^{w}, in this aspect of this embodiment, may be a substituted C₁₋₆ alkyl group. R^{w} may for instance be C₁₋₆ alkyl which is substituted with phenyl or —OC(O)R^{ww}, wherein R^{ww} is is as defined above. Thus R^{ww} may be phenyl, unsubstituted C₁₋₆ alkyl, —N(R^{a})(R^{b}), —C(O)R^{c}, —OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl. R^{w} may for instance be C₁₋₆ alkyl which is substituted with —OC(O)R^{ww}, wherein R^{ww} is is as defined above.

In another aspect of this embodiment, R⁹, R¹⁰ and R^{w}, which may be the same or different, are unsubstituted C₁₋₆ alkyl groups. R⁹, R¹⁰ and R^{w} may for instance be selected from methyl and ethyl groups.

The substituted azine of formula (Ic) may for instance be selected from any one of the following structures

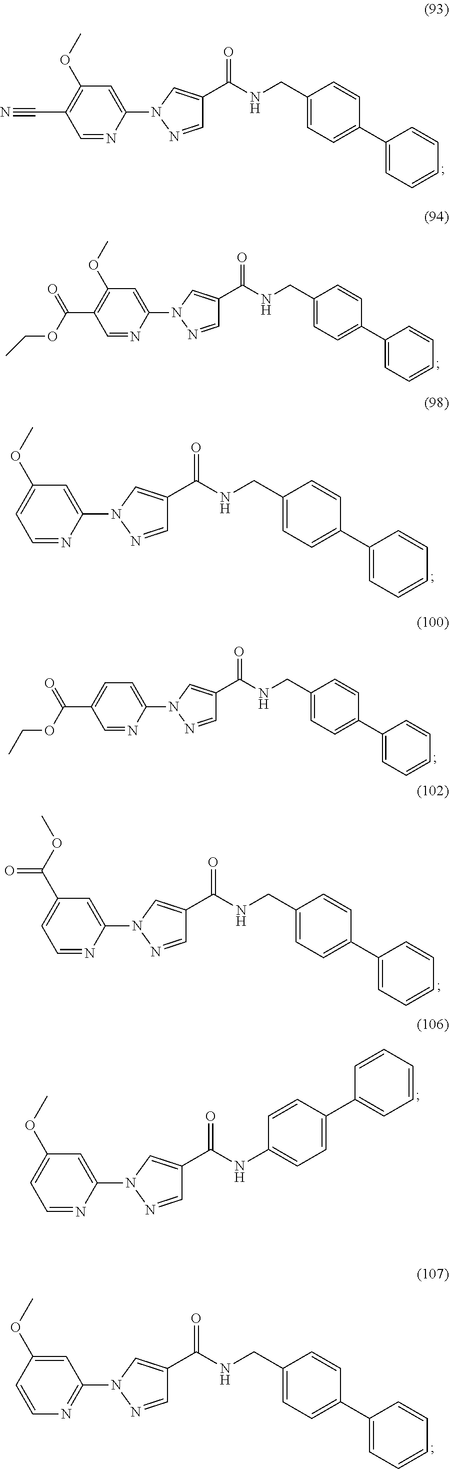

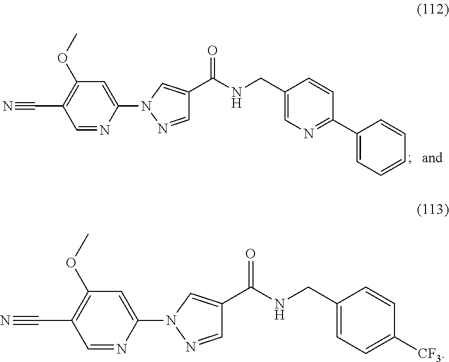 [SMILES: COc1cc(-n2cc(C(=O)NCc3ccc(-c4ccccc4)nc3)cn2)ncc1C#N ; COc1cc(-n2cc(C(=O)NCc3ccc(C(F)(F)F)cc3)cn2)ncc1C#N]

The numbers in parentheses next to the structures listed above correspond to the compound numbers given in the Examples section hereinbelow.

The invention also provides a compound which is a substituted azine of formula (Id) as defined herein, or a pharmaceutically acceptable salt thereof, wherein R⁹ is other than H (and wherein R⁰, R¹, R^{y}, R⁶, R⁷ and R^{x} are as defined herein for formula Id). Such a compound embraces prodrugs. Typically, R⁹ in this embodiment is unsubstituted or substituted C₁₋₆ alkyl. In one aspect of this embodiment, R⁹ is substituted C₁₋₆ alkyl. R⁹ may for instance be C₁₋₆ alkyl which is substituted with phenyl or —OC(O)R⁹⁹, wherein R⁹⁹ is as defined above. Thus R⁹⁹ may be phenyl, unsubstituted C₁₋₆ alkyl, —N(R^{a})(R^{b}), —C(O)R^{c}, —OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl. R⁹ may for instance be C₁₋₆ alkyl which is substituted with —OC(O)R⁹⁹, wherein R⁹⁹ is as defined above. In another aspect of this embodiment, R⁹ is unsubstituted C₁₋₆ alkyl. R⁹ may for instance be methyl. The substituted azine of formula (Id) may for instance have the following structure

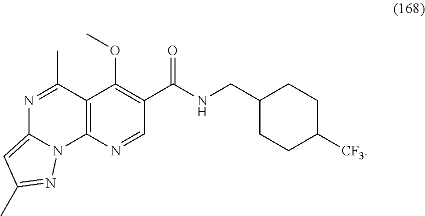 [SMILES: COc1c(C(=O)NCC2CCC(C(F)(F)F)CC2)cnc2c1c(C)nc1cc(C)nn12]

The number in parentheses next to the structure listed above corresponds to the compound numbers given in the Examples section hereinbelow.

The above compounds of formula (Ia), (Ib), (Ic) and (Id) wherein R⁸, R⁹, R¹⁰ or R^{w} are typically unsubstituted or substituted C₁₋₆ have surprising advantages as prodrug structures for compounds of formula (Ia), (Ib), (Ic) or (Id), which are effective HIF-PHD inhibitors. In particular, the compounds with the structures as described above have been surprisingly shown to improve efficacy of the inhibitors in a cellular assay, even when they themselves do not have high potency as HIF-PHD inhibitors. The reduced potency but higher activity in a cellular assay means that these compounds have the potential to provide targeting inhibition with reduced off-target effects.

The invention also provides a compound which is a substituted pyrimidine of formula (IV) or a pharmaceutically acceptable salt thereof

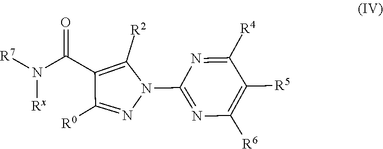

wherein
R⁰ is H or unsubstituted or substituted C₁₋₆ alkyl;
R² is H, —OR^{q} or unsubstituted or substituted C₁₋₆ alkyl;
R⁴ is —OR⁹, wherein R⁹ is selected from H and unsubstituted or substituted C₁₋₆ alkyl;
R⁵ is H, unsubstituted or substituted C₁₋₆ alkyl, unsubstituted or substituted aryl, unsubstituted or substituted heteroaryl, —CN or —C(O)OR^{w};
R⁶ is H or unsubstituted or substituted C₁₋₆ alkyl;
R⁷ is —CH(R¹¹)—Ar, —CH(R¹¹)-Ary-Ar, -Ary-Ar, CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R^{x} is H, unsubstituted or substituted C₁₋₄ alkyl, or unsubstituted or substituted phenyl;
R^{w} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.

Typically, in formula (IV), R⁰ is H or unsubstituted C₁₋₆ alkyl, for instance H or methyl. Often, R⁰ is H.

Usually, in formula (IV), R² is H or unsubstituted C₁₋₆ alkyl, for instance H or methyl. Often, R² is H.

Typically, in formula (IV), R⁰ is H and R² is H.

Often, in formula (IV), R⁵ is —CN.

R⁶ in formula (IV) is often H or unsubstituted C₁₋₆ alkyl, for instance H or methyl. R⁶ in formula (IV) is typically H.

R⁴ in formula (IV) is —OR⁹, and R⁹ is selected from H and unsubstituted or substituted C₁₋₆ alkyl. However, R⁹, in formula (IV), may be as defined anywhere herein for R⁹ formula (I).

Often, in formula (IV), R⁰ is H. When R⁹ is H (i.e. when R⁴ is OH), then R⁵ is typically —CN.

In some embodiments, however, R⁹ in formula (IV) may be unsubstituted or substituted C₁₋₆ alkyl. Such embodiments embrace prodrugs. In one aspect of this embodiment, R⁹ in formula (IV) is substituted C₁₋₆ alkyl. R⁹ may for instance be C₁₋₆ alkyl which is substituted with phenyl or —OC(O)R⁹⁹, wherein R⁹⁹ is as defined above. Thus R⁹⁹ may be phenyl, unsubstituted C₁₋₆ alkyl, —N(R^{a})(R^{b}), —C(O)R^{c}, —OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl. R⁹ in formula (IV) may for instance be C₁₋₆ alkyl which is substituted with —OC(O)R⁹⁹, wherein R⁹⁹ is as defined above. In another aspect of this embodiment, R⁹ in formula (IV) is unsubstituted C₁₋₆ alkyl. R⁹ may for instance be methyl.

R^{x} in formula (IV) is typically H or unsubstituted C₁₋₄ alkyl, for instance H or methyl. Often, R^{x} in formula (IV) is H.

R^{w} in formula (IV) is H, unsubstituted or substituted C₁₋₆ alkyl, or unsubstituted or substituted phenyl. R^{w} may be H, or unsubstituted or substituted C₁₋₆ alkyl. Typically, for instance, R^{w} is H, unsubstituted C₁₋₆ alkyl, or C₁₋₆ alkyl which is substituted with phenyl or —OC(O)R^{ww}, wherein R^{ww} is phenyl, unsubstituted C₁₋₆ alkyl, —N(R^{a})(R^{b}), —C(O)R^{c}, —OR^{d} or an amino acid, wherein R^{a}, R^{b}, R^{c} and R^{d} are each independently selected from H, unsubstituted or substituted C₁₋₆ alkyl and an amino acid. Often, R^{w} in formula (IV) is H.

Typically, R² in formula (IV) is H or unsubstituted C₁₋₄ alkyl, for instance H or methyl. Often, R² in formula (IV) is H.

R^{q} in formula (IV) is typically H or unsubstituted C₁₋₆ alkyl, or unsubstituted phenyl. It is often, for instance, H or unsubstituted C₁₋₄ alkyl, for instance H or methyl. Usually, R^{q} in formula (IV) is H.

Usually, R⁷ in formula (IV) is —CH(R¹¹)—Ar, —CH(R¹¹)-Ary-Ar, or —CH(R¹¹) Cyc, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR² or unsubstituted or substituted C₁₋₄ alkyl. R¹¹ is typically H or unsubstituted C₁₋₄ alkyl, for instance H or methyl. Often, R¹¹ is H.

R⁷ in formula (IV) may for instance be —CH(R¹¹)—Ar, —CH(R¹¹)-Ary-Ar, or CH(R¹¹)-Cyc; wherein Ar is unsubstituted phenyl, unsubstituted pyrimidyl, unsubstituted benzothiazole or phenyl substituted with —C(O)OH, —C(O)OMe, —C(O)OEt, —C(O)NH₂, —C(O)N(H)Me, —OMe or N-morpholino; Ary is unsubstituted phenylene or unsubstituted pyridylene; Cyc is unsubstituted cyclohexyl or cyclohexyl substituted with —CF₃ or —OCF₃; and R¹¹ is as defined above, typically H. Often, R⁷ in formula (IV) is —CH(R¹¹)—Ar, —CH(R¹¹)-Ary-Ar, or —CH(R¹¹) Cyc; wherein Ar is unsubstituted phenyl or phenyl substituted with —C(O)OH or —C(O)OMe; Ary is unsubstituted phenylene or unsubstituted pyridylene; Cyc is unsubstituted cyclohexyl or cyclohexyl substituted with —CF₃; and R¹¹ is as defined above, typically H.

A compound of formula (IV) may be represented by one of the following structures or a pharmaceutically acceptable salt thereof

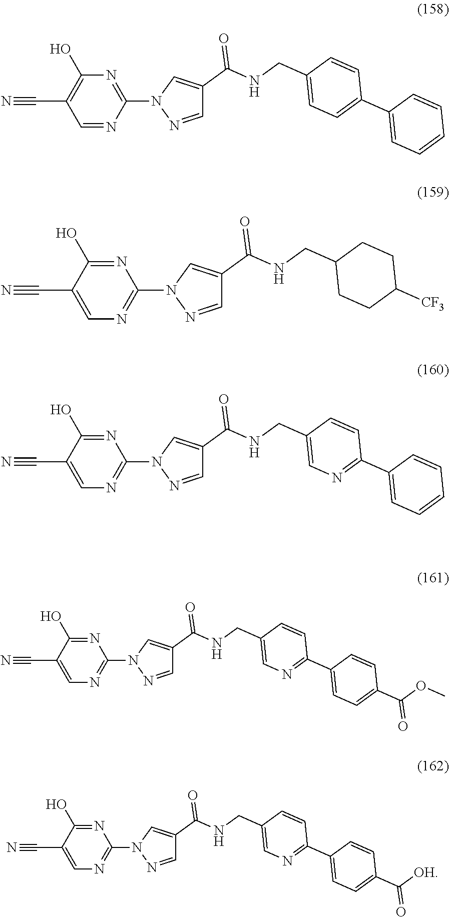 [SMILES: N#Cc1cnc(-n2cc(C(=O)NCc3ccc(-c4ccccc4)cc3)cn2)nc1O ; N#Cc1cnc(-n2cc(C(=O)NCC3CCC(C(F)(F)F)CC3)cn2)nc1O ; N#Cc1cnc(-n2cc(C(=O)NCc3ccc(-c4ccccc4)nc3)cn2)nc1O ; COC(=O)c1ccc(-c2ccc(CNC(=O)c3cnn(-c4ncc(C#N)c(O)n4)c3)cn2)cc1 ; N#Cc1cnc(-n2cc(C(=O)NCc3ccc(-c4ccc(C(=O)O)cc4)nc3)cn2)nc1O]

The numbers in parentheses next to the structures listed above correspond to the compound numbers given in the Examples section hereinbelow.

### General Synthetic Methodology

All compounds described herein can be prepared by any suitable method. Detailed general synthetic routes for compounds of the invention are set out below and in the Examples.

Compounds of formula (II) and pharmaceutically acceptable salts thereof may be prepared as described in WO2007/150011.

Compounds of formula (III) and pharmaceutically acceptable salts thereof may be prepared as described in WO2008/067871.

The substituted azines of formula (I) and the substituted pyrimidines of formula (IV) may for instance be synthesised using the methodology set forth under the headings “General Procedure A”, “General Procedure B”, “General Procedure C” and “General Procedure D” in the Examples section hereinbelow. The application of these General Procedures to produce substituted azines of formula (I) is shown and described below with reference to schemes 2, 3, 4, 5, 6, 7, 8, 9, 10, 11 and 12, and specific synthesis examples are described in the Examples section hereinbelow. Similarly, the application of the General Procedures to produce substituted pyrimidines of formula (IV) is shown and described below with reference to scheme 11.

Substituted azines of formula (I) and substituted pyrimidines of formula (IV) may be synthesised using an amide coupling procedure (General Procedure A or B), which can be used to introduce an amide group (e.g. the amide group of formula-C(O)N(R^{x})R⁷) by coupling it with a carboxylic acid or an ester group in a precursor compound. A Pd-catalysed coupling procedure (General Procedure C) may then be employed to further modify the amide group introduced in the previous step, to arrive at the desired C(O)N(R^{x})R⁷ group in the final compound. An alkoxy-dealkylation step (General Procedure D) may then be employed as a final step to render an OH group in the final compound.

Scheme 10 below, for instance, shows how substituted azines of formula (Ia) may be produced using the General Procedures B, C and D provided in the Example section. The same General Procedures B, C and D can also be used to produce substituted azines of formula (Ib). Scheme 6 below shows how substituted azines of formula (Ic) may be produced using the General Procedures B and D described in the Example section. Schemes 7, 8 and 12 below illustrate the synthesis of the pyrazolo[1,5-a]pyrido[3,2-e]-pyrimidine-7-amido structures of formula (Id) using the general procedures A and D in the Examples. Furthermore, scheme 11 illustrates how the substituted pyrimidines of formula (IV) can be produced using General Procedure B. Schemes 1 and 2 are also provided below, to illustrate general methods for preparation of some of the reference Examples described herein. As the skilled person will appreciate, alternative precursor compounds, with substituent groups that are different from those shown in the schemes below, may be employed in the same methods in order to achieve variation within the scopes of formulae (I), (Ia), (Ib), (Ic), (Id) and (IV) herein.

Thus, compounds described herein can be prepared according to the following reaction schemes:

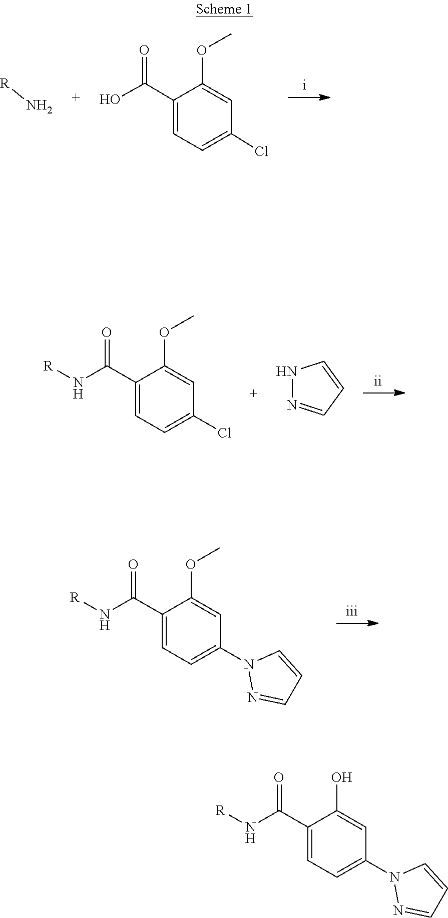

Scheme 1 step (i) may be carried out using treatment with any appropriate peptide coupling reagents. Typically, step (i) comprises treatment with an acid anhydride, for example propanephosphonic acid anhydride in the presence of a base such as N,N-diisopropylethylamine (DIPEA). The reaction typically occurs in a solvent. The solvent may be a polar solvent. Typically the solvent may be a polar, aprotic solvent. The polar aprotic solvent may be dimethylacetamide (DMAc). Typically step (i) occurs at standard atomospheric temperature and pressure (SATP), i.e. approximately 25° C., and 1 atmospheric pressure (around 100,000 Pa). The step may last for between 1 and 24 hours, for example about 16 hours.

Step (ii) of Scheme 1 comprises treatment in the presence of a catalyst. The catalyst may be a palladium catalyst. Typically, the catalyst may be Pd^{t}BuXPhos. The step may also take place in the presence of a base. The base may be a carbonate. Usually the base is cesium carbonate (Cs₂CO₃). The reaction typically occurs in a solvent. The solvent may be a mixture of solvents. Usually, the reaction occurs in tert butanol (^{t}BuOH). Step (ii) typically occurs at a temperature greater than room temperature. For example, step (iv) typically occurs between 60° C. and 100° C. Usually the step occurs at around 80° C. The step may last for between 1 and 24 hours, for example about 16 hours.

Step (iii) of Scheme 1 is a deprotection step that may comprise treatment with any suitable reagents known to the skilled person. In some instances, a silyl halide compound is used. Usually, trimethylsilyl iodide (TMS-I) is used. This step typically takes place in the presence of a solvent. The solvent is typically a polar solvent, and more typically a polar aprotic solvent. The solvent may be dichloromethane (CH₂Cl₂). Step (iii) typically occurs at a temperature greater than room temperature. For example, step (iii) typically occurs between 40° C. and 80° C. Usually the step occurs at around 60° C. The step may last for between 1 and 24 hours, for example about 8 hours.

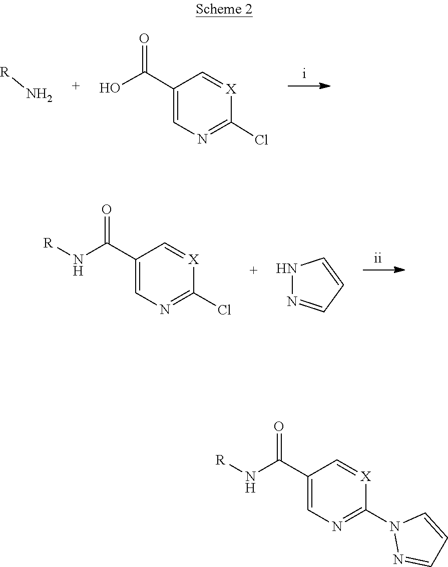

Scheme 2 step (i) may comprise treatment with any appropriate amide coupling reagents. Typically, step (i) comprises treatment with an acid anhydride, for example propanephosphonic acid anhydride (T3P) in the presence of a base such as N,N-diisopropylethylamine (DIPEA). The reaction typically occurs in a solvent. The solvent may be a polar solvent. Typically, the solvent may be a polar, aprotic solvent. The polar aprotic solvent may be dimethylacetamide (DMAc). Typically step (i) occurs at SATP. The step may last for between 1 and 24 hours, for example about 16 hours.

Step (ii) of Scheme 2 comprises treatment in the presence of a catalyst. The catalyst may be a palladium catalyst. Typically, the catalyst is Pd^{t}BuXPhos. The step may also take place in the presence of a base. The base may be a carbonate. Usually, the base is cesium carbonate (Cs₂CO₃). The reaction typically occurs in a solvent. The solvent may be a mixture of solvents. Typically, the solvent may be a mixture of one or more polar solvents, often polar aprotic solvents. Usually, the reaction occurs in a mixture of DMAc and tetrahydrofuran (THF). This mixture may be an approximately 1:1 mixture (1:1). Step (ii) typically occurs at a temperature greater than room temperature. For example, step (ii) typically occurs between 80° C. and 120° C. Usually, the step occurs at around 100° C. The step may last for between 30 minutes and 6 hours, for example about 1 hour.

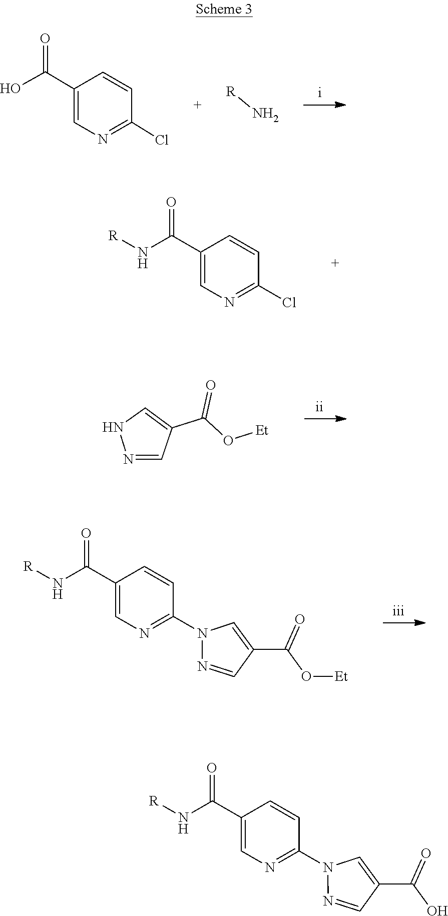

Scheme 3 above shows how certain substituted azines of formula (I) may be produced using the general synthesis procedures set forth in the Examples section hereinbelow (see the General Procedures A, B, C and D in the Examples).

Scheme 3 step (i) may comprise treatment with any appropriate peptide coupling reagents. Typically step (i) comprises treatment with an acid anhydride, for example propanephosphonic acid anhydride in the presence of a base such as N,N-diisopropylethylamine (DIPEA). The reaction typically occurs in a solvent. The solvent may be a polar solvent. Typically, the solvent may be a polar, aprotic solvent. The polar aprotic solvent may be dimethylacetamide (DMAc). Typically step (i) occurs at SATP. The step may last for between 1 and 24 hours, for example about 16 hours.

Step (ii) of Scheme 3 comprises treatment in the presence of a catalyst. The catalyst may be a palladium catalyst. Typically, the catalyst may be Pd^{t}BuXPhos. The step may also take place in the presence of a base. The base may be a carbonate. Typically, the base is Cs₂CO₃, Na₂CO₃ or K₂CO₃. Usually, the base is Cs₂CO₃. The reaction typically occurs in a solvent. The solvent may be a mixture of solvents. Typically, the solvent may be a mixture of one or more polar solvents, often polar aprotic solvents. Usually, the reaction occurs in a mixture of DMAc and tetrahydrofuran (THF). This mixture may be a 1:1 mixture (1:1). Step (ii) typically occurs at a temperature greater than room temperature. For example, step (ii) typically occurs between 80° C. and 120° C. Usually the step occurs at around 100° C. The step may last for between 30 minutes and 6 hours, for example about 1 hour.

Step (iii) may be carried out using any suitable reagents for ester hydrolysis. Typically, the reaction may be carried out in the presence of a hydroxide base, usually lithium hydroxide. The reaction is usually carried out in the presence of a solvent. The solvent may be a mixture of one or more solvents, typically one or more polar solvents. The one or more polar solvents may be protic or aprotic. For example, the solvent may be a mixture of THF and H₂O. Usually, the mixture is approximately a 1:1 mixture. Typically step (iv) occurs at SATP. The step may last for between 1 and 24 hours, for example about 16 hours.

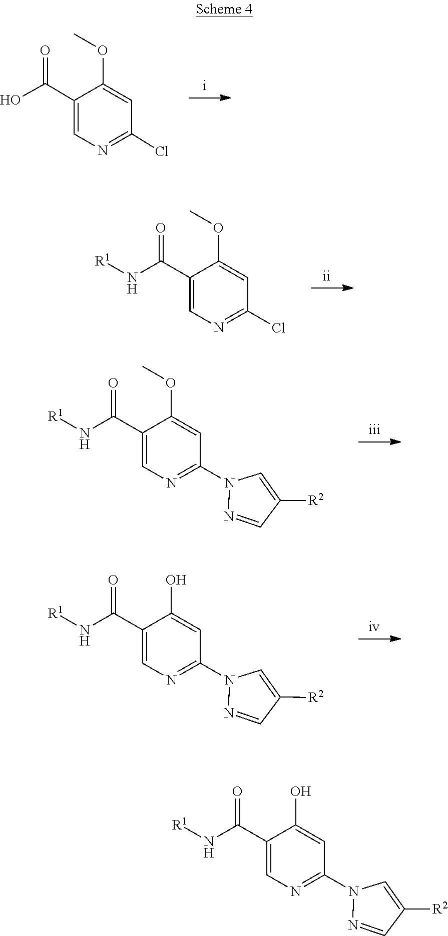

Scheme 4 above shows how the 4-hydroxypyridine/pyridinone structures of formula (Ia) may be produced using the general synthesis procedures set forth in the Examples section hereinbelow (see the General Procedures A and D in the Examples).

Step (i) of Scheme 4 typically comprises treatment with R—NH₂. Scheme 4 step (i) may comprise treatment with any appropriate amide coupling reagents. Typically, step (i) comprises treatment with an acid anhydride, for example propanephosphonic acid anhydride in the presence of a base such as N,N-diisopropylethylamine (DIPEA). The reaction typically occurs in a solvent. The solvent may be a polar solvent. Typically, the solvent may be a polar, aprotic solvent. The polar aprotic solvent may be dimethylacetamide (DMAc). Typically step (i) occurs at SATP. The step may last for between 1 and 24 hours, for example about 16 hours.

Step (ii) of Scheme 4 may comprise treatment with further reagents such as pyrazole and substituted pyrazoles. Step (ii) of Scheme 4 comprises treatment in the presence of a catalyst. The catalyst may be a palladium catalyst. Typically, the catalyst may be Pd^{t}BuxPhos G3. The step may also take place in the presence of a base. The base may be a carbonate. Usually, the base is Cs₂CO₃. The reaction typically occurs in a solvent. The solvent may be a mixture of solvents. Typically, the solvent may be a mixture of one or more polar solvents, often polar aprotic solvents. Usually, the reaction occurs in tert butanol or 1,4-dioxane. Step (ii) typically occurs at a temperature greater than room temperature. For example, step (ii) typically occurs between 40° C. and 80° C. Usually, the step occurs at around 60° C. The step may last for between 1 hour to 48 hours, for example about 16 hours.

Step (iii) of Scheme 4 is a deprotection step that may comprise treatment with any suitable reagents known to the skilled person. In some instances, lithium chloride is used. This step typically takes place in the presence of a solvent. The solvent is typically a polar solvent, and more typically a polar aprotic solvent. The solvent may be DMAc. Step (v) typically occurs at a temperature greater than room temperature. For example, step (v) typically occurs between 80° C. and 120° C. Usually, the step occurs at around 100° C. The step may last for between 30 minutes and 6 hours, for example about 2 hours.

Step (iv) of Scheme 4, if required, may be carried out using any suitable reagents for ester hydrolysis. Typically, the reaction may be carried out in the presence of a hydroxide compound, usually lithium hydroxide. The reaction is usually carried out in the presence of a solvent. The solvent may be a mixture of one or more solvents, typically one or more polar solvents. The one or more polar solvents may be protic or aprotic. For example, the solvent may be a mixture of THF and H₂O. Usually the mixture is 1:1 mixture. Typically step (ii) occurs at SATP. The step may last for between 1 and 24 hours, for example about 16 hours.

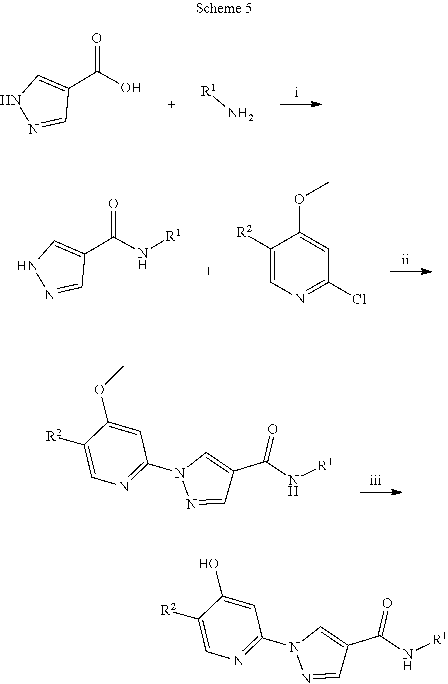

Scheme 5 above shows how the 4-hydroxypyridine/pyridinone structures of formula (Ic) may be produced using the general synthesis procedures set forth in the Examples section hereinbelow (see the General Procedures A and D in the Examples).

Step (i) of Scheme 5 may comprise treatment with any appropriate amide coupling reagents. Typically, step (i) comprises treatment with an acid anhydride, for example propanephosphonic acid anhydride in the presence of a base such as N,N-diisopropylethylamine (DIPEA). The reaction typically occurs in a solvent. The solvent may be a polar solvent. Typically, the solvent may be a polar, aprotic solvent. The polar aprotic solvent may be dimethylacetamide (DMAc). Typically step (i) occurs at SATP. The step may last for between 1 and 24 hours, for example about 16 hours.

Step (ii) of Scheme 5 comprises treatment in the presence of a catalyst. The catalyst may be a palladium catalyst. Typically, the catalyst may be Pd BuxPhos G3. The step may also take place in the presence of a base. The base may be a carbonate. Usually, the base is Cs₂CO₃. The reaction typically occurs in a solvent. The solvent may be a mixture of solvents. Typically, the solvent may be a mixture of one or more polar solvents, often polar aprotic solvents. Usually, the reaction occurs in tert butanol or 1,4-dioxane. Step (ii) typically occurs at a temperature greater than room temperature. For example, step (ii) typically occurs between 40° C. and 80° C. Usually, the step occurs at around 60° C. The step may last for between 1 hour to 48 hours, for example about 16 hours. Step (ii) may also comprise treatment with further reagents such as pyrazole and substituted pyrazoles.

Step (iii) of Scheme 5 is a deprotection step that may comprise treatment with any suitable reagents known to the skilled person. In some instances, lithium chloride is used. This step typically takes place in the presence of a solvent. The solvent is typically a polar solvent, and more typically a polar aprotic solvent. The solvent may be DMAc. Step (v) typically occurs at a temperature greater than room temperature. For example, step (v) typically occurs between 80° C. and 120° C. Usually, the step occurs at around 100° C. The step may last for between 30 minutes and 6 hours, for example about 2 hours.

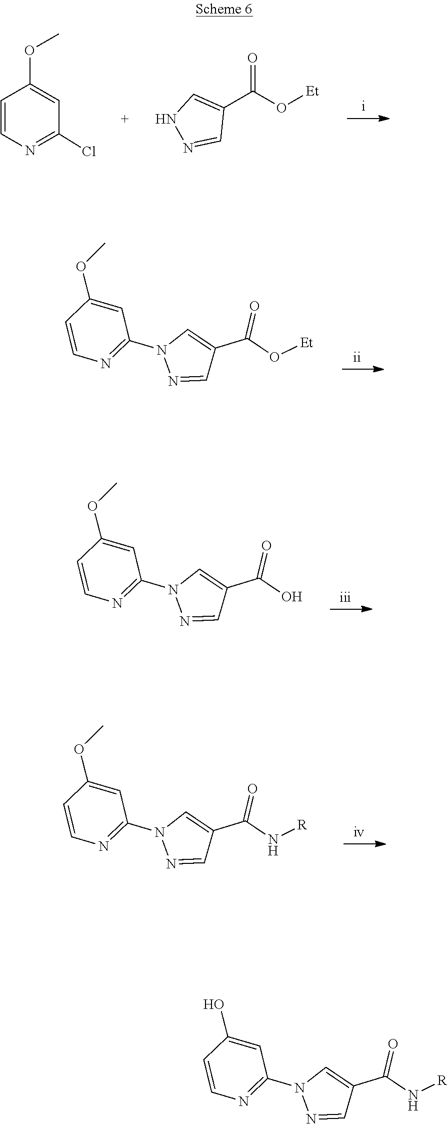

Scheme 6 above shows how substituted azines of formula (Ic) may be produced using the general synthesis procedures set forth in the Examples section hereinbelow (see the General Procedures B and D in the Examples).

Step (i) of Scheme 6 comprises treatment in the presence of a catalyst. The catalyst may be a palladium catalyst. Typically, the catalyst may be RockPhos Pd G3. The step may also take place in the presence of a base. The base may be a carbonate. Typically, the base is Cs₂CO₃. The reaction typically occurs in a solvent. The solvent may be a non-polar solvent. Typically, the solvent is a non-polar protic solvent. Usually, the solvent is ^{t}BuOH. Step (i) typically occurs at a temperature greater than room temperature. For example, step (i) typically occurs between 60° C. and 100° C. Usually the step occurs at around 80° C. The step may last for between 1 hour to 24 hours, for example about 16 hours.

Step (ii) may be carried out using any suitable reagents for ester hydrolysis. Typically, the reaction may be carried out in the presence of a hydroxide base, usually lithium hydroxide. The reaction is usually carried out in the presence of a solvent. The solvent may be a mixture of one or more solvents, typically one or more polar solvents. The one or more polar solvents may be protic or aprotic. For example, the solvent may be a mixture of THF and H₂O. Usually the mixture is 1:1 mixture. Typically step (ii) occurs at SATP. The step may last for between 1 and 24 hours, for example about 16 hours.

In Scheme 6 step (iii) the starting material is treated with a group R—NH₂. For step (iii) any appropriate peptide coupling reagents may be used. Typically, Scheme 6 step (iii) comprises treatment with an acid anhydride, for example propanephosphonic acid anhydride in the presence of a base such as N,N-diisopropylethylamine (DIPEA). The reaction typically occurs in a solvent. The solvent may be a polar solvent. Typically, the solvent may be a polar, aprotic solvent. The polar aprotic solvent may be dimethylacetamide (DMAc). Typically step (iii) occurs at SATP. The step may last for between 1 and 24 hours, for example about 16 hours.

Step (iv) of Scheme 6 is a deprotection step that may comprise treatment with any suitable reagents known to the skilled person. In some instances, lithium chloride is used. This step typically takes place in the presence of a solvent. The solvent is typically a polar solvent, and more typically a polar aprotic solvent. The solvent may be DMAc. Step (iv) typically occurs at a temperature greater than room temperature. For example, step (iv) typically occurs between 80° C. and 120° C. Usually the step occurs at around 100° C. The step may last for between 30 minutes and 6 hours, for example about 2 hours.

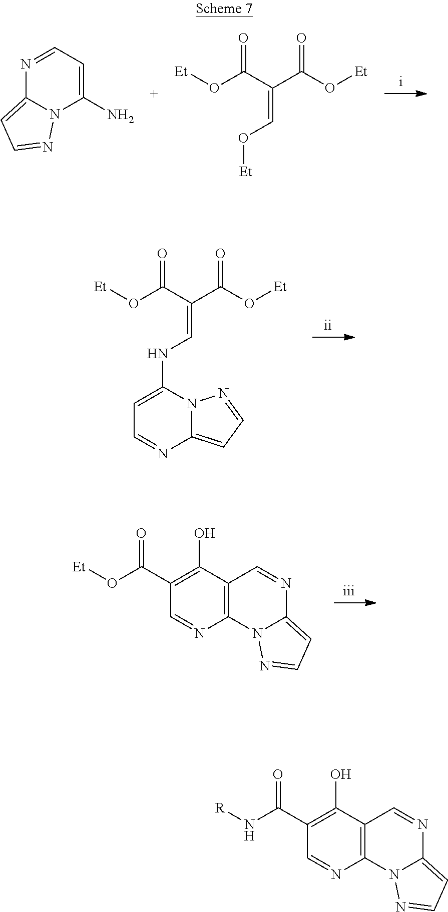

Scheme 7 above shows how the pyrazolo[1,5-a]pyrido[3,2-e]-pyrimidine-7-amido structures of formula (Id) may be produced using the general synthesis procedures set forth.

Step (i) of Scheme 7 is a Michael addition reaction that may compromise treatment with any suitable reagents known to the skilled person. In some instances, sodium ethoxide is used. This step typically takes place in the presence of a solvent. The solvent is typically a polar solvent, and more typically a polar aprotic solvent. The solvent may be EtOH. Step (i) typically occurs at a temperature greater than room temperature. Typically occurs between 80° C. and 120° C. Usually, the step occurs at around 100° C. The step may last for between 1 hour to 48 hours, for example about 2 hours.

Step (ii) of Scheme 7 is an intramolecular cyclisation that may compromise treatment with any suitable reagents known to the skilled person. This step typically takes place in the presence of a solvent. The solvent is typically one of high boiling point. The solvent may be diphenylether. Step (ii) typically occurs at a temperature greater than room temperature. Typically the temperature is between 140° C. and 250° C. Usually, the step occurs at around 250° C. The step may last for between 10 minutes to 8 hours, for example about 30 minutes.

Step (iii) of Scheme 7 is an amide coupling directly from the ethyl ester and comprises treatment in the presence of a catalyst. Typically, the catalyst may be DABCO-(AlMe₃)₂. The reaction typically occurs in a solvent. The solvent may be a mixture of solvents. Usually, the reaction occurs in tetrahydrofuran. Step (iii) typically occurs at a temperature greater than room temperature. For example, step (iii) typically occurs between 40° C. and 150° C. Usually, the step occurs at around 130° C. The step may last for between 10 minutes to 12 hours, for example about 1 hour.

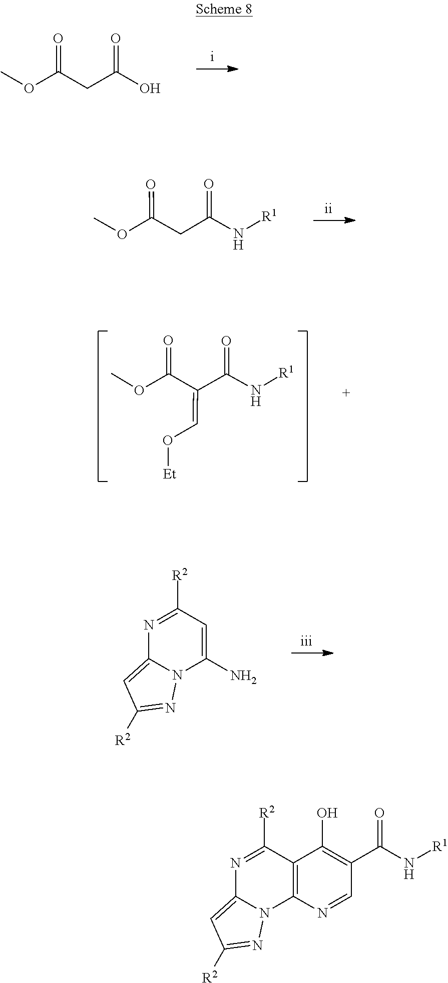

Scheme 8 above shows how the pyrazolo[1,5-a]pyrido[3,2-e]-pyrimidine-7-amido structures of formula (Id) may be produced using the general synthesis procedures set forth in the Examples section hereinbelow (see the General Procedures A and D in the Examples).

In Scheme 8 step (i) the starting material treated with a group R—NH₂. For step (i) any appropriate amide coupling reagents may be used. Typically, Scheme 4 step (i) comprises treatment with an acid anhydride, for example propanephosphonic acid anhydride in the presence of a base such as N,N-diisopropylethylamine (DIPEA). The reaction typically occurs in a solvent. The solvent may be a polar solvent. Typically, the solvent is a polar, aprotic solvent. The polar aprotic solvent may be dimethylacetamide (DMAc). Typically step (i) occurs at SATP. The step may last for between 1 and 24 hours, for example about 16 hours.

Scheme 8 step (ii) comprises treatment with ethyl orthoformate. The reaction typically occurs neat. Typically, step (ii) occurs at a temperature greater than room temperature. For example, step (ii) may occur at between 100° C. and 140° C. Usually this step occurs at around 120° C. The step may last for between 30 minutes and 6 hours, for example about 2 hours.

Step (iii) of Scheme 8 comprises heating the reagents at a temperature greater than room temperature. Typically, the reagents are heated to greater than 200° C., typically to around 240° C. This step may last for between 10 minutes and 2 hours, for example around 30 minutes. The step may also take place in the presence of further reagents, such a diphenylether.

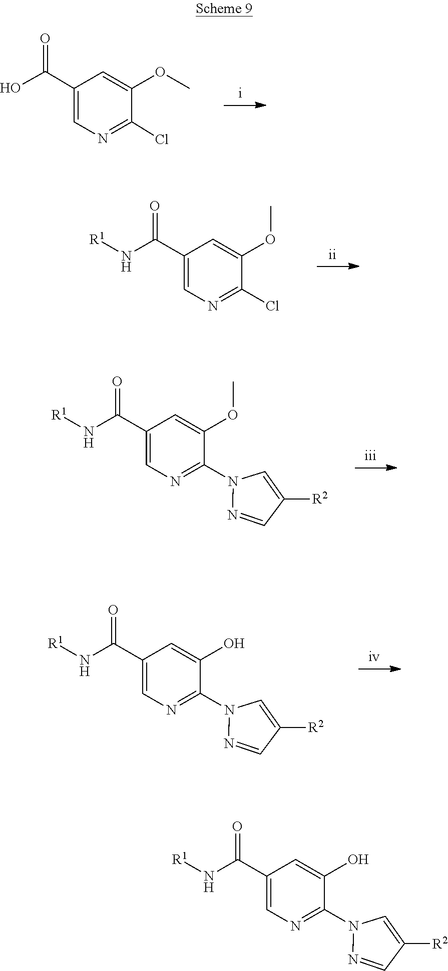

Scheme 9 above shows how the 3-hydroxypyridine structures of formula (Ib) may be produced using the general synthesis procedures set forth in the Examples section hereinbelow (see the General Procedures A and D in the Examples).

Step (i) of Scheme 9 typically comprises treatment with R—NH₂. Scheme 9 step (i) may comprise treatment with any appropriate amide coupling reagents. Typically, step (i) comprises treatment with an acid anhydride, for example propanephosphonic acid anhydride in the presence of a base such as N,N-diisopropylethylamine (DIPEA). The reaction typically occurs in a solvent. The solvent may be a polar solvent. Typically, the solvent may be a polar, aprotic solvent. The polar aprotic solvent may be dimethylacetamide (DMAc). Typically step (i) occurs at SATP. The step may last for between 1 and 24 hours, for example about 16 hours.

Step (ii) of Scheme 9 comprise treatment with further reagents such as pyrazole and substituted pyrazoles. Step (ii) of Scheme 9 comprises treatment in the presence of a catalyst. The catalyst may be a palladium catalyst. Typically, the catalyst may be Pd BuxPhos G3. The step may also take place in the presence of a base. The base may be a carbonate. Usually, the base is Cs₂CO₃. The reaction typically occurs in a solvent. The solvent may be a mixture of solvents. Typically, the solvent may be a mixture of one or more polar solvents, often polar aprotic solvents. Usually, the reaction occurs in tert butanol or 1,4-dioxane. Step (ii) typically occurs at a temperature greater than room temperature. For example, step (ii) typically occurs between 40° C. and 80° C. Usually, the step occurs at around 60° C. The step may last for between 1 hour to 48 hours, for example about 16 hours. Step (ii) may also comprise treatment with further reagents such as pyrazole and substituted pyrazoles.

Step (iii) of Scheme 9 is a deprotection step that may comprise treatment with any suitable reagents known to the skilled person. In some instances, lithium chloride is used. This step typically takes place in the presence of a solvent. The solvent is typically a polar solvent, and more typically a polar aprotic solvent. The solvent may be DMAc. Step (v) typically occurs at a temperature greater than room temperature. For example, step (v) typically occurs between 80° C. and 120° C. Usually, the step occurs at around 100° C. The step may last for between 30 minutes and 6 hours, for example about 2 hours.

Step (iv) of Scheme 9, if required, may be carried out using any suitable reagents for ester hydrolysis. Typically, the reaction may be carried out in the presence of a hydroxide compound, usually lithium hydroxide. The reaction is usually carried out in the presence of a solvent. The solvent may be a mixture of one or more solvents, typically one or more polar solvents. The one or more polar solvents may be protic or aprotic. For example, the solvent may be a mixture of THF and H₂O. Usually the mixture is 1:1 mixture. Typically step (ii) occurs at SATP. The step may last for between 1 and 24 hours, for example about 16 hours.

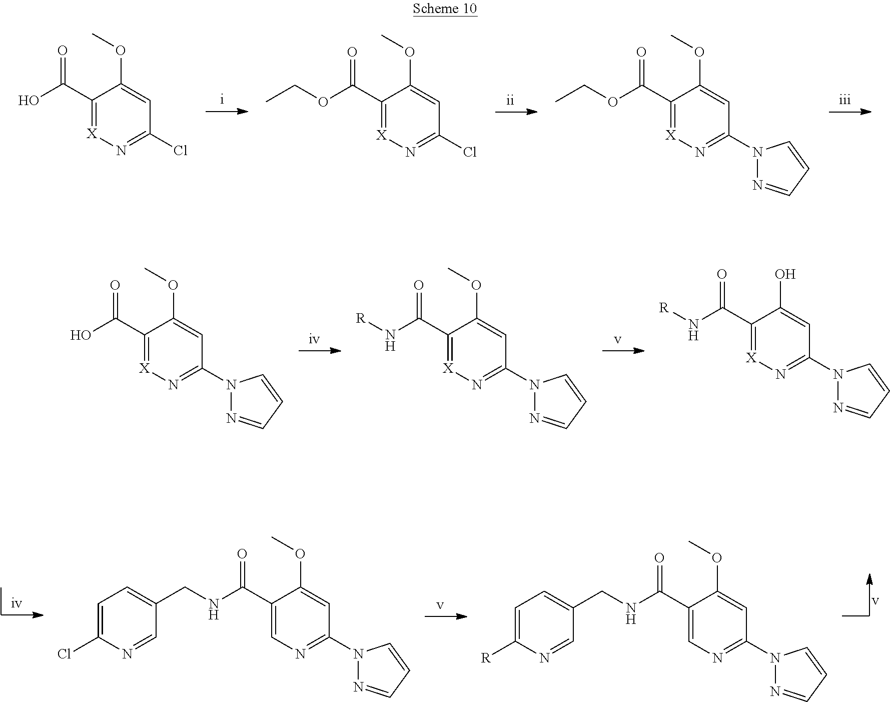

Scheme 10 above shows how the substituted azines of formula (Ia) may be produced using the general synthesis procedures set forth in the Examples section hereinbelow (see the General Procedures B, C and D in the Examples).

Step (i) of Scheme 10 may be carried out using any appropriate esterification reagents known to the skilled person. In some cases N′-ethylcarboiimide hydrochloride (EDC.HCl) is used. Typically, a catalyst is also present. Usually the catalyst present is an organocatalyst. The catalyst may be 4-dimthylaminopyridine (DMAP). A base may also be present in step (i) of Scheme 10. The base may be N,N-diisopropylethylamine (DIPEA). Typically step (i) of Scheme 10 takes place in the presence of a solvent. The solvent may be a mixture of two solvents. Typically, the solvent is a mixture of two polar solvents. Usually, the solvent is a mixture of a polar protic solvent and a polar aprotic solvent. Therefore, the solvent may be a mixture of dimethylformamide (DMF) and ethanol. Typically, step (i) occurs at SATP. This step may last for between 1 hour and 24 hours, for example around 16 hours.

In step (ii) of Scheme 10 the product of step (i) is treated with pyrazole. Step (ii) comprises treatment in the presence of a catalyst. The catalyst may be a palladium catalyst. Typically, the catalyst may be Pd^{t}BuxPhos G3. The step may also take place in the presence of a base. The base may be a carbonate. Usually, the base is Cs₂CO₃. Step (ii) typically occurs at a temperature greater than room temperature. For example, step (ii) typically occurs between 40° C. and 80° C. Usually, the step occurs at around 60° C. The step may last for between 1 hour to 24 hours, for example about 16 hours.

Step (iii) may be carried out using any suitable reagents for ester hydrolysis. Typically, the reaction may be carried out in the presence of a hydroxide, usually lithium hydroxide. The reaction is usually carried out in the presence of a solvent. The solvent may be a mixture of one or more solvents, typically one or more polar solvents. The one or more polar solvents may be protic or aprotic. For example, the solvent may be a mixture of THF and H₂O. Usually, the mixture is an approximately 1:1 mixture. Typically step (iii) occurs at SATP. The step may last for between 1 and 24 hours, for example about 16 hours.

In Scheme 10 step (iv) the product of step (iii) is treated with a group R—NH₂ . . . . For step (iv) any appropriate peptide coupling reagents may be used. Typically, Scheme 10 step (iv) comprises treatment with HATU in the presence of a base such as N, N-diisopropylethylamine (DIPEA). The reaction typically occurs in a solvent. The solvent may be a polar solvent. Typically, the solvent may be a polar, aprotic solvent. The polar aprotic solvent may be N,N-dimethylacetamide (DMAc). Typically step (iv) occurs at SATP. The step may last for between 1 and 24 hours, for example about 16 hours.

Step (v) of Scheme 10 is a deprotection step that may comprise treatment with any suitable reagents known to the skilled person. In some instances, lithium chloride is used. This step typically takes place in the presence of a solvent. The solvent is typically a polar solvent, and more typically a polar aprotic solvent. The solvent may be DMAc. Step (v) typically occurs at a temperature greater than room temperature. For example, step (v) typically occurs between 80° C. and 120° C. Usually, the step occurs at around 100° C. The step may last for between 30 minutes and 6 hours, for example about 2 hours.

Step (vi) of Scheme 10 comprises reaction in the presence of a catalyst. The catalyst may be a palladium catalyst. Typically, the catalyst may be PdAmPhos. The step may also take place in the presence of a base. The base may be a carbonate. Usually, the base is Cs₂CO₃. In step (vi) the product of step (iv) is usually treated with an organoborane compound comprising a group —R. Typically this compound is a compound of the formula RB(OH)₂ or R-B-pinacol ester. Step (vi) typically occurs at a temperature greater than room temperature. For example, step (vi) typically occurs between 80° C. and 120° C. Usually, the step occurs at around 100° C. The step may last for between 30 minutes to 6 hours, for example about 2 hours. This step typically takes place in the presence of a solvent. The solvent is typically a non-polar solvent, and more typically a non-polar aprotic solvent. The solvent may be 1,4-dioxane.

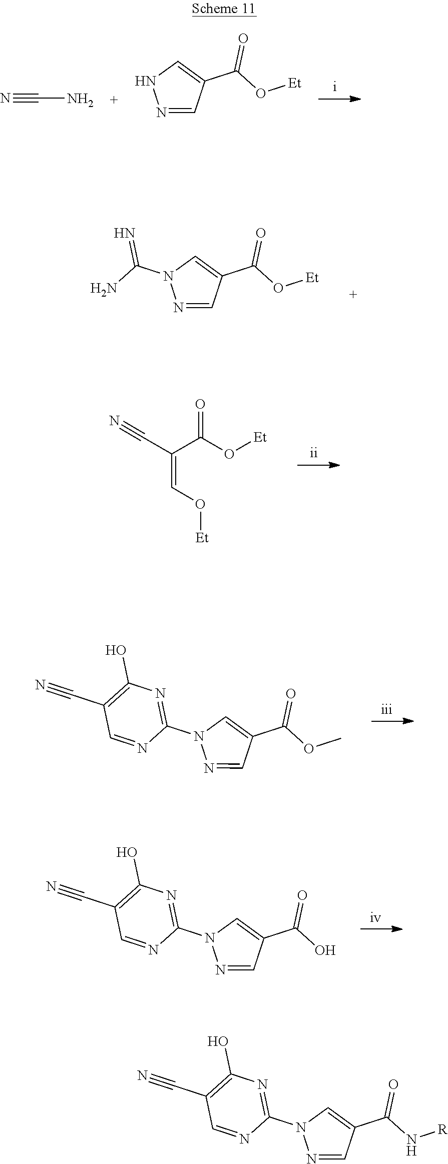

Scheme 11 above shows how the substituted pyrimidines of formula (IV) may be produced using the general synthesis procedure set forth in the Examples section hereinbelow (see the General Procedure B in the Examples).

Step (i) of Scheme 11 typically comprises treatment with an acid. Usually, the acid is a protic acid, such as HCl. For example, 4M HCl in 1,4-dioxane may be used. Step (i) typically occurs at a temperature greater than room temperature. For example, step (i) typically occurs between 80° C. and 120° C. Usually, the step occurs at around 100° C. The step may last for between 1 hour and 24 hours, for example about 16 hours.

Step (ii) of Scheme 11 typically comprises treatment in the presence of a base. Any suitable base may be used. Typically, the base is a carbonate. K₂CO₃ may be used. Step (ii) typically takes place in a solvent. The solvent may be a polar solvent, typically a polar protic solvent. Usually, the solvent is methanol. Typically step (ii) occurs at SATP. The step may last for between 1 and 24 hours, for example about 16 hours.

Step (iii) may be carried out using any suitable reagents for ester hydrolysis. Typically, the reaction may be carried out in the presence of a hydroxide base, usually lithium hydroxide. The reaction is usually carried out in the presence of a solvent. The solvent may be a mixture of one or more solvents, typically one or more polar solvents. The one or more polar solvents may both be protic. For example, the solvent may be a mixture of methanol and H₂O. Usually the mixture is 1:1 mixture. Typically step (iii) occurs at SATP. The step may last for between 1 and 24 hours, for example about 16 hours.

In Scheme 11 step (iv) the product of step (iii) is treated with a group R—NH₂ . . . . For step (iv) any appropriate peptide coupling reagents may be used. Typically, Scheme 11 step (iv) comprises treatment with T3P in the presence of a base such as N,N-diisopropylethylamine (DIPEA). The reaction typically occurs in a solvent. The solvent may be a polar solvent. Typically, the solvent may be a polar, aprotic solvent. The polar aprotic solvent may be N,N-dimethylacetamide (DMAc). Typically, step (iv) occurs at SATP. The step may last for between 1 and 24 hours, for example about 16 hours.

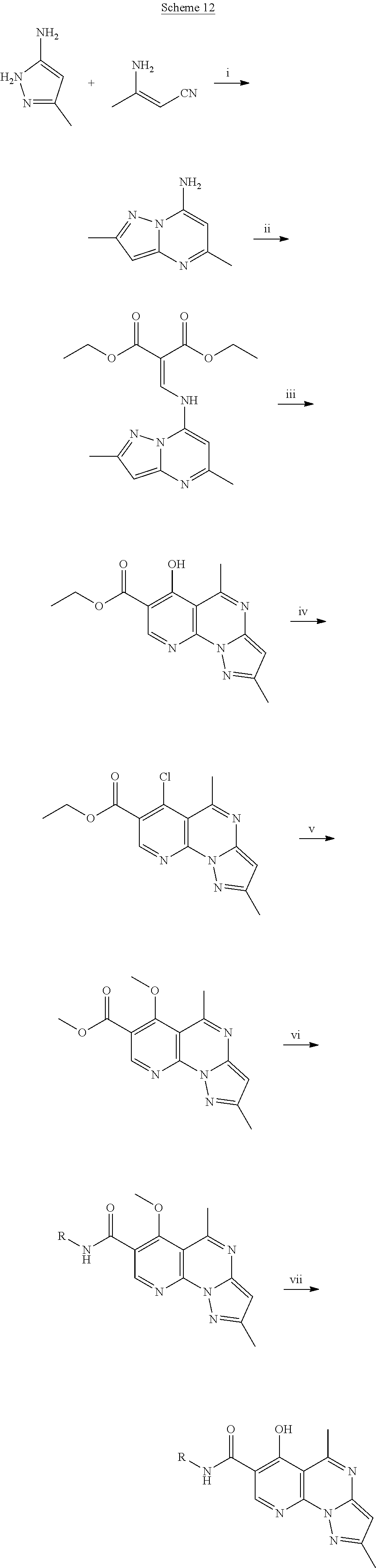

Scheme 12 above shows how the pyrazolo[1,5-a]pyrido[3,2-e]-pyrimidine-7-amido structures of formula (Id) may be produced using the general synthesis procedures set forth in the Examples section hereinbelow (see the General Procedures A and D in the Examples).

In Scheme 12, step (i) usually comprises heating together the starting materials. Typically heating occurs at a temperature of 100° C. to 200° C., more typically heating occurs at from 140° C. to 160° C. Heating may occur at about 150° C. The step may last for between 30 minutes and 6 hours, for example about 1 hour.

Step (ii) of Scheme 12 comprises heating the product of step (i) with diethyl ethoxymethylenemalonate. Step (ii) typically takes place in a solvent. The solvent may be a polar solvent or apolar protic solvent. Usually, the solvent is toluene. Typically heating occurs at a temperature of 80° C. to 160° C., more typically heating occurs at from 100° C. to 140° C. Heating may occur at about 120° C. Step (ii) typically lasts for about 24 hours to 72 hours. For example, step (ii) may last for around 48 hours.

Step (iii) of Scheme 12 typically comprises treating with a reagent to promote ring formation. Any appropriate reagent may be used. Typically, Eaton's reagent (10 wt % phosphorous pentoxide solution in methanesulfonic acid) is used. Step (iii) typically occurs at a temperature greater than room temperature. For example, step (iii) typically occurs between 50° C. and 90° C. Usually, the step occurs at around 70° C. The step may last for between 16 hours and 30 hours, for example about 24 hours.

Step (iv) of Scheme 12 typically comprises treatment with a chlorinating agent. Any appropriate chlorinating agent known to the skilled person may be used. Usually phosphoryl chloride (POCl₃) is used. Step (iv) typically occurs at a temperature greater than room temperature. For example, step (iv) typically occurs between 50° C. and 90° C. Usually, the step occurs at around 70° C. The step may last for between 1 hour and 6 hours, for example about 3 hours.

Step (v) of Scheme 12 typically comprises treatment with sodium methoxide (NaOMe). Step (v) typically takes place in a solvent. The solvent may be a polar solvent, typically a polar protic solvent. Usually, the solvent is methanol. Typically step (v) occurs at SATP. The step may last for between 1 and 10 hours, for example about 4 hours.

Step (vi) of Scheme 12 typically comprises treatment with R—NH₂ in the presence of an agent to promote amide formation. The agent may be an organoaluminium reagent, typically bis(trimethylaluminum)-1,4-diazabicyclo[2.2.2]octane adduct (DABAL-AlMe₃). Typically step (vi) takes place in the presence of a solvent. The solvent is usually a non-polar solvent, and often a non-polar aprotic solvent such as THF. Step (vi) typically occurs at a temperature greater than room temperature. For example, step (vi) typically occurs between 100° C. and 140° C. Usually, the step occurs at around 120° C. The step may last for between 30 minutes and 6 hours, for example about 3 hours.

Scheme 12 step (vii) can comprise treatment with any agent appropriate for converting an ether to a hydroxyl. Typically, lithium chloride can be used. The reaction typically occurs in a solvent. The solvent may be a polar solvent. Typically, the solvent may be a polar, aprotic solvent. The polar aprotic solvent may be N,N-dimethylacetamide (DMAc). Step (vii) typically occurs at a temperature greater than room temperature. For example, step (vii) typically occurs between 80° C. and 120° C. Usually, the step occurs at around 100° C. The step may last for between 30 minutes and 6 hours, for example about 2 hours.

Compounds of the invention containing one or more chiral centres may be used in enantiomerically or diastereoisomerically pure form, or in the form of a mixture of isomers. For the avoidance of doubt, the compounds of the invention can, if desired, be used in the form of solvates. Further, for the avoidance of doubt, the compounds of the invention may be used in any tautomeric form.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines. A substituted azine of formula (I), (Ia), (Ib), (Ic) or (Id), or a substituted pyrimidine of formula (IV), may be converted into a pharmaceutically acceptable salt, and salts may be converted into the free compound, by conventional methods.

### Pharmaceutical Compositions

Also provided by the invention is a pharmaceutical composition comprising a compound of the invention as defined anywhere herein, and a pharmaceutically acceptable carrier or diluent.

Thus, a pharmaceutical composition of the invention may comprise a compound of formula (I), (Ia), (Ib), (Ic) or (Id) as defined herein, or a compound of formula (IV) as defined herein, and a pharmaceutically acceptable carrier or diluent.

Typically, the composition contains up to 85 wt % of a compound of the invention. More typically, it contains up to 50 wt % of a compound of the invention. Preferred pharmaceutical compositions are sterile and pyrogen free. Further, when the pharmaceutical compositions provided by the invention contain a compound of the invention which is optically active, the compound of the invention is typically a substantially pure optical isomer.

The composition of the invention may be provided as a kit comprising instructions to enable the kit to be used as described herein or details regarding which subjects the composition may be used for.

The composition of the invention is typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

The composition of the invention may be formulated for inhaled (aerosolised) administration as a solution or suspension. The compound or combination of the invention may be administered by a metered dose inhaler (MDI) or a nebulizer such as an electronic or jet nebulizer. Alternatively, the compound or combination of the invention may be formulated for inhaled administration as a powdered drug, such formulations may be administered from a dry powder inhaler (DPI). When formulated for inhaled administration, the compound or combination of the invention may be delivered in the form of particles which have a mass median aerodynamic diameter (MMAD) of from 1 to 100 μm, preferably from 1 to 50 μm, more preferably from 1 to 20 μm such as from 3 to 10 μm, e.g. from 4 to 6 μm. When the compound or combination of the invention is delivered as a nebulized aerosol, the reference to particle diameters defines the MMAD of the droplets of the aerosol. The MMAD can be measured by any suitable technique such as laser diffraction.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections or inhalation may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for inhalation, injection or infusion may contain as carrier, for example, sterile water or preferably they may be in the form of sterile, aqueous, isotonic saline solutions. Pharmaceutical compositions suitable for delivery by needleless injection, for example, transdermally, may also be used.

The pharmaceutical composition of the invention may further comprise one or more additional active agents.

The pharmaceutical composition of the invention may further comprise one or more additional active agents. These may be one or more further biologically active agents which are useful in the treatment of blood cancer (including AML, CML and MM). The one or more further biologically active agents are typically a chemotherapeutic agent and may be selected from daunorubicin, doxorubicin, cyclophosphamide, vincristine, asparaginase, pegasparaginase, dexamethasone, prednisone, methotrexate, cytarabine, midostaurin, gemtuzumab ozogamicin, cladribine, venetoclax, and fludarabine. The one or more further biologically active agents may also be selected from all trans retinoic acid (ATRA) and arsenic trioxide. The pharmaceutical composition of the invention may further comprise two further biologically active agents for use in the treatment of blood cancer. For instance, the two further biologically active agents may be cytarabine and an anthracycline (such as daunorubicin or idarubicin). Typically, however, the pharmaceutical composition of the invention further comprises one further biologically active agent for use in the treatment of blood cancer, such as cytarabine or ventetoclax, preferably venetoclax. The pharmaceutical composition of the invention may further comprise a BCL-2 inhibitor. The BCL-2 inhibitor may for instance be venetoclax. A particularly preferred pharmaceutical composition of the invention comprises IOX5 (compound 68 herein), or a pharmaceutically acceptable salt thereof, and venetoclax, or a pharmaceutically acceptable salt thereof.

Additionally or alternatively, the pharmaceutical composition of the invention may further comprise one or more additional active agents selected from ACE inhibitors, angiotensin II receptor agonists, beta receptor blockers, calcium antagonists, PDE inhibitors, mineralocorticoid receptor antagonists, diuretics, aspirin, iron supplements, vitamin B12 and folic acid supplements, statins, digitalis (digoxin) derivatives, tumor chemotherapeutics and antibiotics.

### Therapeutic Uses

Compounds of the invention, of formula (I), (Ia), (Ib), (Ic), (Id) and (IV) as defined herein, have been shown have high efficacy and specificity as hypoxia inducible factor prolyl hydroxylase inhibitors (PHD inhibitors). For instance, some compounds of the invention have been shown to have IC₅₀ for PHD2 of less than 200 nM, which is a substantial improvement compared to known inhibitors (e.g. roxadustat has an IC₅₀ of 2.7 μM in the RF-LC PHD2 hydroxylation assay described in general experimental procedures). As well as their potency, compounds of the invention have been found to be highly selective for the PHDs, with greater than 100-fold selectivity compared to other 2OG oxygenases.

As well as these desirable biochemical properties, compounds of the invention have been shown to have desirable physical properties including good solubility and permeability in cells. These physical properties mean that compounds of the invention of have been found to increase cellular HIF-1α at concentrations in the nM range. The compounds of the invention, of formula (I), (Ia), (Ib), (Ic), (Id) and (IV) as defined herein, and the pharmaceutical compositions of the invention, therefore have potential utility in treating conditions for which HIF-PHD is a therapeutic target, including blood cancer, and in particular acute myeloid leukaemia (AML), chronic myeloid leukaemia (CML) and multiple myeloma (MM).

Accordingly, the invention provides a compound of the invention as defined herein, or a pharmaceutical composition of the invention as defined herein, for use in the treatment of the human or animal body by therapy. As dicussed hereinbefore, the terms “treatment”, “treat” and “treating” herein refer to both therapeutic treatment and prophylactic or preventative measures. The compound of the invention may be a compound of formula (I), (Ia), (Ib), (Ic), (Id) or (IV) as defined herein.

The invention also provides a compound of the invention as defined herein, or a pharmaceutical composition of the invention as defined herein, for use as a modulator of hypoxia inducible factor prolyl hydroxylase activity. The compound of the invention may be a compound of formula (I), (Ia), (Ib), (Ic), (Id) or (IV) as defined herein. Typically, the compound or pharmaceutical composition is for use as an inhibitor of hypoxia inducible factor prolyl hydroxylase activity. Thus, the invention also provides a compound of the invention as defined herein, or a pharmaceutical composition of the invention as defined herein, for use as a PHD inhibitor.

The invention also provides a compound of the invention as defined herein, or a pharmaceutical composition of the invention as defined herein, for use in treating a PHD-related disorder.

The term, “PHD-related disorder”, as used herein, means a disorder that can be treated by modulating hypoxia inducible factor prolyl hydroxylase activity. Typically, the PHD-related disorder is one that can be treated by inhibiting inducible factor prolyl hydroxylase activity.

The invention also provides a compound of the invention as defined herein, or a pharmaceutical composition of the invention as defined herein, for use in treating blood cancer. Typically, the blood cancer is leukaemia, for instance AML or CML. The blood cancer may for instance be acute myeloid leukaemia (AML). The blood cancer may be CML. The blood cancer may be MM.

A therapeutically effective amount of the compound of the invention is administered to a subject, the term “therapeutically effective amount” as used herein meaning a therapeutically or prophylactically effective amount. Similarly, a composition comprising a therapeutically effective amount of the compound of the invention may be administered to a subject. The dose may be determined according to various parameters, especially according to the compound used; the age, weight and condition of the subject to be treated; the route of administration; and the required regimen. Again, a physician will be able to determine the required route of administration and dosage for any particular subject. A typical daily dose is from about 0.01 to 100 mg per kg, preferably from about 0.1 mg/kg to 50 mg/kg, e.g. from about 1 to 10 mg/kg of body weight, according to the activity of the specific inhibitor, the age, weight and conditions of the subject to be treated, the type and severity of the disease and the frequency and route of administration. Preferably, daily dosage levels are from 1 mg to 2 g.

The subject is generally a mammal, and typically a human. However, it may be non-human. Preferred non-human animals include, but are not limited to, primates, such as marmosets or monkeys, commercially farmed animals, such as horses, cows, sheep or pigs, and pets, such as dogs, cats, mice, rats, guinea pigs, ferrets, gerbils or hamsters.

Typically a compound of the invention is for use in the treatment of blood cancer, for instance AML, CML or MM. Indeed, any PHD inhibitor as described herein may be for use in the treatment of blood cancer, for instance AML, CML and MM, in accordance with the present invention.

### Compounds for Use in Methods of Treatment

The Examples section herein provides both genetic and pharmacological evidence that PHD inhibition is a very promising therapeutic strategy for blood cancer.

The present invention also therefore provides a PHD inhibitor for use in the treatment of blood cancer, for instance AML, CML or MM. The PHD inhibitor may for instance be a PHD inhibitor as further defined anywhere herein. It may for instance be any of the known PHD inhibitors described herein, or it may be any of the compounds of the invention of formulae (I), (Ia), (Ib), (Ic), (Id) or (IV) as defined herein.

The invention also provides a method of treating a subject suffering from or susceptible to blood cancer, which method comprises administering to said subject an effective amount of a PHD inhibitor. The PHD inhibitor may be as further defined anywhere herein.

The invention additionally provides the use of a PHD inhibitor in the manufacture of a medicament for use in the treatment of blood cancer. The PHD inhibitor may be as further defined anywhere herein.

Therefore the invention also provides a PHD for use in a method of treating blood cancer. The PHD inhibitor may be as further defined anywhere herein.

In all of these aspects, the blood cancer may for instance be acute myeloid leukaemia (AML), chronic myeloid leukaemia (CML) or multiple myeloma (MM).

Preferably, the blood cancer is acute myeloid leukaemia (AML).

Preferably, the blood cancer is chronic myeloid leukaemia (CML).

Preferably, the blood cancer is multiple myeloma (MM).

The method typically comprises administering to said patient an effective amout of the PHD inhibitor.

The treatment generally involves the PHD inhibitor binding to the active site of hypoxia inducible factor prolyl hydroxylase (PHD). Typically, the PHD inhibitor competes with 2-oxoglutarate for binding to said active site. The PHD inhibitor may or may not compete with HIF-alpha for binding to said active site.

The method may comprise subsequent, sequential or simultaneous administration of the PHD inhibitor with comprise one or more additional active agents.

The one or more additional active agents may be one or more further biologically active agents which are useful in the treatment of blood cancer. The one or more additional active agents may for instance include a BCL-2 inhibitor, such as for example venetoclax. The one or more additional active agents may for instance include cytarabine or venetoclax, and preferably include venetoclax. Thus, the PHD inhibitor may be IOX5 (compound 68 herein), or a pharmaceutically acceptable salt thereof, and the one or more additional active agents may comprise venetoclax.

The one or more further biologically active agents often include a chemotherapeutic agent and thus may include an agent selected from daunorubicin, doxorubicin, cyclophosphamide, vincristine, asparaginase, pegasparaginase, dexamethasone, prednisone, methotrexate, cytarabine, midostaurin, gemtuzumab ozogamicin, cladribine, venetoclax, and fludarabine. The one or more further biologically active agents may also be selected from all trans retinoic acid (ATRA) and arsenic trioxide.

The one or more additional active agents may comprise two further biologically active agents for use in the treatment of blood cancer. For instance, the two further biologically active agents may be cytarabine and an anthracycline (such as daunorubicin or idarubicin). Typically, however, the one or more additional active agents comprise one further biologically active agent for use in the treatment of blood cancer, such as cytarabine or venetoclax, preferably venetoclax. The one or more additional active agents may for instance comprise: (a) a factor inhibiting HIF inhibitor (FIH inhibitor), for instance dimethyl N-oxalyl-D-phenylalanine (DM-NOFD) or a pharmaceutically acceptable salt thereof; and (b) a BCL-2 inhibitor, for instance venetoclax.

Alternatively, the one or more additional active agents may be selected from ACE inhibitors, angiotensin II receptor agonists, beta receptor blockers, calcium antagonists, PDE inhibitors, mineralocorticoid receptor antagonists, diuretics, aspirin, iron supplements, vitamin B12 and folic acid supplements, statins, digitalis (digoxin) derivatives, tumor chemotherapeutics and antibiotics.

The method may also comprise administration of the PHD inhibitor before, during or after non-therapeutic blood cancer treatment. For example, before, during or after leukapheresis or blood transfusion.

Evidence for the efficacy of combination therapies which employ a PHD inhibitor in combination with factor inhibiting HIF inhibitor (FIH inhibitor) and/or a B-cell lymphoma 2 (BCL2) inhibitor is provided in the Examples and Figures herein.

The invention therefore provides a hypoxia inducible factor prolyl hydroxylase inhibitor (PHD inhibitor) for use in the treatment of blood cancer by simultaneous, separate or sequential co-administration with a factor inhibiting HIF inhibitor (FIH inhibitor). Often the blood cancer is acute myeloid leukaemia (AML), chronic myeloid leukaemia (CML) or multiple myeloma (MM).

The PHD inhibitor may be a PHD inhibitor as defined anywhere herein. For example, the PHD inhibitor may be a compound of formula (I), (II), (III) or (IV) as defined anywhere herein.

Often the PHD inhibitor is a compound of formula (I) as defined anywhere herein. Typically, the PHD inhibitor is a compound of formula (Ia) as defined anywhere herein.

Often the PHD inhibitor is a compound of the following formula or a pharmaceutically acceptable salt thereof

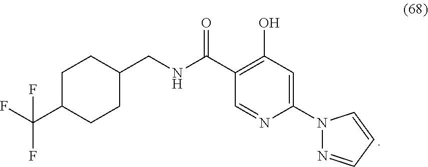 [SMILES: O=C(NCC1CCC(C(F)(F)F)CC1)c1cnc(-n2cccn2)cc1O]

For a PHD inhibitor for use in the treatment of blood cancer by simultaneous, separate or sequential co-administration with a factor inhibiting HIF inhibitor (FIH inhibitor), the FIH inhibitor may be any known FIH inhibitor, and it may for instance be any of the FIH inhibitors described in Corner et al., Chem. Sci., 2023, 14, 12098-12120. The FIH inhibitor is often dimethyl N-oxalyl-D-phenylalanine (DM-NOFD) (which is described in Corner et al., Chem. Sci., 2023, 14, 12098-12120) or a pharmaceutically acceptable salt thereof.

Often, for the PHD inhibitor for use in the treatment of blood cancer by simultaneous, separate or sequential co-administration with a factor inhibiting HIF inhibitor (FIH inhibitor), the treatment of blood cancer further comprises administration of a B-cell lymphoma 2 (BCL2) inhibitor. The BCL2 inhibitor may be venetoclax, or a pharmaceutically acceptable salt thereof.

The present invention also provides a factor inhibiting HIF inhibitor (FIH inhibitor) for use in the treatment of blood cancer by simultaneous, separate or sequential co-administration with a hypoxia inducible factor prolyl hydroxylase inhibitor (PHD inhibitor). The FIH inhibitor for use may be dimethyl N-oxalyl-D-phenylalanine (DM-NOFD) or a pharmaceutically acceptable salt thereof.

For the FIH inhibitor for use in the treatment of blood cancer by simultaneous, separate or sequential co-administration with a hypoxia inducible factor prolyl hydroxylase inhibitor (PHD inhibitor), the PHD inhibitor may be a PHD inhibitor as defined anywhere herein. For example, the PHD inhibitor may be a compound of formula (I), (II), (III) or (IV) as defined anywhere herein.

Often the PHD inhibitor is a compound of formula (I) as defined anywhere herein. Typically, the PHD inhibitor is a compound of formula (Ia) as defined anywhere herein.

Often the PHD inhibitor is a compound of the following formula or a pharmaceutically acceptable salt thereof

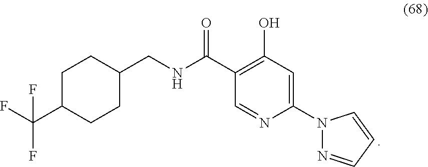 [SMILES: O=C(NCC1CCC(C(F)(F)F)CC1)c1cnc(-n2cccn2)cc1O]

For the FIH inhibitor for use in the treatment of blood cancer by simultaneous, separate or sequential co-administration with a hypoxia inducible factor prolyl hydroxylase inhibitor (PHD inhibitor), the treatment of blood cancer may further comprise administration of a BCL2 inhibitor. The BCL2 inhibitor may be venetoclax, or a pharmaceutically acceptable salt thereof. Often the blood cancer is is acute myeloid leukaemia (AML), chronic myeloid leukaemia (CML) or multiple myeloma (MM).

The invention further provides a method of treatment of a blood cancer, which method comprises administering to a subject in need of such treatment an effective amount of a hypoxia inducible factor prolyl hydroxylase inhibitor (PHD inhibitor) and an effective amount of a factor inhibiting HIF inhibitor (FIH inhibitor). The method may further comprise administering to a subject in need of such treatment an effective amount of a BCL2 inhibitor, optionally wherein the BCL2 inhibitor is venetoclax or a pharmaceutically acceptable salt thereof. Often the blood cancer is is acute myeloid leukaemia (AML), chronic myeloid leukaemia (CML) or multiple myeloma (MM).

The invention also provides a combination comprising a hypoxia inducible factor prolyl hydroxylase inhibitor (PHD inhibitor) and a factor inhibiting HIF inhibitor (FIH inhibitor). In this combination the PHD inhibitor may be a PHD inhibitor as defined anywhere herein. For example, the PHD inhibitor may be a compound of formula (I), (II), (III) or (IV) as defined anywhere herein, or a pharmaceutically acceptable salt thereof. The combination may comprise two separate pharmaceutical compositions, one comprising the PHD inhibitor, and another comprising the FIH inhibitor. Alternatively, it may be a single pharmaceutical composition comprising both inhibitors. Any pharmaceutical composition will typically further comprise a pharmaceutically acceptable carrier or diluent. The combination may be a kit comprising the PHD inhibitor and the FIH inhibitor. The kit may further comprise instuctions for using the inhibitors therein (for instance, the PHD inhibitor and the FIH inhibitor) in combination to treat blood cancer, for instance acute myeloid leukaemia (AML), chronic myeloid leukaemia (CML) or multiple myeloma (MM).

Often the PHD inhibitor is a compound of formula (I) as defined anywhere herein, or a pharmaceutically acceptable salt thereof. Typically, the PHD inhibitor is a compound of formula (Ia) as defined anywhere herein or a pharmaceutically acceptable salt thereof.

Often the PHD inhibitor is a compound of the following formula or a pharmaceutically acceptable salt thereof

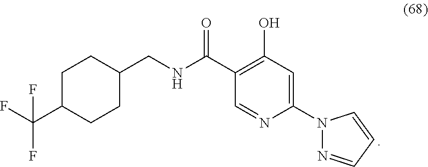 [SMILES: O=C(NCC1CCC(C(F)(F)F)CC1)c1cnc(-n2cccn2)cc1O]

Furthermore, the FIH inhibitor may be dimethyl N-oxalyl-D-phenylalanine (DM-NOFD) or a pharmaceutically acceptable salt thereof.

Said combination often further comprises a BCL2 inhibitor. The BCL2 inhibitor may be venetoclax or a pharmaceutically acceptable salt thereof.

The combination comprising a hypoxia inducible factor prolyl hydroxylase inhibitor (PHD inhibitor) and a factor inhibiting HIF inhibitor (FIH inhibitor) as described herein may be for use in the treatment of blood cancer. Often the blood cancer is acute myeloid leukaemia (AML), chronic myeloid leukaemia (CML) or multiple myeloma (MM).

The invention also provides a pharmaceutical composition comprising a hypoxia inducible factor prolyl hydroxylase inhibitor (PHD inhibitor), a factor inhibiting HIF inhibitor (FIH inhibitor), and a pharmaceutically acceptable carrier or diluent.

In said pharmaceutical composition the PHD inhibitor may be a PHD inhibitor as defined anywhere herein. For example, the PHD inhibitor may be a compound of formula (I), (II), (III) or (IV) as defined anywhere herein or a pharmaceutically acceptable salt thereof.

Often the PHD inhibitor is a compound of formula (I) as defined anywhere herein or a pharmaceutically acceptable salt thereof. Typically, the PHD inhibitor is a compound of formula (Ia) as defined anywhere herein or a pharmaceutically acceptable salt thereof.

Often the PHD inhibitor is a compound of the following formula or a pharmaceutically acceptable salt thereof

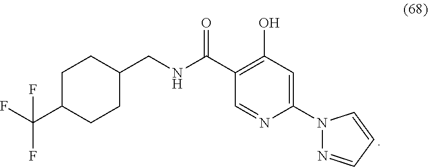 [SMILES: O=C(NCC1CCC(C(F)(F)F)CC1)c1cnc(-n2cccn2)cc1O]

Furthermore, the FIH inhibitor may be dimethyl N-oxalyl-D-phenylalanine (DM-NOFD) or a pharmaceutically acceptable salt thereof.

Said pharmaceutical composition often further comprises a BCL2 inhibitor. The BCL2 inhibitor may be venetoclax or a pharmaceutically acceptable salt thereof.

The invention pharmaceutical composition comprising a hypoxia inducible factor prolyl hydroxylase inhibitor (PHD inhibitor), a factor inhibiting HIF inhibitor (FIH inhibitor), and a pharmaceutically acceptable carrier or diluent as described herein may be for use in the treatment of blood cancer. Often the blood cancer is acute myeloid leukaemia (AML), chronic myeloid leukaemia (CML) or multiple myeloma (MM).

The invention also provides a hypoxia inducible factor prolyl hydroxylase inhibitor (PHD inhibitor) for use in the treatment of blood cancer by simultaneous, separate or sequential co-administration with a BCL2 inhibitor. Often the blood cancer is acute myeloid leukaemia (AML), chronic myeloid leukaemia (CML) or multiple myeloma (MM).

The PHD inhibitor may be a PHD inhibitor as defined anywhere herein. For example, the PHD inhibitor may be a compound of formula (I), (II), (III) or (IV) as defined anywhere herein or a pharmaceutically acceptable salt thereof.

Often the PHD inhibitor is a compound of formula (I) as defined anywhere herein or a pharmaceutically acceptable salt thereof. Typically, the PHD inhibitor is a compound of formula (Ia) as defined anywhere herein or a pharmaceutically acceptable salt thereof.

Often the PHD inhibitor is a compound of the following formula or a pharmaceutically acceptable salt thereof

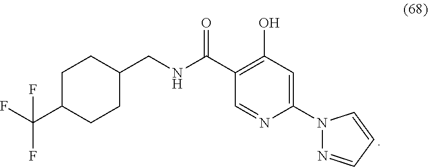 [SMILES: O=C(NCC1CCC(C(F)(F)F)CC1)c1cnc(-n2cccn2)cc1O]

For the PHD inhibitor for use in the treatment of blood cancer by simultaneous, separate or sequential co-administration with a BCL2 inhibitor the BCL2 inhibitor is often venetoclax. Often the blood cancer is acute myeloid leukaemia (AML), chronic myeloid leukaemia (CML) or multiple myeloma (MM).

The invention also provides a BCL2 inhibitor for use in the treatment of blood cancer by simultaneous, separate or sequential co-administration with a hypoxia inducible factor prolyl hydroxylase inhibitor (PHD inhibitor). Often the blood cancer is acute myeloid leukaemia (AML), chronic myeloid leukaemia (CML) or multiple myeloma (MM).

The PHD inhibitor may be a PHD inhibitor as defined anywhere herein. For example, the PHD inhibitor may be a compound of formula (I), (II), (III) or (IV) as defined anywhere herein or a pharmaceutically acceptable salt thereof.

Often the PHD inhibitor is a compound of formula (I) as defined anywhere herein or a pharmaceutically acceptable salt thereof. Typically, the PHD inhibitor is a compound of formula (Ia) as defined anywhere herein or a pharmaceutically acceptable salt thereof.

Often the PHD inhibitor is a compound of the following formula or a pharmaceutically acceptable salt thereof

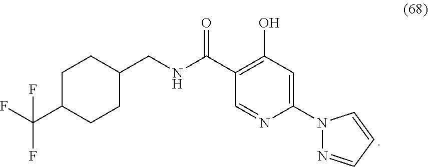 [SMILES: O=C(NCC1CCC(C(F)(F)F)CC1)c1cnc(-n2cccn2)cc1O]

Often, the BCL2 inhibitor is venetoclax or a pharmaceutically acceptable salt thereof.

The invention further provides a method of treatment of a blood cancer, which method comprises administering to a subject in need of such treatment an effective amount of a hypoxia inducible factor prolyl hydroxylase inhibitor (PHD inhibitor) and an effective amount of a BCL2 inhibitor. The BCL2 inhibitor is often venetoclax or a pharmaceutically acceptable salt thereof. Often the blood cancer is acute myeloid leukaemia (AML), chronic myeloid leukaemia (CML) or multiple myeloma (MM).

The invention provides a combination comprising a hypoxia inducible factor prolyl hydroxylase inhibitor (PHD inhibitor) and a BCL2 inhibitor. The combination may comprise two separate pharmaceutical compositions, one comprising the PHD inhibitor, and another comprising the BCL2 inhibitor. Alternatively, it may be a single pharmaceutical composition comprising both inhibitors. Any pharmaceutical composition will typically further comprise a pharmaceutically acceptable carrier or diluent. The combination may be a kit comprising the PHD inhibitor and the BCL2 inhibitor. The kit may further comprise instuctions for using the inhibitors therein (for instance, the PHD inhibitor and the BCL2 inhibitor) in combination to treat blood cancer, for instance acute myeloid leukaemia (AML), chronic myeloid leukaemia (CML) or multiple myeloma (MM).

The PHD inhibitor may be a PHD inhibitor as defined anywhere herein. For example, the PHD inhibitor may be a compound of formula (I), (II), (III) or (IV) as defined anywhere herein or a pharmaceutically acceptable salt thereof.

Often the PHD inhibitor is a compound of formula (I) as defined anywhere herein or a pharmaceutically acceptable salt thereof. Typically, the PHD inhibitor is a compound of formula (Ia) as defined anywhere herein or a pharmaceutically acceptable salt thereof.

Often the PHD inhibitor is a compound of the following formula or a pharmaceutically acceptable salt thereof

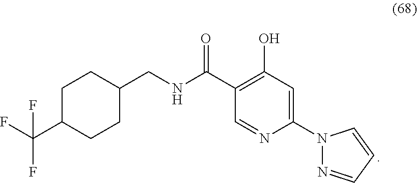 [SMILES: O=C(NCC1CCC(C(F)(F)F)CC1)c1cnc(-n2cccn2)cc1O]

The BCL2 inhibitor is often venetoclax or a pharmaceutically acceptable salt thereof.

A combination comprising a hypoxia inducible factor prolyl hydroxylase inhibitor (PHD inhibitor) and a BCL2 inhibitor may be for use in the treatment of blood cancer. Often the blood cancer is acute myeloid leukaemia (AML), chronic myeloid leukaemia (CML) or multiple myeloma (MM).

The invention further provides a pharmaceutical composition comprising a hypoxia inducible factor prolyl hydroxylase inhibitor (PHD inhibitor), a BCL2 inhibitor, and a pharmaceutically acceptable carrier or diluent.

The PHD inhibitor may be a PHD inhibitor as defined anywhere herein. For example, the PHD inhibitor may be a compound of formula (I), (II), (III) or (IV) as defined anywhere herein.

Often the PHD inhibitor is a compound of formula (I) as defined anywhere herein. Typically, the PHD inhibitor is a compound of formula (Ia) as defined anywhere herein.

Often the PHD inhibitor is a compound of the following formula or a pharmaceutically acceptable salt thereof

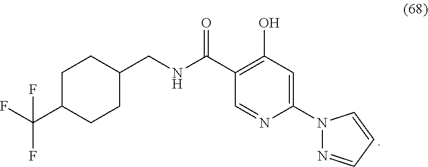 [SMILES: O=C(NCC1CCC(C(F)(F)F)CC1)c1cnc(-n2cccn2)cc1O]

The BCL2 inhibitor is often venetoclax or a pharmaceutically acceptable salt thereof.

The pharmaceutical composition comprising a hypoxia inducible factor prolyl hydroxylase inhibitor (PHD inhibitor), a BCL2 inhibitor, and a pharmaceutically acceptable carrier or diluent may be for use in the treatment of blood cancer.

Often the blood cancer is acute myeloid leukaemia (AML), chronic myeloid leukaemia (CML) or multiple myeloma (MM).

### Increase In HIF

As described in the Examples section herein, given that the primary function of PHD2 is to promote hypoxia inducible factor-1a (HIF-1a) degradation, the inventors measured HIF-1α levels following the treatment of AML cells with PHD inhibitors, and found increased HIF-1α levels in the treated cells. They went on to find that a PHD inhibitor did not compromise the proliferation of AML cells lacking both HIF-1α and HIF-2α and but did compromise the proliferation of control AML cells, implying that PHD inhibition exhibits its anti-leukaemic effect in a HIF-dependent manner. These findings show that the increase of hypoxia inducible factor (HIF) is a promising clinical pathway to the treatment of blood cancer.

The present invention therefore provides a method of treating blood cancer by increasing HIF. One method of increasing HIF in order to treat blood cancer is to employ a PHD inhibitor, but any other method of increasing HIF will also be applicable.

Preferably, the HIF is HIF-alpha (HIF-α). Thus, in a preferred embodiment, the invention provides a method of treating blood cancer by increasing HIF-α.

Thus the invention provides a method of treating a subject suffering from or susceptible to blood cancer, which method comprises increasing HIF in said subject. Preferably, the HIF is HIF-α.

Typically the blood cancer is AML, CML or MM.

In one embodiment, HIF is increased by administering to the subject an effective amount of a HIF increasing agent. The HIF increasing agent may for instance be a PHD inhibitor, which may be a PHD inhibitor as further defined anywhere herein, or a compound of the invention as defined anywhere herein. Alternatively, as will be explained below, HIF may be increased by any other suitable method. For instance HIF may be increased by administering a double stranded RNA, a small interfering RNA, or by using CRISPR and guide RNA, a zinc finger protein, a transcription activator-like effector nuclease, a designer receptor exclusively activated by designer drugs, an amino acid, a signalling molecule, a peptide, a protein, an antibody, a nucleic acid, an oligonucleotide, or a cell.

The invention also provides a HIF increasing agent for use in treating blood cancer. Typically, the blood cancer is AML, CML or MM.

The increase of HIF can comprise treatment with any PHD inhibitor as described herein. The increase of HIF may also comprise treatment with any agent suitable for the Increase of HIF including small molecule drugs and biologics as defined herein.

Increase of HIF can also occur by dimishing or silencing the gene associated with a protein that, when inhibited, increasing HIF. For instance genes related to prolyl hydroxylase or BNIP3 may be targeted to increased HIF. Increase of HIF may for instance be achieved by dimishing or silencing the gene associated with a PHD, for instance PHD1, PHD2 or PHD3.

The term silencing, as used herein, encompasses diminishing, inhibition or downregulation of gene expression; diminishing, inhibition or downregulation of transcription; diminishing, inhibition or downregulation of translation; and/or diminishing, inhibition or downregulation of protein activity. The diminishing, inhibition or downregulation can be direct, or indirect. Methods of determining the level of diminishing, inhibition or downregulation of gene expression; diminishing, inhibition or downregulation of transcription; diminishing, inhibition or downregulation of translation; and/or diminishing inhibition or downregulation of protein activity are known to the skilled person. Examples include in situ hybridisation to determine gene expression, immunoblotting to determine protein expression and electrophysiology to determine protein activity. The diminishing, inhibition or downregulation can be complete or partial.

Silencing can occur via any appropriate method known to the skilled person. Typically, silencing may occur using double stranded RNA, small interfering RNA, CRISPR and guide RNAs, zinc finger proteins, transcription activator-like effector nucleases (TALENs), designer receptor exclusively activated by designer drugs (DREADDs).

Silencing may also occur using antibody, or antigen binding proteins.

Genes which may be targeted to upregulate HIF could be genes related to prolyl hydroxylase, or BNIP3, particularly prolyl hydroxylase.

A HIF increasing agent for use in treating blood cancer may therefore be a PHD inhibitor as defined anywhere herein, a double stranded RNA, a small interfering RNA, CRISPR and guide RNA, a zinc finger protein, a TALEN, a DREADD, an amino acid, a signalling molecule, a peptide, a protein, an antibody, a nucleic acid, an oligonucleotide, or a cell.

The invention also provides a method for the treatment of blood cancer. Often said method comprises administering an effective amount of a HIF increasing agent to a subject in need thereof. Said HIF increasing agent may be a PHD inhibitor as defined anywhere herein, a double stranded RNA, a small interfering RNA, CRISPR and guide RNA, a zinc finger protein, a TALEN, a DREADD, an amino acid, a signalling molecule, a peptide, a protein, an antibody, a nucleic acid, an oligonucleotide, or a cell.

The invention also provides the use of a HIF increasing agent in the manufacture of a medicament for use in treating blood cancer.

The invention will be further described in the Examples and Reference Examples which follow:

### EXAMPLES

### Synthetic Examples

### General Procedures

All reactions involving moisture-sensitive reagents were carried out under a nitrogen atmosphere using standard vacuum line techniques. Glassware was oven dried and cooled under nitrogen before use. Commercial anhydrous solvents used in reactions and HPLC grade solvents were employed for work-up and chromatography. Aqueous solutions were made using de-ionized water, purified using an Elix UV-10 system. Thin layer chromatography (TLC) was carried out using Merck (Darmstadt, Germany) silica gel 60 F254 TLC plates. TLC visualization was carried out under UV light and stained with one of three stains; ninhydrin, potassium permanganate, or anisaldehyde. Chromatography was carried out using a Biotage® (Uppsala, Sweden) Isolera One or Biotage® SP4 flash purification system, using Biotage® pre-packed SNAP columns. Reactions were monitored using an Agilent (Cheshire, UK)₁₂₀₀ series, 6120 quadrupole LC-MS system using a Merck Chromolith® Performance RP-18 HPLC column. Deuterated solvents were from Sigma-Aldrich, and ¹H NMR spectra were obtained using Bruker AVANCE AVIII HD 400 nanobay (400 MHZ) machine or a machine Bruker AV500 (500M Hz) with a ¹³C cryoprobe. All signals are described in 8 ppm with multiplets being denoted as singlet, doublet, triplet, quartet, and multiplet using the abbreviations s, d, t, q, and m, respectively. Chemical shifts in presented NMR spectra were referenced using residual solvent peaks with coupling constants, J, reported in hertz (Hz) to an accuracy of 0.5 Hz. For high-resolution mass spectrometry (HR-MS), a Bruker MicroTOF instrument with an ESI source and Time of Flight (TOF) analyzer was used. MS data are represented as a ratio of mass to charge (m/z) in Daltons. A Bruker Tensor 27 instrument was used to obtain Fourier transform infrared spectra (FT-IR). Spectroscopic grade solvents and a Perkin Elmer 241 Polarimeter were used to obtain optical rotations.

All chemicals, reagents, and solvents were obtained from Sigma-Aldrich (Dorset, UK) and used without further purification. HPLC grade solvents were used for reactions, chromatography, and work-ups.

### General Procedure A

Ethyl ester amide coupling: the relevant ethyl ester (1 equivs), the relevant amine (1 equivs) and DABACO-(AlMe₃)₂ (1.0 equivs) were added; the microwave vial was flushed with N₂ which was removed in vacuo (3 times) before the addition of anhydrous THF. The reaction mixture was then heated at 130° C. for 8 minutes with biotage microwave irradiation (unless stated differently). The reaction mixture was diluted with a mixture of CH₃Cl: IPA (3:1, 20 ml), followed by the addition of KNaC₄H₄O₆·4H₂O_{aq} (50 ml). The resultant mixture was stirred for 1 hr. The phases were then separated, the organic phase was washed with water, brine and dried over Na₂SO₄. The solvent was removed in vacuo. The crude compound was purified by flash column chromatography using (conditions stated per reaction) over 20 column volumes to give the desired compound.

### General Procedure B

Amide coupling: The carboxylic acid (lequiv) and DIPEA (2.5 equiv) were dissolved in DMF. T3P (1.5 equiv, 50% in DMF) or HATU (2 equiv) were then added. The resultant reaction mixture was stirred at room temperature for 30 mins before the addition of the amine (1.2 equiv). The resultant mixture was stirred overnight at room temperature. EtOAc (20 ml) and H₂O (100 ml) was added to the reaction mixture. The organic and aqueous fractions were separated. The aqueous layer was extracted with EtOAc (30 ml) twice more. The organic fractions were combined before washing with brine and drying with anhydrous Na₂SO₄. The crude compound was then purified using flash column chromatography using (cyclohexane 100%-50%, EtOAc 0%-50%) over 20 column volumes to give the desired compound.

### General Procedure C

Pd-catalysed amination: The aryl halide (1 equiv), amine (1.2 equiv), Cs₂CO₃ (2 equiv), Pd-ligand conjugate (0.1 equiv) were put under N₂ before the addition of tert-butanol. The resultant mixture was heated at 80° C. for 16 hr. The reaction mixture was then allowed to cool to room temperature. EtOAc (20 ml) and H₂O (100 ml) was added to the reaction mixture. The organic and aqueous fractions were separated. The aqueous layer was extracted with EtOAc (30 ml) twice more. The organic fractions were combined, then washed with brine and dried using anhydrous Na₂SO₄. The crude compound was then purified using flash column chromatography using (0-100% in EtOAc in cyclohexane) over 20 column volumes to give the desired compound.

### General Procedure D

C-4 Methoxy Demethylation: The methoxy starting material (1 equiv) was dissolved in DMAc (0.1 M); LiCl·H₂O (10 equivs) was then added. The resultant mixture was heated with microwave irradiation at 100° C. for 2 hrs (unless stated). The resultant mixture was diluted with water (100 ml) and extracted with EtOAc (3×20 ml). The organic phases were combined and washed with water, brine and dried with anhydrous Na₂SO₄. The volatiles were then evaporated in vacuo and purified by flash column chromatography using (100%-95% CH₂Cl₂, 0%-20% MeOH) over 15 column volumes (unless otherwise stated) to give the desired compound.

### Reference Example 1—Synthesis of N-([1,1′-Biphenyl]-4-ylmethyl)-4-chloro-2-methoxybenzamide (14)

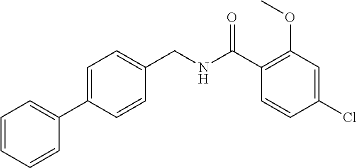 [SMILES: COc1cc(Cl)ccc1C(=O)NCc1ccc(-c2ccccc2)cc1]

Following general procedure B: 4-Chloro-2-methoxybenzoic acid (250 mg, 1.34 mmol), 4-phenylbenzylamine (294 mg, 1.6 mmol), T3P (1.06 g, 3.36 mmol), DIPEA (412 mg, 3.36 mmol) gave 14 (451 mg, 1.24 mmol, 95%).

¹H NMR (400 MHZ, Chloroform-d) δ 8.21 (d, J=8.5 Hz, 1H), 8.12 (t, J=6.0 Hz, 1H), 7.60-7.56 (m, 4H), 7.46-7.41 (m, 4H), 7.37-7.30 (m, 1H), 7.08 (dd, J=8.5, 2.0 Hz, 1H), 6.97 (d, J=2.0 Hz, 1H), 4.71 (d, J=6.0 Hz, 2H), 3.93 (s, 3H). HRMS (ESI-TOF) calcd for C₂₁H₁₉O₂N₃₅Cl [M+H]⁺: 352.1098, found: 352.1098.

### Reference Example 2—Synthesis of N-([1,1′-biphenyl]-4-ylmethyl)-2-methoxy-4-(1H-pyrazol-1-yl)benzamide (15)

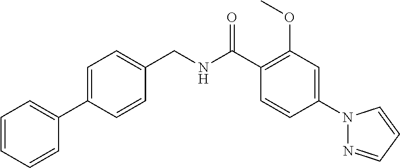 [SMILES: COc1cc(-n2cccn2)ccc1C(=O)NCc1ccc(-c2ccccc2)cc1]

Following general procedure C: 14 (100 mg, 0.284 mmol), Pd^{t}BuXPhos G3 (20 mg, 0.028 mmol), Cs₂CO₃ (185 mg, 0.568 mmol), pyrazole (23 mg, 0.34 mmol) gave 15 (28 mg, 0.073 mmol, 26%).

¹H NMR (400 MHZ, DMSO-d₆) δ 8.77 (t, J=6.0 Hz, 1H), 8.66 (d, J=2.5 Hz, 1H), 7.80 (d, J=1.5 Hz, 1H), 7.69-7.32 (m, 12H), 6.60 (dd, J=2.5, 1.5 Hz, 1H), 4.56 (d, J=6.0 Hz, 2H), 4.01 (s, 3H)

HRMS (ESI-TOF) calcd for C₂₄H₂₂O₂N₃ [M+H]⁺: 384.1704, found: 384.1704.

### Reference Example 3—Synthesis of N-([1,1′-biphenyl]-4-ylmethyl)-2-hydroxy-4-(1H-pyrazol-1-yl)benzamide (16)

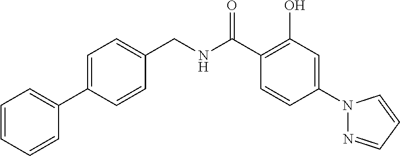 [SMILES: O=C(NCc1ccc(-c2ccccc2)cc1)c1ccc(-n2cccn2)cc1O]

TMS-I (39 mg, 0.195 mmol) was added to a solution of 15 (25 mg, 0.0652 mmol) and CH₂Cl₂ (2.5 ml). The resultant mixture was refluxed at 90° C. for 8 hrs, then cooled to rt, HCl_{aq} (1.5 ml, 1M) was added before being extracted with CH₂Cl₂ (3×10 ml). The organic fractions were combined, dried with anhydrous Na₂SO₄ concentrated in vacuo and the crude compound was then purified using flash column chromatography using (0%-5% MeOH, CH₂Cl₂, 1% NH₃) over 20 column volumes gave 16 (7.5 mg, 0.020 mmol, 31%).

¹H NMR (400 MHZ, DMSO-d₆) δ 12.96 (s, 1H), 9.43 (t, J=6.0 Hz, 1H), 8.61 (dd, J=2.5, 1.0 Hz, 1H), 8.05 (d, J=8.0 Hz, 1H), 7.79 (d, J=1.5 Hz, 1H), 7.70-7.31 (m, 11H), 6.58 (dd, J=2.5, 1.5 Hz, 1H), 4.57 (d, J=6.0 Hz, 2H).

HRMS (ESI-TOF) calcd for C₂₃H₂₀O₂N₃ [M+H]⁺: 370.1548, found: 370.1548.

### Reference Example 4—Synthesis of N-([1,1′-biphenyl]-4-ylmethyl)-2-chloropyrimidine-5-carboxamide (17)

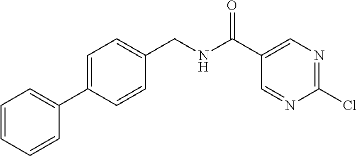 [SMILES: O=C(NCc1ccc(-c2ccccc2)cc1)c1cnc(Cl)nc1]

Following general procedure B: 2-chloro-5-carboxypyrimidine (300 mg, 1.89 mmol), 4-phenylbenzylamine (370 mg, 2.26 mmol), T3P (819 mg, 2.83 mmol) and DIPEA (365 mg, 2.83 mmol) gave 17 (285 mg, 0.88 mmol, 47%).

¹H NMR (400 MHZ, DMSO-d₆) δ 9.46 (t, J=6.0 Hz, 1H), 9.18 (s, 2H), 7.87-6.86 (m, 9H), 4.56 (d, J=6.0 Hz, 2H).

HRMS (ESI-TOF) calcd for C₁₈H₁₅³⁵ClN₃O [M+H]⁺: 324.0898, found: 324.0899.

### Reference Example 5—Synthesis of 2-Chloro-N-(4-phenoxybenzyl)pyrimidine-5-carboxamide (18)

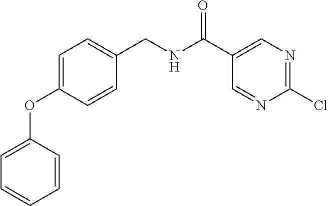 [SMILES: O=C(NCc1ccc(Oc2ccccc2)cc1)c1cnc(Cl)nc1]

Following general procedure B: 2-chloro-5-carboxypyrimidine (300 mg, 1.89 mmol), 4-phenoxybenzyamine (410 μl, 2.26 mmol) and T3P (1.5 g, 4.72 mmol) gave 18 (336 mg, 0.99 mmol, 52%).

¹H NMR (400 MHZ, DMSO-d₆) δ 9.41 (t, J=6.0 Hz, 1H), 9.16 (s, 2H), 7.53-7.27 (m, 4H), 7.19-6.78 (m, 5H), 4.50 (d, J=6.0 Hz, 2H).

HRMS (ESI-TOF) calcd for C₁₈H₁₄³⁵ClN₃O₂ [M+H]⁺: 340.0847, found: 340.1327.

### Reference Example 6—Synthesis of N-([1,1′-Biphenyl]-4-ylmethyl)-6-chloronicotinamide (19)

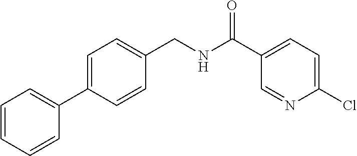 [SMILES: O=C(NCc1ccc(-c2ccccc2)cc1)c1ccc(Cl)nc1]

Following general procedure A: 2-chloroethylnicotinate (83 μl, 0.53 mmol), 4-phenyl-benzylamine (97 mg, 0.53 mmol) and DABCO-(AlMe₃)₂ (108 mg, 0.424 mmol) gave 19 (154 mg, 0.48 mmol, 91%).

¹H NMR (400 MHZ, DMSO-d₆) δ 9.34 (t, J=6.0 Hz, 1H), 8.90 (d, J=2.5 Hz, 1H), 8.30 (dd, J=8.5, 2.5 Hz, 1H), 7.87-7.19 (m, 10H), 4.54 (d, J=6.0 Hz, 2H).

HRMS (ESI-TOF) calcd for C₁₉H₁₅ClN₂O [M+H]⁺: 322.0873, found: 322.0821.

### Reference Example 7—Synthesis of N-([1,1′-Biphenyl]-4-ylmethyl)-2-(1H-pyrazol-1-yl)pyrimidine-5-carboxamide (20)

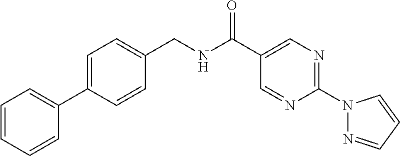 [SMILES: O=C(NCc1ccc(-c2ccccc2)cc1)c1cnc(-n2cccn2)nc1]

Following general procedure C: 17 (57 mg, 0.176 mmol), Pd^{t}BuXPhos G3 (15 mg, 0.0176 mmol), Cs₂CO₃ (201 mg, 0.619 mmol), pyrazole (42 mg, 0.619 mmol) gave 20 (10 mg, 0.0281 mmol, 16%).

¹H NMR (400 MHZ,DMSO-d₆) δ 9.44 (t, J=6.0 Hz, 1H), 9.27 (s, 2H), 8.73 (d, J=3.0 Hz, 1H), 7.93 (d, J=1.5 Hz, 1H), 7.74-7.60 (m, 4H), 7.51-7.40 (m, 4H), 7.39-7.31 (m, 1H), 6.65 (dd, J=3.0, 1.5 Hz, 1H), 4.58 (d, J=5.8 Hz, 2H).

HRMS (ESI-TOF) calcd for C₂₁H₁₈ON₅ [M+H]⁺: 356.1505, found: 356.1504.

### Example 8—Synthesis of N-(4-Phenoxybenzyl)-2-(1H-pyrazol-1-yl)pyrimidine-5-carboxamide (21)

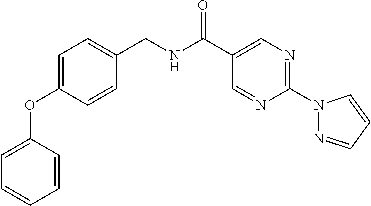 [SMILES: O=C(NCc1ccc(Oc2ccccc2)cc1)c1cnc(-n2cccn2)nc1]

Following general procedure C: 18 (100 mg, 0.294 mmol), pyrazole (40 mg, 0589 mmol), Pd^{t}BuXPhos G3 (23 mg, 0.0294 mmol), Cs₂CO₃ (238 mg, 0.735 mmol) gave 21 (64 mg, 0.172 mmol, 58%).

¹H NMR (400 MHZ, DMSO-d₆) δ 8.96 (d, J=3.0 Hz, 1H), 8.68 (d, J=3.0 Hz, 1H), 8.51-8.37 (m, 1H), 8.03-7.98 (m, 1H), 7.48-6.90 (m, 10H), 6.62-6.61 (m, 1H), 4.51 (d, J=6.0 Hz, 2H).

HRMS (ESI-TOF) calcd for C₂₁H₁₇N₅O₂ [M−H]⁻: 370.1382, found: 370.1309.

### Example 9—Synthesis of N-([1,1′-Biphenyl]-4-ylmethyl)-6-(1H-pyrazol-1-yl) nicotinamide (22)

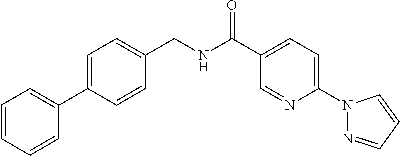 [SMILES: O=C(NCc1ccc(-c2ccccc2)cc1)c1ccc(-n2cccn2)nc1]

Following procedure C: 19 (100 mg, 0.31 mmol), pyrazole (40 mg, 0589 mmol), Pd^{t}BuXPhos G3 (25 mg, 0.031 mmol), Cs₂CO₃ (238 mg, 0.735 mmol) gave 22 (53 mg, 0.028 mmol, 48%).

¹H NMR (400 MHz, DMSO-d₆) δ 9.32 (t, J=6.0 Hz, 1H), 8.97 (d, J=2.0 Hz, 1H), 8.69 (d, J=2.5 Hz, 1H), 8.47 (dd, J=9.0, 2.0 Hz, 1H), 8.30 (dd, J=9.0, 2.0 Hz, 1H), 7.90 (d, J=1.5 Hz, 1H), 7.74-7.22 (m, 9H), 6.63 (dd, J=2.5, 1.5 Hz, 1H), 4.57 (d, J=6.0 Hz, 2H).

HRMS (ESI-TOF) calcd for C₂₂H₁₉ON₄ [M+H]⁺: 355.1553, found: 355.1551.

### Reference Example 10—Synthesis of N-Benzyl-6-chloronicotinamide (28)

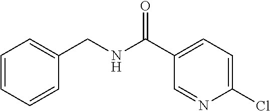 [SMILES: O=C(NCc1ccccc1)c1ccc(Cl)nc1]

Following general procedure A: 2-chloro-ethylnicotinate (200 mg, 1.081 mmol), benzylamine (116 mg, 1.08 mmol) and DABACO-AlMe₃ (221 mg, 0.864 mmol) gave 28 (220 mg, 0.897 mmol, 83%).

Solvent system used for purification: 0%-100% EtOAc in cyclohexane.

¹H NMR (400 MHZ, DMSO-d₆) δ 9.32 (t, J=6.0 Hz, 1H), 8.89 (dd, J=2.5, 1.0 Hz, 1H), 8.29 (dd, J=8.5, 2.5 Hz, 1H), 7.87-7.57 (m, 1H), 7.50-6.86 (m, 5H), 4.51 (d, J=6.0 Hz, 2H).

HRMS (ESI-TOF) calcd for C₁₃H₁₀ON₂³⁵Cl [M−H]⁻: 245.0487, found: 245.0482.

### Example 11—Synthesis of Ethyl 1-(5-(([1,1′-Biphenyl]-4-ylmethyl) carbamoyl)pyridin-2-yl)-1H-pyrazole-4-carboxylate (29)

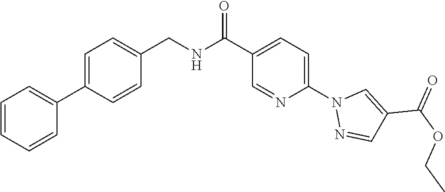 [SMILES: CCOC(=O)c1cnn(-c2ccc(C(=O)NCc3ccc(-c4ccccc4)cc3)cn2)c1]

Following general procedure C: 19 (125 mg, 0.388 mmol), pyrazole-4-carboxylate ethyl ester (65 mg, 0.465 mmol), PdtBuxPhos G3 (31 mg, 0.0388 mmol), Cs₂CO₃ (252 mg, 0.776 mmol) and ^{t}BuOH (3 ml) gave 29 (97 mg, 0.227 mmol, 59%).

¹H NMR (400 MHZ, DMSO-d₆) δ 9.38 (t, J=6.0 Hz, 1H), 9.04 (s, 1H), 9.02 (d, J=2.5 Hz, 1H), 8.51 (dd, J=8.5, 2.5 Hz, 1H), 8.27 (s, 1H), 8.07 (d, J=8.5 Hz, 1H), 7.69-7.30 (m, 9H), 4.54 (d, J=6.0 Hz, 2H), 4.21 (q, J=7.0 Hz, 2H), 1.31 (t, J=7.0 Hz, 3H).

HRMS (ESI-TOF) calcd for C₂₅H₂₁O₃N₄ [M−H]^{±}: 425.1619, found: 425.1622.

### Example 12—Synthesis of Ethyl 1-(5-(Benzylcarbamoyl)pyridin-2-yl)-1H-pyrazole-4-carboxylate (30)

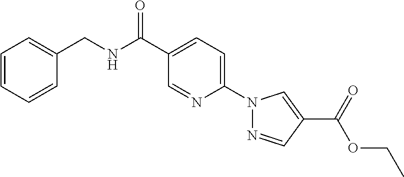 [SMILES: CCOC(=O)c1cnn(-c2ccc(C(=O)NCc3ccccc3)cn2)c1]

Following general procedure C: 28 (80 mg, 0.325 mmol), Pd^{t}BuXPhos G3 (25 mg, 0.0325 mmol), Cs₂CO₃ (317 mg, 0.97 mmol), pyrazole-4-carboxylate ethyl ester (67 mg, 0.48 mmol) and ^{t}BuOH (3 ml) gave 30 (41 mg, 0.117 mmol, 36%).

¹H NMR (400 MHZ, DMSO-d₆) δ 9.33 (t, J=6.0 Hz, 1H), 9.04 (d, J=1.0 Hz, 1H), 8.99 (dd, J=2.5, 1.0 Hz, 1H), 8.49 (dd, J=8.5, 2.5 Hz, 1H), 8.27 (d, J=1.0 Hz, 1H,), 8.06 (dd, J=8.5, 1.0 Hz, 1H), 7.38-7.32 (m, 5H), 4.53 (d, J=6.0 Hz, 2H), 4.28 (q, J=7.0 Hz, 2H), 1.31 (t, J=7.0 Hz, 3H).

HRMS (ESI-TOF) calcd for C₁₉H₁₇O₃N₄ [M−H]⁻: 349.1306, found: 349.1302.

### Example 13—Synthesis of 1-(5-(([1,1′-Biphenyl]-4-ylmethyl) carbamoyl)pyridine-2-yl)-1H-pyrazole-4-carboxylic acid (31)

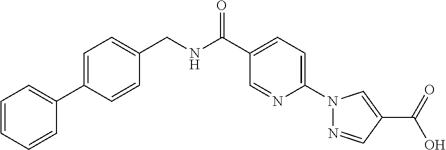 [SMILES: O=C(O)c1cnn(-c2ccc(C(=O)NCc3ccc(-c4ccccc4)cc3)cn2)c1]

29 (63 mg, 0.147 mmol) was dissolved in a mixture of THF and water (10 ml (10:1)) before the addition of LiOH-monohydrate (19 mg, 0.45 mmol). The resultant mixture was allowed to stir for 16 hr and confirmed to have gone to completion by TLC. HCl_{aq} (10 ml, 1 M) was added the reaction mixture and the resulting mixture was extracted with EtOAc (3×20 ml). The organic fractions were combined and washed with brine, dried with Na₂SO₄ and purified by flash column chromatography using (CH₂Cl₂, MeOH 0-5%, formic acid 1%) over 20 column volumes gave 31 (15 mg, 0.037 mmol, 26%).

¹H NMR (400 MHZ, DMSO-d₆) δ 9.37-9.34 (m, 1H), 9.01 (d, J=2.5 Hz, 1H), 8.92 (d, J=2.5 Hz, 1H), 8.53-8.47 (m, 1H), 8.09-8.01 (m, 1H), 7.70-7.62 (m, 4H), 7.46 (t, J=7.8 Hz, 4H), 7.39-7.30 (m, 2H), 4.56 (d, J=6.0 Hz, 2H).

HRMS (ESI-TOF) calcd for C₂₃H₁₇O₃N₄ [M−H]⁻: 397.1306, found: 397.1310.

### Example 14—Synthesis of 1-(5-(Benzylcarbamoyl)pyridin-2-yl)-1H-pyrazole-4-carboxylic acid (32)

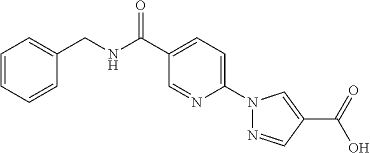 [SMILES: O=C(O)c1cnn(-c2ccc(C(=O)NCc3ccccc3)cn2)c1]

30 (20 mg, 0.043 mmol) was dissolved in THF (2 ml), MeOH (2 ml) and water (0.5 ml) was added to the reaction mixture before the addition of LiOH-monohydrate (16 mg, 0.40 mmol). The resultant mixture was stirred overnight at room temperature. The reaction mixture was acidified to pH 3 with HCl (1M) solution and extracted with EtOAc (3×25 ml), washed with brine dried with anhydrous Na₂SO₄. The crude compound was then purified using flash column chromatography using (CH₂Cl₂, MeOH 0-5%, formic acid 1%) over 20 column volumes gave 32 (13 mg, 0.040 mmol, 94%).

¹H NMR (400 MHZ, DMSO-d₆) δ 9.34 (t, J=6.0 Hz, 1H), 8.99 (dd, J=2.5, 1.0 Hz, 1H), 8.96 (s, 1H), 8.48 (dd, J=8.5, 2.5 Hz, 1H), 8.19 (s, 1H), 8.08-8.02 (m, 1H), 7.48-7.21 (m, 5H), 4.54 (d, J=6.0 Hz, 2H).

HRMS (ESI-TOF) calcd for C₁₇H₁₃O₃N₄[M−H]⁻: 321.0993, found: 321.0994.

### Example 15—Synthesis of N-([1,1′-Biphenyl]-4-ylmethyl)-6-chloro-4-methoxynicotinamide (33)

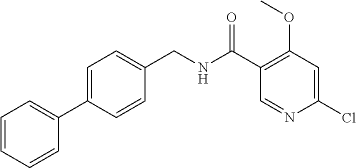 [SMILES: COc1cc(Cl)ncc1C(=O)NCc1ccc(-c2ccccc2)cc1]

Following general procedure B: 6-chloro-4-methoxy-nicotinic acid (300 mg, 1.60 mmol), 4-phenylbenzyl amine (439 mg, 2.4 mmol), T3P (1.27 g, 4 mmol) and DIPEA (825 μl, 4.8 mmol) gave 33 (432 mg, 1.22 mmol, 76%).

¹H NMR (400 MHZ, DMSO-d₆) δ 8.82 (t, J=6.0 Hz, 1H), 8.51 (s, 1H), 7.68-7.60 (m, 4H), 7.57-7.27 (m, 6H), 4.53 (d, J=6.0 Hz, 2H), 3.99 (s, 3H).

HRMS (ESI-TOF) calcd for C₂₀H₁₈O₂N₂³⁵Cl [M+H]⁺: 353.1051, found: 353.1048.

### Reference Example 16—Synthesis of N-([1,1′-Biphenyl]-4-yl)-6-chloro-4-methoxynicotinamide (34)

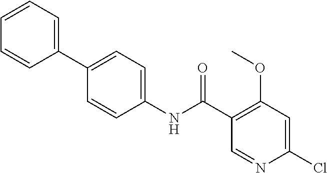 [SMILES: COc1cc(Cl)ncc1C(=O)Nc1ccc(-c2ccccc2)cc1]

Following general procedure B: 6-chloro-4-methoxy-nicotinic acid (500 mg, 2.67 mmol), 4-aminobiphenyl (540 mg, 3.2 mmol), T3P (2.12 g, 6.68 mmol) and DIPEA (846 μl, 6.68 mmol) gave 34 (311 mg, 0.92 mmol, 34%).

¹H NMR (400 MHZ, DMSO-d₆) δ 10.36 (s, 1H), 8.46 (s, 1H), 7.87-7.77 (m, 2H), 7.71-7.62 (m, 4H), 7.52-7.30 (m, 4H), 3.99 (s, 3H).

HRMS (ESI-TOF) calcd for C₁₉H₁₆O₂N₂³⁵Cl [M+H]⁺: 339.0894, found: 339.0893.

### Reference Example 17—Synthesis of 6-Chloro-4-methoxy-N-(3-(trifluoromethyl)benzyl) nicotinamide (35)

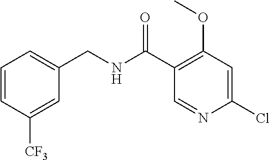 [SMILES: COc1cc(Cl)ncc1C(=O)NCc1cccc(C(F)(F)F)c1]

Following general procedure B: 6-chloro-4-methoxy-nicotinic acid (500 mg, 2.67 mmol), 3-trifluoromethylbenzyl amine (566 mg, 3.2 mmol), T3P (2.12 g, 6.68 mmol) and DIPEA (846 μl, 6.68 mmol) gave 35 (518 mg, 1.505 mmol, 56%).

¹H NMR (400 MHZ, DMSO-d₆) δ 8.91 (t, J=6.0 Hz, 1H), 8.48 (s, 1H), 7.75-7.53 (m, 4H), 7.35 (s, 1H), 4.57 (d, J=6.0 Hz, 2H), 3.98 (s, 3H).

HRMS (ESI-TOF) calcd for C₁₅H₁₃O₂N₂³⁵ClF₃ [M+H]⁺: 345.0612, found: 345.0613.

### Reference Example 18—6-Chloro-N-(cyclohexylmethyl)-4-methoxynicotinamide (36)

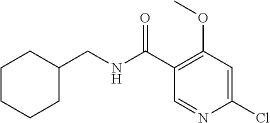 [SMILES: COc1cc(Cl)ncc1C(=O)NCC1CCCCC1]

Following general procedure B: 6-chloro-4-methoxy-nicotinic acid (500 mg, 2.67 mmol), cyclohexane-methylamine (361 mg, 3.2 mmol), T3P (2.12 g, 6.68 mmol) and DIPEA (846 μl, 6.68 mmol) gave 36 (438 mg, 1.55 mmol, 58%).

¹H NMR (400 MHZ, DMSO-d₆) δ 8.40 (s, 1H), 8.17 (t, J=6.0 Hz, 1H), 7.30 (s, 1H), 3.95 (s, 3H,), 3.12-3.06 (m, 2H), 1.87-0.75 (m, 11H).

HRMS (ESI-TOF) calcd for C₁₄H₂₀O₂N₂³⁵Cl [M+H]⁺: 283.1207, found: 283.1208.

### Example 19—N-([1,1′-Biphenyl]-4-ylmethyl)-4-methoxy-6-(1H-pyrazol-1-yl) nicotinamide (37)

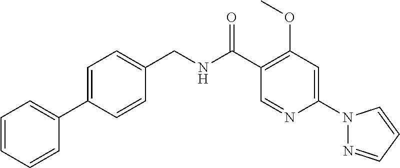 [SMILES: COc1cc(-n2cccn2)ncc1C(=O)NCc1ccc(-c2ccccc2)cc1]

Following general procedure C: 33 (50 mg, 0.142 mmol), Pd^{t}BuXPhos G3 (11.2 mg, 0.0142 mmol), Cs₂CO₃ (138 mg, 0.426 mmol), pyrazole (24 mg, 0.355 mmol) gave 37 (30 mg, 0.078 mmol, 55%).

¹H NMR (400 MHZ, DMSO-d₆) δ 8.79 (t, J=6.0 Hz, 1H), 8.68-8.65 (m, 2H), 7.88 (dd, J=1.5, 1.0 Hz, 1H), 7.67-7.46 (m, 9H), 7.39-7.33 (m, 1H), 6.61 (dd, J=2.5, 1.5 Hz, 1H), 4.55 (d, J=6.0 Hz, 2H), 4.08 (s, 3H).

HRMS (ESI-TOF) calcd for C₂₃H₂₁O₂N₄ [M+H]⁺: 385.1659, found: 385.1658.

### Example 20—Synthesis of N-([1,1′-Biphenyl]-4-yl)-4-methoxy-6-(1H-pyrazol-1-yl) nicotinamide (38)

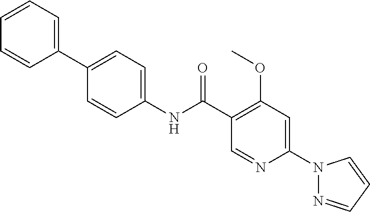 [SMILES: COc1cc(-n2cccn2)ncc1C(=O)Nc1ccc(-c2ccccc2)cc1]

Following general procedure C: 34 (150 mg, 0.43 mmol), Pd^{t}BuXPhos G3 (34 mg, 0.043 mmol), Cs₂CO₃ (354 mg, 1.09 mmol), pyrazole (74 mg, 1.09 mmol) gave 38 (67 mg, 0.181 mmol, 40%).

¹H NMR (400 MHZ, DMSO-d₆) δ 8.68 (d, J=2.5 Hz, 1H), 8.59 (s, 1H), 7.90 (d, J=1.5 Hz, 1H), 7.70-7.63 (m, 6H), 7.52-7.42 (m, 4H), 6.63 (dd, J=2.5, 1.5 Hz, 1H), 4.08 (s, 3H).

HRMS (ESI-TOF) calcd for C₂₂H₁₉O₂N₄ [M+H]⁺: 371.1502, found: 371.1501.

### Example 21—Synthesis of 6-Chloro-4-methoxy-N-(3-(trifluoromethyl)benzyl) nicotinamide (39)

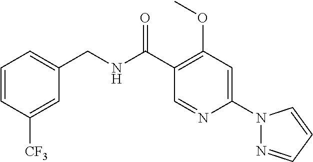 [SMILES: COc1cc(-n2cccn2)ncc1C(=O)NCc1cccc(C(F)(F)F)c1]

Following general procedure C: 35 (150 mg, 0.43 mmol), Pd^{t}BuXPhos G3 (34 mg, 0.043 mmol), Cs₂CO₃ (354 mg, 1.09 mmol), Pyrazole (74 mg, 1.09 mmol) gave 39 (72 mg, 0.191 mmol, 44%).

¹H NMR (400 MHZ, DMSO-d₆) δ 9.26 (t, J=6.0 Hz, 1H), 8.66 (dd, J=2.5, 1.0 Hz, 1H), 8.62 (s, 1H), 8.33 (d, J=2.5 Hz, 1H), 7.88 (dd, J=1.5, 1.0 Hz, 1H), 7.68-7.57 (m, 4H), 6.61 (dd, J=2.5, 1.5 Hz, 1H), 4.59 (d, J=6.0 Hz, 2H), 4.07 (s, 3H). HRMS (ESI-TOF) calcd for C₁₈H₁₆O₂N₄F₃ [M+H]⁺: 377.1219, found: 377.1220.

### Example 22—Synthesis of N-(Cyclohexylmethyl)-4-methoxy-6-(1H-pyrazol-1-yl) nicotinamide (40)

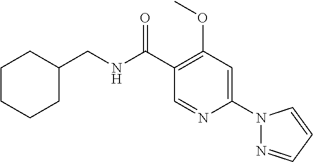 [SMILES: COc1cc(-n2cccn2)ncc1C(=O)NCC1CCCCC1]

Following general procedure C: 36 (150 mg, 0.43 mmol), Pd^{t}BuXPhos G3 (34 mg, 0.043 mmol), Cs₂CO₃ (354 mg, 1.09 mmol), pyrazole (74 mg, 1.09 mmol) gave 40 (67 mg, 0.21 mmol, 40%).

¹H NMR (400 MHZ, DMSO-d₆) δ 8.68 (dd, J=2.5, 1.0 Hz, 1H), 8.56 (s, 1H), 7.90 (dd, J=1.5, 1.0 Hz, 1H), 7.57 (s, 1H), 6.64 (dd, J=2.5, 1.5 Hz, 1H), 3.95 (s, 3H), 3.06-3.03 (m, 2H), 1.79-1.58 (m, 5H), 1.28-1.09 (m, 4H), 1.00-0.83 (m, 2H).

HRMS (ESI-TOF) calcd for C₁₇H₂₃O₂N₄ [M+H]⁺: 315.1815, found: 315.1816.

### Example 23—Synthesis of N-([1,1′-Biphenyl]-4-ylmethyl)-4-hydroxy-6-(1H-pyrazol-1-yl) nicotinamide (41)

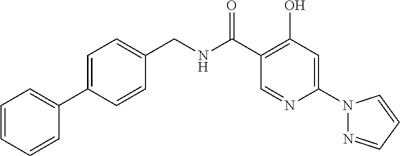 [SMILES: O=C(NCc1ccc(-c2ccccc2)cc1)c1cnc(-n2cccn2)cc1O]

TMS-I (17 mg,0.0858 mmol) was added to the mixture of 37 (11 mg, 0.0286 mmol) and CH₂Cl₂ (1 ml). The resultant mixture was heated at 90° C. for 90 minutes, then cooled to rt, HCl_{aq} (1.5 ml, 1M) was added before being extracted with CH₂Cl₂ (3×10 ml). The organic fractions were combined, dried with anhydrous Na₂SO₄ concentrated in vacuo and the crude compound was then purified using flash column chromatography using (0%-5% MeOH, CH₂Cl₂, 1% NH₃) over 20 column volumes gave 41 (2 mg, 0.0054 mmol, 20%).

¹H NMR (400 MHZ, DMSO-d₆) δ 8.76 (s, 1H), 8.64 (d, J=2.5 Hz, 1H), 7.86 (d, J=1.5 Hz, 1H), 7.70-7.60 (m, 5H), 7.52-7.29 (m, 6H), 6.60 (dd, J=2.5, 1.5 Hz, 1H), 4.59 (d, J=6.0 Hz, 2H).

HRMS (ESI-TOF) calcd for C₂₂H₁₉O₂N₄ [M+H]⁺: 371.1502, found: 371.1502.

### Example 24—Synthesis of N-([1,1′-Biphenyl]-4-yl)-4-hydroxy-6-(1H-pyrazol-1-yl) nicotinamide (42)

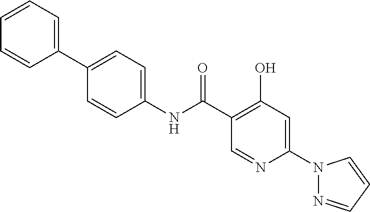 [SMILES: O=C(Nc1ccc(-c2ccccc2)cc1)c1cnc(-n2cccn2)cc1O]

TMS-I (66 mg, 0.33 mmol) was added to the reaction mixture of 38 (41 mg, 0.11 mmol) and CH₂Cl₂ (2.5 ml). The resultant mixture was heated at 90° C. for 90 minutes, then cooled to rt, HCl_{aq} (1.5 ml, 1M) was added before being extracted with CH₂Cl₂ (3× 10 ml). The organic fractions were combined, dried with anhydrous Na₂SO₄ concentrated in vacuo and the crude compound was then purified using flash column chromatography using (0%-5% MeOH, CH₂Cl₂, 1% NH₃) over 20 column volumes gave 42 (7 mg, 0.0196 mmol, 18%).

¹H NMR (400 MHZ, DMSO-d₆) δ 8.70 (d, J=2.0 Hz, 1H), 8.64 (s, 1H), 7.92 (d, J=2.0 Hz, 1H), 7.72-7.62 (m, 5H), 7.55-7.27 (m, 6H), 6.65 (dd, J=2.0 Hz, 1H). HRMS (ESI-TOF) calcd for C₂₁H₁₇O₂N₄ [M+H]⁺: 357.1346, found: 357.1343.

### Example 25—Synthesis of 4-Hydroxy-6-(1H-pyrazol-1-yl)-N-(3-(trifluoromethyl)benzyl) nicotinamide (43)

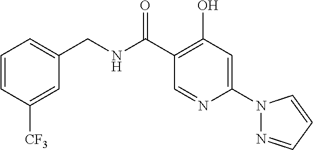 [SMILES: O=C(NCc1cccc(C(F)(F)F)c1)c1cnc(-n2cccn2)cc1O]

TMS-I (59 mg, 0.295 mmol) was added to the reaction mixture of 39 (37 mg, 0.0986 mmol) and CH₂Cl₂ (2.5 ml). The resultant mixture was heated at 90° C. for 90 minutes, then cooled to rt, HCl_{aq} (1.5 ml, 1M) was added before being extracted with CH₂Cl₂ (3×10 ml). The organic fractions were combined, dried with anhydrous Na₂SO₄ concentrated in vacuo and the crude compound was then purified using flash column chromatography using (0%-5% MeOH, CH₂Cl₂, 1% NH₃) over 20 column volumes gave 43 (20 mg, 0.055 mmol, 57%).

¹H NMR (400 MHZ, DMSO-d₆) δ 8.66 (s, 1H), 8.59 (dd, J=2.5, 1.0 Hz, 1H), 7.82 (dd, J=1.5, 1.0 Hz, 1H), 7.71-7.49 (m, 4H), 7.26 (s, 1H), 6.55 (dd, J=2.5, 1.5 Hz, 1H), 4.59 (d, J=6.0 Hz, 2H).

HRMS (ESI-TOF) calcd for C₁₇H₁₄O₂N₄F₃ [M+H]⁺: 363.1063, found: 363.1067.

### Example 26—Synthesis of N-(Cyclohexylmethyl)-4-hydroxy-6-(1H-pyrazol-1-yl) nicotinamide (44)

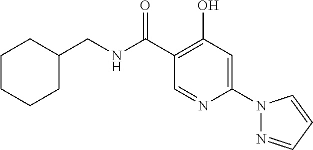 [SMILES: O=C(NCC1CCCCC1)c1cnc(-n2cccn2)cc1O]

TMS-I (128 mg, 0.64 mmol) was added to the reaction mixture of 40 (67 mg, 0.214 mmol) and CH₂Cl₂ (2.5 ml). The resultant mixture was heated at 90° C. for 90 minutes, then cooled to rt, HCl_{aq} (1.5 ml, 1M) was added before being extracted with CH₂Cl₂ (3×10 ml). The organic fractions were combined, dried with anhydrous Na₂SO₄ concentrated in vacuo and the crude compound was then purified using flash column chromatography using (0%-5% MeOH, CH₂Cl₂, 1% NH₃) over 20 column volumes gave 44 (17 mg, 0.056 mmol, 26%).

¹H NMR (400 MHZ, DMSO-d₆) δ 8.63 (d, J=2.5 Hz, 1H), 8.47 (s, 1H), 8.29 (d, J=2.5 Hz, 1H), 8.21 (s, 1H), 6.65-6.63 (m, 1H), 3.05 (t, J=6.0 Hz, 2H), 1.75-1.44 (m, 5H), 1.27-1.10 (m, 3H), 1.05-0.80 (m, 2H).

HRMS (ESI-TOF) calcd for C₁₋₆H₂₁O₂N₄ [M+H]⁺: 301.1659, found: 301.1656.

### Reference Example 27—Synthesis of 6-Chloro-4-methoxy-N-(4-(trifluoromethyl)benzyl) nicotinamide (45)

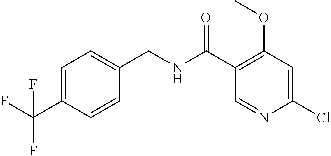 [SMILES: COc1cc(Cl)ncc1C(=O)NCc1ccc(C(F)(F)F)cc1]

Following general procedure B: 6-chloro-5-methoxy-nicotinic acid (350 mg, 1.87 mmol), 4-trifluoromethylbenzylamine (448 mg, 2.56 mmol), T3P (1.48 g, 4.67 mmol) and DIPEA (803 μl, 4.67 mmol) gave 45 (437 mg, 1.27 mmol, 66%).

¹H NMR (400 MHZ, Chloroform-d) δ 9.02 (s, 1H), 7.81 (t, J=6.0 Hz, 1H), 7.58 (d, J=8.0 Hz, 2H,), 7.43 (d, J=8.0 Hz, 2H), 6.93 (s, 1H), 4.69 (d, J=6.0 Hz, 2H), 4.01 (s, 3H). HRMS (ESI-TOF) calcd for C₁₅H₁₃O₂N₂³⁵ClF₃ [M+H]⁺: 345.0612, found: 345.0608.

### Reference Example 28—Synthesis of N-([1,1′-Biphenyl]-3-ylmethyl)-6-chloro-4-methoxynicotinamide (46)

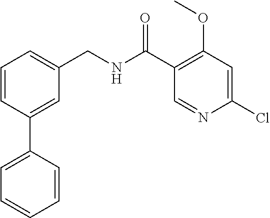 [SMILES: COc1cc(Cl)ncc1C(=O)NCc1cccc(-c2ccccc2)c1]

Following general procedure B: 6-chloro-5-methoxy-nicotinic acid (400 mg, 2.13 mmol), 3-phenylbenzyl amine (469 mg, 2.56 mmol), T3P (1.62 g, 5.12 mmol) and DIPEA (880 μl, 5.12 mmol) gave 46 (570 mg, 1.61 mmol, 76%).

¹H NMR (400 MHZ, DMSO-d₆) δ 8.85 (t, J=6.0 Hz, 1H), 8.48 (s, 1H), 7.70-7.30 (m, 10H), 4.56 (d, J=6.0 Hz, 2H), 3.96 (s, 3H).

HRMS (ESI-TOF) calc'd for C₂₀H₁₈O₂N₂³⁵Cl [M+H]⁺: 353.1051, found: 353.1053.

### Reference Example 29—Synthesis of N-([1,1′-Biphenyl]-3-yl)-6-chloro-4-methoxynicotinamide (47)

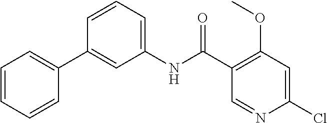 [SMILES: COc1cc(Cl)ncc1C(=O)Nc1cccc(-c2ccccc2)c1]

Following general procedure B: 6-chloro-5-methoxy-nicotinic acid (350 mg, 1.87 mmol), 3-aminobiphenyl (411 mg, 2.43 mmol), T3P (1.48 g, 4.67 mmol) and DIPEA (803 μl, 4.67 mmol) gave 47 (421 mg, 1.24 mmol, 66%).

¹H NMR (400 MHZ, Chloroform-d) δ 9.24 (s, 1H), 9.05 (s, 1H), 7.90-7.87 (m, 1H), 7.66-7.30 (m, 8H), 6.91 (s, 1H), 4.06 (s, 3H).

HRMS (ESI-TOF) calcd for C₁₉H₁₆O₂N₂³⁵Cl [M+H]⁺: 339.0894, found: 339.0894.

### Reference Example 30—Synthesis of 6-Chloro-4-methoxy-N-(4-(trifluoromethoxy)benzyl) nicotinamide (48)

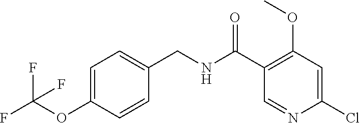 [SMILES: COc1cc(Cl)ncc1C(=O)NCc1ccc(OC(F)(F)F)cc1]

Following general procedure B: 6-chloro-5-methoxy-nicotinic acid (300 mg, 1.60 mmol), 4-trifluoromethoxybenzylamine (367 mg, 1.92 mmol), T3P (1.01 g, 3.2 mmol) and DIPEA (803 μl, 4.67 mmol) gave 48 (434 mg, 1.20 mmol, 75%).

¹H NMR (400 MHZ, Chloroform-d) δ 8.97 (s, 1H), 7.78 (t, J=6.0 Hz, 1H), 7.37-7.29 (m, 2H), 7.17-7.11 (m, 2H), 6.90 (s, 1H), 4.61 (d, J=6.0 Hz, 2H), 3.98 (s, 3H). HRMS (ESI-TOF) calcd for C₁₅H₁₃O₃N₂³⁵ClF₃ [M+H]⁺: 361.0561, found: 361.0563.

### Reference Example 31—Synthesis of 6-Chloro-4-methoxy-N-((4-(trifluoromethyl)cyclohexyl)methyl) nicotinamide (49)

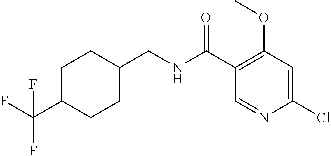 [SMILES: COc1cc(Cl)ncc1C(=O)NCC1CCC(C(F)(F)F)CC1]

Following general procedure B: 6-chloro-5-methoxy-nicotinic acid (250 mg, 1.33 mmol), C-(4-trifluoromethyl-cyclohexylamine (255 mg, 1.59 mmol), T3P (827 mg, 2.6 mmol) and DIPEA (803 μl, 4.67 mmol) gave 49 (402 mg, 1.14 mmol, 86%).

¹H NMR (400 MHZ, CDCl₃) δ 8.89 (s, 1H), 7.31 (s, 1H), 6.82 (s, 1H), 3.94 (s, 3H), 3.23 (t, J=6.2 Hz, 2H), 1.95-1.74 (m, 5H), 1.57-1.42 (m, 1H), 1.21 (qd, J=12.7, 2.9 Hz, 2H), 0.93 (qd, J=12.8, 3.1 Hz, 2H).

HRMS (ESI-TOF) calc'd for C₁₅H₁₉O₂N₂³⁵ClF₃ [M+H]⁺: 351.1081, found: 351.1078.

### Reference Example 32—Synthesis of tert-Butyl ((6-chloropyridin-3-yl)methyl) carbamate (50)

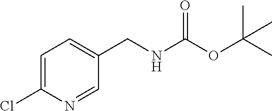 [SMILES: CC(C)(C)OC(=O)NCc1ccc(Cl)nc1]

6-Chloropyridin-3-yl) methamine (4.0 g, 0.0281 mol) was dissolved in CH₂Cl₂ (50 ml) before the addition of DIPEA (36.9 ml, 0.049 mol). Di tert-butyl dicarbonate (7.6 g, 0.035 mol) was added slowly to the reaction mixture and the resultant mixture was allowed to stir for 16 hrs at room temperature. The reaction mixture was then washed with water (3×50 ml), brine (50 ml) and dried over Na₂SO₄. The organic phase was reduced in vacuo before being purified by flash column chromatography using (Cyclohexane 100%-50%, EtOAc 0%-50%) over 20 column volumes gave 50 (6.08 g, 0.0251 mmol, 89%).

¹H NMR (400 MHZ, Chloroform-d) δ 8.25 (dd, J=2.5, 1.0 Hz, 1H), 7.58 (dd, J=8.5, 2.5 Hz, 1H,), 7.24 (d, J=1.0 Hz, 1H), 5.08 (s, 1H), 4.26 (d, J=6.0 Hz, 2H), 1.41 (s, 9H). HRMS (ESI-TOF) calcd for C₁H₁₆O₂N₂³⁵Cl [M+H]⁺: 243.0894, found: 243.0895.

### Reference Example 33—Synthesis of tert-Butyl ((6-phenylpyridin-3-yl)methyl) carbamate (51)

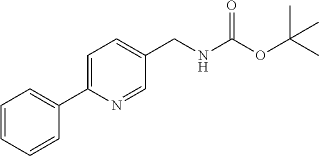 [SMILES: CC(C)(C)OC(=O)NCc1ccc(-c2ccccc2)nc1]

50 (500 mg, 2.06 mmol), phenyl boronic acid (249 mg, 2.06 mmol), Pd tetrakis (118 mg, 0.103 mmol) and Cs₂CO₃ (1.33 g, 4.12 mmol) were dissolved in anhydrous dioxane (5 ml). The resultant mixture was heated under microwave irradiation at 100° C. for 30 mins. The reaction mixture was filtered through a celite pad, water (25 ml) was added to the reaction mixture and was extracted with CH₂Cl₂ (3×25 ml). The organic fractions were combined, washed with water (3×50 ml), brine (50 ml) and dried over Na₂SO₄. The organic phase was removed in vacuo before being purified by flash column chromatography using (cyclohexane 100%-50%, EtOAc 0%-50%) over 20 column volumes gave 51 (520 mg, 1.83 mmol, 89%).

¹H NMR (400 MHZ, Chloroform-d) δ 8.58 (t, J=1.5 Hz, 1H), 7.98-7.93 (m, 2H,), 7.67 (d, J=1.5 Hz, 2H), 7.48-7.38 (m, 3H), 5.08 (s, 1H), 4.34 (d, J=6.0 Hz, 2H), 1.46 (s, 9H).

HRMS (ESI-TOF) calcd for C₁₇H₂₁O₂N₂ [M+H]⁺: 285.1597, found: 285.1594.

### Reference Example 34—Synthesis of (6-Phenylpyridin-3-yl) methanamine (52)

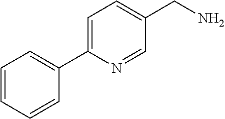 [SMILES: NCc1ccc(-c2ccccc2)nc1]

51 (500 mg, 1.76 mmol) was dissolved in CH₂Cl₂ (5 ml) and HCl ((2M) in ether (3 ml)) was added to the solution. The resultant mixture was put under vacuo and flushed with N₂; this was repeated 3 times. The resultant mixture was stirred at room temperature for 16 hrs. The volatiles were evaporated in vacuo to give 52 (312 mg, 1.69 mmol, 96%).

¹H NMR (400 MHZ, DMSO-d₆) δ 8.85 (s, 2H), 8.79 (dd, J=2.5, 1.0 Hz, 1H), 8.15-8.07 (m, 3H,), 8.02 (dd, J=8.0, 1.0 Hz, 1H), 7.55-7.41 (m, 3H), 4.08 (s, 2H).

HRMS (ESI-TOF) calcd for C₁₂H₁₃N₂ [M+H]⁺: 185.1073, found: 185.1073.

### Reference Example 35—Synthesis of 6-Chloro-4-methoxy-N-((6-phenylpyridin-3-yl)methyl) nicotinamide (53)

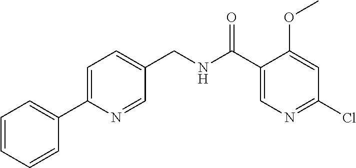 [SMILES: COc1cc(Cl)ncc1C(=O)NCc1ccc(-c2ccccc2)nc1]

Following general procedure B: 52 (100 mg, 0.54 mmol), 6-chloro-4-methoxy-nicotinic acid (85 mg, 0.45 mmol) and T3P (358 mg, 1.125 mmol) gave 53 (144 mg, 0.41 mmol, 90%).

¹H NMR (400 MHZ, DMSO-d₆) δ 8.91 (t, J=6.0 Hz, 1H,), 8.51 (s, 1H), 8.13-7.79 (m, 4H), 7.63-7.37 (m, 4H), 7.32 (s, 1H), 4.54 (d, J=6.0 Hz, 2H), 3.98 (s, 3H). HRMS (ESI-TOF) calcd for C₁₉H₁₇O₂N₃³⁵Cl [M+H]⁺: 354.1003, found: 354.1002.

### Example 36—Synthesis of 4-Methoxy-6-(1H-pyrazol-1-yl)-N-(4-(trifluoromethyl)benzyl) nicotinamide (56)

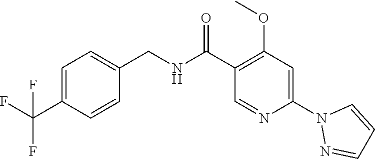 [SMILES: COc1cc(-n2cccn2)ncc1C(=O)NCc1ccc(C(F)(F)F)cc1]

Following general procedure C: 45 (200 mg, 0.58 mmol), Pd^{t}BuXPhos G3 (46 mg, 0.058 mmol), Cs₂CO₃ (377 mg, 1.16 mmol), pyrazole (42 mg, 0.63 mmol) and ^{t}BuOH (2 ml) gave 56 (69 mg, 0.185 mmol, 32%).

¹H NMR (400 MHZ, DMSO-d₆) δ 8.88 (t, J=6.0 Hz, 1H), 8.66 (dd, J=2.5, 1.0 Hz, 1H), 8.64 (s, 1H), 7.89 (t, J=1.5, 1.0 Hz, 1H), 7.72 (d, J=7.0 Hz, 2H), 7.60 (s, 1H), 7.56 (d, J=7.0 Hz, 2H), 6.62 (dd, J=2.5, 1.5 Hz, 1H), 4.59 (d, J=6.0 Hz, 2H), 4.08 (s, 3H).

HRMS (ESI-TOF) calcd for C₁₈H₁₆O₂N₄F₃ [M+H]⁺: 377.1221, found: 377.1221.

### Example 37—Synthesis of N-([1,1′-Biphenyl]-3-ylmethyl)-4-methoxy-6-(1H-pyrazol-1-yl) nicotinamide (57)

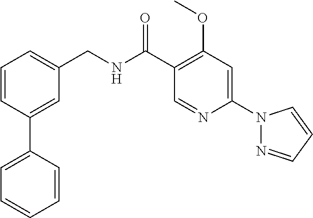 [SMILES: COc1cc(-n2cccn2)ncc1C(=O)NCc1cccc(-c2ccccc2)c1]

Following general procedure C: 46 (150 mg, 0.42 mmol), Pd^{t}BuXPhos G3 (33 mg, 0.0426 mmol), Cs₂CO₃ (341 mg, 1.05 mmol), pyrazole (42 mg, 0.63 mmol) and ^{t}BuOH (2 ml) gave 57 (78 mg, 0.203 mmol, 48%).

¹H NMR (400 MHz, Chloroform-d) δ 9.03 (d, J=2.0 Hz, 1H), 8.53 (d, J=2.5 Hz, 1H,), 7.85-7.76 (m, 1H), 7.70-7.63 (m, 1H), 7.56-7.20 (m, 9H), 7.20-7.15 (m, 1H), 6.40 (dd, J=2.5, 2.0 Hz, 1H), 4.66 (d, J=6.0 Hz, 2H), 3.97 (s, 3H).

HRMS (ESI-TOF) calcd for C₂₃H₂₁O₂N₄ [M+H]⁺: 385.1659, found: 385.1656.

### Example 38—Synthesis of N-([1,1′-Biphenyl]-3-yl)-4-methoxy-6-(1H-pyrazol-1-yl) nicotinamide (58)

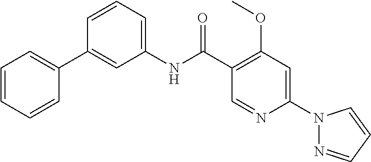 [SMILES: COc1cc(-n2cccn2)ncc1C(=O)Nc1cccc(-c2ccccc2)c1]

Following general procedure C: 47 (100 mg, 0.295 mmol), Pd^{t}BuXPhos G3 (20 mg, 0.028 mmol), Cs₂CO₃ (185 mg, 0.568 mmol), pyrazole (23 mg, 0.34 mmol) and ^{t}BuOH (2 ml) gave 58 (50 mg, 0.13 mmol, 46%).

¹H NMR (400 MHZ, Chloroform-d) δ 9.42 (s, 1H), 9.18 (s, 1H), 8.64 (dd, J=2.5, 1.0 Hz, 1H), 7.91 (m, 1H), 7.78 (dd, J=1.5, 1.0 Hz, 1H,), 7.69-7.33 (m, 9H), 6.51 (dd, J=2.5, 1.5 Hz, 1H,), 4.23 (s, 3H).

HRMS (ESI-TOF) calcd for C₂₂H₁₉O₂N₄ [M+H]⁺: 371.1502, found: 371.1497.

### Example 39—Synthesis of 4-Methoxy-6-(1H-pyrazol-1-yl)-N-(4-(trifluoromethoxy)benzyl) nicotinamide (59)

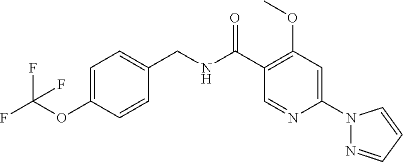 [SMILES: COc1cc(-n2cccn2)ncc1C(=O)NCc1ccc(OC(F)(F)F)cc1]

Following general procedure C: 48 (100 mg, 0.277 mmol), Pd^{t}BuXPhos G3 (19 mg, 0.027 mmol), Cs₂CO₃ (180 mg, 0.554 mmol), pyrazole (23 mg, 0.33 mmol) and ^{t}BuOH (2 ml) gave 59 (79 mg, 0.201 mmol, 73%).

¹H NMR (400 MHZ, Chloroform-d) δ 9.12 (s, 1H), 8.63 (dd, J=2.5, 1.0 Hz, 1H), 7.76 (t, J=1.5, 1.0 Hz, 1H), 7.61 (s, 1H), 7.40 (d, J=7.5 Hz, 2H), 7.20 (d, J=7.5 Hz, 2H), 6.50 (dd, J=2.5, 1.5 Hz, 1H,), 4.68 (d, J=6.0 Hz, 2H), 4.10 (s, 3H).

HRMS (ESI-TOF) calcd for C₁₈H₁₆O₃N₄F₃ [M+H]⁺: 393.1169, found: 393.1163.

### Example 40—Synthesis of 4-Methoxy-6-(1H-pyrazol-1-yl)-N-((4-(trifluoromethyl)cyclohexyl)methyl) nicotinamide (60)

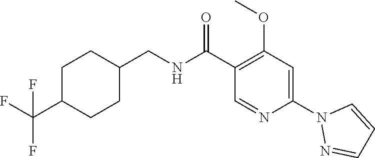 [SMILES: COc1cc(-n2cccn2)ncc1C(=O)NCC1CCC(C(F)(F)F)CC1]

Following general procedure C: 49 (100 mg, 0.277 mmol), Pd^{t}BuXPhos G3 (19 mg, 0.027 mmol), Cs₂CO₃ (180 mg, 0.554 mmol), pyrazole (23 mg, 0.33 mmol) and ^{t}BuOH (2 ml) gave 60 (49 mg, 0.127 mmol, 46%).

¹H NMR (400 MHZ, CDCl₃) δ 9.07 (s, 1H), 8.62 (dd, J=2.6, 0.7 Hz, 1H), 7.75 (d, J=1.6 Hz, 1H), 7.60 (s, 1H), 7.54 (d, J=6.2 Hz, 1H), 6.49 (dd, J=2.7, 1.7 Hz, 1H), 4.13 (s, 3H), 3.35 (t, J=6.4 Hz, 2H), 2.06-1.90 (m, 5H), 1.69-1.57 (m, 1H), 1.43-1.20 (m, 2H), 1.05 (qd, J=12.8, 2.6 Hz, 2H).

HRMS (ESI-TOF) calcd for C₁₈H₂₂O₂N₄F₃ [M+H]⁺: 383.1689, found: 383.1689.

### Example 41—Synthesis of 4-Methoxy-N-((6-phenylpyridin-3-yl)methyl)-6-(1H-pyrazol-1-yl) nicotinamide (61)

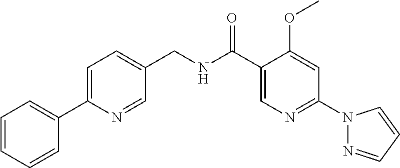 [SMILES: COc1cc(-n2cccn2)ncc1C(=O)NCc1ccc(-c2ccccc2)nc1]

Following general procedure C: 53 (80 mg, 0.226 mmol), Pd^{t}BuXPhos G1 (14 mg, 0.0226 mmol), Cs₂CO₃ (145 mg, 0.452 mmol), pyrazole (15 mg, 0.226 mmol) and ^{t}BuOH (2 ml) gave 61 (16 mg, 0.043 mmol, 19%).

¹H NMR (400 MHZ, Chloroform-d) δ 9.11 (s, 1H), 8.69 (dd, J=2.5, 1.0 Hz, 1H), 8.62 (dd, J=2.5, 1.0 Hz, 1H), 8.01-7.68 (m, 7H), 7.52-7.38 (m, 3H), 6.48 (dd, J=2.5, 1.5 Hz, 1H), 4.73 (d, J=6.0 Hz, 2H), 4.10 (s, 3H).

HRMS (ESI-TOF) calcd for C₂₂H₂₀O₂N₄ [M+H]⁺: 386.1611, found: 386.1604.

### Example 42—Synthesis of 4-Hydroxy-6-(1H-pyrazol-1-yl)-N-(4-(trifluoromethyl)benzyl) nicotinamide (64)

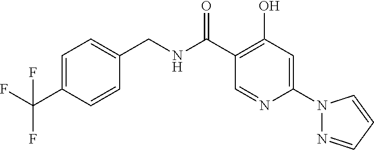 [SMILES: O=C(NCc1ccc(C(F)(F)F)cc1)c1cnc(-n2cccn2)cc1O]

Following general procedure D: 56 (12 mg, 0.0319 mmol) and LiCl (19 mg, 0.319 mmol) in DMAc (2 ml) gave 64 (6 mg, 0.016 mmol, 52%).

¹H NMR (400 MHZ, DMSO-d₆) δ 10.12 (s, 1H), 8.68 (s, 1H), 8.64-8.58 (m, 1H), 7.81 (s, 1H,), 7.71 (d, J=8.0 Hz, 2H), 7.56 (d, J=8.0 Hz, 2H), 7.20 (m, 1H), 6.60-6.53 (m, 1H), 4.61 (d, J=6.0 Hz, 2H).

HRMS (ESI-TOF) calcd for C₁₇H₁₂O₂N₄F₃ [M−H]⁻: 361.0917, found: 361.0917.

### Example 43—Synthesis of N-([1,1′-Biphenyl]-3-ylmethyl)-4-hydroxy-6-(1H-pyrazol-1-yl) nicotinamide (65)

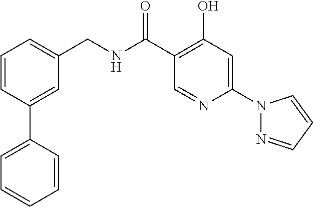 [SMILES: O=C(NCc1cccc(-c2ccccc2)c1)c1cnc(-n2cccn2)cc1O]

Following general procedure D: 57 (50 mg, 0.129 mmol) and LiCl (78 mg, 1.29 mmol) in DMAc (5 ml) gave 65 (30 mg, 0.081 mmol, 63%).

¹H NMR (400 MHZ, DMSO-d₆) δ 13.21 (s, 1H), 9.26 (t, J=6.0 Hz, 1H), 8.69 (dd, J=2.5, 1.0 Hz, 1H), 8.64 (d, J=2.5 Hz, 1H), 8.28 (d, J=2.5 Hz, 1H), 7.91 (dd, J=1.5, 1.0 Hz, 1H), 7.59-7.29 (m, 9H), 6.64 (dd, J=2.5, 1.5 Hz, 1H), 4.63 (d, J=6.0 Hz, 2H,).

HRMS (ESI-TOF) calcd for C₂₂H₁₉O₂N₄ [M+H]⁺: 371.1502, found: 371.1507

### Example 44—Synthesis of N-([1,1′-Biphenyl]-3-yl)-4-hydroxy-6-(1H-pyrazol-1-yl) nicotinamide (66)

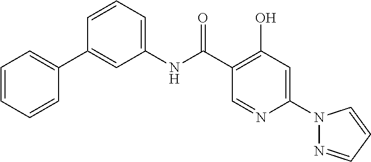 [SMILES: O=C(Nc1cccc(-c2ccccc2)c1)c1cnc(-n2cccn2)cc1O]

Following general procedure D: 58 (40 mg, 0.107 mmol) and LiCl (45 mg, 1.07 mmol) in DMAc (2 ml) gave 66 (19 mg, 0.053 mmol, 50%).

¹H NMR (400 MHZ, DMSO-d₆) δ 8.74-8.64 (m, 2H), 8.04 (s, 1H), 7.93 (s, 1H), 7.76-7.63 (m, 3H), 7.56-7.29 (m, 7H), 6.68-6.63 (m, 1H).

HRMS (ESI-TOF) calcd for C₂₁H₁₇O₂N₄ [M+H]⁺: 357.1346, found: 357.1345.

### Example 45—Synthesis of 4-Hydroxy-6-(1H-pyrazol-1-yl)-N-(4-(trifluoromethyloxy)benzyl) nicotinamide (67)

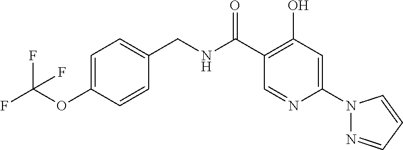 [SMILES: O=C(NCc1ccc(OC(F)(F)F)cc1)c1cnc(-n2cccn2)cc1O]

Following general procedure D: 59 (70 mg, 0.178 mmol) and LiCl (74 mg, 1.78 mmol) in DMAc (2 ml) gave 67 (43 mg, 0.114 mmol, 64%).

¹H NMR (400 MHZ, DMSO-d₆) δ 13.16 (s, 1H), 9.41 (s, 1H), 8.76 (s, 1H), 8.67-8.61 (m, 1H), 7.89-7.84 (m, 1H), 7.51-7.45 (m, 2H), 7.35-7.31 (m, 3H), 6.61-6.58 (m, 1H), 4.57 (d, J=6.0 Hz, 2H).

HRMS (ESI-TOF) calcd for C₁₇H₁₂O₃N₄F₃ [M−H]⁻: 377.0867, found: 377.865.

### Example 46—Synthesis of 4-Hydroxy-6-(1H-pyrazol-1-yl)-N-((4-(trifluoromethyl)cyclohexyl)methyl) nicotinamide (68)

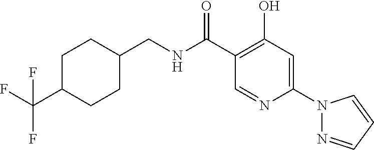 [SMILES: O=C(NCC1CCC(C(F)(F)F)CC1)c1cnc(-n2cccn2)cc1O]

Following general procedure D: 60 (30 mg, 0.078 mmol) and LiCl (33 mg, 0.78 mmol) in DMAc (2 ml) gave 68 (8.5 mg, 0.023 mmol, 30%).

¹H NMR (400 MHZ, DMSO) δ 13.33 (s, 1H), 9.10 (s, 1H), 8.72 (s, 1H), 8.63 (d, J=2.6 Hz, 1H), 7.86 (d, J=1.6 Hz, 1H), 7.28 (s, 1H), 6.60 (dd, J=2.7, 1.7 Hz, 1H), 3.20 (t, J=6.3 Hz, 2H), 2.29-2.13 (m, 1H), 1.96-1.78 (m, 4H), 1.65-1.47 (m, 1H), 1.32-0.96 (m, 4H).

HRMS (ESI-TOF) calcd for C₁₇H₂₀O₂N₄F₃ [M+H]⁺: 369.1532, found: 369.1533.

### Example 47—Synthesis of 4-Hydroxy-N-((6-phenylpyridin-3-yl)methyl)-6-(1H-pyrazol-1-yl) nicotinamide (69)

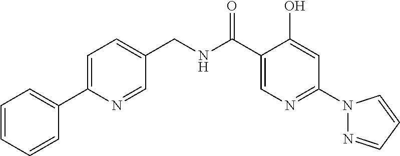 [SMILES: O=C(NCc1ccc(-c2ccccc2)nc1)c1cnc(-n2cccn2)cc1O]

Following general procedure D: 61 (16 mg, 0.041 mmol) and LiCl (17 mg, 0.41 mmol) in DMAc (2 ml) gave 69 (6 mg, 0.0161 mmol, 40%).

¹H NMR (400 MHZ, DMSO-d₆) δ 8.80-8.62 (m, 3H), 8.15-7.86 (m, 6H), 7.56-7.43 (m, 4H), 6.62-6.59 (m, 1H), 4.62 (d, J=6.0 Hz, 2H).

HRMS (ESI-TOF) calcd for C₂₁H₁₈O₂N₅ [M+H]⁺: 372.1455, found: 372.1447.

### Reference Example 48—Synthesis of N-([1,1′-Biphenyl]-4-ylmethyl)-6-chloro-5-methoxynicotinamide (72)

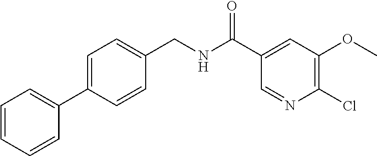 [SMILES: COc1cc(C(=O)NCc2ccc(-c3ccccc3)cc2)cnc1Cl]

Following general procedure B: 6-chloro-5-methoxy-nicotinic acid (850 mg, 4.55 mmol), 4-phenylbenzyl amine (1 g, 5.46 mmol), T3P (4.32 g, 13.6 mmol) and DIPEA (2.2 ml, 6.68 mmol) gave 72 (502 mg, 1.42 mmol, 31%).

¹H NMR (400 MHZ, DMSO-d₆) δ 9.32 (t, J=6.0 Hz, 1H), 8.50 (d, J=2.0 Hz, 1H), 7.96 (d, J=2.0 Hz, 1H), 7.74-7.31 (m, 9H), 4.56 (d, J=6.0 Hz, 2H), 3.96 (s, 3H). HRMS (ESI-TOF) calcd for C₂₀H₁₈O₂N₂³⁵Cl [M+H]⁺: 353.1051, found: 353.1049.

### Example 49—Synthesis of N-([1,1′-Biphenyl]-4-ylmethyl)-5-methoxy-6-(1H-pyrazol-1-yl) nicotinamide (73)

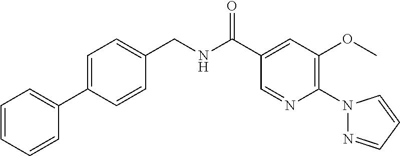 [SMILES: COc1cc(C(=O)NCc2ccc(-c3ccccc3)cc2)cnc1-n1cccn1]

Following general procedure C: 72 (100 mg, 0.284 mmol), Pd^{t}BuXPhos G3 (22 mg, 0.0284 mmol), Cs₂CO₃ (279 mg, 0.852 mmol), pyrazole (38 mg, 0.568 mmol) and ^{t}BuOH (2 ml) gave 73 (23 mg, 0.0598 mmol, 20%).

¹H NMR (400 MHz, Chloroform-d) δ 8.46 (d, J=2.0 Hz, 1H), 8.30 (dd, J=2.5, 1.0 Hz, 1H), 7.97 (d, J=2.0 Hz, 1H), 7.76 (dd, J=1.5, 1.0 Hz, 1H), 7.64-7.32 (m, 9H), 6.46 (dd, J=2.5, 1.5 Hz, 1H), 4.67 (d, J=6.0 Hz, 2H), 3.93 (s, 3H).

HRMS (ESI-TOF) calcd for C₂₃H₂₁O₂N₄ [M+H]⁺: 385.1659, found: 385.1661.

### Example 50—Synthesis of N-([1,1′-Biphenyl]-3-ylmethyl)-5-methoxy-6-(1H-pyrazol-1-yl) nicotinamide (74)

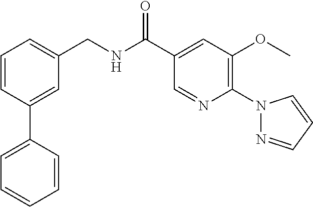 [SMILES: COc1cc(C(=O)NCc2cccc(-c3ccccc3)c2)cnc1-n1cccn1]

Following general procedure B: 6-chloro-5-methoxy-nicotinic acid (250 mg, 1.33 mmol), 3-phenylbenzyl amine (285 mg, 1.56 mmol), T3P (1.08 g, 3.4 mmol) and DIPEA (574 μl, 3.34 mmol) gave [N-([1,1′-biphenyl]-3-ylmethyl)-6-chloro-5-methoxynicotinamide. The crude material was taken onto the next step without purification and following general procedure C: N-([1,1′-biphenyl]-3-ylmethyl)-6-chloro-5-methoxynicotinamide (100 mg, 0.284 mmol), Pd^{t}BuXPhos G3 (20 mg, 0.028 mmol), Cs₂CO₃ (185 mg, 0.568 mmol), pyrazole (23 mg, 0.34 mmol) gave 74 (54 mg, 0.14 mmol, 49%).

¹H NMR (400 MHZ, DMSO-d₆) δ 9.37 (t, J=6.0 Hz, 1H), 8.62 (d, J=2.0 Hz, 1H), 8.28 (dd, J=2.5, 1.0 Hz, 1H), 8.10 (d, J=2.0 Hz, 1H), 7.77 (t, J=1.5, 1.0 Hz, 1H), 7.69-7.32 (m, 9H), 6.53 (dd, J=2.5, 1.5 Hz, 1H), 4.62 (d, J=6.0 Hz, 2H), 3.94 (s, 3H).

HRMS (ESI-TOF) calcd for C₂₃H₂₁O₂N₄ [M+H]⁺: 385.1659, found: 385.1661.

### Example 51—Synthesis of N-([1,1′-Biphenyl]-4-ylmethyl)-5-hydroxy-6-(1H-pyrazol-1-yl) nicotinamide (75)

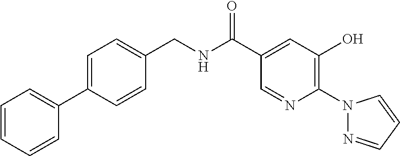 [SMILES: O=C(NCc1ccc(-c2ccccc2)cc1)c1cnc(-n2cccn2)c(O)c1]

Following general procedure D: 73 (12 mg, 0.031 mmol) and LiCl (19 mg, 0.31 mmol) in DMAc (2 ml) gave 75 (4 mg, 0.01 mmol, 35%).

¹H NMR (500 MHZ, DMSO-d₆) δ 11.68 (s, 1H), 9.28 (t, J=6.0 Hz, 1H), 8.76 (d, J=2.5 Hz, 1H), 8.52 (d, J=2.0 Hz, 1H), 7.96 (d, J=2.0 Hz, 1H), 7.72-7.60 (m, 4H), 7.48-7.41 (m, 4H), 7.39-7.30 (m, 1H), 6.74-6.72 (m, 1H), 4.55 (d, J=6.0 Hz, 2H).

HRMS (ESI-TOF) calcd for C₂₂H₁₉O₂N₄ [M+H]⁺: 371.1502, found: 371.1503.

### Example 52—Synthesis of N-([1,1′-Biphenyl]-3-ylmethyl)-5-hydroxy-6-(1H-pyrazol-1-yl) nicotinamide (76)

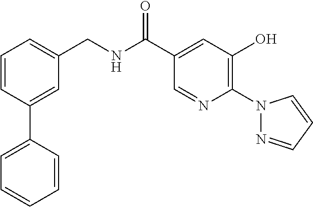 [SMILES: O=C(NCc1cccc(-c2ccccc2)c1)c1cnc(-n2cccn2)c(O)c1]

Following general procedure D: 74 (45 mg, 0.117 mmol) and LiCl (71 mg, 1.17 mmol) in DMAc (3.5 ml) gave 76 (14 mg, 0.037 mmol, 32%).

¹H NMR (400 MHZ,DMSO-d₆) δ 11.68 (s, 1H), 9.28 (t, J=6.0 Hz, 1H), 8.76 (d, J=2.5 Hz, 1H), 8.52 (d, J=2.0 Hz, 1H), 7.97 (d, J=2.0 Hz, 1H), 7.71-7.32 (m, 10H), 6.75-6.71 (m, 1H), 4.59 (d, J=6.0 Hz, 2H).

HRMS (ESI-TOF) calcd for C₂₂H₁₉O₂N₄ [M+H]⁺: 371.1502, found: 371.1502.

### Example 53—Synthesis of Ethyl 1-(5-(([1,1′-biphenyl]-4-ylmethyl) carbamoyl)-4-methoxypyridin-2-yl)-1H-pyrazole-4-carboxylate (77)

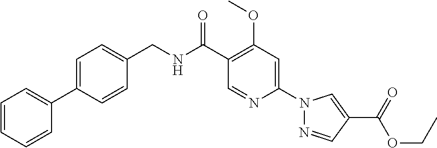 [SMILES: CCOC(=O)c1cnn(-c2cc(OC)c(C(=O)NCc3ccc(-c4ccccc4)cc3)cn2)c1]

Following general procedure C: 33 (150 mg, 0.43 mmol), Pd^{t}BuXPhos G3 (34 mg, 0.043 mmol), Cs₂CO₃ (354 mg, 1.09 mmol), pyrazole-4-carboxylate ethyl ester (89 mg, 0.649 mmol) and ^{t}BuOH (2 ml) gave 77 (93 mg, 0.20 mmol, 48%).

¹H NMR (400 MHz, DMSO-d₆) δ 9.37 (s, 1H), 9.00 (s, 1H), 8.85 (s, 1H), 8.24 (s, 1H), 7.69-7.62 (m, 4H), 7.54-7.29 (m, 6H), 4.59 (d, J=6.0 Hz, 2H), 4.28 (q, J=7.0 Hz, 2H), 4.09 (s, 3H), 1.31 (t, J=7.0 Hz, 3H).

HRMS (ESI-TOF) calcd for C₂₆H₂₅O₄N₄ [M+H]⁺: 457.1870, found: 457.1876.

### Example 54—Synthesis of N-([1,1′-Biphenyl]-4-ylmethyl)-6-(4-cyano-1H-pyrazol-1-yl)-4-methoxynicotinamide (78)

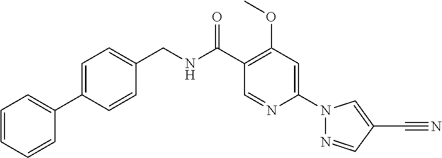 [SMILES: COc1cc(-n2cc(C#N)cn2)ncc1C(=O)NCc1ccc(-c2ccccc2)cc1]

Following general procedure C: 33 (150 mg, 0.426 mmol), Pd^{t}BuXPhos G3 (16 mg, 0.0213 mmol), Cs₂CO₃ (276 mg, 0.852 mmol), pyrazole-4-nitrile (47 mg, 0.511 mmol) and ^{t}BuOH (4 ml) gave 78 (9 mg, 0.022 mmol, 5%).

¹H NMR (400 MHZ, DMSO-d₆) δ 9.47 (d, J=0.1 Hz, 1H), 8.87 (t, J=6.0 Hz, 1H), 8.66 (s, 1H), 8.48 (d, J=1.0 Hz, 1H), 7.70-7.31 (m, 10H), 4.55 (d, J=6.0 Hz, 2H), 4.09 (s, 3H).

HRMS (ESI-TOF) calcd for C₂₄H₁₈O₂N₅ [M−H]⁻: 408.1466, found: 408.1467.

### Example 55—Synthesis of Ethyl 1-(5-(([1,1′-biphenyl]-4-ylmethyl) carbamoyl)-3-methoxypyridin-2-yl)-1H-pyrazole-4-carboxylate (79)

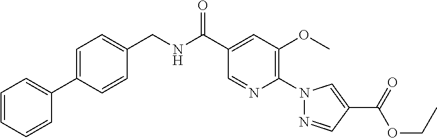 [SMILES: CCOC(=O)c1cnn(-c2ncc(C(=O)NCc3ccc(-c4ccccc4)cc3)cc2OC)c1]

Following general procedure C: 72 (150 mg, 0.43 mmol), Pd^{t}BuXPhos G3 (34 mg, 0.043 mmol), Cs₂CO₃ (354 mg, 1.09 mmol), pyrazole-4-carboxylate ethyl ester (89 mg, 0.649 mmol) and ^{t}BuOH (4 ml) gave 79 (44 mg, 0.096 mmol, 23%).

¹H NMR (400 MHZ, DMSO-d₆) δ 9.41 (d, J=6.0 Hz, 1H), 8.78-8.75 (m, 1H), 8.64 (d, J=2.5 Hz, 1H), 8.15 (d, J=2.5 Hz, 1H), 7.67-7.61 (m, 5H), 7.53-7.42 (m, 4H), 7.39-7.34 (m, 1H), 4.59 (d, J=6.0 Hz, 2H), 4.27 (q, J=7.0 Hz, 2H), 4.00-3.94 (s, 3H), 1.30 (t, J=7.0 Hz, 3H).

HRMS (ESI-TOF) calcd for C₂₆H₂₅O₄N₄ [M+H]⁺: 457.1870, found: 457.1874.

### Example 56—Synthesis of Ethyl 1-(4-Methoxy-5-((4-(trifluoromethyl)benzyl) carbamoyl)pyridin-2-yl)-1H-pyrazole-4-carboxylate (80)

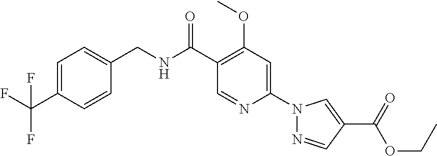 [SMILES: CCOC(=O)c1cnn(-c2cc(OC)c(C(=O)NCc3ccc(C(F)(F)F)cc3)cn2)c1]

Following general procedure C: 45 (100 mg, 0.29 mmol), RockPhos Pd G3 (20 mg, 0.029 mmol), Cs₂CO₃ (188 mg, 0.58 mmol), 4-ethyl ester pyrazole (24 mg, 0.63 mmol) and BuOH (4 ml) gave 80 (86 mg, 0.192 mmol, 66%).

¹H NMR (400 MHZ, Chloroform-d) δ 9.12 (s, 1H), 9.07 (d, J=1.0 Hz, 1H), 8.11 (d, J=1.0 Hz, 1H), 7.89 (t, J=6.0 Hz, 1H), 7.63 (s, 1H), 7.62-7.58 (m, 2H), 7.50-7.45 (m, 2H), 4.73 (d, J=6.0 Hz, 2H), 4.35 (q, J=7.0 Hz, 2H), 4.11 (s, 3H), 1.37 (t, J=7.0 Hz, 3H).

HRMS (ESI-TOF) calcd for C₂₁H₁₈O₄N₄F₃ [M−H]⁻: 447.1285, found: 447.1281.

### Example 57—Synthesis of 6-(4-Cyano-1H-pyrazol-1-yl)-4-methoxy-N-(4-(trifluoromethyl)benzyl) nicotinamide (81)

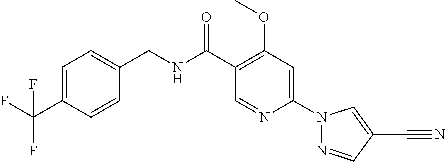 [SMILES: COc1cc(-n2cc(C#N)cn2)ncc1C(=O)NCc1ccc(C(F)(F)F)cc1]

Following general procedure C: 45 (100 mg, 0.29 mmol), RockPhos Pd G3 (24 mg, 0.029 mmol), Cs₂CO₃ (180 mg, 0.554 mmol), pyrazole-4-nitrile (24 mg, 0.35 mmol) and ^{t}BuOH (4 ml) gave 81 (23 mg, 0.057 mmol, 20%).

¹H NMR (400 MHZ, Chloroform-d) δ 9.13 (s, 1H), 9.04 (d, J=1.0 Hz, 1H), 7.99 (d, J=1.0 Hz, 1H), 7.88 (t, J=6.0 Hz, 1H), 7.67-7.56 (m, 3H), 7.51-7.42 (m, 2H), 4.74 (d, J=6.0 Hz, 2H), 4.03 (s, 3H).

HRMS (ESI-TOF) calcd for C₁₈H₁₅O₄N₄F₃ [M+H]⁺: 402.1172, found: 402.1172.

### Example 58—Synthesis of Ethyl 1-(4-methoxy-5-(((6-phenylpyridin-3-yl)methyl) carbamoyl)pyridin-2-yl)-1H-pyrazole-4-carboxylate (82)

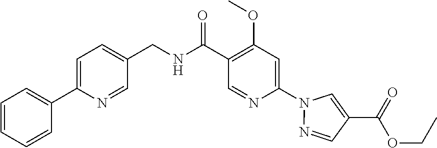 [SMILES: CCOC(=O)c1cnn(-c2cc(OC)c(C(=O)NCc3ccc(-c4ccccc4)nc3)cn2)c1]

Following general procedure C: 53 (80 mg, 0.226 mmol), RockPhosPd G3 (19 mg, 0.0226 mmol), Cs₂CO₃ (145 mg, 0.452 mmol), 4-ethyl ester pyrazole (31 mg, 0.226 mmol) and ^{t}BuOH (2.5 ml) gave 82 (11 mg, 0.024 mmol, 11%).

¹H NMR (400 MHZ, Chloroform-d) δ 9.13 (s, 1H), 9.08 (s, 1H), 8.70-8.68 (m, 1H), 8.12 (s, 1H), 8.01-7.94 (m, 2H), 7.90 (t, J=6.0 Hz, 1H), 7.84-7.37 (m, 6H), 4.73 (d, J=6.0 Hz, 2H), 4.32 (q, J=7.0 Hz, 2H), 4.11 (s, 3H), 1.37 (t, J=7.0 Hz, 3H).

HRMS (ESI-TOF) calcd for C₂₅H₂₄O₄N₅ [M+H]⁺: 458.1822, found: 458.1816.

### Example 59—Synthesis of Ethyl 1-(5-(([1,1′-biphenyl]-4-ylmethyl) carbamoyl)-4-hydroxypyridin-2-yl)-1H-pyrazole-4-carboxylate (83)

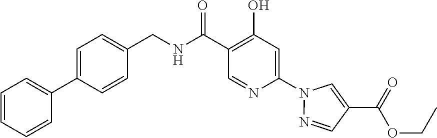 [SMILES: CCOC(=O)c1cnn(-c2cc(O)c(C(=O)NCc3ccc(-c4ccccc4)cc3)cn2)c1]

Following general procedure D: 80 (82 mg, 0.179 mmol) and LiCl (mg, 1.79 mmol) in DMAc (35 ml) gave 83 (70 mg, 0.158 mmol, 90%).

¹H NMR (400 MHZ, DMSO-d₆) δ 9.42 (s, 1H), 9.00 (s, 1H), 8.85 (s, 1H), 8.24 (s, 1H), 7.72-7.58 (m, 4H), 7.54-7.39 (m, 5H), 7.38-7.29 (m, 1H), 4.59 (d, J=6.0 Hz, 2H,), 4.27 (q, J=7.0 Hz, 2H), 1.30 (t, J=7.0 Hz, 3H).

HRMS (ESI-TOF) calcd for C₂₅H₂₃O₄N₄ [M+H]⁺: 443.1713, found: 443.1713.

### Example 60—Synthesis of 1-(5-(([1,1′-Biphenyl]-4-ylmethyl) carbamoyl)-4-hydroxypyridin-2-yl)-1H-pyrazole-4-carboxylic acid (84)

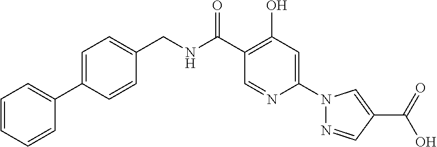 [SMILES: O=C(O)c1cnn(-c2cc(O)c(C(=O)NCc3ccc(-c4ccccc4)cc3)cn2)c1]

83 (65 mg, 0.147 mmol) was dissolved in THF (5 ml) and water (0.5 ml) was added to the reaction mixture before the addition of LiOH-monohydrate (16 mg, 0.40 mmol). The resultant mixture was stirred overnight at room temperature. The reaction mixture was acidified to pH 3 with HCL_{aq} (1M) and extracted with EtOAc (3×25 ml), washed with brine dried with anhydrous Na₂SO₄. The crude compound was then purified using flash column chromatography using (CH₂Cl₂: MeOH 0-20%: Formic Acid 1%) over 20 columns volumes gave 84 (52 mg, 0.125 mmol, 85%) as an off-white solid.

¹H NMR (400 MHZ, DMSO-d₆) δ 13.34 (s, 1H), 9.42 (s, 1H), 9.00 (s, 1H), 8.96 (s, 1H), 8.19 (s, 1H), 7.71-7.31 (m, 11H), 4.59 (d, J=6.0 Hz, 2H).

HRMS (ESI-TOF) calcd for C₂₃H₁₉O₄N₄ [M+H]⁺: 415.1400, found: 415.1401.

### Example 61—Synthesis of N-([1,1′-Biphenyl]-4-ylmethyl)-6-(4-cyano-1H-pyrazol-1-yl)-4-hydroxynicotinamide (85)

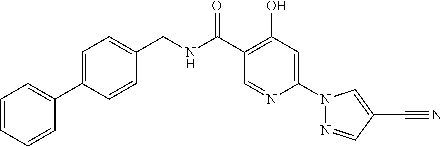 [SMILES: N#Cc1cnn(-c2cc(O)c(C(=O)NCc3ccc(-c4ccccc4)cc3)cn2)c1]

78 (6 mg, 0.0146 mmol) was dissolved in DMAc (1.5 ml) in a microwave vial, before the addition of Cs₂CO₃ (14.3 mg, 0.044 mmol). The resultant reaction mixture was heated under microwave irradiation at 130° C. for 1 hr. H₂O (50 ml) was added, and the resultant mixture was extracted with EtOAc (3×15 ml). The combined organic fractions were washed with brine, dried over Na₂SO₄ and the solvents were removed in vacuo. The crude compound was then purified using flash column chromatography using (CH₂Cl₂: MeOH 0-10%) over 20 column volumes to give 85 (2.5 mg, 0.006 mmol, 44%).

¹H NMR (400 MHZ, DMSO-d₆) δ 9.45 (s, 1H), 8.87 (d, J=2.0 Hz, 1H), 8.44 (s, 1H), 7.73-7.42 (m, 11H), 4.60 (d, J=6.0 Hz, 2H).

HRMS (ESI-TOF) calcd for C₂₃H₁₆O₂N₅ [M−H]⁻: 394.1309, found: 394.1309.

### Example 62—Synthesis of 1-(5-(([1,1′-Biphenyl]-4-ylmethyl) carbamoyl)-3-hydroxypyridin-2-yl)-1H-pyrazole-4-carboxylic acid (86)

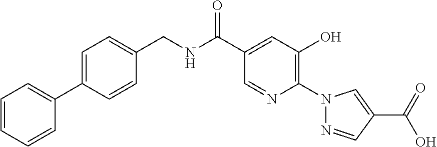 [SMILES: O=C(O)c1cnn(-c2ncc(C(=O)NCc3ccc(-c4ccccc4)cc3)cc2O)c1]

79 (20 mg, 0.043 mmol) was dissolved in THF (2 ml), MeOH (2 ml) and water (0.5 ml) was added to the reaction mixture before the addition of LiOH-monohydrate (16 mg, 0.40 mmol). The resultant mixture was stirred overnight at room temperature. The reaction mixture was acidified to PH3 with HCl_{aq} (1M) solution and extracted with EtOAc (3×25 ml), washed with brine dried with anhydrous Na₂SO₄. The crude compound was then purified using flash column chromatography using (CH₂Cl₂: MeOH 0-20%: formic acid 1%) over 20 column volumes to give 86 (8 mg, 0.019 mmol, 45%).

¹H NMR (400 MHZ, DMSO-d₆) δ 9.09 (s, 1H), 8.90 (d, J=6.0 Hz, 1H), 7.92-7.09 (m, 12H), 4.47 (d, J=6.0 Hz, 2H).

HRMS (ESI-TOF) calcd for C₂₃H₁₇O₄N₄ [M−H]⁻: 413.1255, found: 413.1252.

### Example 63—Synthesis of Ethyl 1-(4-hydroxy-5-((4-(trifluoromethyl)benzyl) carbamoyl)pyridin-2-yl)-1H-pyrazole-4-carboxylate (87)

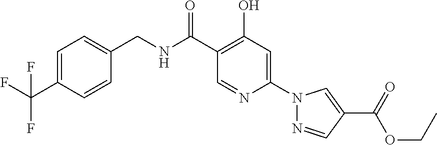 [SMILES: CCOC(=O)c1cnn(-c2cc(O)c(C(=O)NCc3ccc(C(F)(F)F)cc3)cn2)c1]

Following general procedure D: 80 (68 mg, 0.151 mmol) and LiCl (91 mg, 1.51 mmol) in DMAc (3.5 ml) gave 87 (48 mg, 0.110 mmol, 73%).

¹H NMR (400 MHZ, THF-d₈) δ 13.46 (s, 1H), 8.99 (d, J=1.0 Hz, 1H), 8.93-8.85 (m, 1H), 8.74 (s, 1H), 8.07 (d, J=1.0 Hz, 1H), 7.65 (d, J=7.0 Hz, 2H), 7.58 (d, J=7.0 Hz, 2H), 7.48 (s, 1H), 4.71 (d, J=6.0 Hz, 2H), 4.29 (q, J=7.0 Hz, 2H), 1.33 (t, J=7.0 Hz, 3H).

HRMS (ESI-TOF) calcd for C₂₀H₁₆O₄N₄F₃ [M−H]⁻: 433.1129, found: 433.1125.

### Example 64—Synthesis of Ethyl 1-(4-hydroxy-5-(((6-phenylpyridin-3-yl)methyl) carbamoyl)pyridin-2-yl)-1H-pyrazole-4-carboxylate (88)

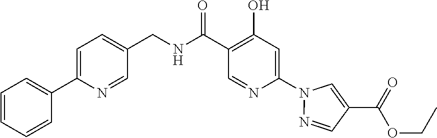 [SMILES: CCOC(=O)c1cnn(-c2cc(O)c(C(=O)NCc3ccc(-c4ccccc4)nc3)cn2)c1]

Following general procedure D: 82 (7 mg, 0.015 mmol) and LiCl (9 mg, 0.15 mmol) in DMAc (2 ml) gave 88 (3.5 mg, 0.0079 mmol, 53%).

¹H NMR (400 MHZ, DMSO-d₆) δ 9.00 (s, 1H), 8.80 (s, 1H), 8.67 (d, J=2.0 Hz, 1H), 8.24 (s, 1H), 8.07 (dd, J=8.0, 2.0 Hz, 2H), 7.95 (d, J=8.0 Hz, 1H), 7.85 (dd, J=8.0, 2.0 Hz, 1H), 7.54-7.39 (m, 5H), 4.61 (d, J=6.0 Hz, 2H), 4.26 (q, J=7.0 Hz, 2H), 1.30 (t, J=7.0 Hz, 3H).

HRMS (ESI-TOF) calcd for C₂₄H₂₂O₄N₅[M+H]⁺: 444.1666, found: 444.1659.

### Example 65—Synthesis of 6-(4-Cyano-1H-pyrazol-1-yl)-4-hydroxy-N-(4-(trifluoromethyl)benzyl) nicotinamide (89)

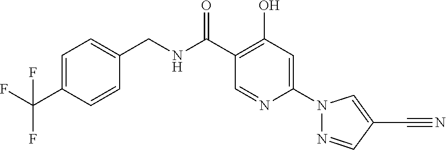 [SMILES: N#Cc1cnn(-c2cc(O)c(C(=O)NCc3ccc(C(F)(F)F)cc3)cn2)c1]

Following general procedure D: 81 (20 mg, 0.049 mmol) and LiCl (30 mg, 0.50 mmol) in DMAc (2 ml) gave 89 (5 mg, 0.012 mmol, 26%).

¹H NMR (400 MHZ, DMSO-d₆) δ 9.43 (d, J=1.0 Hz, 1H), 8.81 (s, 1H), 8.43 (d, J=1.0 Hz, 1H), 7.71 (d, J=8.0 Hz, 2H), 7.57 (d, J=8.0 Hz, 2H), 7.42 (s, 1H), 4.63 (d, J=6.0 Hz, 2H).

HRMS (ESI-TOF) calcd for C₁₈H₁₁O₄N₄F₃ [M−H]⁻: 386.0870, found: 386.0867.

### Example 66—Synthesis of 1-(4-Hydroxy-5-((4-(trifluoromethyl)benzyl) carbamoyl)pyridin-2-yl)-1H-pyrazole-4-carboxylic acid (90)

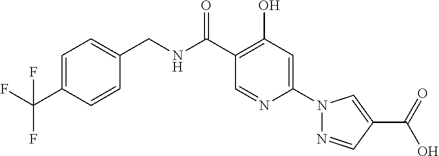 [SMILES: O=C(O)c1cnn(-c2cc(O)c(C(=O)NCc3ccc(C(F)(F)F)cc3)cn2)c1]

87 (40 mg, 0.092 mmol) was dissolved in THF (2 ml), MeOH (2 ml) and water (0.5 ml) was added to the reaction mixture before the addition of LiOH-monohydrate (19 mg, 0.46 mmol). The resultant mixture was stirred overnight at room temperature. The reaction mixture was acidified to pH 3 with HCL_{aq} (1M) and extracted with EtOAc (3×25 ml), washed with brine, dried with anhydrous Na₂SO₄. The crude compound was then purified using flash column chromatography using (CH₂Cl₂: MeOH 0-20%: Formic acid 1%) over 20 column volumes gave 90 (17 mg, 0.042 mmol, 46%).

¹H NMR (400 MHZ, DMSO-d₆) δ 9.56 (s, 1H), 8.94 (s, 1H), 8.78 (s, 1H), 8.17 (s, 1H), 7.70 (d, J=8.0 Hz, 2H), 7.56 (d, J=8.0 Hz, 2H), 7.38 (s, 1H), 4.63 (d, J=6.0 Hz, 2H).

HRMS (ESI-TOF) calcd for C₁₈H₁₂O₄N₄F₃ [M−H]⁻: 405.0816, found: 405.0815.

### Example 67—Synthesis of 1-(4-Hydroxy-5-(((6-phenylpyridin-3-yl)methyl) carbamoyl)pyridin-2-yl)-1H-pyrazole-4-carboxylic acid (91)

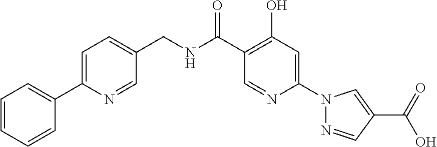 [SMILES: O=C(O)c1cnn(-c2cc(O)c(C(=O)NCc3ccc(-c4ccccc4)nc3)cn2)c1]

88 (3 mg, 0.0072 mmol) was dissolved in THF (0.8 ml), MeOH (0.15 ml) and water (0.05 ml) was added to the reaction mixture before the addition of LiOH-monohydrate (1 mg, 0.023 mmol). The resultant mixture was stirred overnight at room temperature. The reaction mixture was acidified to pH 3 with HCl aq (1M) and was using preparative HPLC to give 91 (1 mg, 0.0024 mmol, 33%).

¹H NMR (500 MHZ, DMSO-d₆) δ 8.94 (s, 1H), 8.68 (s, 1H), 8.19 (s, 1H), 8.13-8.05 (m, 2H), 7.96 (d, J=8.5 Hz, 1H), 7.87 (d, J=8.5 Hz, 1H), 7.55-7.39 (m, 6H), 4.62 (d, J=6.0 Hz, 2H).

HRMS (ESI-TOF) calcd for C₂₂H₁₈O₄N₅[M+H]⁺: 416.1353, found: 416.1352.

### Reference Example 68—Synthesis of N-([1,1′-Biphenyl]-4-ylmethyl)-1H-pyrazole-4-carboxamide (92)

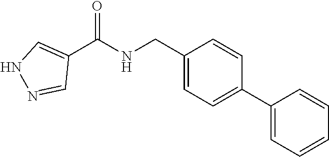 [SMILES: O=C(NCc1ccc(-c2ccccc2)cc1)c1cn[nH]c1]

Following general procedure B: 1H-pyrazole-4-carboxylic acid (1 g, 8.9 mmol), 4-phenylbenzylamine (2.44 g, 13.35 mmol), T3P (7.05 g, 22.2 mmol) and DIPEA (4.6 g, 35.7 mmol) gave 92 (462 mg, 1.66 mmol, 19%).

¹H NMR (400 MHZ, DMSO-d₆) δ 8.66 (t, J=6.0 Hz, 1H), 8.09 (s, 2H), 7.70-7.58 (m, 4H), 7.50-7.30 (m, 5H,), 4.47 (d, J=6.0 Hz, 2H).

HRMS (ESI-TOF) calcd for C₁₇H₁₆ON₃ [M+H]⁺: 278.1287, found: 278.1290.

### Example 69—Synthesis of N-([1,1′-Biphenyl]-4-ylmethyl)-1-(5-cyano-4-methoxypyridin-2-yl)-1H-pyrazole-4-carboxamide (93)

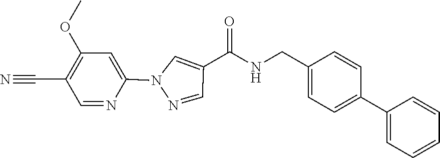 [SMILES: COc1cc(-n2cc(C(=O)NCc3ccc(-c4ccccc4)cc3)cn2)ncc1C#N]

General Procedure C: 6-chloro-4-methoxynicotinonitrile (40 mg, 0.238 mmol), 92 (65 mg, 0.238 mmol), Pd^{t}BuXPhos Pd G3 (19 mg, 0.023 mmol) and Cs₂CO₃ (232 mg, 0.714 mmol) in gave 93 (14 mg, 0.034 mmol, 12%).

¹H NMR (400 MHZ, DMSO-d₆) δ 9.03 (t, J=6.0 Hz, 1H), 8.82 (s, 1H), 8.30-8.29 (m, 1H), 7.70-7.59 (m, 5H), 7.50-7.31 (m, 6H), 4.50 (d, J=6.0 Hz, 2H), 4.12 (s, 3H).

HRMS (ESI-TOF) calcd for C₂₄H₂₀O₂N₅ [M+H]⁺: 410.1622, found: 410.1611.

### Example 70—Synthesis of Ethyl 6-(4-(([1,1′-Biphenyl]-4-ylmethyl) carbamoyl)-1H-pyrazol-1-yl)-4-methoxynicotinate (94)

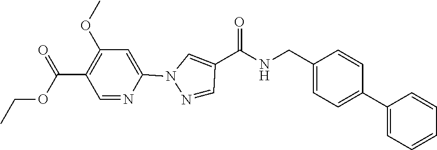 [SMILES: CCOC(=O)c1cnc(-n2cc(C(=O)NCc3ccc(-c4ccccc4)cc3)cn2)cc1OC]

Following general procedure C: Ethyl 6-chloro-4-methoxynicotinate (39 mg, 0.18 mmol), 92 (50 mg, 0.18 mmol), Pd^{t}BuXPhos Pd G3 (25 mg, 0.009 mmol) and dioxane (3 mL) gave 94 as a clear oil (23 mg, 0.05 mmol, 28%).

¹H NMR (400 MHZ, THF-d₈) δ 9.08 (d, J=1.0 Hz, 1H), 8.68 (s, 1H), 8.13 (d, J=1.0 Hz, 1H), 8.05 (t, J=6.0 Hz, 1H), 7.69 (s, 1H), 7.64-7.23 (m, 9H), 4.59 (d, J=6.0 Hz, 2H), 4.30 (q, J=7.0 Hz, 2H), 4.03 (s, 3H), 1.34 (t, J=7.0 Hz, 3H).

HRMS (ESI-TOF) calcd for C₂₆H₂₅O₄N₄ [M+H]⁺: 457.1870, found: 457.1868.

### Example 71—Synthesis of N-([1,1′-Biphenyl]-4-ylmethyl)-1-(5-cyano-4-hydroxypyridin-2-yl)-1H-pyrazole-4-carboxamide (95)

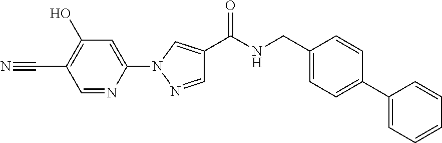 [SMILES: N#Cc1cnc(-n2cc(C(=O)NCc3ccc(-c4ccccc4)cc3)cn2)cc1O]

Following general procedure D: 93 (6 mg, 0.014 mmol) and LiCl (8.5 mg, 0.14 mmol) in DMAc (1 ml) gave 95 (1.9 mg, 0.0048 mmol, 35%).

¹H NMR (400 MHz, DMSO-d₆) δ 9.14 (s, 1H), 8.95 (t, J=6.0 Hz, 1H), 8.45 (s, 1H), 8.16 (s, 1H), 7.70-7.59 (m, 4H), 7.53-7.29 (m, 5H), 7.16 (s, 1H), 4.48 (d, J=6.0 Hz, 2H).

HRMS (ESI-TOF) calcd for C₂₃H₁₈O₂N₅ [M+H]⁺: 396.1466, found: 396.1456.

### Example 72—Synthesis of 6-(4-(([1,1′-Biphenyl]-4-ylmethyl) carbamoyl)-1H-pyrazol-1-yl)-4-hydroxynicotinic acid (96)

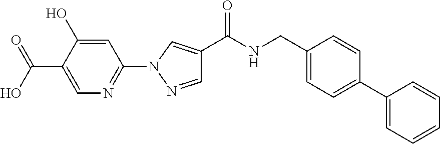 [SMILES: O=C(NCc1ccc(-c2ccccc2)cc1)c1cnn(-c2cc(O)c(C(=O)O)cn2)c1]

Following general procedure D: 94 (23 mg, 0.0504 mmol) and LiCl (30 mg, 0.504 mmol) in DMAc (3 ml) gave 96 as a yellow oil (9 mg, 0.0216 mmol, 43%).

¹H NMR (600 MHZ, DMSO-d₆) δ 9.21 (s, 1H), 8.96 (t, J=6.0 Hz, 1H), 8.71 (s, 1H), 8.22 (s, 1H), 7.64 (m, 5H), 7.51-7.32 (m, 5H), 4.49 (d, J=6.0 Hz, 2H).

HRMS (ESI-TOF) calcd for C₂₃H₁₉O₄N₄ [M+H]⁺: 415.1400, found: 415.1401.

### Example 73—Synthesis of N-([1,1′-Biphenyl]-4-ylmethyl)-1-(5-cyanopyridin-2-yl)-1H-pyrazole-4-carboxamide (97)

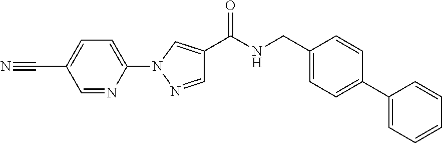 [SMILES: N#Cc1ccc(-n2cc(C(=O)NCc3ccc(-c4ccccc4)cc3)cn2)nc1]

Following general procedure C: 6-chloro-nicotinonitrile (30 mg, 0.216 mmol), 92 (30 mg, 0.108 mmol), Pd^{t}BuXPhos G3 (17 mg, 0.0216 mmol), Cs₂CO₃ (175 mg, 0.432 mmol) gave 97 (6 mg, 0.015 mmol, 16%).

¹H NMR (400 MHZ, DMSO-d₆) δ 9.27 (d, J=1.0 Hz, 1H), 9.10-8.96 (m, 2H), 8.51 (dd, J=8.5, 2.0 Hz, 1H), 8.29 (d, J=1.0 Hz, 1H), 8.10 (dd, J=8.5, 1.0 Hz, 1H), 7.67-7.59 (m, 4H), 7.51-7.31 (m, 5H), 4.50 (d, J=6.0 Hz, 2H).

HRMS (ESI-TOF) calcd for C₂₃H₁₆ON₅ [M−H]⁻: 378.1360, found: 378.1354.

### Example 74—Synthesis of N-([1,1′-Biphenyl]-4-ylmethyl)-1-(4-methoxypyridin-2-yl)-1H-pyrazole-4-carboxamide (98)

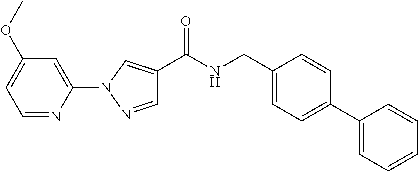 [SMILES: COc1ccnc(-n2cc(C(=O)NCc3ccc(-c4ccccc4)cc3)cn2)c1]

Following general procedure C: 2-chloro-4-methoxypyridine (16 mg, 0.114 mmol), 92 (31 mg, 0.114 mmol), RockPhos Pd G3 (9.5 mg, 0.0114 mmol) and Cs₂CO₃ (92 mg, 0.285 mmol) gave 98 (28 mg, 0.072 mmol, 63%).

¹H NMR (400 MHZ, DMSO-d₆) δ 9.18 (d, J=1.0 Hz, 1H), 8.95 (t, J=6.0 Hz, 1H), 8.32 (d, J=6.0 Hz, 1H), 8.19 (d, J=1.0 Hz, 1H), 7.69-7.31 (m, 10H), 7.00 (dd, J=6.0, 2.5 Hz, 1H), 4.49 (d, J=6.0 Hz, 2H), 3.93 (s, 3H).

HRMS (ESI-TOF) calcd for C₂₃H₂₁O₂N₄ [M+H]⁺: 385.1690, found: 385.1659.

### Example 75—Synthesis of N-([1,1′-Biphenyl]-4-ylmethyl)-1-(4-hydroxypyridin-2-yl)-1H-pyrazole-4-carboxamide (99)

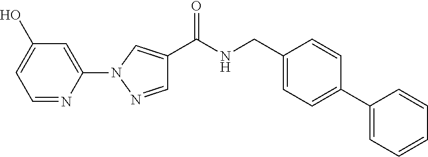 [SMILES: O=C(NCc1ccc(-c2ccccc2)cc1)c1cnn(-c2cc(O)ccn2)c1]

Following general procedure D: 92 (7 mg, 0.018 mmol) and LiCl (11 mg, 0.18 mmol) dissolved in DMAc (1 ml) gave 99 (3 mg, 0.008 mmol, 45%).

¹H NMR (400 MHZ, DMSO-d₆) δ 9.14 (d, J=1.0 Hz, 1H), 8.89 (t, J=6.0 Hz, 1H), 8.73-8.66 (m, 1H), 8.33-8.29 (m, 1H), 7.91-7.89 (m, 1H), 7.50-7.28 (m, 9H), 6.77 (dd, J=6.0, 2.5 Hz, 1H), 4.48 (d, J=6.0 Hz, 2H).

HRMS (ESI-TOF) calcd for C₂₂H₁₉O₂N₄ [M+H]⁺: 371.1506, found: 371.1506.

### Example 76—Synthesis of Ethyl 6-(4-(([1,1′-biphenyl]-4-ylmethyl) carbamoyl)-1H-pyrazol-1-yl) nicotinate (100)

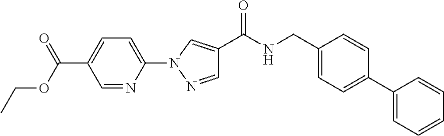 [SMILES: CCOC(=O)c1ccc(-n2cc(C(=O)NCc3ccc(-c4ccccc4)cc3)cn2)nc1]

Following general procedure C: Ethyl 6-chloronicotinate (42 mg, 0.23 mmol), 92 (30 mg, 0.108 mmol), Pd^{t}BuXPhos G3 (18 mg, 0.023 mmol), Cs₂CO₃ (149 mg, 0.46 mmol) in dioxane (5 ml) gave 100 as a (10 mg, 0.023 mmol, 21%).

¹H NMR (400 MHZ, THF-d₈) δ 9.09 (d, J=1.0 Hz, 1H), 9.00 (dd, J=2.0, 1.0 Hz, 1H), 8.70-8.60 (m, 1H), 8.14-8.10 (m, 1H), 7.96 (t, J=6.0 Hz, 1H), 7.67-7.35 (m, 10H,), 4.59 (d, J=6.0 Hz, 2H), 4.40 (q, J=7.0 Hz, 2H), 1.38 (t, J=7.0 Hz, 3H).

HRMS (ESI-TOF) calcd for C₂₅H₂₃O₃N₄ [M+H]⁺: 427.1766, found: 427.1766.

### Example 77—Synthesis of 6-(4-(([1,1′-Biphenyl]-4-ylmethyl) carbamoyl)-1H-pyrazol-1-yl) nicotinic acid (101)

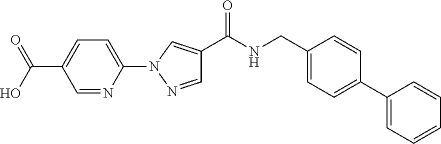 [SMILES: O=C(O)c1ccc(-n2cc(C(=O)NCc3ccc(-c4ccccc4)cc3)cn2)nc1]

100 (8 mg, 0.0187 mmol) was dissolved in a mixture of THF and water (3 ml (10:1). Lithium hydroxide monohydrate (1.5 mg, 0.0374 mmol) was added to the reaction mixture and the resulting mixture was allowed to stir at room temperature for 16 hrs. The reaction was confirmed to have gone to completion by TLC and HCl aq (5 ml, 1M) was added to the reaction mixture. The resulting mixture was extracted with EtOAc (3×10 ml), then washed with brine, then dried with anhydrous Na₂SO₄ and purified using flash column chromatography using (CH₂Cl₂, MeOH 0-5%, 1% formic acid) gave 101 (2 mg, 0.005 mmol, 27%).

¹H NMR (400 MHZ, DMSO-d₆) δ 9.26 (d, J=1.0 Hz, 1H), 8.97 (t, J=6.0 Hz, 1H), 8.87 (s, 1H), 8.35 (d, J=2.0 Hz, 1H), 8.19 (d, J=7.0 Hz, 1H), 7.89 (d, J=7.0 Hz, 1H), 7.68-7.55 (m, 4H), 7.52-7.29 (m, 5H), 4.49 (d, J=6.0 Hz, 2H).

HRMS (ESI-TOF) calcd for C₂₃H₁₇O₃N₄ [M−H]⁻: 397.1304, found: 397.1304.

### Example 78—Synthesis of Methyl 2-(4-(([1,1′-Biphenyl]-4-ylmethyl) carbamoyl)-1H-pyrazol-1-yl) isonicotinate (102)

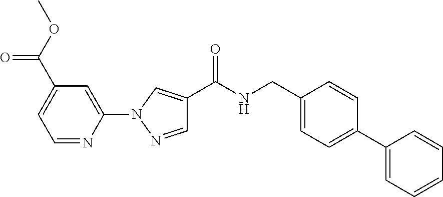 [SMILES: COC(=O)c1ccnc(-n2cc(C(=O)NCc3ccc(-c4ccccc4)cc3)cn2)c1]

Following general procedure C: Methyl 2-chloroisonictinate (37 mg, 0.216 mmol), 92 (30 mg, 0.108 mmol), 1BuXPhos Pd G3 (17 mg, 0.0216 mmol) and Cs₂CO₃ (175 mg, 0.54 mmol) in dioxane (3 ml) gave 102 (6 mg, 0.014 mmol, 13%).

¹H NMR (400 MHz, DMSO-d₆) δ 9.24 (d, J=1.0 Hz, 1H), 8.99 (t, J=6.0 Hz, 1H), 8.73 (dd, J=5.0, 1.0 Hz, 1H), 8.34 (dd, J=1.5, 1.0 Hz, 1H), 8.26 (d, J=1.0 Hz, 1H), 7.85 (dd, J=5.0, 1.5 Hz, 1H), 7.66-7.61 (m, 4H), 7.51-7.29 (m, 5H), 4.50 (d, J=6.0 Hz, 2H), 3.95 (s, 3H).

HRMS (ESI-TOF) calcd for C₂₄H₂₁O₃N₄ [M+H]⁺: 413.1608, found: 413.1608.

### Example 79—Synthesis of 2-(4-(([1,1′-Biphenyl]-4-ylmethyl) carbamoyl)-1H-pyrazol-1-yl) isonicotinic acid (103)

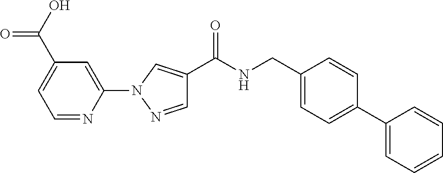 [SMILES: O=C(O)c1ccnc(-n2cc(C(=O)NCc3ccc(-c4ccccc4)cc3)cn2)c1]

102 (4 mg, 0.0097 mmol) was dissolved in a mixture of THF and water (3 ml (10:1). Lithium hydroxide monohydrate (1 mg, 0.0194 mmol) was added to the reaction mixture and the resulting mixture was allowed to stir at room temperature for 16 hrs. The reaction was confirmed to have gone to completion by LCMS and HCl_{aq} (5 ml, 1M) was added to the reaction mixture. The resultant mixture was extracted with EtOAc (3×10 ml), then washed with brine, dried with anhydrous Na₂SO₄ and purified using flash column chromatography using (CH₂Cl₂, MeOH 0-5%, formic acid 1%) over 20 column volumes gave 103 (3 mg, 0.0075 mmol, 78%).

¹H NMR (400 MHZ, DMSO-d₆) δ 9.24 (s, 1H), 8.98 (t, J=6.0 Hz, 1H), 8.68 (d, J=5.0 Hz, 1H), 8.33 (t, J=1.5 Hz, 1H), 8.24 (s, 1H), 7.81 (dd, J=5.0, 1.5 Hz, 1H), 7.69-7.61 (m, 4H), 7.50-7.37 (m, 5H), 4.50 (d, J=6.0 Hz, 2H).

HRMS (ESI-TOF) calcd for C₂₃H₁₇O₃N₄ [M−H]⁻: 397.1304, found: 397.1304.

### Reference Example 80—Synthesis of Ethyl 1-(4-methoxypyridin-2-yl)-1H-pyrazole-4-carboxylate (104)

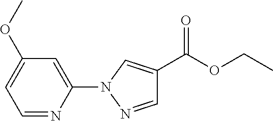 [SMILES: CCOC(=O)c1cnn(-c2cc(OC)ccn2)c1]

Following general procedure C: 4-methoxy-2-chloropyridine (500 mg, 3.49 mmol), 4-ethyl ester pyrazole (725 mg, 5.2 mmol), RockPhos Pd G3 (244 mg, 0.349 mmol), Cs₂CO₃ (2.8 g, 8.7 mmol) and ^{t}BuOH (10 ml) gave 104 (190 mg, 0.769 mmol, 22%).

¹H NMR (400 MHZ, THF-d₈) δ 8.23 (d, J=5.5 Hz, 1H), 8.13 (d, J=5.5 Hz, 1H), 7.57 (d, J=2.0 Hz, 1H), 6.94 (d, J=2.0 Hz, 2H), 4.29 (q, J=7.0 Hz, 2H), 3.94 (s, 3H), 1.33 (t, J=7.0 Hz, 3H).

HRMS (ESI-TOF) calcd for C₁₂H₁₄O₃N₃ [M+H]⁺: 248.1029, found: 248.1033.

### Reference Example 81—Synthesis of 1-(4-Methoxypyridin-2-yl)-1H-pyrazole-4-carboxylic acid (105)

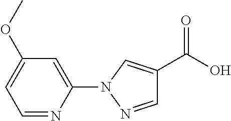 [SMILES: COc1ccnc(-n2cc(C(=O)O)cn2)c1]

104 (190 mg, 0.769 mmol) was dissolved in a mixture of THF and water (11 ml (10:1)). Lithium hydroxide monohydrate (78 mg, 2.19 mmol) was added to the reaction mixture and the resultant mixture was allowed to stir at room temperature for 16 hrs. The reaction was confirmed to have gone to completion by LCMS and HCl (1M, 5 ml) was added to the reaction mixture. The resultant mixture was extracted with EtOAc (3×10 ml), then washed with brine, then dried over Na₂SO₄. The solvents were removed in vacuo to give 105 (73 mg, 0.33 mmol, 44%).

¹H NMR (400 MHZ, DMSO-d₆) δ 8.90 (s, 1H), 8.34 (d, J=6.0 Hz, 1H), 8.15 (s, 1H), 7.45 (d, J=2.5 Hz, 1H), 7.03 (dd, J=6.0, 2.5 Hz, 1H), 3.93 (s, 3H).

HRMS (ESI-TOF) calcd for C₁₀H₈O₃N₃ [M−H]⁻: 218.0569, found: 218.0571.

### Example 82—Synthesis of N-([1,1′-Biphenyl]-4-yl)-1-(4-methoxypyridin-2-yl)-1H-pyrazole-4-carboxamide (106)

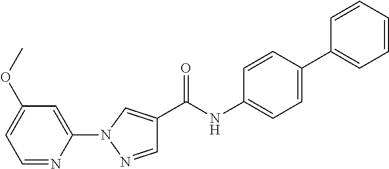 [SMILES: COc1ccnc(-n2cc(C(=O)Nc3ccc(-c4ccccc4)cc3)cn2)c1]

Following general procedure B: 105 (30 mg, 0.136 mmol), 4-aminobiphenyl (35 mg, 0.204 mmol), T3P (108 mg, 0.34 mmol) and DIPEA (116 μl, 0.68 mmol) gave 106 (27 mg, 0.072 mmol, 54%).

¹H NMR (400 MHz, THF-d₈) δ 9.29 (s, 1H), 9.18 (d, J=1.0 Hz, 1H), 8.24 (d, J=6.0 Hz, 1H), 8.17 (d, J=1.0 Hz, 1H), 7.91-7.84 (m, 2H), 7.68-7.21 (m, 8H), 6.87 (d, J=6.0 Hz, 1H), 3.95 (s, 3H).

HRMS (ESI-TOF) calcd for C₂₂H₁₉O₂N₄ [M+H]⁺: 371.1502, found: 371.1396.

### Example 83—Synthesis of N-([1,1′-Biphenyl]-3-ylmethyl)-1-(4-methoxypyridin-2-yl)-1H-pyrazole-4-carboxamide (107)

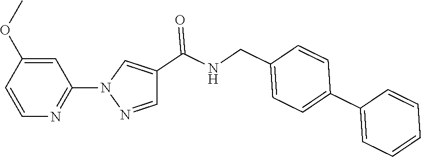 [SMILES: COc1ccnc(-n2cc(C(=O)NCc3ccc(-c4ccccc4)cc3)cn2)c1]

Following general procedure B: 105 (30 mg, 0.136 mmol), 3-aminomethylbiphenyl (36 mg, 0.204 mmol), T3P (108 mg, 0.34 mmol) and DIPEA (116 μl, 0.68 mmol) gave 107 (32 mg, 0.083 mmol, 61%).

¹H NMR (400 MHZ, Chloroform-d) δ 8.97 (d, J=1.0 Hz, 1H), 8.15 (d, J=6.0 Hz, 1H), 8.07 (d, J=1.0 Hz, 1H), 7.58-7.28 (m, 10H), 6.73 (d, J=6.0 Hz, 1H), 6.59 (t, J=6.0 Hz, 1H), 4.64 (d, J=6.0 Hz, 2H), 3.90 (s, 3H).

HRMS (ESI-TOF) calcd for C₂₃H₂₁O₂N₄ [M+H]⁺: 385.1659, found: 385.1656.

### Example 84—Synthesis of N-([1,1′-Biphenyl]-4-yl)-1-(4-hydroxypyridin-2-yl)-1H-pyrazole-4-carboxamide (108)

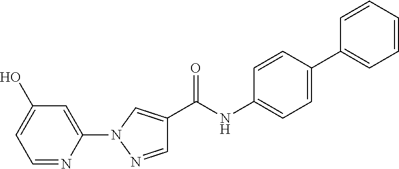 [SMILES: O=C(Nc1ccc(-c2ccccc2)cc1)c1cnn(-c2cc(O)ccn2)c1]

Following general procedure D: 106 (27 mg, 0.073 mmol) and LiCl (29 mg, 0.73 mmol) in DMAc (2 ml) for 8 hrs gave 108 (15 mg, 0.0421 mmol, 57%).

¹H NMR (400 MHZ, DMSO-d₆) δ 10.18 (s, 1H), 9.39 (s, 1H), 8.32-8.18 (m, 2H), 7.91-7.82 (m, 2H), 7.70-7.63 (m, 4H), 7.50-7.31 (m, 4H), 6.82 (dd, J=5.5, 2.5 Hz, 1H).

HRMS (ESI-TOF) calcd for C₂₁H₁₇O₂N₄ [M+H]⁺: 357.1346, found: 357.1348.

### Example 85—Synthesis of N-([1,1′-Biphenyl]-3-ylmethyl)-1-(4-hydroxypyridin-2-yl)-1H-pyrazole-4-carboxamide (109)

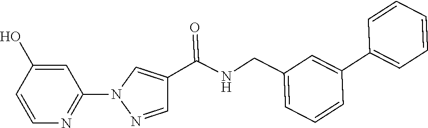 [SMILES: O=C(NCc1cccc(-c2ccccc2)c1)c1cnn(-c2cc(O)ccn2)c1]

Following general procedure D: 107 (32 mg, 0.083 mmol) and LiCl (35 mg, 0.83 mmol) in DMAc (2 ml) gave 109 (11 mg, 0.029 mmol, 36%).

¹H NMR (400 MHZ, DMSO-d₆) δ 11.23 (s, 1H), 9.14 (d, J=1.0 Hz, 1H), 8.93 (t, J=6.0 Hz, 1H), 8.19 (d, J=5.5 Hz, 1H), 8.15 (d, J=1.0 Hz, 1H), 7.70-7.27 (m, 10H), 6.78 (dd, J=5.5, 2.5 Hz, 1H), 4.52 (d, J=6.0 Hz, 2H).

HRMS (ESI-TOF) calcd for C₂₂H₁₉O₂N₄ [M+H]⁺: 371.1502, found: 371.1497.

### Reference Example 86—Synthesis of Ethyl 1-(5-Cyano-4-methoxypyridin-2-yl)-1H-pyrazole-4-carboxylate (110)

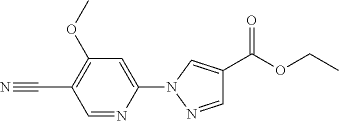 [SMILES: CCOC(=O)c1cnn(-c2cc(OC)c(C#N)cn2)c1]

Following general procedure C: 6-chloro-4-methoxynicotinonitrile (400 mg, 2.38 mmol), pyrazole-4-carboxylate ethyl ester (500 mg, 3.57 mmol), ^{t}BuxPhos Pd G3 (188 mg, 0.238 mmol) and Cs₂CO₃ (2.3 g, 7.14 mmol) in dioxane (10 ml) gave 110 (110 mg, 0.40 mmol, 17%).

¹H NMR (400 MHZ, DMSO-d₆) δ 9.02 (s, 1H), 8.83 (s, 1H), 8.32 (s, 1H), 7.69 (s, 1H), 4.28 (q, J=7.0 Hz, 2H), 4.13 (s, 3H), 1.30 (t, J=7.0 Hz, 3H).

HRMS (ESI-TOF) calcd for C₁₃H₁₃O₃N₄ [M+H]⁺: 273.0978, found: 273.0978.

### Reference Example 87—Synthesis of 1-(5-Cyano-4-methoxypyridin-2-yl)-1H-pyrazole-4-carboxylic acid (111)

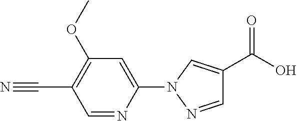 [SMILES: COc1cc(-n2cc(C(=O)O)cn2)ncc1C#N]

110 (95 mg, 0.399 mmol) was dissolved in a mixture of THF and water (11 ml (10:1)). Lithium hydroxide monohydrate (14 mg, 0.399 mmol) was added to the reaction mixture and the resulting mixture was allowed to stir at room temperature for 16 hrs. The reaction was confirmed to have gone to completion by TLC and HCl (10 ml, 1M) was added to the reaction mixture. The resultant mixture was extracted with EtOAc (3×10 ml), then washed with brine, then dried with anhydrous Na₂SO₄ and concentrated under vacuo gave 111 (90 mg, 0.368 mmol, 93%).

¹H NMR (400 MHZ, DMSO-d₆) δ 8.89 (d, J=1.0 Hz, 1H), 8.76 (s, 1H), 8.20 (d, J=1.0 Hz, 1H), 7.63 (s, 1H), 4.08 (s, 3H).

HRMS (ESI-TOF) calcd for C₁₁H₇O₃N₄ [M−H]⁻: 243.0523, found: 243.0520.

### Example 88—Synthesis of 1-(5-Cyano-4-methoxypyridin-2-yl)-N-((6-phenylpyridin-3-yl)methyl)-1H-pyrazole-4-carboxamide (112)

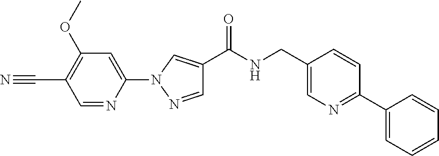 [SMILES: COc1cc(-n2cc(C(=O)NCc3ccc(-c4ccccc4)nc3)cn2)ncc1C#N]

Following general procedure B: 111 (40 mg, 0.164 mmol), 52 (56 mg, 0.306 mmol), T3P (130 mg, 0.409 mmol) and DIPEA (116 μl, 0.68 mmol) gave 112 (28 mg, 0.068 mmol, 35%).

¹H NMR (400 MHZ, THF-d₈) δ 9.06 (d, J=1.0 Hz, 1H), 8.57 (s, 1H), 8.34 (dd, J=8.5, 1.5 Hz, 1H), 8.18 (t, J=6.0 Hz, 1H), 8.12-8.04 (m, 2H), 7.81 (d, J=1.5 Hz, 2H), 7.73 (s, 1H), 7.44-7.31 (m, 4H), 4.59 (d, J=6.0 Hz, 2H), 4.12 (s, 3H).

HRMS (ESI-TOF) calcd for C₂₃H₁₉O₂N₆ [M+H]⁺: 411.1564, found: 411.1555.

### Example 89—Synthesis of 1-(5-Cyano-4-methoxypyridin-2-yl)-N-(4-(trifluoromethyl)benzyl)-1H-pyrazole-4-carboxamide 113

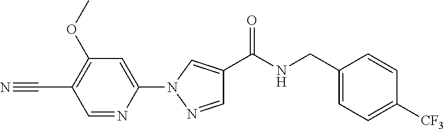 [SMILES: COc1cc(-n2cc(C(=O)NCc3ccc(C(F)(F)F)cc3)cn2)ncc1C#N]

Following general procedure B: 111 (40 mg, 0.164 mmol), 4-trifluoromethyl benzylamine (54 mg, 0.306 mmol), T3P (130 mg, 0.409 mmol) and DIPEA (116 μl, 0.68 mmol) gave 113 (38 mg, 0.0947 mmol, 47%).

¹H NMR (400 MHZ, THF-d₈) δ 8.82 (s, 1H), 8.41 (dd, J=4.5, 1.5 Hz, 1H), 8.11 (dd, J=8.5, 1.5 Hz, 1H), 7.50 (s, 1H), 7.39-7.29 (m, 4H), 7.23 (t, J=6.0 Hz, 1H), 4.39 (d, J=6.0 Hz, 2H), 3.89 (s, 3H).

HRMS (ESI-TOF) calcd for C₁₉H₁₃O₂N₅F₃ [M−H]⁻: 400.1026, found: 400.1022.

### Example 90—Synthesis of 1-(5-Cyano-4-hydroxypyridin-2-yl)-N-((6-phenylpyridin-3-yl)methyl)-1H-pyrazole-4-carboxamide (114)

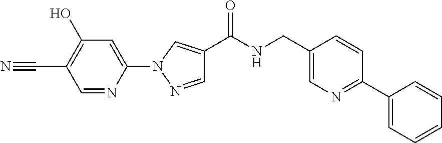 [SMILES: N#Cc1cnc(-n2cc(C(=O)NCc3ccc(-c4ccccc4)nc3)cn2)cc1O]

Following general procedure D: 112 (20 mg, 0.0487 mmol) and LiCl (20.5 mg, 0.487 mmol) in DMAc (2 ml) gave 114 (8 mg, 0.020 mmol, 42%).

¹H NMR (400 MHZ, DMSO-d₆) δ 9.11 (d, J=1.0 Hz, 1H), 9.10-9.04 (m, 1H), 8.64-8.61 (m, 1H), 8.37 (s, 1H), 8.16 (s, 1H), 8.09-8.03 (m, 2H), 7.93 (dd, J=8.0, 1.0 Hz, 1H), 7.81 (dd, J=8.0, 2.5 Hz, 1H), 7.53-7.38 (m, 3H), 7.15 (s, 1H), 4.49 (d, J=6.0 Hz, 2H).

HRMS (ESI-TOF) calcd for C₂₂H₁₇O₂N₆ [M+H]⁺: 397.1407, found: 397.1407.

### Example 91—Synthesis of 1-(5-Cyano-4-hydroxypyridin-2-yl)-N-(4-(trifluoromethyl)benzyl)-1H-pyrazole-4-carboxamide (115)

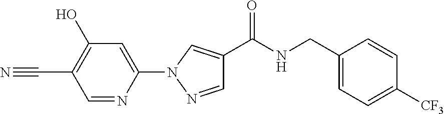 [SMILES: N#Cc1cnc(-n2cc(C(=O)NCc3ccc(C(F)(F)F)cc3)cn2)cc1O]

Following general procedure D: 113 (25 mg, 0.062 mmol) and LiCl (26 mg, 0.62 mmol) in DMAc (2 ml) gave 115 (5 mg, 0.0129 mmol, 21%).

¹H NMR (400 MHZ, DMSO-d₆) δ 9.06 (s, 1H), 8.96 (t, J=6.0 Hz, 1H), 8.13 (s, 1H), 8.08 (s, 1H), 7.70 (d, J=8.0 Hz, 2H), 7.53 (d, J=8.0 Hz, 2H), 6.78 (s, 1H), 4.51 (d, J=6.0 Hz, 2H).

HRMS (ESI-TOF) calcd for C₁₈H₁₁O₂N₅F₃ [M−H]⁻: 386.0864, found: 386.0867.

### Reference Example 92—Synthesis of Ethyl 6-hydroxypyrazolo[1,5-a]pyrido[3,2-e]pyrimidine-7-carboxylate (116)

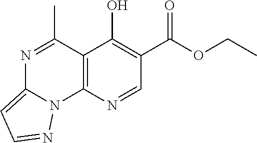 [SMILES: CCOC(=O)c1cnc2c(c(C)nc3ccnn32)c1O]

Pyrazolo[1,5-a]pyrimidin-7-amine (250 mg, 1.86 mmol), diethyl ethoxymethylenemalonate (402 mg, 1.86 mmol) were added to a microwave vial and dissolved in anhydrous EtOH (5 ml) before the addition of NaOEt (189 mg, 2.79 mmol). The reaction mixture was heated under microwave irradiation at 100° C. for 2 hrs before allowing to cool back to room temperature. The reaction mixture was then filtered, dried and before the addition of diphenyl ether (2 ml). The resultant mixture was then heated to 240° C. for 30 minutes and then allowed to cool to room temperature. The reaction mixture was then filtered, washed with Et₂O (4×50 ml) and dried gave 116 (210 mg, 0.81 mmol, 43%).

¹H NMR (400 MHZ, DMSO-d₆) δ 8.97 (s, 1H), 8.62 (s, 1H), 8.15 (d, J=2.0 Hz, 1H), 6.68 (d, J=2.0 Hz, 1H), 4.20 (q, J=7.0 Hz, 2H), 1.28 (t, J=7.0 Hz, 3H).

HRMS (ESI-TOF) calcd for C₁₂H₁₁O₃N₄ [M+H]⁺: 259.0825, found: 259.0826.

### Example 93—Synthesis of N-([1,1′-Biphenyl]-4-ylmethyl)-6-hydroxypyrazolo[1,5-a]pyrido[3,2-e]pyrimidine-7-carboxamide (117)

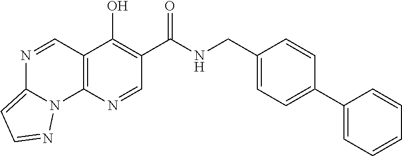 [SMILES: O=C(NCc1ccc(-c2ccccc2)cc1)c1cnc2c(cnc3ccnn32)c1O]

General Procedure A: 116 (100 mg, 0.38 mmol), 4-phenylbenzylamine (70 mg, 0.38 mmol), DABCO-(AlMe₃)₂ (100 mg, 0.38 mmol) gave 117 (4 mg, 0.01 mmol, 3%). Solvent system used for purification: 0%-5% MeOH in CH₃Cl.

¹H NMR (400 MHZ, DMSO-d₆) δ 9.04 (s, 1H), 8.72 (s, 1H), 8.26 (d, J=2.0 Hz, 1H), 7.74-7.59 (m, 4H), 7.54-7.25 (m, 5H), 6.78 (d, J=2.0 Hz, 1H), 4.60 (d, J=6.0 Hz, 2H).

HRMS (ESI-TOF) calcd for C₂₃H₁₆O₂N₅ [M−H]⁻: 394.1309, found: 394.1303.

### Reference Example 94—Synthesis of Methyl 3-([1,1′-biphenyl]-4-ylamino)-3-oxopropanoate (118)

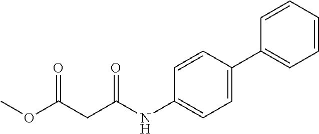 [SMILES: COC(=O)CC(=O)Nc1ccc(-c2ccccc2)cc1]

Following General Procedure B: Methyl hydrogen malonate (585 mg, 4.97 mmol), 4-aminobiphenyl (600 mg, 3.55 mmol), T3P (3.16 g, 9.94 mmol) and DIPEA (1.8 g, 14.2 mmol) gave 118 (230 mg, 0.855 mmol, 24%).

¹H NMR (400 MHZ, Chloroform-d) δ 9.27 (s, 1H), 7.68-7.30 (m, 9H), 3.82 (s, 3H), 3.52 (s, 2H).

HRMS (ESI-TOF) calcd for C₁₋₆H₁₆O₃N [M+H]⁺: 270.1124, found: 270.1122.

### Example 95—Synthesis of N-([1,1′-Biphenyl]-4-yl)-6-hydroxypyrazolo[1,5-a]pyrido[3,2-e]pyrimidine-7-carboxamide (119)

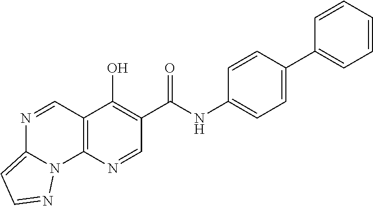 [SMILES: O=C(Nc1ccc(-c2ccccc2)cc1)c1cnc2c(cnc3ccnn32)c1O]

118 (100 mg, 0.37 mmol) were added to a microwave vial and flushed with N₂ and removed in vacuo (3 times) before the addition of anhydrous ethyl orthoformate (3 ml). The reaction mixture was heated at 120° C. for 3 hrs. The excess ethyl orthoformate was removed in vacuo and the resultant mixture was taken up in diphenyl ether (5 ml). Pyrazolo[1,5-a]pyrimidin-7-amine (75 mg, 0.55 mmol) was added, and the resultant mixture was heated to 240° C. for 30 minutes, allowed to cool to room temperature and directly purified by flash column chromatography using (CH₂Cl₂, MeOH 0-10%, 1% HCOOH)₂₀ column volumes gave 119 (5.5 mg, 0.015 mmol, 4%).

¹H NMR (400 MHZ, DMSO-d₆) δ 13.31 (s, 1H), 9.11 (s, 1H), 8.95 (s, 1H), 8.11 (d, J=2.0 Hz, 1H), 7.85-7.27 (m, 9H), 6.68 (d, J=2.0 Hz, 1H).

HRMS (ESI-TOF) calcd for C₂₂H₁₄O₂N₅ [M−H]⁻: 380.1153, found: 380.1150.

### Reference Example 96—Synthesis of Methyl 3-(([1,1′-biphenyl]-4-ylmethyl)amino)-3-oxopropanoate (120)

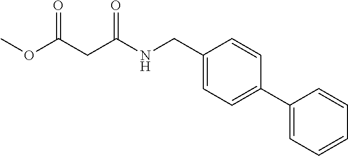 [SMILES: COC(=O)CC(=O)NCc1ccc(-c2ccccc2)cc1]

Following General Procedure B: Methyl hydrogen malonate (1.00 g, 8.9 mmol), 4-aminomethyl biphenyl (1.83 g, 10.0 mmol), T3P (6.7 g, 21.1 mmol) and DIPEA (2.7 g, 21.1 mmol) gave 120 (1.5 g, 5.3 mmol, 60.0%).

¹H NMR (400 MHZ, THF-d₈) δ 7.71 (s, 1H), 7.41-7.03 (m, 9H), 4.23 (d, J=6.0 Hz, 2H), 3.46 (s, 3H), 3.10 (s, 2H).

HRMS (ESI-TOF) calcd for C₁₇H₁₈O₃N [M+H]⁺: 284.1281, found: 284.1281.

### Reference Example 97—Synthesis of Methyl 3-oxo-3-((4-(trifluoromethyl)benzyl)amino) propanoate (121)

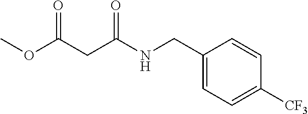 [SMILES: COC(=O)CC(=O)NCc1ccc(C(F)(F)F)cc1]

Following General Procedure B: Methyl hydrogen malonate (1.00 g, 8.9 mmol), 4-trifluorobenzyl amine (1.75 g, 10.0 mmol), T3P (6.7 g, 21.1 mmol) and DIPEA (2.7 g, 21.1 mmol) gave 121 (736 mg, 2.67 mmol, 30%).

¹H NMR (400 MHZ, Chloroform-d) δ 7.86 (s, 1H), 7.52 (d, J=8.0 Hz, 2H), 7.35 (d, J=8.0 Hz, 2H), 4.46 (d, J=6.0 Hz, 2H), 3.68 (s, 3H), 3.33 (s, 2H).

HRMS (ESI-TOF) calcd for C₁₂H₁₃O₃NF₃ [M+H]⁺: 276.0842, found: 276.0843.

### Example 98—Synthesis of N-([1,1′-Biphenyl]-4-ylmethyl)-6-hydroxy-2,5-dimethylpyrazolo[1,5-a]pyrido[3,2-e]pyrimidine-7-carboxamide (122)

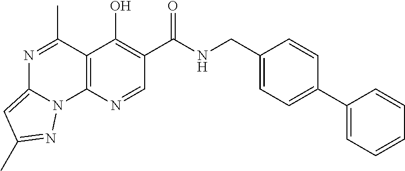 [SMILES: Cc1cc2nc(C)c3c(O)c(C(=O)NCc4ccc(-c5ccccc5)cc4)cnc3n2n1]

120 (200 mg, 0.70 mmol) were added to a microwave vial and flushed with N₂ and removed in vacuo (3 times) before the addition of anhydrous ethyl orthoformate (5 ml). The reaction mixture was heated under microwave irradiation at 120° C. for 3 hrs. The excess ethyl orthoformate was removed in vacuo and the resultant mixture was taken up in diphenyl ether (5 ml). 2,5-dimethylpyrazolo[1,5-a]pyrimidin-7-amine (114 mg, 0.70 mmol) was added and the resultant mixture was heated to 240° C. for 30 minutes, allowed to cool to room temperature and directly purified by by flash column chromatography using (CH₂Cl₂: MeOH 0-10%, 1% formic acid) over 20 column volumes to give 122 (10 mg, 0.024 mmol, 3%).

¹H NMR (400 MHZ, DMSO-d₆) δ 10.54 (s, 1H), 8.59 (s, 1H), 7.69-7.30 (m, 9H), 6.44 (s, 1H), 4.59 (d, J=6.0 Hz, 2H), 2.88 (s, 3H), 2.45 (s, 3H).

HRMS (ESI-TOF) calcd for C₂₅H₂₂O₂N₅ [M+H]⁺: 424.1768, found: 424.1768.

### Example 99—Synthesis of 6-Hydroxy-2,5-Dimethyl-N-(4-(trifluoromethyl)benzyl) pyrazolo[1,5-a]pyrido[3,2-e]pyrimidine-7-carboxamide (123)

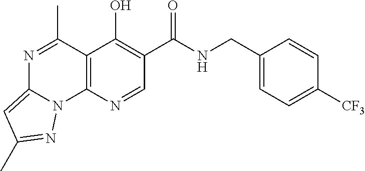 [SMILES: Cc1cc2nc(C)c3c(O)c(C(=O)NCc4ccc(C(F)(F)F)cc4)cnc3n2n1]

121 (200 mg, 0.70 mmol) were added to a microwave vial and flushed with N₂ and removed in vacuo 3 times before the addition of anhydrous ethyl orthoformate (5 ml). The reaction mixture was heated under microwave irradiation at 120° C. for 3 hrs. The excess ethyl orthformate was removed in vacuo and the resultant mixture was taken up in diphenyl ether (5 mL). 2,5-dimethylpyrazolo[1,5-a]pyrimidin-7-amine (114 mg, 0.70 mmol) was added and the resultant mixture was heated to 240° C. for 30 minutes, allowed to cool to room temperature and directly purified by flash column chromatography using (CH₂Cl₂: MeOH 0-10%, 1% formic acid) over 20 column volumes gave 123 (8 mg, 0.020 mmol, 3%).

¹H NMR (400 MHZ, DMSO-d₆) δ 8.52 (s, 1H), 7.71 (d, J=8.1 Hz, 2H), 7.55 (d, J=8.0 Hz, 2H), 6.50 (s, 1H), 4.64 (d, J=6.0 Hz, 2H), 2.89 (s, 3H), 2.47 (s, 3H).

HRMS (ESI-TOF) calcd for C₂₀H₁₅O₂N₅F₃ [M−H]⁻: 414.1183, found: 414.1182.

### Example 100—Synthesis of N-([1,1′-biphenyl]-4-ylmethyl)-6-(4-cyano-1H-pyrazol-1-yl)-5-methoxynicotinamide (124)

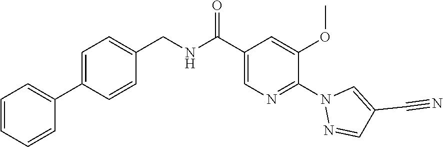 [SMILES: COc1cc(C(=O)NCc2ccc(-c3ccccc3)cc2)cnc1-n1cc(C#N)cn1]

Following general procedure C: 72 (147 mg, 0.42 mmol) Pd^{t}BuXPhos G3 (33 mg, 0.04 mmol), Cesium Carbonate (273 mg, 0.84 mmol) and 1H-pyrazole-4-carbonitrile (70 mg, 0.5 mmol) gave the titled compound (56 mg, 0.137 mmol, 33%).

¹H NMR (400 MHZ, DMSO) δ 9.41 (t, J=6.0 Hz, 1H), 9.11 (d, J=1.0 Hz, 1H), 8.65 (d, J=2.0 Hz, 1H), 8.36 (d, J=1.0 Hz, 1H), 8.18 (d, J=2.0 Hz, 1H), 7.67-7.62 (m, 4H), 7.49-7.42 (m, 4H), 7.40-7.32 (m, 1H), 4.59 (d, J=6.0 Hz, 2H), 3.97 (s, 3H).

### Example 101—Synthesis of N-([1,1′-biphenyl]-4-ylmethyl)-6-(4-cyano-1H-pyrazol-1-yl)-5-hydroxynicotinamide (125)

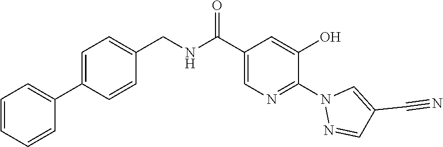 [SMILES: N#Cc1cnn(-c2ncc(C(=O)NCc3ccc(-c4ccccc4)cc3)cc2O)c1]

Following general procedure D: 125 (56 mg, 0.14 mmol) and lithium chloride (58 mg, 1.37 mmol) were combined before the addition of NN-Dimethylacetamide (3 mL). The resultant mixture was microwaved at 150° C. for 8 hrs. The crude material was then purified using flash reverse phase chromatography (H₂O 0.1% formic acid (0-100% ACN, 0.1% formic acid)) to give the titled compound (3 mg, 0.007 mmol, 5%).

¹H NMR (400 MHZ, DMSO) δ 9.35 (t, J=5.9 Hz, 1H), 9.24 (s, 1H), 8.53 (d, J=1.9 Hz, 1H), 8.42 (s, 1H), 7.95 (d, J=1.9 Hz, 1H), 7.67-7.62 (m, 4H), 7.45 (qd, J=7.1, 1.9 Hz, 4H), 7.38-7.32 (m, 1H), 4.55 (d, J=6.0 Hz, 2H).

### Reference Example 102—Synthesis of 6-chloro-5-methoxy-N-((4-(trifluoromethyl)cyclohexyl)methyl) nicotinamide (126)

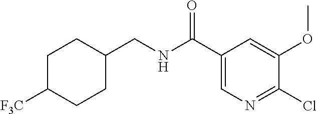 [SMILES: COc1cc(C(=O)NCC2CCC(C(F)(F)F)CC2)cnc1Cl]

Following general procedure B: 6-chloro-5-methoxynicotinic acid (1000 mg, 5.35 mmol), HATU (4064 mg, 10.70 mmol), C-4 trifluorocyclohexyl amine (1209 μL, 8.02 mmol) & DIPEA (2759 μL, 16.04 mmol) gave the title compound (1625 mg, 4.64 mol, 86%). LRMS m/z calcd. For C₁₅H₁₉ClF₃N₂O₂ [M+H]⁺: 351.1, found: 351.1

### Example 103—Synthesis of Ethyl 1-(3-methoxy-5-(((4-(trifluoromethyl)cyclohexyl)methyl) carbamoyl)pyridin-2-yl)-1H-pyrazole-4-carboxylate (127)

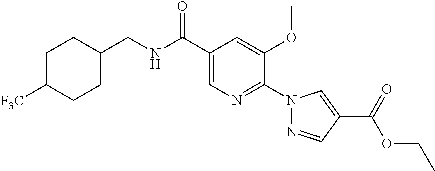 [SMILES: CCOC(=O)c1cnn(-c2ncc(C(=O)NCC3CCC(C(F)(F)F)CC3)cc2OC)c1]

Following general procedure C: 127 (300 mg, 0.86 mmol), Pd^{t}BuXPhos G3 (68 mg, 0.09 mmol), ethyl 1H-pyrazole-4-carboxylate (180 mg, 1.29 mmol) and Cs₂CO₃ (835 mg, 2.57 mmol) gave the targeted compound (92 mg, 0.20 mmol, 23%).

¹H NMR (400 MHZ, DMSO) δ 8.80 (t, J=6.0 Hz, 1H), 8.74 (d, J=0.7 Hz, 1H), 8.57 (d, J=1.8 Hz, 1H), 8.14 (d, J=0.6 Hz, 1H), 8.07 (d, J=1.8 Hz, 1H), 4.27 (q, J=7.0 Hz, 2H), 3.96 (s, 3H), 3.19 (t, J=6.0 Hz, 2H), 2.35-2.16 (m, 1H), 1.94-1.82 (m, 4H), 1.63-1.50 (m, 1H), 1.30 (t, J=7.0 Hz, 3H), 1.27-0.98 (m, 4H).

LRMS m/z calcd. For C₂₁H₂₆F₃N₄O₄ [M+H]⁺: 455.19, found: 455.30

### Example 104—Synthesis of Ethyl 1-(3-hydroxy-5-(((4-(trifluoromethyl)cyclohexyl)methyl) carbamoyl)pyridin-2-yl)-1H-pyrazole-4-carboxylate (128)

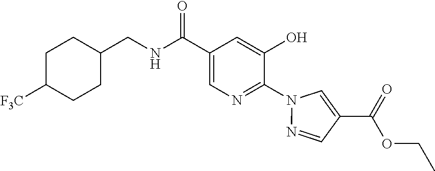 [SMILES: CCOC(=O)c1cnn(-c2ncc(C(=O)NCC3CCC(C(F)(F)F)CC3)cc2O)c1]

Follow general procedure D: 128 (92 mg, 0.20 mmol), LiCl (85 mg, 2.03 mmol) and DMSO (2 mL) gave the title compound (20 mg, 0.04 mmol, 20%).

¹H NMR (400 MHZ, DMSO) δ 8.95 (s, 1H), 8.69 (t, J=6.0 Hz, 1H), 8.42 (d, J=2.0 Hz, 1H), 8.24 (s, 1H), 7.87 (d, J=2.0 Hz, 1H), 4.28 (q, J=7.0 Hz, 2H), 3.15 (t, J=6.3 Hz, 2H), 1.92-1.79 (m, 5H), 1.55 (dp, J=18.8, 7.4, 5.7 Hz, 1H), 1.29 (t, J=7.0 Hz, 3H), 1.25-1.16 (m, 2H), 1.02 (qd, J=13.8, 13.1, 3.9 Hz, 2H).

### Example 105—Synthesis of 1-(3-hydroxy-5-(((4-(trifluoromethyl)cyclohexyl)methyl) carbamoyl)pyridin-2-yl)-1H-pyrazole-4-carboxylic acid (129)

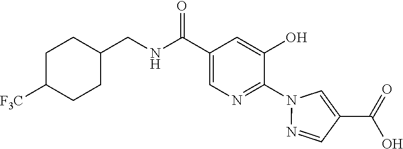 [SMILES: O=C(O)c1cnn(-c2ncc(C(=O)NCC3CCC(C(F)(F)F)CC3)cc2O)c1]

129 (20 mg, 0.04 mmol) was dissolved in THF (10 mL) before the addition of LiOH aq solution (500 μL, 2 M). The resultant mixture was stirred at room temperature of 16 hr.

The reaction mixture was neutralized with formic acid before the addition of celite. The solvent was then removed under vacuum and the crude mixture was purified using flash reverse column chromatography (0%-100% ACN (0.1% formic acid) in H₂O (0.1% formic acid)) gave the title compound (6 mg, 0.014 mmol, 36%).

¹H NMR (400 MHZ, DMSO) δ 8.82 (s, 1H), 8.65 (t, J=6.0 Hz, 1H), 8.40 (d, J=2.0 Hz, 1H), 8.09 (s, 1H), 7.88 (d, J=2.0 Hz, 1H), 3.14 (t, J=6.0 Hz, 2H), 2.28-2.15 (m, 1H), 1.92-1.80 (m, 5H), 1.21 (qt, J=13.9, 6.7 Hz, 3H), 1.02 (qd, J=13.7, 12.9, 4.0 Hz, 2H). LRMS m/z calcd. For C₁₈H₁₈F₃N₄O₄ [M−H]⁻: 411.13, found: 411.0

### Reference Example 106—Synthesis of Ethyl 6-chloro-4-methoxynicotinate (130)

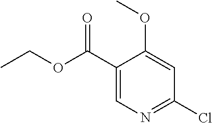

6-chloro-4-methoxynicotinic acid (5 g, 0.03 mol), EDC.HCl (6.22 g, 0.04 mol), DMAP (0.05 g, 1.2 mmol) were dissolved in DMF (50 mL). DIPEA (4.60 mL, 0.03 mmol) and EtOH (5 mL) were then added to the reaction mixture. The resultant mixture was then stirred at room temperature for 16 hr. EtOAc (50 mL), 1M HCl (50 mL) and H₂O (100 mL) were added to the reaction mixture. The organic and aqueous layers were separated. This process was repeat 2 more times. The organic layers were combined before washed with brine and then dried with anhydrous Na₂SO₄. The crude compound was then purified using flash column chromatography using (cyclohexane 100%-50%, EtOAc 0%-50%) over 15 column volumes to give the desired compound (2.02 g, 9.39 mmol, 31%).

LRMS m/z calcd. For C₉H₁₁ClNO₃ [M+H]⁺: 216.0, found: 216.2

### Reference Example 107—Synthesis of Ethyl 4-methoxy-6-(1H-pyrazol-1-yl) nicotinate (131)

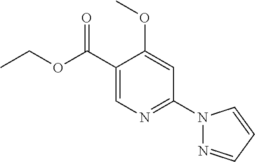 [SMILES: CCOC(=O)c1cnc(-n2cccn2)cc1OC]

Following the general procedure C: 130 (1.90 g, 0.088 mol), Pd^{t}BuXPhos G3 (0.35 g, 0.00044 mol), Cesium carbonate (4.30 g, 0.0132 mol) and pyrazole (0.90 g, 0.0132 mol) and anhydrous1,4-dioxane (50 mL) gave the titled compound (1.9 g, 7.65 mmol, 87%). LRMS m/z calcd. For C₁₂H₁₄N₃O₃ [M+H]⁺: 248.10, found: 248.10

### Reference Example 108—Synthesis of 4-methoxy-6-(1H-pyrazol-1-yl) nicotinic acid (132)

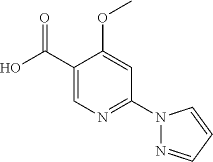 [SMILES: COc1cc(-n2cccn2)ncc1C(=O)O]

131 (1.9 g, 7.65 mmol) was dissolved in a mixture of THF (65 mL), MeOH (35 mL), H₂O (10 mL) before the addition of lithium hydroxide monohydrate (3.7 g, 90.5 mmol). The resultant mixture was stirred at room temperature for 16 hr. The reaction was confirmed to go to completion via TLC. The reaction mixture was acidified, cooled to 5° C. and filtered to collect the precipitate. The precipitate was washed with diethyl ether to give the tilted compound (1.4 g, 6.39 mmol, 84%).

LRMS m/z calcd. For C₁₀H₁₀N₃O₄ [M+H]⁺: 220.07, found: 220.2.

### Example 109—Synthesis of N-(but-2-yn-1-yl)-4-methoxy-6-(1H-pyrazol-1-yl) nicotinamide (133)

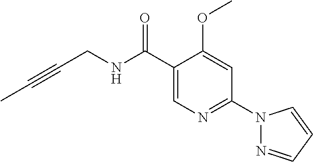 [SMILES: CC#CCNC(=O)c1cnc(-n2cccn2)cc1OC]

Following general procedure B: 132 (187 mg, 0.85 mmol), HATU (646 mg, 1.7 mmol), but-2-yn-1-amine hydrogen chloride (135 mg, 1.27 mmol) and DIPEA (438 L, 2.55 mmol) gave the titled compound (148 mg, 0.54 mmol, 63%).

LRMS m/z calcd. For C₁₄H₁₅N₄O₂ [M+H]⁺: 271.1, found: 271.2

### Example 110—Synthesis of N-(but-2-yn-1-yl)-4-hydroxy-6-(1H-pyrazol-1-yl) nicotinamide (134)

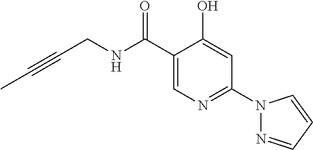 [SMILES: CC#CCNC(=O)c1cnc(-n2cccn2)cc1O]

Following general procedure D: 133 (143 mg, 0.53 mmol) and lithium chloride (111 mg, 2.64 mmol) gave the titled compound (44 mg, 0.171 mmol, 33%).

¹H NMR (400 MHZ, DMSO) δ 11.38 (t, J=5.4 Hz, 1H), 8.48-8.39 (m, 2H), 7.73-7.62 (m, 1H), 6.73 (d, J=3.1 Hz, 1H), 6.42 (q, J=2.1 Hz, 1H), 4.01 (dq, J=5.2, 2.5 Hz, 2H), 1.78 (t, J=2.7 Hz, 3H).

LRMS m/z calcd. For C₁₃H₁₃N₄O₂ [M+H]⁺: 257.10, found: 257.30

### Example 111—Synthesis of N-((6-chloropyridin-3-yl)methyl)-4-methoxy-6-(1H-pyrazol-1-yl) nicotinamide (135)

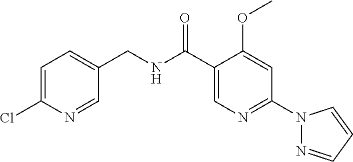 [SMILES: COc1cc(-n2cccn2)ncc1C(=O)NCc1ccc(Cl)nc1]

Following general procedure B: 132 (900 mg, 4.11 mmol), HATU (3123 mg, 8.22 mmol), (6-chloropyridin-3-yl) methanamine (700 mg, 4.93 mmol) and DIPEA (2.1 mL, 12.33 mmol) gave the titled compound (1.315 g, 3.83 mmol, 93%).

LRMS m/z calcd. For C₁₆H₁₅ClN₅O₂ [M+H]⁺: 344.1, found: 344.2

### Example 112—Synthesis of Ethyl 4-(5-((4-methoxy-6-(1H-pyrazol-1-yl) nicotinamido)methyl)pyridin-2-yl)benzoate (136)

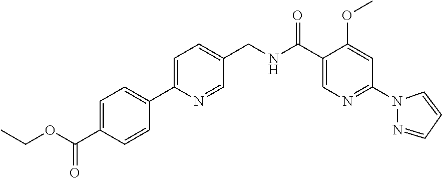 [SMILES: CCOC(=O)c1ccc(-c2ccc(CNC(=O)c3cnc(-n4cccn4)cc3OC)cn2)cc1]

Following general procedure C: 135 (44 mg, 0.128 mmol), (4-ethoxycarbonyl)phenyl) boronic acid (42 mg, 0.22 mmol), Pd Amphos (5.16 mg, 0.01 mmol) and cesium carbonate (142.13 mg, 0.44 mmol) gave the titled compound (50 mg, 0.109 mmol, 54%).

LRMS m/z calcd. For C₂₅H₂₄N₅O₄ [M+H]⁺: 458.18, found: 458.3, [M+2H]/2 was observed 229.7.

### Example 113—Synthesis of 4-methoxy-6-(1H-pyrazol-1-yl)-N-((6-(pyrimidin-5-yl) pyridin-3-yl)methyl)-nicotinamide (137)

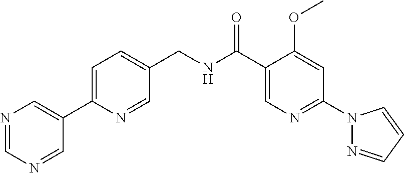 [SMILES: COc1cc(-n2cccn2)ncc1C(=O)NCc1ccc(-c2cncnc2)nc1]

Following general procedure C: 135 (27 mg, 0.078 mmol), pyrimidin-5-ylboronic acid (27 mg, 0.22 mmol), Pd Amphos (5.16 mg, 0.01 mmol) and cesium carbonate (142.13 mg, 0.44 mmol) gave the titled compound (9 mg, 0.012 mmol, 15%).

LRMS m/z calcd. For C₂₀H₁₈N₇O₂ [M+H]⁺: 388.15, found: 388.00. [M+2H]/2 was observed 194.8

### Example 114—Synthesis of 4-methoxy-N-((6-(4-(methylcarbamoyl)phenyl)pyridin-3-yl)methyl)-6-(1H-pyrazol-1-yl) nicotinamide (138)

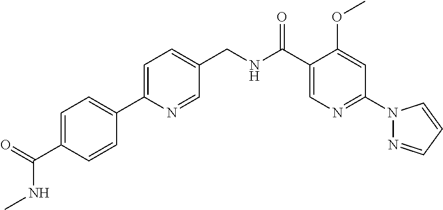 [SMILES: CNC(=O)c1ccc(-c2ccc(CNC(=O)c3cnc(-n4cccn4)cc3OC)cn2)cc1]

Following general procedure C: 135 (50 mg, 0.15 mmol), (4-(methylcarbamoyl)phenyl) boronic acid (39 mg, 0.22 mmol), Pd Amphos (5.16 mg, 0.01 mmol) and cesium carbonate (142.13 mg, 0.44 mmol) gave the titled compound (39 mg, 0.088 mmol, 59%).

LRMS m/z calcd. For C₂₄H₂₃N₆O₃ [M+H]⁺: 443.18, found: 443.1, [M+2H]/2 was observed 222.20.

### Example 115—Synthesis of N-((6-(4-carbamoylphenyl)pyridin-3-yl)methyl)-4-methoxy-6-(1H-pyrazol-1-yl) nicotinamide (139)

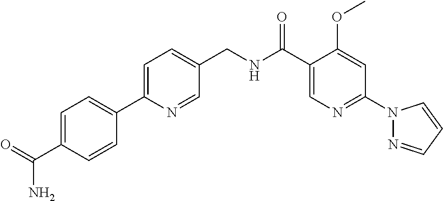 [SMILES: COc1cc(-n2cccn2)ncc1C(=O)NCc1ccc(-c2ccc(C(N)=O)cc2)nc1]

Following general procedure C: 135 (50 mg, 0.15 mmol), (4-(carbamoyl)phenyl) boronic acid (36 mg, 0.22 mmol), Pd Amphos (5.16 mg, 0.01 mmol) and cesium carbonate (142.13 mg, 0.44 mmol) gave the titled compound (22 mg, 0.051 mmol, 34%).

LRMS m/z calcd. For C₂₃H₂₁N₆O₃ [M+H]⁺: 429.17, found: 429.2

### Example 116—Synthesis of 4-methoxy-N-((6-(4-morpholinophenyl)pyridin-3-yl)methyl)-6-(1H-pyrazol-1-yl) nicotinamide (140)

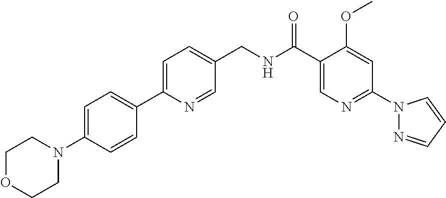 [SMILES: COc1cc(-n2cccn2)ncc1C(=O)NCc1ccc(-c2ccc(N3CCOCC3)cc2)nc1]

Following general procedure C: 135 (50 mg, 0.15 mmol), (4-morpholinophenyl) boronic acid (59 mg, 0.22 mmol), Pd Amphos (5.16 mg, 0.01 mmol) and cesium carbonate (142.13 mg, 0.44 mmol) gave the titled compound (50 mg, 0.106 mmol, 71%).

LRMS m/z calcd. For C₂₆H₂₇N₆O₃ [M+H]⁺: 471.21, found: 471.20, [M+2H]/2 was observed 236.20.

### Example 117—Synthesis of N-((6-(benzo[d]thiazol-5-yl) pyridin-3-yl)methyl)-4-methoxy-6-(1H-pyrazol-1-yl) nicotinamide (141)

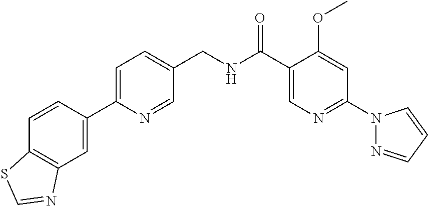 [SMILES: COc1cc(-n2cccn2)ncc1C(=O)NCc1ccc(-c2ccc3scnc3c2)nc1]

Following general procedure C: 135 (50 mg, 0.15 mmol), (5-(4,4,5,5-tetramethyl-1,3,2-dioxaborolan-2-yl)benzo[d]thiazole (57.07 mg, 0.22 mmol), Pd Amphos (5.16 mg, 0.01 mmol) and cesium carbonate (142.13 mg, 0.44 mmol) gave the titled compound (22 mg, 0.0497 mmol, 33%).

LRMS m/z calcd. For C₂₃H₁₉N₆O₂S [M+H]⁺: 443.13, found [M+2H]²⁺/2:222.0

### Example 118—Synthesis of Ethyl 4-(5-((4-hydroxy-6-(1H-pyrazol-1-yl) nicotinamido)methyl)pyridin-2-yl)benzoate (142)

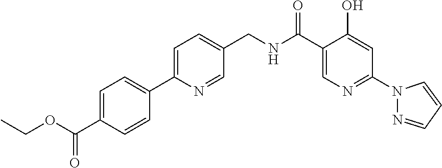 [SMILES: CCOC(=O)c1ccc(-c2ccc(CNC(=O)c3cnc(-n4cccn4)cc3O)cn2)cc1]

Following general procedure D: 140 (50 mg, 0.11 mmol) and lithium chloride (46 mg, 1.09 mmol) gave the titled compound (10 mg, 0.022 mmol, 22%) and was purified via reverse flash chromatography (0-100% ACN (0.1% formic acid) in H₂O (0.1% formic acid)) gave the titled compound (10 mg, 0.0225 mmol, 22%).

LRMS m/z calcd. For C₂₄H₂₂N₅O₄ [M+H]⁺: 444.10, found: 444.2, [M+2H]²⁺/2 was observed 222.7.

### Example 119—Synthesis of 4-(5-((4-hydroxy-6-(1H-pyrazol-1-yl) nicotinamido)methyl)pyridin-2-yl)benzoic acid (143)

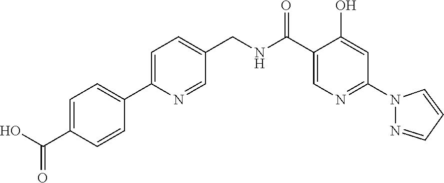 [SMILES: O=C(O)c1ccc(-c2ccc(CNC(=O)c3cnc(-n4cccn4)cc3O)cn2)cc1]

143 (6 mg, 0.0135 mmol) was dissolved in a mixture of MeOH (5 mL) and H₂O (1 mL) before the addition of lithium hydroxide (2.84 mg, 0.07 mmol). The resultant mixture was stirred at 16 hr at room temperature. 1M HCl (1 mL) was added to the reaction mixture and the resultant precipitate was filtered, washed with Et₂O, and allowed to dry to give the title compound (2 mg, 0.0048 mmol, 35%).

¹H NMR (400 MHZ, DMSO) δ 11.88 (t, J=5.8 Hz, 1H), 8.65 (d, J=2.3 Hz, 1H), 8.48 (s, 1H), 8.44 (s, 1H), 8.15 (d, J=8.2 Hz, 2H), 8.01 (dd, J=8.3, 6.3 Hz, 3H), 7.82 (dd, J=8.0, 2.5 Hz, 1H), 7.65 (d, J=1.5 Hz, 1H), 6.68 (s, 1H), 6.41 (dd, J=2.1, 2.1 Hz, 1H), 4.55 (d, J=5.8 Hz, 2H).

LRMS m/z calcd. For C₂₂H₁₈N₅O₄ [M+H]⁺: 416.14, found: 416.20, [M+2H]⁺/2 was observed 208.6

### Example 120—Synthesis of 4-hydroxy-6-(1H-pyrazol-1-yl)-N-((6-(pyrimidin-5-yl) pyridin-3-yl)methyl) nicotinamide (144)

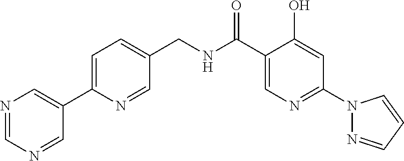 [SMILES: O=C(NCc1ccc(-c2cncnc2)nc1)c1cnc(-n2cccn2)cc1O]

Following general procedure D: 137 (9 mg, 0.02 mmol) and lithium chloride (9.77 mg, 0.23 mmol) gave the titled compound (6 mg, 0.0160 mmol, 81%).

¹H NMR (400 MHZ, DMSO) δ 9.43 (d, J=1.9 Hz, 2H), 9.24 (s, 1H), 8.75 (d, J=2.2 Hz, 1H), 8.64 (d, J=2.7 Hz, 1H), 8.15-8.10 (m, 1H), 7.97-7.85 (m, 2H), 7.35 (s, 1H), 6.61 (q, J=2.1 Hz, 1H), 4.63 (d, J=5.9 Hz, 2H).

LRMS m/z calcd. For C₁₉H₁₆N₇O₂ [M+H]⁺: 374.14, found: 374.00, [M+2H]⁺/2=187.5

### Example 121—Synthesis of 4-hydroxy-N-((6-(4-(methylcarbamoyl)phenyl)pyridin-3-yl)methyl)-6-(1H-pyrazol-1-yl) nicotinamide (145)

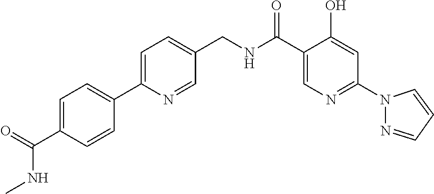 [SMILES: CNC(=O)c1ccc(-c2ccc(CNC(=O)c3cnc(-n4cccn4)cc3O)cn2)cc1]

Following general procedure D: 138 (35 mg, 0.09 mmol) and lithium chloride (37 mg, 0.88 mmol) gave the titled compound (6 mg, 0.0160 mmol, 81%).

¹H NMR (400 MHZ, DMSO) δ 9.57 (s, 1H), 8.74 (s, 1H), 8.70 (d, J=2.2 Hz, 1H), 8.64 (d, J=2.6 Hz, 1H), 8.50 (q, J=4.5 Hz, 1H), 8.21-8.09 (m, 2H), 8.02 (d, J=8.2 Hz, 1H), 7.98-7.90 (m, 2H), 7.90-7.79 (m, 2H), 7.33 (s, 1H), 6.60 (t, J=2.7 Hz, 1H), 4.62 (d, J=5.9 Hz, 2H), 2.81 (d, J=4.5 Hz, 3H).

LRMS m/z calcd. For C₂₃H₂₁N₆O₃ [M+H]⁺: 429.17, found: 429.10, [M+2H]²⁺/2=215.1

### Example 122—Synthesis of N-((6-(4-carbamoylphenyl)pyridin-3-yl)methyl)-4-hydroxy-6-(1H-pyrazol-1-yl) nicotinamide (146)

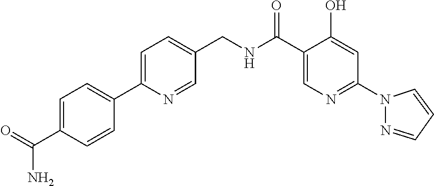 [SMILES: NC(=O)c1ccc(-c2ccc(CNC(=O)c3cnc(-n4cccn4)cc3O)cn2)cc1]

Following general procedure D: 139 (22 mg, 0.05 mmol) and lithium chloride (21.59 mg, 0.51 mmol) gave the titled compound (8 mg, 0.019 mmol, 38%).

¹H NMR (400 MHZ, DMSO) δ 9.72 (s, 1H), 8.75-8.66 (m, 2H), 8.62 (d, J=2.6 Hz, 1H), 8.18-8.10 (m, 2H), 8.06-7.94 (m, 4H), 7.92-7.78 (m, 2H), 7.40 (s, 1H), 7.30 (s, 1H), 6.58 (dd, J=2.7, 1.7 Hz, 1H), 4.61 (d, J=5.9 Hz, 2H).

LRMS m/z calcd. For C₂₂H₁₉N₆O₃ [M+H]⁺: 415.15, found: 415.20, [M+2H]²⁺/2=208.40

### Example 123—Synthesis of 4-hydroxy-N-((6-(4-morpholinophenyl)pyridin-3-yl)methyl)-6-(1H-pyrazol-1-yl) nicotinamide (147)

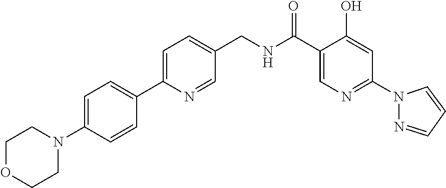 [SMILES: O=C(NCc1ccc(-c2ccc(N3CCOCC3)cc2)nc1)c1cnc(-n2cccn2)cc1O]

Following general procedure D: 140 (35 mg, 0.11 mmol) and lithium chloride (44.68 mg, 1.06 mmol) gave the titled compound (6 mg, 0.0160 mmol, 81%).

¹H NMR (400 MHZ, DMSO) δ 9.65 (s, 1H), 8.72 (s, 1H), 8.63 (dd, J=2.6, 0.7 Hz, 1H), 8.58 (dd, J=2.3, 0.9 Hz, 1H), 8.00-7.92 (m, 2H), 7.88-7.80 (m, 2H), 7.77 (dd, J=8.2, 2.3 Hz, 1H), 7.29 (s, 1H), 7.06-6.98 (m, 2H), 6.59 (dd, J=2.6, 1.7 Hz, 1H), 4.56 (d, J=5.9 Hz, 2H), 3.75 (dd, J=5.8, 3.9 Hz, 4H), 3.22-3.15 (m, 4H).

LRMS m/z calcd. For C₂₅H₂₅N₆O₃ [M+H]⁺: 457.20, found: 457.40, [M+2H]²⁺/2=229.2

### Example 123a—Synthesis of N-((6-(benzo[d]thiazol-5-yl) pyridin-3-yl)methyl)-4-hydroxy-6-(1H-pyrazol-1-yl) nicotinamide (148)

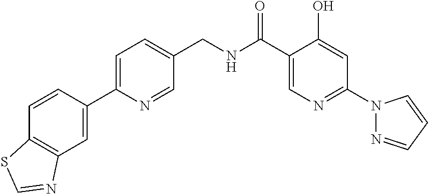 [SMILES: O=C(NCc1ccc(-c2ccc3scnc3c2)nc1)c1cnc(-n2cccn2)cc1O]

Following general procedure D: 141 (22 mg, 0.05 mmol) and lithium chloride (21 mg, 0.5 mmol) gave the titled compound (14 mg, 0.032 mmol, 65%).

¹H NMR (400 MHZ, DMSO) δ 9.45 (s, 1H), 8.84-8.59 (m, 3H), 8.35-8.19 (m, 2H), 8.17-8.07 (m, 2H), 7.95-7.82 (m, 2H), 7.34 (s, 1H), 6.60 (dd, J=3.8, 3.0 Hz, 1H), 4.62 (d, J=6.2 Hz, 2H).

LRMS m/z calcd. For C₂₂H₁₇N₆O₂S [M+H]⁺: 429.11, found: 429.10, [M+2H]²⁺/2=215.2

### Reference Example 124—Synthesis of methyl 6-chloro-4-methoxypyridazine-3-carboxylate (149)

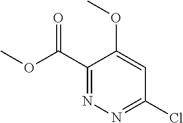

Methyl 4,6-dichloropyridazine-3-carboxylate (1000 mg, 4.93 mmol) was dissolved in THF (10 mL). The resultant mixture was cooled to 0° C. before the slow addition of sodium methoxide (319 mg, 5.91 mmol). The resultant mixture was allowed to warm up to room temperature and stirred for 6 hours. The solvent was then removed under vacuum and the resultant residue was taken up in a mixture of EtOAc (50 mL) and H₂O (50 mL). The organic and aqueous layers were separated, and the aqueous layer was washed with EtOAc (50 mL) twice more. The organic layers were combined, dried (Na₂SO₄) and purified using flash column chromatography (0%-100% EtOAc, cyclohexane 100%-0%) to give the titled compound (103 mg, 0.509 mmol, 10%).

LRMS m/z calcd. For C₇H₈ClN₂O₃ [M+H]⁺: 203.02, found: 203.1

### Reference Example 125—Synthesis of methyl 4-methoxy-6-(1H-pyrazol-1-yl)pyridazine-3-carboxylate (150)

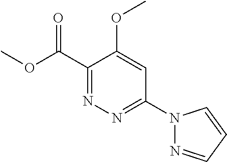 [SMILES: COC(=O)c1nnc(-n2cccn2)cc1OC]

Following general procedure C: 149 (103 mg, 0.29 mmol), pyrazole (30 mg, 0.44 mmol), Pd ^{t}BuXPhos G3 (23 mg, 0.03 mmol) and cesium carbonate (284 mg, 0.88 mmol) gave the titled compound (37 mg, 0.16 mmol, 55%).

LRMS m/z calcd. For C₁₀H₁₁N₄O₃ [M+H]⁺: 235.08, found: 235.2

### Reference Example 126—Synthesis of 4-methoxy-6-(1H-pyrazol-1-yl)pyridazine-3-carboxylic acid (151)

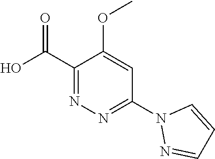 [SMILES: COc1cc(-n2cccn2)nnc1C(=O)O]

150 (37 mg, 0.16 mmol) was taken up in a mixture of methanol (5 mL) and H₂O (0.5 mL) before the addition of lithium hydroxide (33.21 mg, 0.79 mmol). The resultant mixture was stirred at room temperature for 16 hrs. 1M HCl (5 mL) was added to the reaction mixture. The precipitate was then collected and washed with Et₂O to give the titled compound (22 mg, 0.099 mmol, 62%).

LRMS m/z calcd. For C₉H₉N₄O₄ [M+H]⁺: 221.07, found: 221.1

### Example 127—Synthesis of 4-methoxy-6-(1H-pyrazol-1-yl)-N-((4-(trifluoromethyl)cyclohexyl)methyl)pyridazine-3-carboxamide (152)

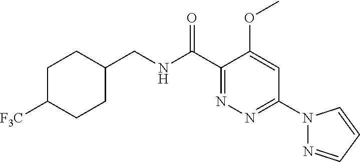 [SMILES: COc1cc(-n2cccn2)nnc1C(=O)NCC1CCC(C(F)(F)F)CC1]

Following general procedure B: 151 (22 mg, 0.10 mmol), HATU (76 mg, 0.20 mmol), (4-trifluoromethyl)cyclohexyl) methanamine (15 μL, 0.1 mmol) and DIPEA (51 μL, 0.3 mmol) gave the titled compound (34 mg, 0.088 mmol, 89%).

LRMS m/z calcd. For C₁₇H₂₁F₃N₅O₂ [M+H]⁺: 384.16, found: 384.2

### Example 128—Synthesis of 4-hydroxy-6-(1H-pyrazol-1-yl)-N-((4-(trifluoromethyl)cyclohexyl)methyl)pyridazine-3-carboxamide (153)

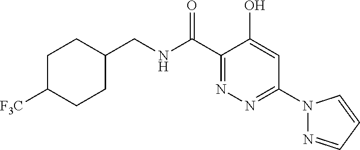 [SMILES: O=C(NCC1CCC(C(F)(F)F)CC1)c1nnc(-n2cccn2)cc1O]

Following general procedure D: 152 (34 mg, 0.08 mmol) and lithium chloride (78 mg, 1.86 mmol) gave the titled compound (8 mg, 0.0216 mmol, 11%).

¹H NMR (400 MHZ, DMSO) δ 9.92 (s, 1H), 8.79-8.74 (m, 1H), 7.94 (d, J=1.7 Hz, 1H), 7.46 (s, 1H), 6.68 (dd, J=2.7, 1.7 Hz, 1H), 3.22 (t, J=6.5 Hz, 2H), 2.32-2.14 (m, 1H), 1.93-1.79 (m, 4H), 1.63 (ddt, J=11.7, 8.3, 4.4 Hz, 1H), 1.32-0.99 (m, 4H).

LRMS m/z calcd. For C₁₋₆H₁₉N₅O₂ [M+H]⁺: 370.15, found: 370.2

### Example 129—Synthesis of Methyl 1-(3-hydroxy-5-(((4-(trifluoromethyl)cyclohexyl)methyl) carbamoyl)pyridin-2-yl)-1H-pyrazole-4-carboxylate (154)

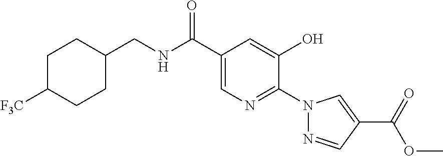 [SMILES: COC(=O)c1cnn(-c2ncc(C(=O)NCC3CCC(C(F)(F)F)CC3)cc2O)c1]

128 (10 mg, 0.02 mmol) was added to a 10 mL round bottom flask and flushed with N₂ before the addition of anhydrous MeOH (3 mL). 10% sodium methoxide in methanol solution (500 μL) was added to the reaction mixture and the resultant mixture was stirred at room temperature of 16 hr. Formic acid was added to neutralise the reaction mixture before the addition of celite. The solvent was removed under vacuum and the crude mixture was purified using flash column chromatography (0-10% MeOH in CH₂Cl₂) to give the titled compound (4.5 mg, 0.010 mmol, 52%).

¹H NMR (400 MHZ, DMSO) δ 8.93 (d, J=0.7 Hz, 1H), 8.71 (t, J=6.0 Hz, 1H), 8.47 (d, J=1.9 Hz, 1H), 8.28 (d, J=0.7 Hz, 1H), 7.90 (d, J=1.9 Hz, 1H), 3.82 (s, 3H), 3.15 (t, J=6.5 Hz, 2H), 1.95-1.78 (m, 5H), 1.30-0.96 (m, 5H).

LRMS m/z calcd. For C₁₉H₂₂F₃N₄O₄ [M+H]⁺: 427.16, found: 427.2

### Reference Example 130—Synthesis of Ethyl 1-carbamimidoyl-1H-pyrazole-4-carboxylate (155)

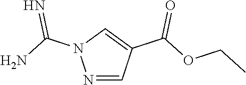

To a solution of 1H-pyrazole-4-carboxylic acid ethyl ester (6.66 g, 47.62 mmol), cyanamide (2.g, 47.62 mmol) and dioxane (30 mL) was added a solution of 4M HCl in dioxane (20 mL). The reaction mixture was heated to 100° C. for 3 hrs. The reaction was cooled to room temperature and Et₂O (30 mL was added. The precipitate was collected to yield the title compound (3 g, 16.4 mmol, 35%).

LRMS m/z calcd. For C₇H₁₁N₄O₂ [M+H]⁺: 183.09, found: 183.1

### Reference Example 131—Synthesis of Methyl 1-(5-cyano-4-hydroxypyrimidin-2-yl)-1H-pyrazole-4-carboxylate (156)

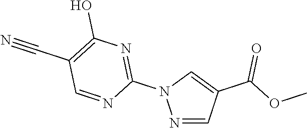 [SMILES: COC(=O)c1cnn(-c2ncc(C#N)c(O)n2)c1]

Ethyl (E)-2-cyano-3-ethoxyacrylate (3.0 g, 17.75 mmol) was added to a solution of 155 (3.23 g, 17.75 mmol), K₂CO₃ (4.9 g, 35.50 mmol) and methanol (100 mL). The resultant mixture was stirred at room temperature for 1 hr. H₂O (50 mL) was added, and the white precipitate was filtered to give the titled compound (3.52 g, 13.59 mmol, 76%).

LRMS m/z calcd. For C₁₀H₈N₅O₃ [M+H]⁺: 246.06, found: 246.1

### Reference Example 132—Synthesis of 1-(5-cyano-4-hydroxypyrimidin-2-yl)-1H-pyrazole-4-carboxylic acid (157)

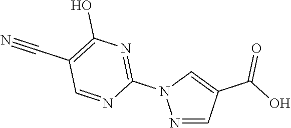 [SMILES: N#Cc1cnc(-n2cc(C(=O)O)cn2)nc1O]

A solution of 156 (3.0 g, 13.1 mmol) in MeOH (15 mL) and H₂O (10 mL) was prepared, before the addition of lithium hydroxide monohydrate (904 mg, 39.3 mmol). The reaction mixture was stirred for 4 hrs. The reaction mixture was neutralised with 1N HCl and the MeOH was removed under vacuum. The precipitate was collected to give the titled compound (2.49 g, 10.8 mmol, 83%).

LRMS m/z calcd. For C₉H₄NO₃ [M−H]⁻: 230.03, found: 230.4

### Example 133—Synthesis of N-([1,1′-biphenyl]-4-ylmethyl)-1-(5-cyano-4-hydroxypyrimidin-2-yl)-1H-pyrazole-4-carboxamide (158)

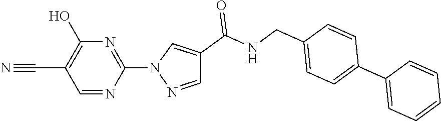 [SMILES: N#Cc1cnc(-n2cc(C(=O)NCc3ccc(-c4ccccc4)cc3)cn2)nc1O]

Following general procedure B: 157 (10 mg, 0.04 mmol), HATU (33 mg, 0.09 mmol), 4-phenylbenzylamine (11.88 mg, 0.06 mmol) and DIPEA (22 μL, 0.13 mmol) gave the titled compound (8 mg, 0.020 mmol, 57%).

¹H NMR (400 MHZ, DMSO) δ 9.15 (s, 1H), 8.99 (t, J=6.0 Hz, 1H), 8.59 (s, 1H), 8.23 (s, 1H), 7.64 (dd, J=7.9, 4.7 Hz, 4H), 7.51-7.31 (m, 5H), 4.48 (d, J=6.0 Hz, 2H).

LRMS m/z calcd. For C₂₂H₁₇N₆O₂ [M+H]⁺: 397.14, found: 397.2

### Example 134—Synthesis of 1-(5-cyano-4-hydroxypyrimidin-2-yl)-N-((4-(trifluoromethyl)cyclohexyl)methyl)-1H-pyrazole-4-carboxamide (159)

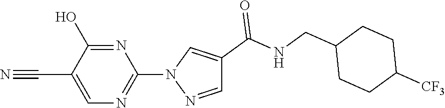 [SMILES: N#Cc1cnc(-n2cc(C(=O)NCC3CCC(C(F)(F)F)CC3)cn2)nc1O]

Following general procedure B: 157 (37 mg, 0.16 mmol), C-4 trifluoromethyl-cyclohexyl-methamine (28 μL, 0.19 mmol), T3P (190 μL, 0.32 mmol) DIPEA (83 UL, 0.48 mmol) gave the title compound (9 mg, 0.022 mmol, 14%).

¹H NMR (400 MHZ, DMSO) δ 9.05 (s, 1H), 8.27 (d, J=6.7 Hz, 2H), 7.98 (s, 1H), 3.07 (t, J=6.0 Hz, 2H), 2.29-2.12 (m, 1H), 1.85 (t, J=16.4 Hz, 4H), 1.64-1.42 (m, 1H), 1.27-1.13 (m, 2H), 1.05-0.91 (m, 2H).

LRMS m/z calcd. For C₁₇H₁₈F₃N₆O₂ [M+H]⁺: 395.14, found: 395.2

### Example 135—Synthesis of 1-(5-cyano-4-hydroxypyrimidin-2-yl)-N-((6-phenylpyridin-3-yl)methyl)-1H-pyrazole-4-carboxamide (160)

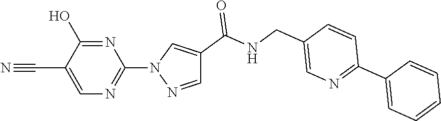 [SMILES: N#Cc1cnc(-n2cc(C(=O)NCc3ccc(-c4ccccc4)nc3)cn2)nc1O]

Following general procedure B: 157 (100 mg, 0.43 mmol), (6-phenylpyridin-3-yl) methanamine (119 mg, 0.65 mmol), T3P (515 μL, 0.87 mmol) DIPEA (223 μL, 0.130 mmol) gave the title compound (65 mg, 0.163 mmol, 38%).

¹H NMR (400 MHz, DMSO) δ 9.28-8.99 (m, 2H), 8.87-8.60 (m, 2H), 8.43-8.19 (m, 1H), 8.09-8.03 (m, 2H), 7.95 (d, J=8.1 Hz, 1H), 7.82 (ddd, J=16.8, 8.3, 2.4 Hz, 1H), 7.54-7.39 (m, 3H), 4.46 (d, J=5.5 Hz, 2H).

LRMS m/z calcd. For C₂₁H₁₋₆N₇O₂ [M+H]⁺: 398.14, found: 398.1

### Example 136—Synthesis of Methyl4-(5-((1-(5-cyano-4-hydroxypyrimidin-2-yl)-1H-pyrazole-4-carboxamido)-methyl)-pyridin-2-yl)-benzoate (161)

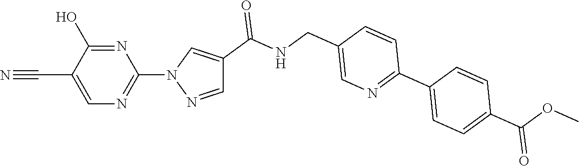 [SMILES: COC(=O)c1ccc(-c2ccc(CNC(=O)c3cnn(-c4ncc(C#N)c(O)n4)c3)cn2)cc1]

Following general procedure B: 157 (100 mg, 0.43 mmol), methyl 4-(5-(aminomethyl)pyridin-2-yl)benzoate (157 mg, 0.65 mmol), T3P (515 μL, 0.87 mmol) DIPEA (223 μL, 0.130 mmol) gave the title compound (11 mg, 0.0241 mmol, 6%).

¹H NMR (400 MHZ, DMSO) δ 9.05 (s, 1H), 8.27 (d, J=6.7 Hz, 2H), 7.98 (s, 1H), 3.07 (t, J=6.0 Hz, 2H), 2.29-2.12 (m, 1H), 1.85 (t, J=16.4 Hz, 4H), 1.64-1.42 (m, 1H), 1.27-1.13 (m, 2H), 1.05-0.91 (m, 2H).

LRMS m/z calcd. For C₂₃H₁₈N₇O₄ [M+H]⁺: 456.14, found: 456.2.

### Example 137—Synthesis of 4-(5-((1-(5-cyano-4-hydroxypyrimidin-2-yl)-1H-pyrazole-4-carboxamido)methyl)pyridin-2-yl)benzoic acid (162)

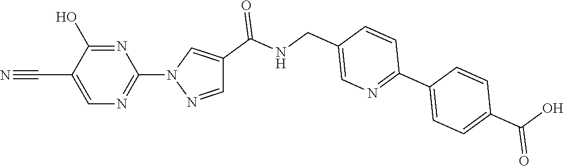 [SMILES: N#Cc1cnc(-n2cc(C(=O)NCc3ccc(-c4ccc(C(=O)O)cc4)nc3)cn2)nc1O]

Following general procedure D: 161 (5 mg, 0.01 mmol), LiCl (4.51 mg, 0.11 mmol) in DMF (1 mL) gave the titled compound (2 mg, 0.0045 mmol, 45%).

¹H NMR (400 MHZ, DMSO) δ 9.13 (s, 1H), 9.04 (t, J=5.5 Hz, 1H), 8.68 (d, J=2.2 Hz, 1H), 8.57 (s, 1H), 8.23-8.16 (m, 3H), 8.04 (dd, J=8.3, 4.0 Hz, 3H), 7.86 (dd, J=8.3, 2.3 Hz, 1H), 4.52 (d, J=5.5 Hz, 2H).

LRMS m/z calcd. For C₂₂H₁₄N₇O₄ [M−H]⁻: 440.11, found: 439.3, [M−2H]²⁻/2=220.0

### Example 138—Synthesis of Ethyl 6-(4-(([1,1′-biphenyl]-4-ylmethyl) carbamoyl)-1H-pyrazol-1-yl)-5-methoxynicotinate (163a)

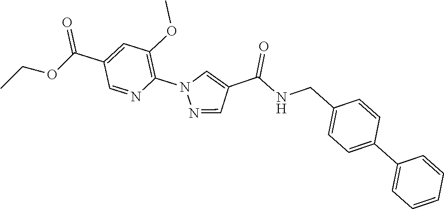 [SMILES: CCOC(=O)c1cnc(-n2cc(C(=O)NCc3ccc(-c4ccccc4)cc3)cn2)c(OC)c1]

Ethyl 6-chloro-5-methoxynicotinate (30 mg, 0.14 mmol), 92 (30 mg, 0.108 mmol), RockPhos G3 (10 mg, 0.014 mmol), Cs₂CO₃ (77 mg, 0.238 mmol) in dioxane (5 ml) for 7 days gave 163a (5 mg, 0.01 mmol, 9%) as a clear oil.

¹H NMR (400 MHZ, THF-d₈) δ 8.71 (s, 1H,), 8.65 (d, J=1.0 Hz, 1H), 8.07-8.05 (m, 1H), 7.91 (t, J=6.0 Hz, 1H), 7.75 (d, J=1.0 Hz, 1H), 7.46-7.24 (m, 9H), 4.58 (d, J=6.0 Hz, 2H), 4.40 (q, J=7.0 Hz, 2H), 3.98 (s, 3H), 1.42 (t, J=7.0 Hz, 3H).

HRMS (ESI-TOF) calcd for C₂₆H₂₅O₄N₄ [M+H]⁺: 457.1870, found: 457.1867.

### Example 139—Synthesis of 6-(4-(([1,1′-biphenyl]-4-ylmethyl) carbamoyl)-1H-pyrazol-1-yl)-5-methoxynicotinic acid (164a)

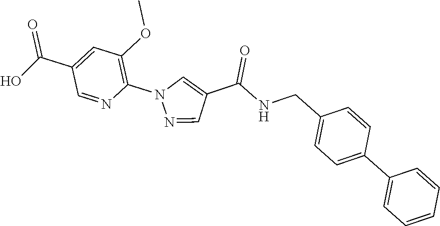 [SMILES: COc1cc(C(=O)O)cnc1-n1cc(C(=O)NCc2ccc(-c3ccccc3)cc2)cn1]

163a (5 mg, 0.0109 mmol) was dissolved in a mixture of THF and water (1.5 ml (10:1). Lithium hydroxide monohydrate (1.0 mg, 0.021 mmol) was added to the reaction mixture and the resulting mixture was allowed to stir at room temperature for 16 hrs. The reaction was confirmed to have gone to completion by TLC and HCL_{aq} (5 ml, 1M) was added to the reaction mixture. The resultant mixture was extracted with EtOAc (3×10 ml), then washed with brine, then dried with anhydrous Na₂SO₄ and purified using flash column chromatography using (CH₂Cl₂, MeOH 0-5%, 1% formic acid) over 20 column volumes gave 164a (3.5 mg, 0.0082 mmol, 76%) as a clear oil.

¹H NMR (400 MHZ, DMSO-d₆) δ 8.69 (s, 1H), 8.47 (s, 1H), 8.12 (s, 1H), 7.99 (s, 1H), 7.70-7.61 (m, 4H), 7.50-7.32 (m, 5H), 4.50 (d, J=6.0 Hz, 2H), 3.89 (s, 3H).

HRMS (ESI-TOF) calcd for C₂₄H₂₁O₄N₄ [M+H]⁺: 429.1557, found: 429.1557.

### Reference Example 140—Synthesis of 2,5-dimethylpyrazolo[1,5-a]pyrimidin-7-amine (163)

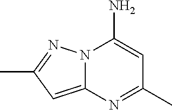

3-aminocrotononitrile (10 g, 0.12 mol) and 3-methyl-1H-pyrazol-5-amine (11.83 g, 0.12 mol) were combined and heated to 140° C. for 2 hrs. The resultant mixture was allowed to cool. The crude mixture was then recrystallised with EtOH to give the titled compound (6.68 g, 0.041 mol, 35%).

LRMS m/z calcd. For C₈H₁₁N₄ [M+H]⁺: 163.1, found: 163.10

### Reference Example 141—Synthesis of Diethyl 2-(((2,5-dimethylpyrazolo[1,5-a]pyrimidin-7-yl)amino)methylene) malonate (164)

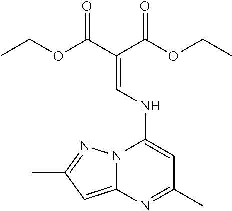 [SMILES: CCOC(=O)C(=CNc1cc(C)nc2cc(C)nn12)C(=O)OCC]

163 (1.50 g, 0.01 mmol) was taken up in toluene (10 mL) before the addition of diethyl 2-(ethoxymethylene) malonate (2.24 ml, 0.01 mmol). The resultant mixture was heated to 120° C. for 48 hr. The reaction mixture was cooled to room temperature. Celite was added to the reaction mixture before the solvent was removed under vacuum. The crude compound was purified using flash chromatography (0-10% MeOH) to give the titled compound (2.26 g, 0.0068 mmol, 74%).

LRMS m/z calcd. For C₁₋₆H₂₁N₄O₄ [M+H]⁺: 333.16, found: 333.2

### Reference Example 142—Synthesis of Ethyl 6-hydroxy-2,5-dimethylpyrazolo[1,5-a]pyrido[3,2-e]pyrimidine-7-carboxylate (165)

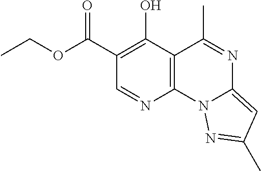 [SMILES: CCOC(=O)c1cnc2c(c(C)nc3cc(C)nn32)c1O]

164 (6.15 g, 18.52 mmol) was dissolved in Eaton's reagent (10 mL). The resultant mixture was heated to 70° C. for 4 hrs, and the progress was followed by LCMS. Once the starting material had be consumed, the reaction mixture was cooled to 0° C. and slowly poured into a saturated NaHCO₃ solution to quench the Eaton's reagent. The resultant mixture was then extracted with a CHCl₃ and IPA mixture (3×100 mL (3:1)). The organic fractions were combined, dried (Na₂SO₄), the solvent was removed under vacuum. The crude compound was purified using flash column chromatography (CHCl₃: MeOH (0-20%) to give the titled compound (4.67 g, 16.32 mmol, 88%).

LRMS m/z calcd. For C₁₄H₁₅N₄O₃ [M+H]⁺: 287.11, found: 287.20

### Reference Example 143—Synthesis of Ethyl 6-chloro-2,5-dimethylpyrazolo[1,5-a]pyrido[3,2-e]pyrimidine-7-carboxylate (166)

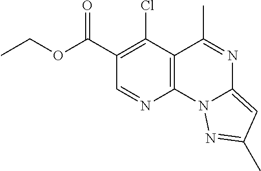 [SMILES: CCOC(=O)c1cnc2c(c(C)nc3cc(C)nn32)c1Cl]

165 (4.67 g, 16.32 mmol) was dissolved in phosphorus oxychloride (25 mL) and refluxed at 100° C. for 3 hrs. The reaction mixture was cooled to 0° C. and slowly quenched with saturated NaHCO₃ solution. The resultant mixture was extracted with a CHCl₃: IPA mixture (3:1 (3×100 mL)). The organic fractions were combined, dried (Na₂SO₄) and the solvents removed under vacuum. The crude mixture was then taken onto the next step without further purification (4.54 g, 14.93 mmol, 91%).

LRMS m/z calcd. For C₁₄H₁₄ClN₄O₄ [M+H]⁺: 305.08, found: 305.2

### Reference Example 144—Synthesis of Methyl 6-methoxy-2,5-dimethylpyrazolo[1,5-a]pyrido[3,2-e]pyrimidine-7-carboxylate (167)

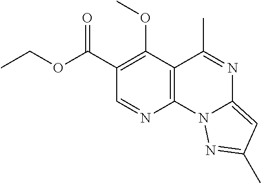 [SMILES: CCOC(=O)c1cnc2c(c(C)nc3cc(C)nn32)c1OC]

166 (156 mg, 0.51 mmol) was dissolved in MeOH (10 mL) before the addition of sodium methoxide solution (5 mL, 0.5 M). The resultant mixture was stirred at room temperature for 4 hrs. Celite was added to the reaction mixture before the solvent was removed under vacuum. The crude mixture was then purified using flash column chromatography (MeOH in CHCl₃ (0-10%) to give the titled compound (38 mg, 0.13 mmol, 26%).

LRMS m/z calcd. For C₁₄H₁₅NO₃ [M+H]⁺: 287.11, found: 287.2

### Example 145—Synthesis of 6-methoxy-2,5-dimethyl-N-((4-(trifluoromethyl)cyclohexyl)methyl) pyrazolo[1,5-a]pyrido[3,2-e]pyrimidine-7-carboxamide (168)

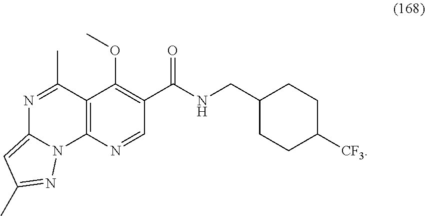 [SMILES: COc1c(C(=O)NCC2CCC(C(F)(F)F)CC2)cnc2c1c(C)nc1cc(C)nn12]

Following general procedure A: 167 (30 mg, 0.1 mmol), 4-trifluoromethyl-cyclohexylamine (19 μL, 0.16 mmol) and DABAL (26.85 mg, 0.1 mmol) gave the titled compound (9 mg, 0.02 mmol, 20%).

Solvent system used for purification: 0%-15% MeOH in CH₃Cl.

LRMS m/z calcd. For C₂₁H₂₅F₃N₅O₂ [M+H]⁺: 436.20, found: 436.2.

### Example 146—Synthesis of N-([1,1′-Biphenyl]-4-ylmethyl)-6-hydroxypyrazolo[1,5-a]pyrido[3,2-e]pyrimidine-7-carboxamide (169)

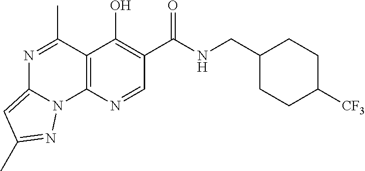 [SMILES: Cc1cc2nc(C)c3c(O)c(C(=O)NCC4CCC(C(F)(F)F)CC4)cnc3n2n1]

Following general procedure D: 168 (9 mg, 0.02 mmol), LiCl (8.6 mg, 0.21 mmol) in DMSO (1 mL) gave the titled compound (1.5 mg, 0.0035 mmol, 18%).

Solvent system used for purification: Reverse phase (0%-100% ACN (0.1% formic acid)) in H₂O (0.1% formic acid).

¹H NMR (400 MHZ, DMSO) δ 8.91 (s, 1H), 6.42 (s, 1H), 3.23 (d, J=5.1 Hz, 2H), 2.66 (s, 3H), 2.41 (s, 3H), 2.07 (s, 1H), 1.83-1.75 (m, 2H), 1.65 (d, J=12.7 Hz, 2H), 1.48 (d, J=9.6 Hz, 1H), 1.25-1.08 (m, 2H), 0.95-0.81 (m, 2H).

LRMS m/z calcd. For C₂₀H₂₃F₃N₅O₂ [M+H]⁺: 422.18, found⁺: 422.2

### Example 147—Synthesis of N-((6-chloropyridin-3-yl)methyl)-6-methoxy-2,5-dimethylpyrazolo[1,5-a]pyrido[3,2-e]pyrimidine-7-carboxamide (170)

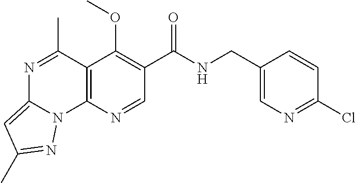 [SMILES: COc1c(C(=O)NCc2ccc(Cl)nc2)cnc2c1c(C)nc1cc(C)nn12]

Following general procedure A: 167 (900 mg, 3.15 mmol), (6-chloropyridin-3-yl) methanamine (532 mg, 3.78 mmol) and DABAL (805 mg, 3.15 mmol) gave the titled compound (364 mg, 0.91 mmol, 29%).

Solvent system used for purification: 0%-15% MeOH in CH₃Cl.

LRMS m/z calcd. For C₁₉H₁₈ClN₆O₂ [M+H]⁺: 397.12, found: 397.10

### Example 148—Synthesis of N-((6-(4-carbamoylphenyl)pyridin-3-yl)methyl)-6-hydroxy-2,5-dimethylpyrazolo[1,5-a]pyrido[3,2-e]pyrimidine-7-carboxamide (171)

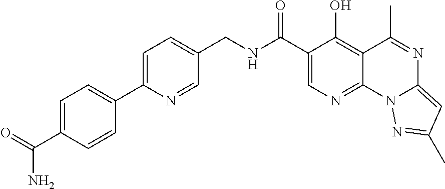 [SMILES: Cc1cc2nc(C)c3c(O)c(C(=O)NCc4ccc(-c5ccc(C(N)=O)cc5)nc4)cnc3n2n1]

Following general procedure C: 170 (15 mg, 0.04 mmol), (4-(carbamoyl)phenyl) boronic acid (9.75 mg, 0.06 mmol), Pd Amphos (2.77 mg, 0.0039 mmol) and cesium carbonate (38.19 mg, 0.12 mmol) gave the titled compound (2 mg, 0.0042 mmol, 11%).

¹H NMR (400 MHZ, DMSO) δ 8.77 (s, 1H), 8.69-8.66 (m, 1H), 8.31 (s, 1H), 8.14 (t, J=4.2 Hz, 3H), 7.99 (dd, J=17.9, 8.4 Hz, 3H), 7.86 (dd, J=8.1, 2.5 Hz, 1H), 6.53 (s, 2H), 6.20 (s, 1H), 4.59 (d, J=6.0 Hz, 2H), 2.87 (s, 3H), 2.36 (s, 3H).

LRMS m/z calcd. For C₂₅H₂₂N₇O₃ [M+H]⁺: 468.18, found: 468.1, [M+2H]²⁺/2=234.70

### Example 149—Synthesis of 6-hydroxy-2,5-dimethyl-N-((6-(pyrimidin-5-yl) pyridin-3-yl)methyl) pyrazolo[1,5-a]pyrido[3,2-e]pyrimidine-7-carboxamide (172)

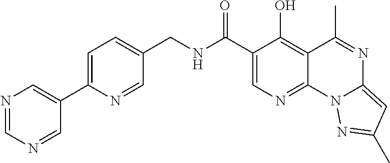 [SMILES: Cc1cc2nc(C)c3c(O)c(C(=O)NCc4ccc(-c5cncnc5)nc4)cnc3n2n1]

Following general procedure C: 170 (15 mg, 0.04 mmol), 5-(4,4,5,5-tetramethyl-1,3,2-dioxaborolan-2-yl)pyrimidine (12.1 mg, 0.06 mmol), Pd Amphos (1.39 mg, 0.0019 mmol) and cesium carbonate (38.19 mg, 0.12 mmol) gave the titled compound (2.5 mg, 0.0058 mmol, 15%).

¹H NMR (400 MHZ, DMSO) δ 10.69 (s, 1H), 9.42 (s, 2H), 9.24 (s, 1H), 8.74 (d, J=2.2 Hz, 1H), 8.59 (s, 1H), 8.14-8.09 (m, 1H), 7.93 (dd, J=8.2, 2.3 Hz, 1H), 6.52 (s, 1H), 6.42 (s, 1H), 4.63 (d, J=6.0 Hz, 2H), 2.89 (s, 3H), 2.45 (s, 3H).

LRMS m/z calcd. For C₂₂H₁₉N₈O₂ [M+H]⁺: 427.16, found: 427.1, [M+2H]²⁺/2=214.20

### Example 150—Synthesis of 6-hydroxy-2,5-dimethyl-N-((6-(4-morpholinophenyl)pyridin-3-yl)methyl) pyrazolo[1,5-a]pyrido[3,2-e]pyrimidine-7-carboxamide (173)

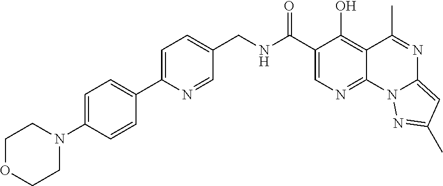 [SMILES: Cc1cc2nc(C)c3c(O)c(C(=O)NCc4ccc(-c5ccc(N6CCOCC6)cc5)nc4)cnc3n2n1]

Following general procedure C: 170 (15 mg, 0.04 mmol), 4-morpholine-phenylboronic acid (12.1 mg, 0.06 mmol), Pd Amphos (1.39 mg, 0.0019 mmol) and cesium carbonate (38.19 mg, 0.12 mmol) gave the titled compound (5 mg, 0.009 mmol, 25%).

¹H NMR (400 MHZ, DMSO) δ 10.50 (s, 1H), 8.59-8.48 (m, 2H), 7.98-7.93 (m, 2H), 7.83 (dd, J=8.3, 0.9 Hz, 1H), 7.76 (dd, J=8.2, 2.3 Hz, 1H), 7.05-6.99 (m, 2H), 6.53 (s, 1H), 6.45 (s, 1H), 4.57 (d, J=5.9 Hz, 2H), 3.77-3.71 (m, 4H), 3.23-3.14 (m, 4H), 2.88 (s, 3H), 2.46 (s, 3H).

LRMS m/z calcd. For C₂₈H₂₈N₇O₃ [M+H]⁺: 510.22, found [M+2H]²⁺/2=255.70

### Example 151—Synthesis of 6-hydroxy-2,5-dimethyl-N-((6-(4-(methylcarbamoyl)phenyl)pyridin-3-yl)methyl) pyrazolo[1,5-a]pyrido[3,2-e]pyrimidine-7-carboxamide (174)

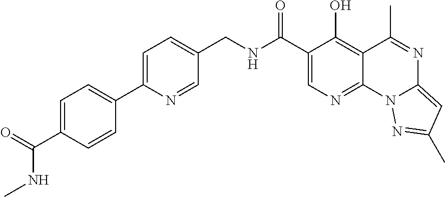

Following general procedure C: 170 (15 mg, 0.04 mmol), 4—N methylcarbonyl-phenylboronic acid acid (10.52 mg, 0.06 mmol), Pd Amphos (2.77 mg, 0.0039 mmol) and cesium carbonate (38.19 mg, 0.12 mmol) gave the titled compound (1 mg, 0.0021 mmol, 5%).

¹H NMR (400 MHZ, DMSO) δ 8.67 (d, J=6.2 Hz, 2H), 8.51 (d, J=5.0 Hz, 1H), 8.18-8.10 (m, 2H), 8.02 (d, J=8.2 Hz, 1H), 7.93 (d, J=8.4 Hz, 2H), 7.88-7.78 (m, 1H), 6.52 (s, 1H), 6.34 (s, 1H), 4.61 (d, J=5.8 Hz, 2H), 2.88 (s, 3H), 2.80 (d, J=2.3 Hz, 3H), 2.41 (s, 3H).

LRMS m/z calcd. For C₂₆H₂₄N₇O₃ [M+H]⁺: 482.19, found: [M+2H]²⁺/2=241.50

### Example 152—Synthesis of 6-hydroxy-2,5-dimethyl-N-((6-phenylpyridin-3-yl)methyl) pyrazolo[1,5-a]pyrido[3,2-e]pyrimidine-7-carboxamide (175)

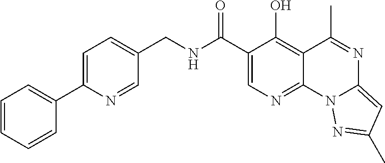 [SMILES: Cc1cc2nc(C)c3c(O)c(C(=O)NCc4ccc(-c5ccccc5)nc4)cnc3n2n1]

Following general procedure C: 170 (50 mg, 0.13 mmol), phenylboronic acid (46.21 mg, 0.38 mmol), Pd Amphos (8.94 mg, 0.01 mmol) and cesium carbonate (123 mg, 0.38 mmol) gave the titled compound (9 mg, 0.0212 mmol, 22%).

¹H NMR (400 MHZ, DMSO) δ 9.42 (s, 1H), 8.95 (s, 1H), 8.49 (s, 1H), 8.09-8.00 (m, 2H), 7.89 (d, J=8.2 Hz, 1H), 7.69-7.62 (m, 1H), 7.51-7.36 (m, 3H), 6.47 (s, 1H), 4.64 (s, 2H), 2.84 (s, 3H), 2.43 (s, 3H).

LRMS m/z calcd. For C₂₄H₂₁N₆O₂ [M+H]⁺: 425.17, found: 425.2, [M+2H]²⁺/2=213.2

### Example 153—Synthesis of 6-hydroxy-2,5-dimethyl-N-((6-(3-morpholinophenyl)pyridin-3-yl)methyl) pyrazolo[1,5-a]pyrido[3,2-e]pyrimidine-7-carboxamide (176)

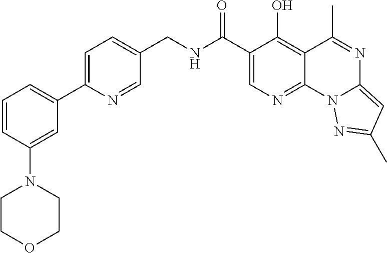 [SMILES: Cc1cc2nc(C)c3c(O)c(C(=O)NCc4ccc(-c5cccc(N6CCOCC6)c5)nc4)cnc3n2n1]

Following general procedure C: 170 (40 mg, 0.10 mmol), 3-morpholo-phenylboronic acid (63 mg, 0.30 mmol), Pd Amphos (7.15 mg, 0.01 mmol) and cesium carbonate (99 mg, 0.30 mmol) gave the titled compound (14 mg, 0.275 mmol, 28%).

¹H NMR (400 MHZ, DMSO) δ 9.52 (s, 1H), 8.95 (s, 1H), 8.46 (d, J=2.2 Hz, 1H), 7.88 (d, J=8.2 Hz, 1H), 7.68-7.56 (m, 2H), 7.46 (d, J=7.6 Hz, 1H), 7.31 (t, J=7.9 Hz, 1H), 7.00 (dd, J=8.3, 2.5 Hz, 1H), 6.46 (s, 1H), 4.67-4.60 (m, 2H), 3.75 (t, J=4.7 Hz, 4H), 3.16 (dd, J=6.6, 3.2 Hz, 4H), 2.84 (s, 3H), 2.43 (s, 3H).

LRMS m/z calcd. For C₂₈H₂₈N₇O₃ [M+H]⁺: 510.22, found: 510.30, [M+2H]²⁺/2=255.80

### Example 154—Synthesis of N-((6-(3-carbamoylphenyl)pyridin-3-yl)methyl)-6-hydroxy-2,5-dimethylpyrazolo[1,5-a]pyrido[3,2-e]pyrimidine-7-carboxamide (177)

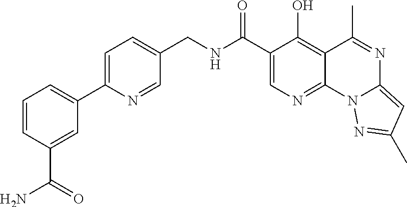 [SMILES: Cc1cc2nc(C)c3c(O)c(C(=O)NCc4ccc(-c5cccc(C(N)=O)c5)nc4)cnc3n2n1]

Following general procedure C: 170 (40 mg, 0.10 mmol), 3-amido-phenylboronic acid (50 mg, 0.30 mmol), Pd Amphos (7.15 mg, 0.01 mmol) and cesium carbonate (99 mg, 0.30 mmol) gave the titled compound (11 mg, 0.0235 mmol, 24%).

¹H NMR (400 MHZ, DMSO) δ 9.41 (s, 1H), 8.95 (s, 1H), 8.56-8.49 (m, 2H), 8.21-8.06 (m, 2H), 7.97 (d, J=8.2 Hz, 1H), 7.91 (d, J=7.7 Hz, 1H), 7.71 (dd, J=8.3, 2.3 Hz, 1H), 7.55 (t, J=7.7 Hz, 1H), 7.43 (s, 1H), 6.47 (s, 1H), 4.66 (d, J=4.1 Hz, 2H), 2.85 (s, 3H), 2.43 (s, 3H).

LRMS m/z calcd. For C₂₅H₂₂N₇O₃ [M+H]⁺: 468.18, found: 468.2, [M+2H]²⁺/2=234.80

### Example 155—Synthesis of N-((6-(benzo[d]thiazol-5-yl) pyridin-3-yl)methyl)-6-hydroxy-2,5-dimethylpyrazolo[1,5-a]pyrido[3,2-e]pyrimidine-7-carboxamide (178)

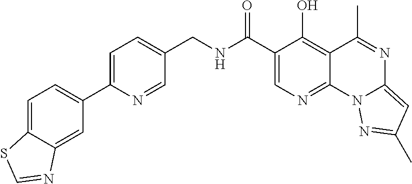 [SMILES: Cc1cc2nc(C)c3c(O)c(C(=O)NCc4ccc(-c5ccc6scnc6c5)nc4)cnc3n2n1]

Following general procedure C: 170 (50 mg, 0.13 mmol), 5-(4,4,5,5-tetramethyl-1,3,2-dioxaborolan-2-yl)benzo[d]thiazole (98.86 mg, 0.38 mmol), Pd Amphos (8.94 mg, 0.01 mmol) and cesium carbonate (123 mg, 0.38 mmol) gave the titled compound (5 mg, 0.0103 mmol, 8%).

¹H NMR (400 MHZ, DMSO) δ 9.44 (s, 1H), 8.96 (s, 1H), 8.73 (d, J=1.6 Hz, 1H), 8.54 (d, J=2.3 Hz, 1H), 8.27-8.19 (m, 2H), 8.08 (d, J=8.2 Hz, 1H), 7.71 (dd, J=8.3, 2.3 Hz, 1H), 6.46 (s, 1H), 4.65 (d, J=4.6 Hz, 2H), 2.85 (s, 3H), 2.43 (s, 3H).

LRMS m/z calcd. For C₂₅H₂₀N₇O₂S [M+H]⁺: 482.14, found 482.20, [M+2H]²⁺/2=241.70

### Example 156—Synthesis of 6-hydroxy-2,5-dimethyl-N-((6-(3-(methylcarbamoyl)phenyl)pyridin-3-yl)methyl) pyrazolo[1,5-a]pyrido[3,2-e]pyrimidine-7-carboxamide (179)

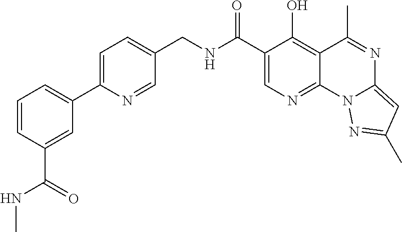 [SMILES: CNC(=O)c1cccc(-c2ccc(CNC(=O)c3cnc4c(c(C)nc5cc(C)nn54)c3O)cn2)c1]

Following general procedure C: 170 (29 mg, 0.07 mmol), 3-(methylcarbamoyl)phenyl) boronic acid (39.33 mg, 0.22 mmol), Pd Amphos (5.18 mg, 0.01 mmol) and cesium carbonate (71 mg, 0.22 mmol) gave the titled compound (11 mg, 0.0228 mmol, 32%).

¹H NMR (400 MHZ, DMSO) δ 9.96 (s, 1H), 8.96 (s, 1H), 8.60-8.44 (m, 3H), 8.16 (d, J=7.9 Hz, 1H), 7.95 (d, J=8.2 Hz, 1H), 7.86 (d, J=7.7 Hz, 1H), 7.70 (dd, J=8.2, 2.3 Hz, 1H), 7.54 (t, J=7.7 Hz, 1H), 6.45 (s, 1H), 4.64 (s, 2H), 2.84 (s, 3H), 2.80 (d, J=4.4 Hz, 3H), 2.42 (s, 3H).

LRMS m/z calcd. for C₂₆H₂₃N₇O₃ [M+H]⁺: 482.19, found: 482.2, [M+2H]²⁺/2=241.60

### Example 157—Synthesis of(S)-6-hydroxy-N-(1-(4-methoxyphenyl)ethyl)-2,5-dimethylpyrazolo[1,5-a]pyrido[3,2-e]pyrimidine-7-carboxamide (180)

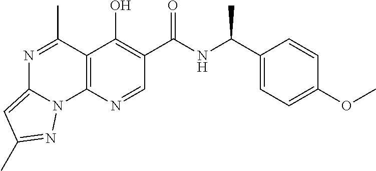

Following general procedure A: 167 (50 mg, 0.17 mmol), (S)-1-(4-methoxyphenyl) ethan-1-amine (31 μL, 0.21 mmol) and DABAL-Me₃ (44.76 mg, 0.17 mmol) gave the titled compound (7 mg, 0.0179 mmol, 11%).

Solvent system used for purification: 0%-15% MeOH in CH₃Cl.

¹H NMR (400 MHZ, DMSO) δ 10.40 (d, J=7.9 Hz, 1H), 8.51 (s, 1H), 7.35-7.28 (m, 2H), 6.91 (dd, J=8.5, 1.9 Hz, 2H), 6.47 (s, 1H), 5.16-5.05 (m, 1H), 3.74 (s, 3H), 2.89 (s, 3H), 2.46 (s, 3H), 1.47 (d, J=6.9 Hz, 3H).

LRMS m/z calcd. For C₂₁H₂₂N₅O₃ [M+H]⁺: 392.17, found: 392.20

### Activity Examples

### General Experimental Methods

### Protein Production

Recombinant forms PHD2⁷¹, FIH¹³⁰, JMJD6¹²³, KDM4A¹⁴⁰, KDM5B^{34,141} and KDM6B¹²⁶ were produced and purified from E. coli as described.

### Inhibition Assays

PHD2⁷¹, FIH⁷¹, JMJD6 (described in Example 172), KDM4A (described in Example 172), KDM5B (described in Example 172), KDM6B (described in Example 172), OGFOD1 (described in Example 172).

Preparation of tPHD2 (Residues 181-426)

In brief, cDNA encoding for the catalytic domain of tPHD2 (181-426 residues) was cloned into the pET28a (+)/pET24a (+) vectors (Novagen), to enable production of tPHD2 (residues 181-426) protein with/without an N-terminal His6-tag. The tPHD2 (residues 181-426) encoding construct was transformed into the Escherichia coli BL21 DE3 cell line; protein production was induced with 0.5 mM isopropyl-b-D-thiogalactosidase (3-5 hr at 28° C.). Cells were harvested and lysed by sonication in 20 mM Tris-HCl (pH 7.0) and 0.3 M NaCl; soluble protein (about 5% total soluble extract) was purified by immobilized Ni ion affinity chromatography using pentadentate tris-carboxymethyl ethylene diamine resin followed by cleavage of the His6-tag by thrombin (or alternately by cation exchange chromatography) with a final purification by gel filtration chromatography. The protein was exchanged into 50 mM Tris-HCl buffer (pH 7.5) and concentrated to % 40 mg/ml. The protein was of >95% purity, as determined by SDS-PAGE analysis and electrospray ionization mass spectrometric analysis.

### LC-MS PHD2 Hydroxylation Assays

The PHD2 RF-MS RapidFire liquid chromatography mass spectrometry (RF-MS) assay monitors turnover of a C-terminal oxygenase dependent domain (CODD) peptide substrate DLDLEMLAPYIPMDDDFQL-CONH2 and appearance of the hydroxylated peptide product (Pro564) in an endpoint type assay format (typical enzyme incubation time of 15 minutes). Tris(hydroxymethyl)aminomethane was from Fisher. Ferrous ammonium sulfate (FAS), 2-oxoglutarate (2OG) and L-ascorbic acid were from Sigma Aldrich; solutions of these were prepared freshly each day. All inhibition assays were carried out in 384-well polypropylene plates (Greiner Bio-One). PHD2 assays were performed in assay buffer (50 mM Tris. Cl pH 7.5, 50 mM NaCl). Titrations of compounds for IC₅₀ determinations (3-fold and 11-point IC₅₀) were prepared using an ECHO 550 acoustic dispenser (Labcyte) and dry dispensed into 384-well polypropylene assay plates. The final assay concentration of DMSO was kept constant at 0.5%. PHD2 protein was prepared at a concentration of 300 nM in assay buffer and 25 μl dispensed across each 384-well assay plate. The PHD2 solution was allowed to equilibrate with the inhibitors for 15 minutes at room temperature; the enzyme reaction then initiated by dispense of 25 μl of substrate (20 μM FAS, 200 UM L-ascorbic acid, 10 μM CODD peptide and 20 μM 2OG in the assay buffer. Enzyme reactions were allowed to proceed for 20 minutes at room temperature and the reaction terminated by addition of 10% formic acid (5 μl). Assay plates were then transferred to a RapidFire RF360 sampling robot (Agilent) connected to an Agilent 6530 accurate mass quadrupole-time-of-flight (Q-TOF) mass spectrometer. Assay samples were aspirated under vacuum and loaded onto a C₄ solid phase extraction (SPE) cartridge. After loading the C4 SPE was washed with 0.1% formic acid in water to remove non-volatile buffer salts and then peptide was eluted from the SPE with 85% acetonitrile, 15% water containing 0.1% formic acid onto the mass spectrometer. Peptide charge states were monitored in positive mode. Ion chromatogram data were extracted for the +2 charge state and peak area data integrated using RapidFire Integrator software (Agilent). % conversion of the CODD peptide substrate to the +16 hydroxylated peptide was calculated using the equation: % conversion=100×hydroxylated/(hydroxylated+non-hydroxylated peptide). IC₅₀ values were determined from non-linear regression plots using GraphPad prism.

Cell Culture and Immunoblotting with Hep3B cells

These were carried out as reported in T. L. Yeh et al, Chem Sci, 2017, 8, 7651-7668

Cell Culture with HEK293T Cells:

HEK293 T cells were grown in Dulbecco's Modified Eagle's Medium DMEM (high glucose, pyruvate, no glutamine, Gibco) supplemented with 10% of FBS and 1% GlutaMAX. in a 37° C. incubator at 5% CO₂. Cells were grown to 90% confluency.

### Inhibitor Addition and Incubation:

Cells were plated at a density of 1.2×10⁶ in T75 flasks. Cells were exposed to the inhibitor at a final concentration at 1% DMSO and were incubated at 37° C. for 3-18 hours.

Protein Extraction and Analysis with HEK293T Cells:

Cells were washed twice with ice-cold PBS (Sigma, D8537). 1× RIPA buffer [Sigma, R0278] buffer, and a protease inhibitor [cOmplete™, Mini, EDTA-free Protease Inhibitor Cocktail, Roche]) were used for protein extraction. Adherent cells were scraped using a scraper and transferred to an ice-cooled microcentrifuge tube. The cell suspension was either frozen at −20° C. or incubated for 45 min on ice and sonicated for 3 cycles of 10 seconds pulse with 5 seconds intervals. After centrifuging (16,000 g, 15 min,4° C.), the cell supernatant was collected in a new microcentrifuge tube. The protein concentrations were determined using a BCA protein assay kit (Thermo Scientific™ Pierce™ BCA Protein Assay Kit).

SDS-PAGE Analyses with HEK293T Cells

To perform the Polyacrylamide gel electrophoresis all the samples were loaded into a pre-cast NuPAGE 4-12% Bis-Tris Protein Gels (Life Tech). The gels were run in 1× Tris/glycine/SDS running buffer (20× NuPAGE MES SDS Running Buffer, Life tech) at 180 V for 45 min (Mini Gel Tank, Life Technologies). 5 μl of protein ladder marker (Page Ruler Prestained Protein Ladder, Thermo Scientific) was used to compare protein sizes.

Western Blot Analyses with HEK293T Cells

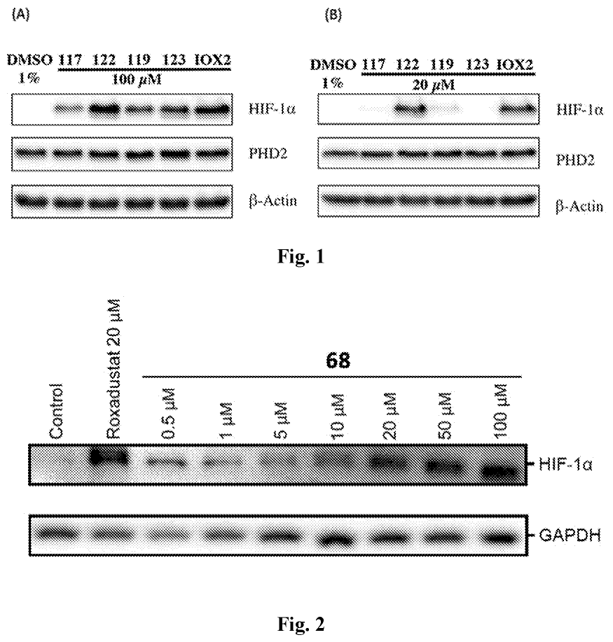

For the Western blot analyes with HEK293T cells as shown in FIGS. 1, 2, 3 and 8 a gel with the resolved proteins was then transferred onto a nitrocellulose membrane (Amersham Protran Premium 0.2 NC 300 mm, GE Healthcare) by using a 1× transfer buffer (20× NuPAGE Transfer Buffer, Invitrogen). Mini Protean Tetra Cell, Bio-Rad used for the transfer. The transfer was performed at 100 volts for 1 hour. Membranes were blocked with 5% milk powder in 1× PBS-T for 30 mins then incubated overnight at 4° C. with primary antibodies (in 1:1000 dilutions) which were prepared in 1% milk powder in 1× PBS-T buffer. The membranes were washed three times for 10-minutes with 1× PBS-T, then incubated for the second time for 1 hour at RT in a horseradish peroxidase (HRP) conjugated secondary antibody (in 1:5000 dilutions), prepared with 1% milk powder in 1× PBS-T. Later the blots were washed three times 10-min with 1×PBS-T and followed by GE Healthcare Amersham™ ECL™ Prime Western Blotting Detection Reagent (RPN2236) and protein levels were measured by densitometric analysis using Bio-Rad Universal Hood iii. Values were normalized to glyceraldehyde 3-phosphate dehydrogenase (GAPDH) and the corresponding controls such as 1% (v/v) dimethyl sulfoxide (DMSO), 20 μM of Roxadustat. The primary antibodies used were Purified Mouse Anti-Human HIF-1α [Catalog Number 610959]. Secondary antibodies used were Rabbit Anti-Mouse IgG (D3V2A) mAb (HRP Conjugate).

Cells were harvested in urea/SDS buffer (6.7 M urea, 10 mM Tris-Cl pH 6.8, 10% glycerol, and 1% SDS) supplemented with 1 mM dithiothreitol following a phosphate-buffered saline (PBS) rinse. Cell extracts were analyzed with SDS-PAGE, electroblotted to polyvinylidene difluoride (PVDF) membrane (Millipore) and probed with the corresponding primary antibody for 1 hour at room temperature or overnight at 4° C. The secondary antibody specific to primary antibody species conjugated with HRP were applied at room temperature for 1 hour. The antibodies used for immonoblotting were as follows: pan-anti-HIF-1α (clone 54, BD Transduction Laboratories™), anti-HIF-1α Hyp402 (catalogue number 07-1585, Merck Millipore), anti-HIF-1α Hyp564 (D43B5, Cell Signaling), anti-HIF-1α HyN803 (a generous gift from Lee et al. 15), PHD216, PHD316, and HRP-conjugated anti-β-actin (clone AC15, Abcam). Details of antibodies used are given in Tian et al. Detection of signal was achieved using SuperSignal™ West Dura Extended Duration Substrate kit (Thermo Scientific 34075, unless otherwise stated) either with film or ChemiDoc™ MP (BIO-RAD) imaging system. Quantification of the immunobloting signal was performed with Image Lab™ software from BIO-RAD.

### Haemato-Oncology Experimental Methods

### Mouse Strains

NBSGW and Vav-iCre mice were purchased from Jackson Laboratory. Vav1-iCre, also known as Vav-iCre mice, were originally developed by De Boer et al (Eur. J. Immunol. 2003. 33: 314-325). Phd1^{fl/fl}, Phd2^{fl/fl} mice were donated by Prof. Sir Peter J Ratcliffe (Mazzone et al., Cell 136, 839-851, 2009). Phd1^{fl/fl}; Vav-iCre and Phd2^{fl/fl}; Vav-iCre mice were generated by crossing Phd1^{fl/fl} and Phd2^{fl/fl} males to Vav-iCre females.

Double heterozygous, double transgenic CAG-rtTA+/−; TRE-shPhd2+/− (referred to as shPhd2) and CAG-rtTA+/−; TRE−/− (referred to as Control) mice were donated by Prof Chris W Pugh and Prof. Sir Peter J Ratcliffe, and were bred to generate CAG-rtTA+/−; TRE-shPhd2+/− and CAG-rtTA+/−; TRE-shPhd2−/− mice used in all experiments. Upon doxycycline (Dox) treatment, there is activation of the tetracycline response element (TRE) controlled GFP-shPhd2 cassette (TRE-shPhd2), which is downstream of the collagen type I gene (Colla1). This results in transcription of both GFP and shPHD2 in the shPhd2 mice, but not control mice.

iMLL-AF9 mice were obtained from Juerg Schwaller (Cancer Cell 30, 43-58, 2006) and crossed to shPhd2 and control mice (as described above) to generate iMLL-AF9;Control and iMLL-AF9;shPhd2 mice.

All mice were on the C₅₇BL/6 genetic background. All transgenic and knockout mice were CD45.2⁺. Congenic recipient mice were CD45.1⁺/CD45.2⁺.

Mice used for support BM cells during transplantation experiments were CD45.1+.

All experiments on animals were performed under UK Home Office authorisation (Project Licence number: pp 4153,210).

### Human Tissue & Ethical Approvals

All use of human tissue was in compliance with the ethical and legal framework of the United Kingdom's Human Tissue Act, 2004. Primary human AML samples were from Barts Cancer Institute Biobank (instituted with approval of the Research Ethics Committee). Their use was authorised following ethical review by the Tissue Biobank's scientific sub-committee, and with the informed consent of the donor.

### Flow Cytometry of Haematopoietic Cells

Bone marrow (BM) cells were isolated by crushing tibias and femurs using a pestle and mortar. Peripheral blood (PB) was collected in EDTA-coated tubes and lysed by incubation with NH₄Cl (Stem Cell Technologies-07850) prior to staining. FL cells were prepared by mashing the tissue and passing through a 70 μm strainer. Single cell suspensions from BM, PB or FL were incubated with Fc block and then stained with antibodies. For haematopotic stem and progenitor cell analyses (HSPC), following incubation with Fc block, unfractionated BM cells were stained with lineage markers containing biotin-conjugated anti-CD4, anti-CD5, anti-CD8a, anti-CD11b, anti-B220, anti-Gr-1 and anti-Ter119 antibodies together with BV711-conjugated anti-c-Kit, APC-Cy7-conjugated anti-Sca-1, PE-conjugated anti-CD48 and PE-Cy7-conjugated anti-CD150 antibodies. Biotin-conjugated antibodies were then stained with PB-conjugated streptavidin. For analyses of differentiated BM, PB or FL cells, following incubation with Fc block, spleen or BM cell suspensions were stained with PerCP-conjugated anti-B220 and APC-Cy7-conjugated anti-CD19 antibodies for B cells; APC-conjugated anti-CD11b and PE-Cy7-conjugated anti-Gr-1 for myeloid cells; PE-conjugated anti-CD4 and anti-CD8 antibodies for T cells.

To distinguish CD45.2⁺-donor derived cells in PB or BM of transplanted mice, BV711-conjugated anti-CD45.1 and Pacific Blue-conjugated anti-CD45.2 antibodies were used. For HSC and progenitor staining in transplanted mice, APC-conjugated anti-c-Kit and PerCp-conjugated streptavidin was used; the remainder of the staining was as described above. For analyses of differentiated cells in PB and BM of transplanted mice, myeloid cells and lymphoid cells were stained as described above. TO-PRO-3 or DAPI were used for dead cell exclusion.

To assess human AML burden, cells were stained with anti-human FITC-conjugated anti-CD45, APC-conjugated anti-CD33 and anti-human PE-conjugated CD14.

Flow cytometry analyses were performed using a LSRFortessa (BD). Cell sorting was performed on a FACSAria Fusion (BD).

### Production of Meis1/Hoxa9 Leukaemic Cells

Foetal livers (FLs) were dissected from 14.5 dpc embryos lysed by incubation with NH₄Cl (Stem Cell Technologies-07850). Cells were incubated with immunomagnetic microbeads, conjugated to monoclonal anti-mouse CD117 (cKit) antibody (isotype: rat IgG2b) (Miltenyi Biotec-130091224) for 25 min at 4° C. Cells were added to a MACS LS column (Miltenyi Biotec-130042401) attached to a QuadroMACS (Miltenyi Biotec) magnetic separator, and washed four times with PBS (2% FCS, 2 mM EDTA). To collect the cKit enriched cells, PBS (2% FCS, 2 mM EDTA) was added, the column was removed from the magnet, and the cKit enriched fraction was flushed out.

cKit⁺ FL cells were incubated overnight in Iscove's Modified Dulbecco's Medium (IMDM) with 10% FCS, 40 ng/ml of mouse recombinant stem cell factor (SCF) (carrier-free) (Biolegend, Cat. 579702), 20 ng/ml of recombinant mouse IL-3 (carrier-free) (Biolegend, Cat. 575502) and IL-6 (Biolegend, Cat. 575702). Non-tissue culture treated 24 well plates (Nunc, 144530) were coated with RetroNectin® (Takara, T100A) and left overnight at 4° C. The next day, RetroNectin® was removed from each well and 1 mL (500 μl of Meis1, 500 μl Hoxa9) of retroviral supernatant was added to the wells. The plate was centrifuged at 2000×g for 2 h at 32° C. After centrifugation, the viral supernatant was removed and 250 000 c-Kit⁺ cells in IMDM with 10% FCS, and 40 ng/mL SCF, 20 ng/ml IL-3 and 20 ng/ml IL-6 were seeded onto the virus-coated plate and left overnight at 37° C., 5% CO₂. 12 h later, a second RetroNectin®-coated plate was similarly prepared with virus. The c-Kit⁺ cells were then washed off the first plate and transferred onto the new viral plate. This sequence was repeated 12 h later, after which the c-Kit⁺ cells were transferred onto a non-coated plate to recover and start expressing antibiotic resistance genes. 24 h later, cells were seeded in media (IMDM with 10% FCS, and 40 ng/mL SCF, 20 ng/ml IL-3 and 20 ng/mL IL-6) containing 1.5 μg/mL puromycin and 1 mg/mL neomycin. Antibiotic selection lasted for 3 days.

Transduced cells were subjected to three rounds of CFC assays in MethoCult™ M3231 (STEMCELL Technologies) supplemented with 20 ng/ml SCF, 10 ng/ml IL-3, 10 ng/ml IL-6 and 10 ng/ml GM-SCF. Colonies were counted 5-7 days after plating, and 2,500 cells were re-plated.

### Syngeneic Transplantation Assays

CD45.1⁺/CD45.2+ recipient mice were lethally irradiated using a split dose of either; a 11 Gy (two doses of 5.5 Gy administered at least 4 hours apart) at an average rate of 0.58 Gy/min using a Cesium 137 GammaCell 40 irradiator, or 8 Gy (two doses of 4 Gy administered at least 4 hours apart) at an average rate of 1.086 Gy/min using a RADSOURCE X-ray irradiator.

For primary transplantations of leukemic cells, 100,000 Meis1/Hoxa9-transduced c-Kit⁺ cells or 2,000 iMLL-AF9 Lin Sca-1⁺c-Kit⁺ (LSK) cells were transplanted into lethally irradiated CD45.1⁺/CD45.2⁺ recipient mice (together with 200,000 unfractionated support CD45.1⁺ wild-type BM cells). All recipient were culled upon reaching their humane endpoint as recorded in survival curves.

For secondary transplantations of leukaemic cells, 50,000 cells harvested from primary recipients were transplanted into lethally irradiated CD45.1+/CD45.2+recipient mice (together with 200,000 unfractionated support CD45.1+wild-type BM cells). Recipients were culled upon reaching their humane endpoint as recorded in survival curves. For LDA analyses, increasing doses (10,000, 50,000, 100,000) of CD45.2+BM cells from primary transplantation were re-transplanted into lethally irradiated CD45.1+/CD45.2+recipient mice (together with 200,000 unfractionated support CD45.1+wild-type BM cells). LSC frequency was calculated using ELDA software¹²¹.

For primary transplantations of healthy total BM cells, 500,000 total BM cells were mixed with 200,000 support CD45.1⁺ BM cells. For primary transplantations of HSCs, 200 LSKCD48⁻CD150⁺ HSCs (per recipient) sorted from BM of the donor mice were mixed with 200,000 support CD45.1⁺ BM cells and transferred into lethally irradiated CD45.1⁺/CD45.2⁺ recipients. All recipient mice were culled and analyzed 16-20 weeks post-transplantation.

For animals treated with Doxycycline (Dox), mice were provided ad libitum access to drinking water containing 2 mg/mL DOX with 30% sucrose.

### Xenotransplantation Experiments

100,000 THP-1 cells, 100,000 OCI-AM3 cells or 100,000 MV411 cells (for combination experiments) were tail vein injected into non-irradiated 10-to 12-week-old mixed gender NBSGW mice and began drug treatment 14 days after transplantation. Mice were injected intraperitoneally twice daily for 21 days with 30 mg/kg Daprodustat (Dap), 30 mg/kg IOX5 (68, example 46) or vehicle control. Mice were culled 5 weeks after transplantation.

Mice in combination treatment experiments with Venetoclax (ABT-199) (MCE) were dosed once daily with 100 mg/kg via oral gavage (o.g.).

For steady-state analyses, 8-to 10-week-old mixed gender C₅₇B16 mice were injected intraperitoneally twice daily for 14 days with 30 mg/kg IOX5 or vehicle control. Mice were bled before and after treatment, and culled 12 hrs after the final dosing.

### Cell Proliferation and Cell Death Analyses

Cells were cultured with 50 μM Dap, 50 μM IOX5 (68, example 46), 50 μM Molidustat, 50 μM Roxadustat, 100 μM DM-NOFD, 0.01 μM Venetoclax (MV411 and MOLM13), 0.1 μM Venetoclax (THP-1 and OCI-AML3) or vehicle control. Viable cells were counted by trypan blue exclusion at the indicated time points. To analyse cells undergoing apoptosis, cells were suspended in binding buffer containing PE-conjugated Annexin-V or FITC-conjugated Annexin-V, and either PI or DAPI.

### Primary Human AML Patient Derived Samples

Frozen AML samples were retrieved from Barts Cancer Institute Biobank and quickly thawed at 37° C. Upon thawing, T cells were depleted from all samples using EasySep™ Human TCR Alpha/Beta Depletion Kit (Stem Cell Technologies, #17847). Enriched samples were plated in concentration 0.4-1.0×10⁶/mL in Myelocult H5100 medium (Stem Cell Technologies, #05150) supplemented with 20 ng/ml IL-3, G-CSF and TPO (Biolegend) in co-culture with irradiated MS-5 cells and treated with 50 μM Dap, 50 μM IOX5 (68, example 46), 1 μM Venetoclax or vehicle control for 7 days. After 3 days of treatment, half the sample was used for plating CFU assays and initial analyses, the other half was given fresh medium containing the corresponding agent. After 7 days, viable cell numbers were counted, viability was assessed using Annexin V FITC/PI stain, cell cycle was measured using PI, and the expression of CD45, CD34, CD11b and CD14 was determined. CFC assays were counted 11 days after plating. Samples were deemed sensitive to treatment if either a decrease in viability or a decrease in proliferation, as validated by cell cycle phase distribution, was detected.

Western Blotting with Human AML Cell Lines

MOLM13, OCI-AML3, MV411 and THP-1 cells treated with 50 μM Dap, 50 μM IOX5 (68, example 46) or vehicle control using cell lysis buffer (Cell Signalling Technology, #9803) supplemented with protease and phosphatase inhibitors (Merck, #20-201, #524624). Total protein extracts (45 μg) were subjected to SDS-PAGE (NuPAGE™ 4-12% Bis-Tris Plus Gel, ThermoFisher Scientific, NP0323BOX) and then transferred onto a polyvinylidene difluoride membranes using wet transfer. Membranes were blocked in 5% milk-TBST (TBS with 0.1% Tween20) and probed with anti-HIF1-α (Novus Biological, NB100-105, 1:1000, ON at 4° C.) and anti-β-actin (Cell Signalling Technology, #3700, 1:10 000, ON at 4° C.). After incubation with appropriate horseradish peroxidase- coupled secondary antibody (Cell Signalling Technology, Anti-mouse IgG, #7076, 1:2000, 2 h at room temperature), proteins were detected with Clarity Western ECL Substrate (BioRad, #1705061) and acquired on the Amersham Imager 600 (GE Healthcare Life Sciences).

### Real Time Quantitative PCR

RNA was isolated using Direct-zol RNA Miniprep kit (Zymo Research, #R2051) and reverse transcribed using High-Capacity cDNA Reverse Transcription Kit (Applied Biosystems). For real-time quantitative PCR (qPCR), 5 ng of cDNA, 5 μL of PowerUp SYBR Green MasterMix (Applied Biosystems) and 2 pmol of primers were used per well of 384-well plate. Reactions were performed in triplicate using C1000 Thermocycler 384well (BioRad). Gene expression was quantified using comparative AA-Ct method and ACTB was used as the housekeeping gene. Data is expressed as log 2 fold change in comparison to control sample and represents results of three independent experiments measured in duplicate.

shRNA-Mediated BNIP3 Knockdown

MOLM13 cells were transduced with lentiviruses expressing shRNAs, shRNA BNIP3, 5′-GCCTCGGTTTCTATTTATAAT-3′; (TRCN0000007831, Sigma-Aldrich); and shRNA CTL, 5′-TTCTCCGAACGTGTCACGTT-3′; (GE Healthcare). Selection of efficiently transduced cells was achieved by treatment with puromycin (2 μg/mL final concentration).

### RNA Extraction, Sequencing and Analysis

Total RNA was extracted from 500,000 leukaemic cells using RNeasy Plus Universal Mini Kit (QIAGEN, Cat. No. 73404) following the manufacture's protocol. The RNA integrity number (RIN) was determined by High Sensitivity RNA Screen Tape analysis (Agilent, Cat. No. 5067) and all RIN was >8. High sensitivity Libraries for RNA-seq were prepared from 500 ng of total RNA using the NEBNext® Ultra™ II Directional RNA Library Prep Kit for Illumina® (NEB, Cat. No. E7760) with NEBNext Poly(A) mRNA Magnetic Isolation Module (NEB, Cat. No. E7490) following the manufacture's protocol. Nine cycles of PCR were performed to amplify libraries and the pooled library was sequenced on illumina NextSeq 500 with 85 bp single-end mode at EMBL GeneCore facility.

For analysis of differentially expressed genes, adapters were trimmed with cutadapt (version 1.18) using options: -a AGATCGGAAGAGC-m 50, and processed reads were mapped to GRCm38 genome_tran (release 84) with HISAT2 (version 2.2.1 (Nature Methods volume 12, pages 357-360 (2015), https://www.nature.com/nmeth)) using following option: —qc-filter. Mapped reads per gene were counted with htseq-count (HTSeq version 2.0.1 (Bioinformatics, Volume 38, Issue 10, 15 May 2022, Pages 2943-2945)) providing GTF file and differentially expressed genes were analysed using DESeq2 (version 1.30.1).

### GSEA and IPA

GSEA was performed using GSEA software version 3.0 with 1,000 permutations and default parameters. Gene differential expression, computed by the edgeR package in R, was ranked by moderated t statistics, which takes into account variability between genes in the ranking. Ranked genes were compared with gene lists in the Hallmark subset of the MSigDB database, version 7.0. IPA was performed using the Core Analysis Function offered by Qiagen's Ingenuity Pathway Analysis software. The interrogated RNA-seq and mass spectrometry datasets were filtered for adjusted P values of differential expression (FDR <0.05), and the threshold for significant activation or inhibition was defined by an absolute Z-score value >2.

### Quantification and Statistical Analysis

Statistical analyses were performed using GraphPad Prism 9 software (GraphPad Software, Inc.) P values were calculated using a two-tailed Mann-Whitney U test or paired student t-test, unless stated otherwise. Kaplan-Meier survival curve statistics were determined using the Log-rank (Mantel Cox) test.

Example 158—Structure activity relationship (SAR) studies for PHD inhibition SAR studies for PHD inhibition were carried out on the compounds of the invention. Compounds were screened against PHD2 using the RF-MS hydroxylation assay. The results are displayed in Table 1 and show the compounds of the invention have high potency as PHD2 inhibitors.

TABLE 1

| Cpd No | Example | IC₅₀ μM |
|---|---|---|
| 16 | 3 | + |
| 20 | 7 | + |
| 22 | 9 | + |
| 31 | 13 | +++ |
| 32 | 14 | ++ |
| 41 | 23 | +++ |
| 42 | 24 | +++ |
| 43 | 25 | ++ |
| 44 | 26 | + |
| 64 | 42 | +++ |
| 65 | 43 | +++ |
| 66 | 44 | +++ |
| 67 | 45 | +++ |
| 68 | 46 | +++ |
| 69 | 47 | +++ |
| 75 | 51 | ++ |
| 76 | 52 | ++ |
| 84 | 60 | +++ |
| 85 | 61 | +++ |
| 86 | 62 | +++ |
| 89 | 65 | +++ |
| 90 | 66 | +++ |
| 91 | 67 | +++ |
| 93 | 69 | + |
| 95 | 71 | +++ |
| 96 | 72 | ++ |
| 97 | 73 | + |
| 99 | 75 | ++ |
| 101 | 77 | ++ |
| 103 | 79 | + |
| 108 | 84 | + |
| 109 | 85 | ++ |
| 117 | 93 | +++ |
| 119 | 95 | +++ |
| 122 | 98 | +++ |
| 123 | 99 | +++ |
| 124 | 100 | +++ |
| 125 | 101 | +++ |
| 129 | 105 | +++ |
| 158 | 133 | +++ |
| 162 | 137 | ++ |
| 176 | 153 | ++ |
| 180 | 157 | +++ |

Key:
+++ = 1 nM-1 μM,
++ = 1 μM-10 μM,
+ = >10 μM

Example 159—Immunoblots of Hep3B cells treated with compounds of the invention Hep3B cells were treated with 117, 119, 122 and 123 of the invention (Examples 93, 95, 98 and 99) at 100 μM (A) and 20 μM (B) PHD inhibitors for 3 hours. The blots show protein levels of HIF1-α and β-actin at the 3^{rd} hour after treatment. Protocols for cell culture and immunoblotting were as performed in T. L. Yeh et al, Chem Sci, 2017, 8, 7651-7668. The results of this example show, as can be seen in FIG. 1, that the compounds of the invention increase HIF-1α.

### Example 160—Immunoblots of HEK293 Cells Treated with a Compound of the Invention at Lower Concentrations

HEK293 T cells were treated with the compound 68 of the invention (Example 46). Cells were treated at 0.5, 1, 5, 10, 20, 50 and 100 μM for 18 hours. The blots show protein levels of HIF1-α and GAPDH at the identified hour after treatment. Protocols for cell culture and immunoblotting were performed using the established methods. The results of this example show, as can be seen in FIG. 2, that the compounds of the invention increase cellular HIF-1α.

### Example 161—Immunoblots of HEK293 Cells Treated with Known PHD HIF Inhibitors

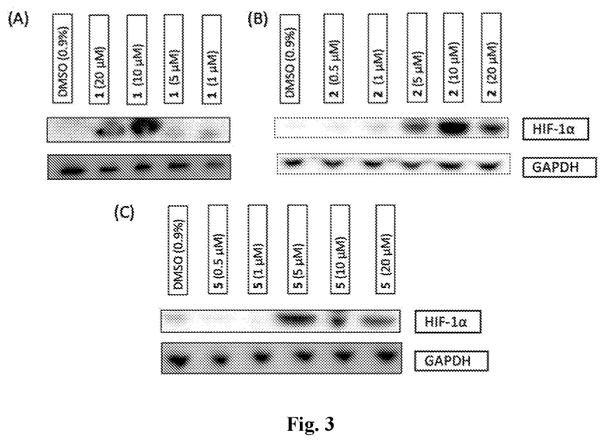

HEK293 T cells were treated with clinically used HIF PHD inhibitors Roxadustat (1), Daprodustat (2) and Molidustat (5) at 1, 5, 10 and 20 μM for 18 hours. The blots show protein levels of HIF1-α and GAPDH at the identified hour after treatment. Protocols for cell culture and immunoblotting were performed using the established methods. The results of this example are shown in FIG. 3.

### Example 162—2OG Oxygenase Selectivity 68 (Example 46)

Thee selectivity of 68 (example 46) against the following purified human 2OG dependent oxygenases was examined using reported assay procedures (T. L. Yeh et al, Chem Sci, 2017, 8, 7651-7668): FIH (factor inhibiting HIF) (IC₅₀ for 68=>100 μM), KDM4A: lysine specific demethylase 4A (IC₅₀ for 68=>100 μM), KDM5B: lysine specific demethylase 5B (IC₅₀ for 68=>100 μM) and KDM6B: lysine specific demethylase 6B (IC₅₀ for 68=>100 μM). These results show 68 is selective for the PHDs.

### Examples Demonstrating HIF PHD Relationship with Leukaemia

### Example 163—Demonstrating Relationship of PHD and AML Driven by Meis1 and Hoxa9

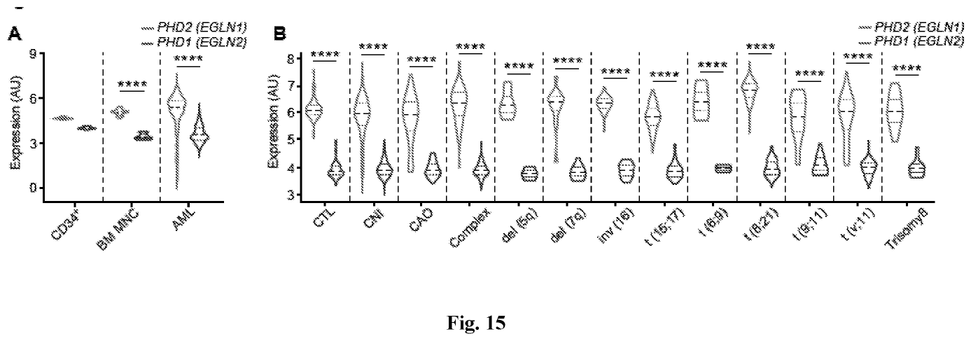

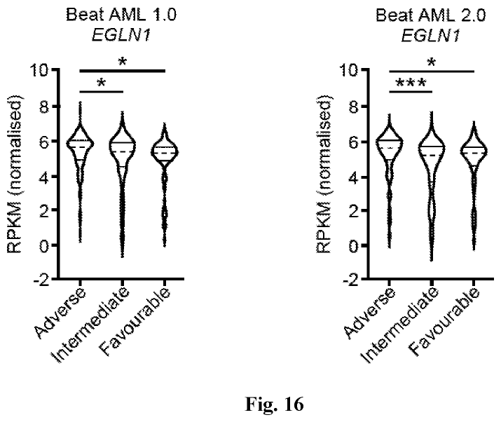

Initially, the relative abundance of PHD1 (EGLN2) and PHD2 (EGLN1) was investigated in normal human BM CD34+ cells and BM mononuclear cells from healthy donors, and human AML cells (FIG. 15A). Both PHD1 and PHD2 were expressed in all cell populations, with the relative expression of PHD2 being higher compared to PHD1 in healthy mononuclear and AML cells (FIG. 15A). Furthermore, while PHD1 and PHD2 were largely unchanged in AML subsets with diverse cytogenetic abnormalities compared to non-leukaemic controls, the relative PHD2 expression was overall higher within all AML subsets compared to PHD1 (FIG. 15B). Notably, the expression level of PHD2 directly correlated with adverse AML prognosis (FIG. 16).

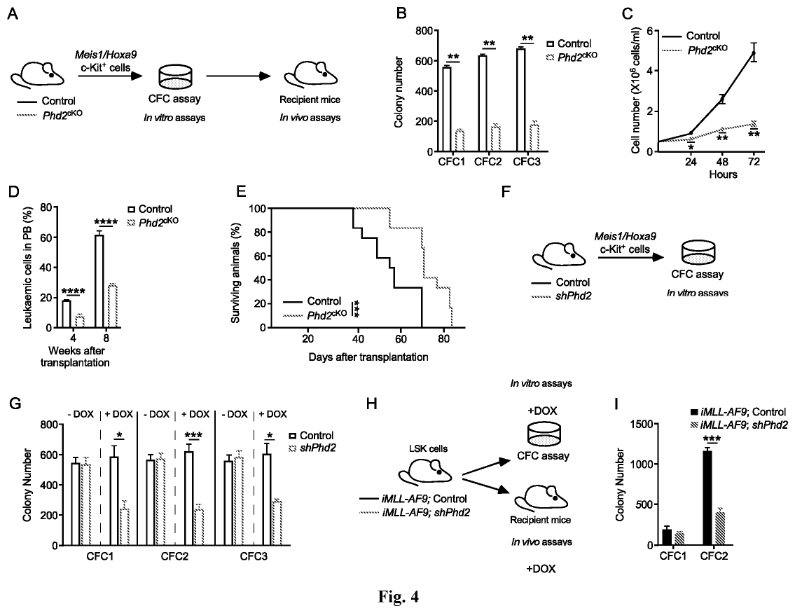

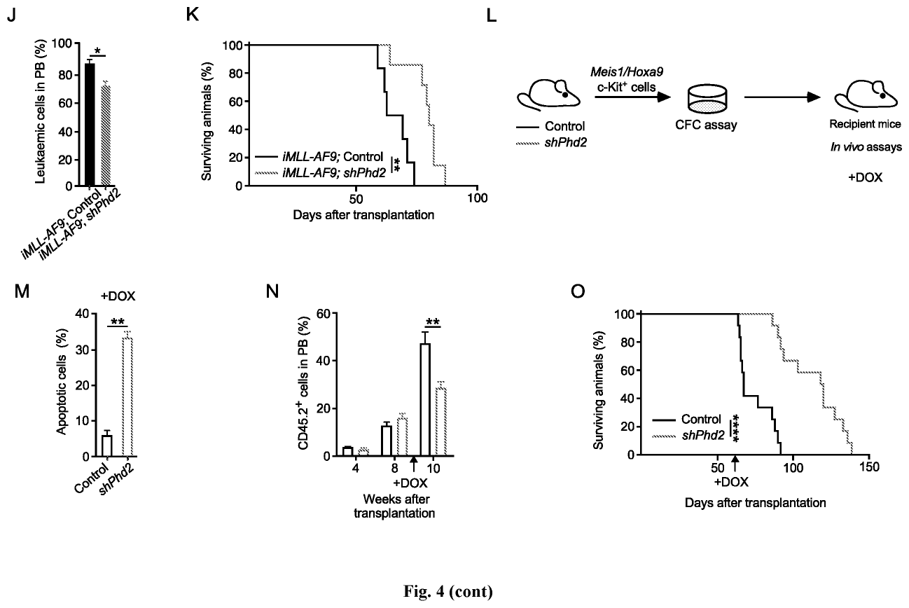

To reveal the requirement for Phd2 in AML initiation, conditional genetics and a mouse model of AML in which Meis1 and Hoxa9, oncogenes frequently overexpressed in human AML (Drabkin et al., 2002; Lawrence et al., 1999), drive leukaemic transformation and the subsequent disease (Guitart et al., 2017; Menendez-Gonzalez et al., 2019; Paris et al., 2019; Vukovic et al., 2015) (FIG. 4A) were used. The Phd2^{fl} allele in which exon 2 and exon 3 are flanked by LoxP sites (Mazzone et al., 2009) was combines with Vav-iCre (de Boer et al., 2003) to generate Phd2^{fl/fl}, Vav-iCre (Phd2^{CKO}) mice in which Phd2 is specifically deleted from the hematopoietic system shortly after the emergence of HSCs. Phd2^{CKO} and Phd2^{fl/fl} (Phd2^{CTL}) foetal liver (FL) HSPCs were transduced with Meis1/Hoxa9 retroviruses and serial replating assays were performed (FIG. 4A).

Phd2^{CKO} Meis1/Hoxa9-expressing cells were found to generate significantly less colonies upon serial replating (FIG. 4B), and display compromised proliferation (FIG. 4C). Notably, while Phd2^{CTL} cells efficiently established leukaemic engraftment upon transplantation (FIG. 4D) and generated aggressive AML (FIG. 4E), those lacking Phd2 showed weaker leukemic burden and initiated AML with a significantly longer latency (FIG. 4D-E). Thus, the loss of Phd2 prior to transformation cripples Meis1/Hoxa9-mediated leukaemogenesis. This indicates that Phd2 is required for AML development.

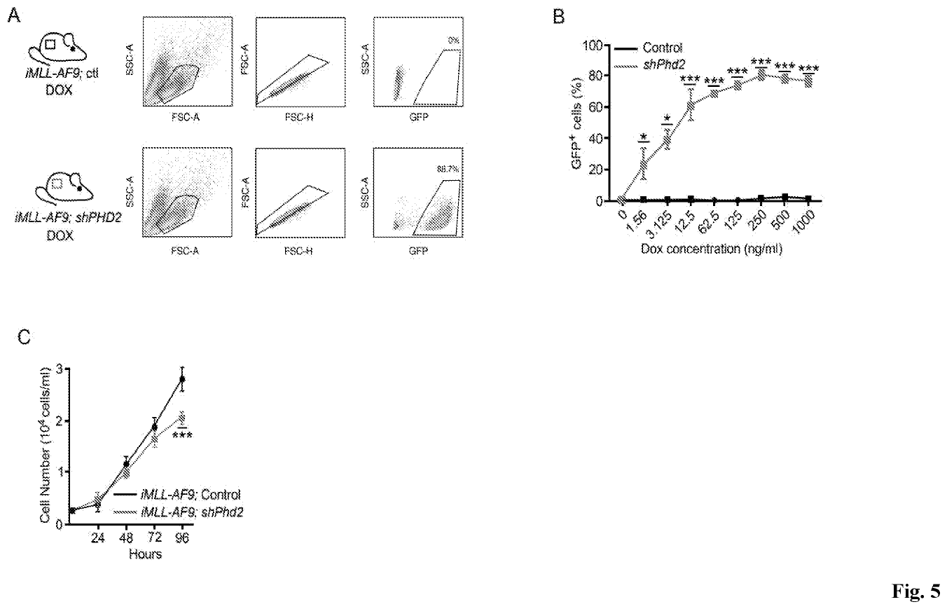

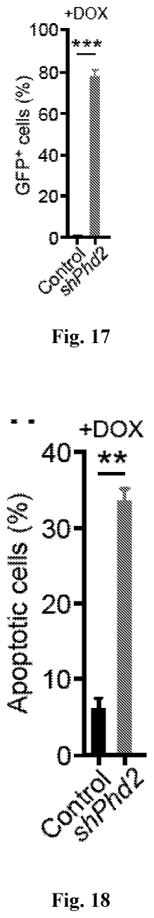

The impact of Phd2 depletion during leukaemic transformation and AML initiation was also examined. To achieve this, a mouse harbouring tetracycline-controlled transactivator (rtTA) knocked into the Rosa26 locus and (DOX)-inducible Phd2 shRNA driven by the TRE (Yamamoto et al., 2019) (referred to as shPhd2 mice) was utilised. Using this model, Meis1/Hoxa9-expressing shPhd2 cells were generated, which in the presence of DOX express shPhd2 coupled with a GFP reporter. Control cells (without rtTA) were also generated (FIG. 4F; FIG. 5B). Serial replating assays were carried out in the absence and presence of DOX (FIG. 4G), and in concordance with the impact of Phd2 deletion (FIG. 4B), inducible Phd2 knockdown during transformation significantly compromised this process (FIG. 6G), thus underscoring the requirement for Phd2 in leukemic transformation. Furthermore, shPhd2 activation evidenced by strong GFP expression (FIG. 17) induced apoptosis of AML cells (FIG. 18). Taken together, Phd2 is required for efficient AML cell survival and initiation of disease driven by the Meis1 and Hoxa9 oncogenes.

### Example 164—Demonstrating Relationship of MLL-AF9-Driven Leukaemogenesis

To validate the requirement for Phd2 in AML initiation using a distinct AML-driving oncogene, an inducible model, in which MLL-AF9 expression is activated concurrently with the loss of Phd2 expression was employed. To achieve this, a doxycycline (DOX)-inducible MLL-AF9 (iMLLL-AF9) mouse, containing tetracycline-controlled transactivator (rtTA) knocked into the Rosa26 locus and MLL-AF9 driven by the TET-responsive element (TRE) was utilised (Stavropoulou et al., 2016). The iMLL-AF9 mice were combined with shPhd2 mice (Yamamoto et al., 2019) to generate iMLL-AF9;shPhd2 and iMLL-AF9;control mice (FIG. 4H; FIG. 5A). Upon DOX treatment, iMLL-AF9;shPhd2 cells activate the expression of iMLL-AF9, shPhd2 and GFP, while iMLL-AF9;control cells activate iMLL-AF9 expression but Phd2 expression was not affected (FIG. 4H).

To test the requirement of Phd2 in MLL-AF9-induced leukaemic transformation in vitro, LSK cells from these mice were sorted and CFC and proliferation assays were performed in the presence of DOX. PHD2 knockdown reduced the number of leukaemic colonies (FIG. 4I) and slowed down AML cell proliferation (FIG. 5C), thus indicating the requirement for PHD2 in leukaemic transformation driven by the MLL-AF9 oncogene in vitro. To investigate the impact of Phd2 knockdown on MLL-AF9-mediated leukemogenesis in vivo, LSK cells from iMLL-AF9;shPhd2 and iMLL-AF9;control mice were transplanted into recipient mice, which were continuously treated with DOX. Phd2 knockdown compromised AML engraftment (FIG. 4J) and significantly impaired disease progression (FIG. 4K). Therefore, PHD2 is necessary for efficient MLL-AF9-driven leukaemogenesis.

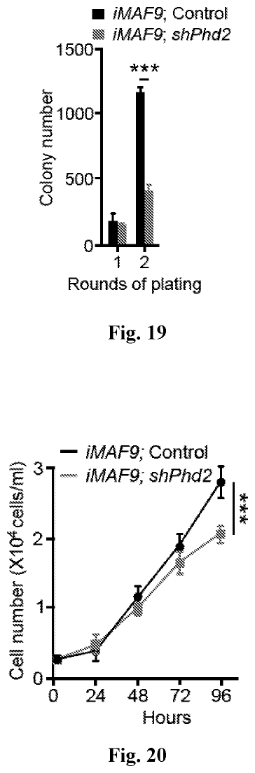

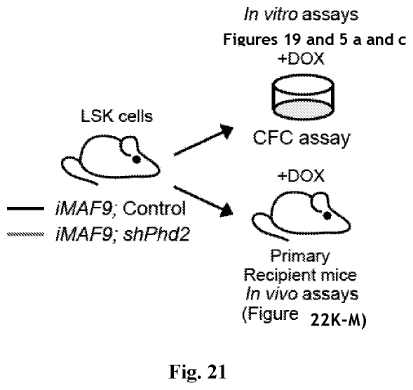

It was observed that while LSK (Lin-Sca-1 c-Kit⁺) HSPCs from iMLL-AF9;Control mice undergo efficient transformation upon DOX treatment, those from iMLL-AF9;shPhd2 mice display defective transformation and compromised proliferative capacity (FIGS. 19 and 20). These results imply that PHD2 promotes MLL-AF9-driven leukaemic transformation in vitro.

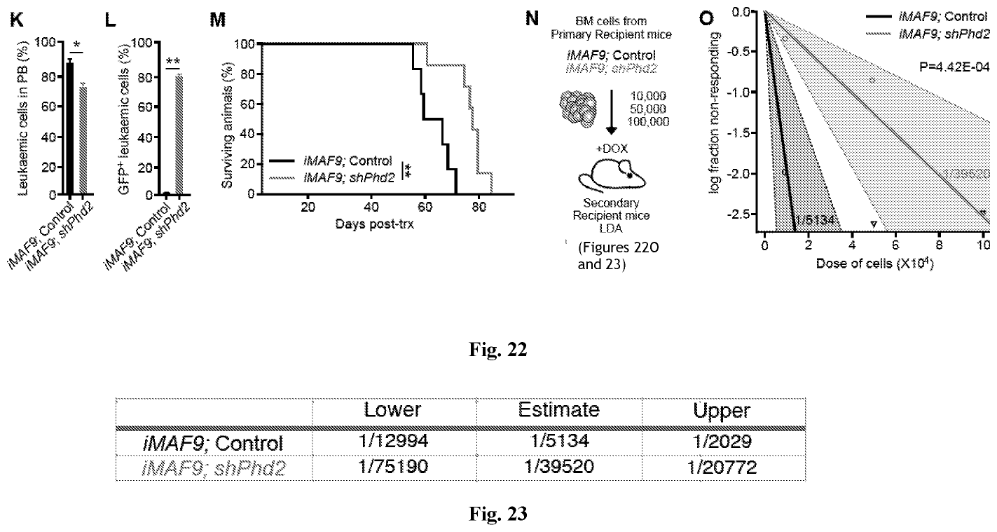

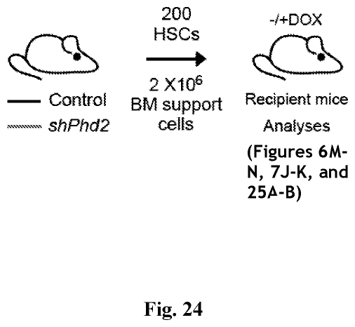

To investigate the role of PHD2 in MLL-AF9-driven AML in vivo, iMLL-AF9; Control and iMLL-AF9;shPhd2 LSK cells were transplanted into DOX-treated recipient mice (FIG. 21). It was found that iMLL-AF9 cells with Phd2 knockdown reduced leukaemic engraftment (FIGS. 22K-L) and caused AML with significantly increased disease latency compared to those engrafted with control cells (FIG. 22M). To enumerate LSCs in the leukaemic recipients of iMLL-AF9;Control and iMLL-AF9;shPhd2 LSK cells, a limiting dilution assay (LDA) was performed with donor-derived CD45.2+BM cells from primary recipients (FIG. 22N). LSC frequency in recipients of iMLL-AF9;shPhd2 cells was significantly decreased compared to that in recipients of iMLL-AF9;Control cells (FIGS. 22O and 23). Therefore, Phd2 inactivation compromises LSC development and/or function and hinders MLL-AF9-driven leukaemogenesis.

### Example 165—Demonstrating that Acute PHD2 Knockdown in Established AML Cells Compromises Leukaemia Progression

This experiment was carried out to determine whether acute depletion of Phd2 from established AML cells impacts leukaemic cell survival and disease outcome. To address this, Meis1/Hoxa9-expressing shPhd2 and control cells were utilised and the effect of inducible PHI)₂ knockdown in vivo was investigated. shPhd2 and control cells were transformed and serially replated in the absence of DOX (FIG. 4L). Upon treatment of DOX, acute Phd2 knockdown resulted in significant apoptosis, indicating that Phd2 is essential for AML cell survival (FIG. 4M). Furthermore, to determine how acute Phd2 knockdown impacts the progression of newly diagnosed AML, Meis1/Hoxa9-transformed shPhd2 and control cells (which were not treated with DOX) were transplanted into recipient mice and who were allowed to establish AML (FIG. 4N). Upon detection of equal leukaemic engraftment by cells of both genotypes (FIG. 4N), DOX was continually administered to induce Phd2 knockdown. While control cells continued to cause aggressive AML, Phd2 knockdown in AML cells significantly compromised disease progression and substantially extended AML latency (FIG. 4N-O). Taken together, these data show that PHD2 is required for AML cell survival and efficient disease progression, highlighting its value as a therapeutic target in AML.

### Example 166—Inactivation of PHD2 has No Major Impact on Mouse Survival and Normal Haematopoiesis

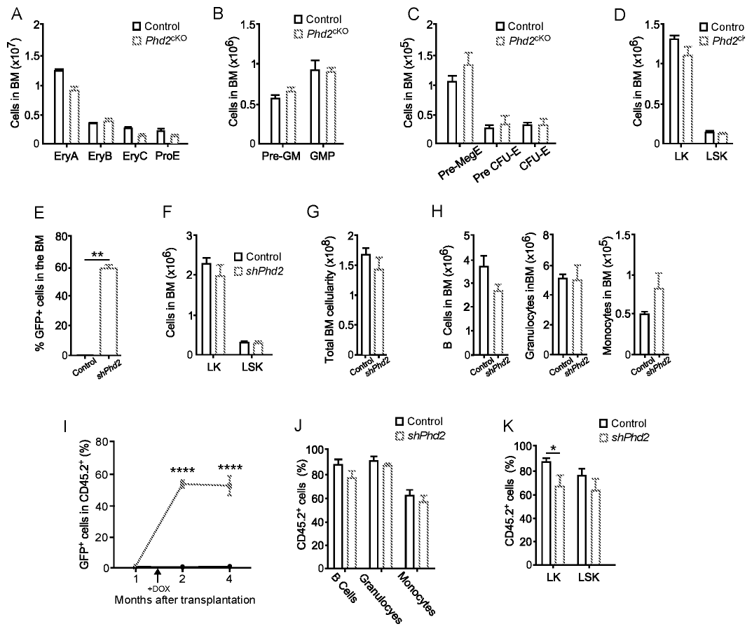

Efficient targeting of AML cells without disrupting normal haematopoiesis is a prerequisite for the development of non-toxic AML therapies. Thus, it was investigated whether Phd2 deletion has any detrimental effects on HSC functions and multilineage haematopoiesis. Phd2^{CKO} mice had normal numbers of HSCs, as well as primitive and committed progenitor cells at different levels of the haematopoietic differentiation hierarchy (FIG. 6A-B, FIG. 7A-D), normal total BM cellularity (FIG. 6C) and sustained unaffected steady-state haematopoiesis (FIG. 6D-E). To test the repopulation capacity of HSCs lacking Phd2 (FIG. 6F), BM cells from Phd2^{CKO} and control mice were competitively transplanted into lethally irradiated recipients and it was found that cells of both genotypes were able to establish and sustain long-term reconstitution (FIGS. 6G-H), and equally contributed to long-term multilineage haematopoiesis (6H). Thus, deletion of Phd2 does not have a significant impact on the steady-state and post-transplantation multilineage haematopoiesis.

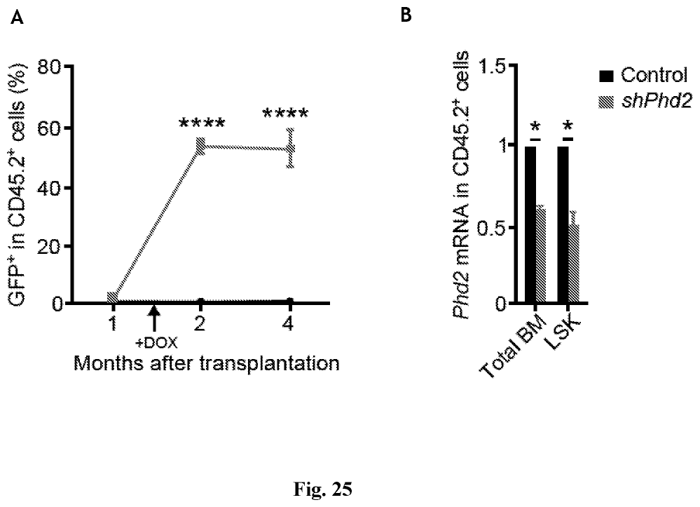

The consequences of global inducible Phd2 knockdown on mouse survival and haematopoiesis were also determined. 8-week-old shPhd2 and control mice were treated with DOX for 8 weeks (FIG. 61) and it was found that mice of both genotypes survived normally without any adverse effects. Expression of the short-hairpin targeting Phd2 was validated by measuring GFP⁺ cells in the BM of DOX treated mice (FIG. 7E). Despite a slight reduction in HSC and MPP numbers (FIG. 6J), DOX-treated shPhd2 mice had normal numbers of myeloid progenitors (FIG. 7F), unaffected total BM cellularity (FIG. 7G) and displayed normal steady state multilineage hematopoiesis (FIG. 6K, FIG. 7H). To test the fitness of HSCs upon Phd2 knockdown in a transplantation setting, HSCs from shPhd2 and control mice (which have not been treated with DOX) were competitively transplanted into recipients and upon engraftment (6 weeks after transplantation), DOX was continuously administered for 10 weeks (FIG. 6L). This knocked down gene Phd2 expression (FIGS. 24 and 25A-B). HSCs of both genotypes equally contributed to multilineage haematopoiesis (FIG. 6M, FIGS. 7I-J) and stem and progenitor cell compartments of the recipient mice (FIG. 6N, FIG. 7K). Thus, inducible Phd2 depletion has no significant detrimental effects on multilineage haematopoiesis. Taken together, while Phd2 inactivation compromises AML establishment and progression (FIG. 4), it has minimal impact of normal haematopoiesis, implying an ample therapeutic window for targeting PHD2 in AML.

### Example 167—Pharmacological Inhibition of PHDs Compromises Genetically Diverse Leukaemia Cells

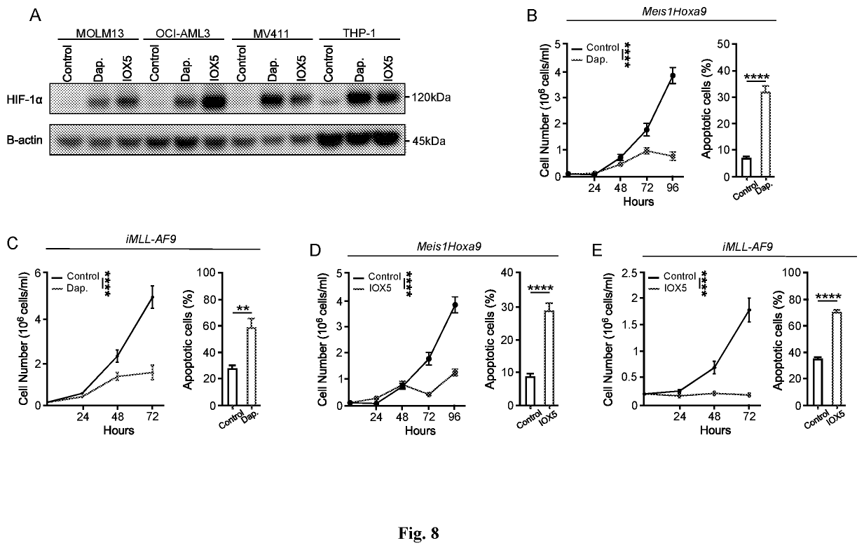

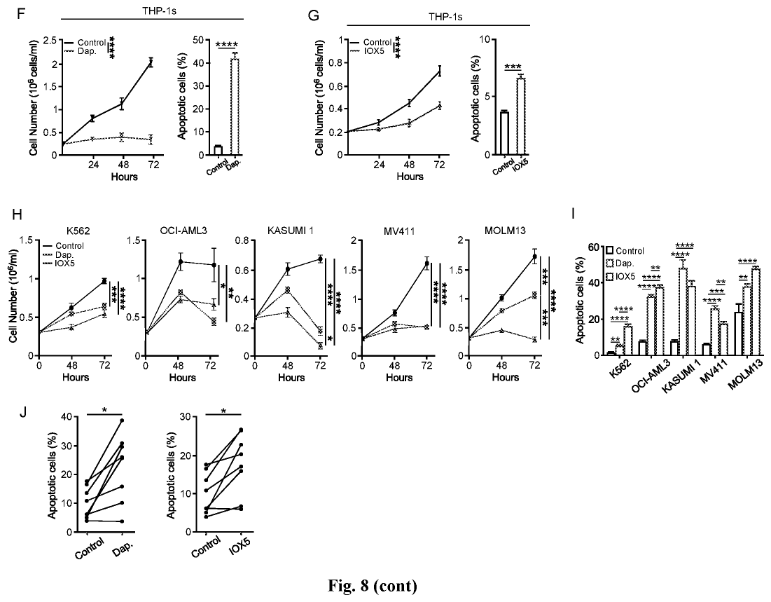

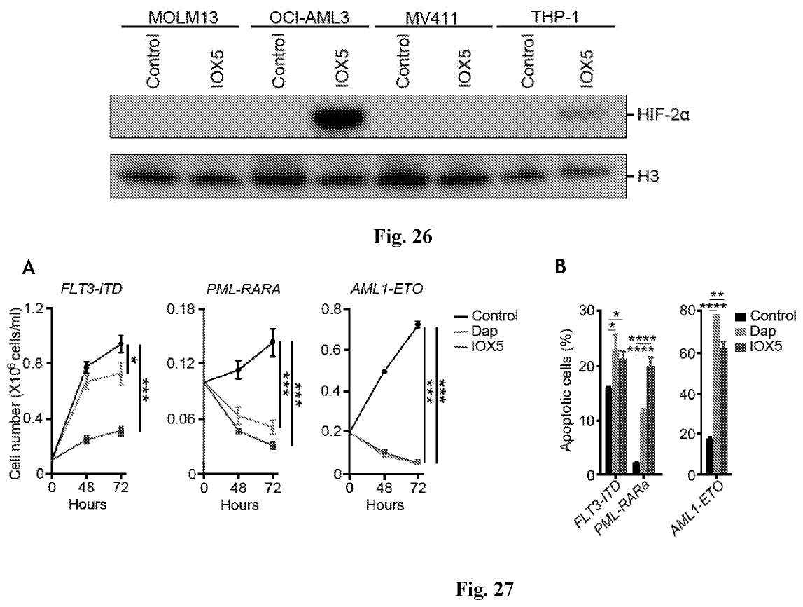

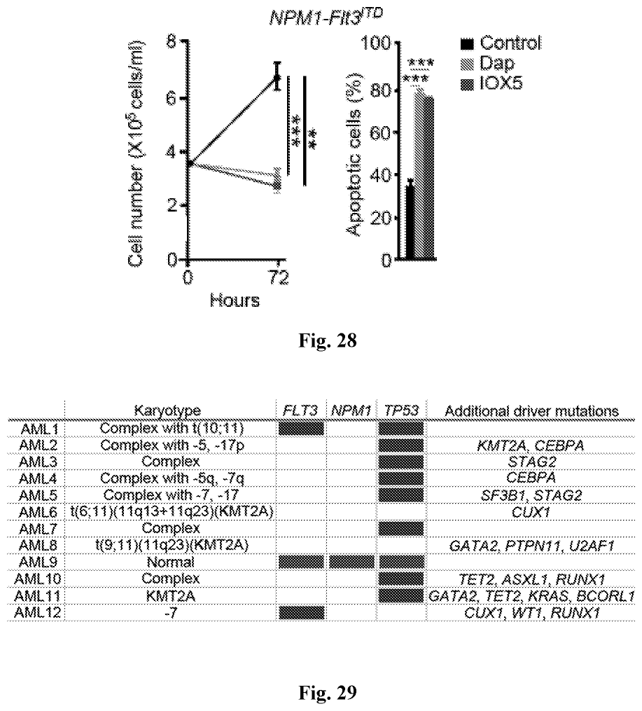

Equipped with currently available PHD inhibitors and newly generated IOX5 (68, example 46), the impact of pharmacological PHD inhibition on AML cells was assessed. As expected, Daprodustat and IOX5 (68, example 46) strongly stabilised HIF-1a across human AML cell lines (FIG. 8A). Furthermore, IOX5 (68, example 46) stabilised HIF-2α protein levels in OCI-AML3 and THP-1 cells, but did not induce any HIF-2α stabilisation in MOLM13 and MV411 (FIG. 26). Thus, while PHD inhibition consistently stabilises HIF-1α protein across all tested AML cell lines, HIF-2α stabilisation is variable and cell line-specific.

To determine consequences of Daprodustat (2) and IOX5 (68, example 46) treatment on AML cells driven by the MLL-AF9→MEIS1/HOXA9 axis, mouse iMLL-AF9-and Meis1Hoxa9-transformed AML cells were employed, as well as human THP-1 cells, which harbour the MIL-AF9 translocation. Both Daprodustat (2) and IOX5 (68, example 46) compromised their proliferative capacity and induced their apoptosis (FIG. 8B-G). Thus, providing compelling evidence that PHD inhibition compromises AML cells driven by the MLL-AF9→MEIS1/HOXA9 axis.

To validate the anti-leukaemic effect of PHD inhibition on AML cells transformed by drivers independent from the MLL/Meis1/Hoxa9 axis, murine AML cells harbouring FLT3-ITD mutations were employed (with and without NPM1 mutations), PML-RARα and AML1-ETO. It was found that Dap and IOX5 (68, example 46) reduced the proliferative rate and induced apoptosis in these cells (FIGS. 27A-B and 28).

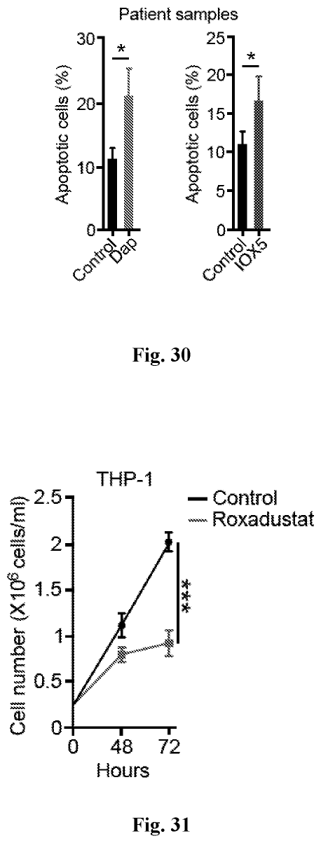

To investigate the clinical utility of PHD inhibition in human AML, it was next determined the impact of Dap or IOX5 (68, example 46) on AML patient samples (FIG. 29), focusing on poor-risk AML, which is characterised by a particularly low overall patient survival rate⁷³. It was found that treatment of independent patient samples with Dap or IOX5 (68, example 46) clearly increased apoptosis of primary human AML cells (FIGS. 29 and 30).

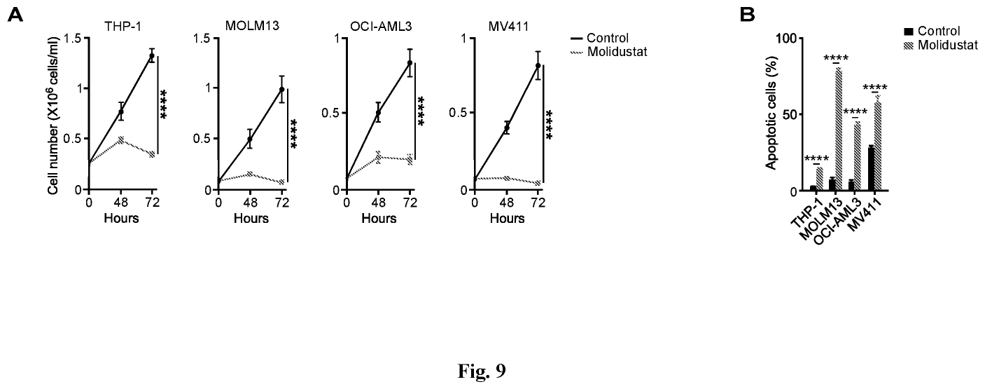

To examine the impact of PHD inhibition in human leukaemia cells with other mutational backgrounds, different myeloid leukaemia cells were used, namely K562 (CML, with BCR-ABL translocation), OCL-AML3 cells (AML with DNMT3A, NRAS and NPM1 mutations), KASUMI1 cells (AML with AML-ETO translocation), MV-4-11 (AML with FLT3-ITD mutation), and MOLM13 (AML with FLT3-ITD mutation and MLL-AF9 translocation). Notably, Daprodustat (2) and IOX5 (68, example 46) treatment consistently compromised the proliferation of all leukaemic cell lines and induced their apoptosis (FIG. 8H-I). Notably, molidustat (5, Bay 85-3934), another commercially available PHD inhibitor, also induced cell death and reduced proliferation of multiple human leukaemic cell lines (FIG. 9). In agreement with these results, other chemically distinct PHD inhibitors, Roxadustat, also manifested strong anti-leukaemic activity in established human AML cells, further supporting the fact that the effects are mediated via PHD inhibition (FIG. 31). Thus, multiple potent PHD inhibitors, with distinct mode of action, compromise leukaemic cells independently from their mutational status.

Finally, the clinical applicability of PHD inhibition in AML was validated by treating independent patient-derived primary AML cells with Daprodustat (2) and IOX5 (68, example 46). In concordance with studies in murine AML models and human AML cells, both compounds significantly increased primary human AML cell apoptosis (FIG. 8J). Taken together, Examples 163 to 167 provide genetic and pharmacological evidence that PHD inhibition is a plausible therapeutic strategy in AML.

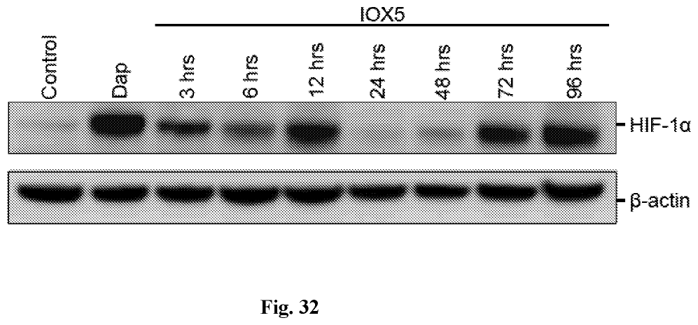

It was also investigated whether prolonged PHD inhibition in AML cells stabilises HIF-1a transiently or in a sustained manner to compromise AML cells. THP-1 AML cells were continuously treated with IOX5 (68, example 46) for 96 hours and it was found that the HIF-1α was substantially stabilised after 3-12 hours, its levels decreased between 24-48 hours, then increased 72-96 hours after treatment initiation (FIG. 32). This finding is consistent with previous reports indicating that HIF-1α levels peak and subsequently decline upon prolonged hypoxic exposure^{74,75} and the fact that HIF activity may oscillate^{76,77}. While future investigations are warranted to provide mechanistic details underpinning this expression pattern, it is concluded that IOX5 (68, example 46)-induced HIF-1α stabilisation is sufficient to significantly compromise AML cells.

### Example 168—Inhibition of PHD Upregulates HIF Signalling

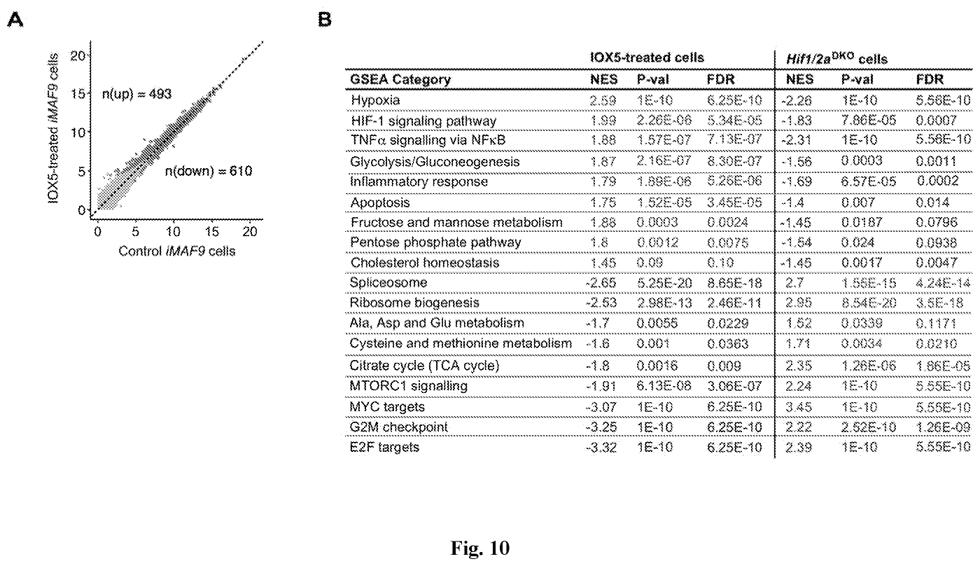

To understand the mechanism by which IOX5 (68, example 46) compromises AML cells, RNA-seq analysis of iMLL-AF9 AML cells treated with IOX5 (68, example 46) vs control was carried out. This revealed that 493 upregulated and 610 downregulated genes (FDR<0.05) (FIG. 10A). Gene set enrichment analysis (GSEA) of deregulated genes revealed a significant up-regulation of hypoxia signature, HIF-1 signalling, and activation of HIF-inducible pathways (including glycolysis, pentose phosphate pathway, fructose and mannose metabolism) as well as apoptosis (FIG. 10B). Furthermore, consistent with IOX5's (68, example 46) anti-leukaemic effect, downregulation of multiple pathways which normally promote or are required for leukaemic transformation was observed (including MYC and E2F targets, TCA cycle, ribosome biogenesis, MTORC1 signalling) (FIG. 10B). Notably, comparing the dysregulated pathways in AML cells treated with IOX5 (68, example 46) with those dysregulated in AML cells lacking Hif-1α and Hif-2α (Hif½α^{DKO} cells) exposed to hypoxia, a clear inverse correlation was observed, indicating that IOX5 (68, example 46) regulates gene expression in a HIF-dependent manner (FIG. 10B).

### Example 169—Inhibition of PHD Activity Ablates AML Through HIF-α Stabilisation

Given that the primary function of PHD2 is to promote HIF-α degradation, HIF-1α levels were measured following the treatment of AML cells with PHD inhibitors. It was found that Daprodustat (2) and IOX5 (68, example 46) increased HIF-1α levels across all treated human AML cell lines versus the control (FIG. 10A). To investigate whether HIF-α mediates the anti-leukaemic effects of PHD inhibition, Meis1/Hoxa9-transformed AML cells lacking both Hif1α and Hif2α and control cells were employed (Vukovic et al., 2015). Notably, it was found that 68 (Example 46) compromised proliferation of control but not Hif1α/Hif2α-deficient cells (FIG. 11A), thus implying that PHD inhibition exhibits its anti-leukaemic effect in the HIF-dependent manner. Next, to investigate a molecular mechanism behind the anti-leukaemic effect of PHD inhibition, HIF-dependent transcripts that were dysregulated were interrogated via (68, example 46)-treatment in murine cells. Amongst other HIF-dependent transcripts, the pro-apoptotic Bcl-2 family member BNIP3 was significantly upregulated following IOX5 (68, example 46) treatment (FIG. 11B). To confirm this, expression analysis on human AML MOLM-13 cells treated with Daprodustat (2) and IOX5 (68, example 46) was performed and upregulation of BNIP3 was found, as well as its downstream effectors BAX and BAK (FIG. 11C).

### Example 170—Combining PHD2 Inhibition with Existing AML Treatments

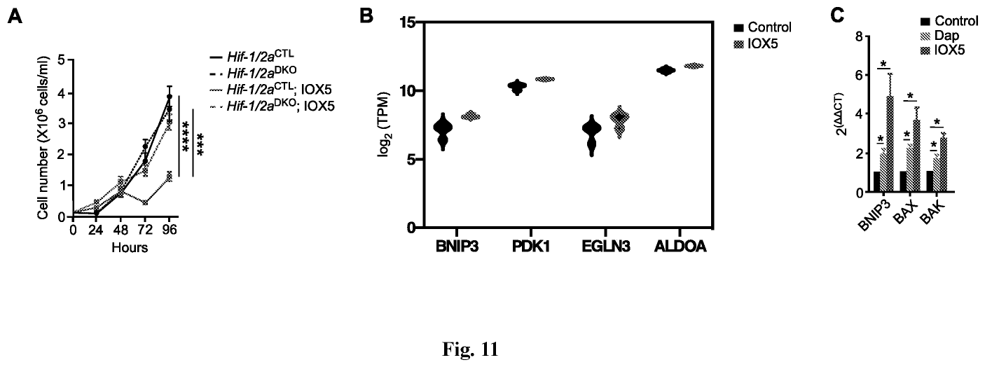

Following the finding that PHD inhibition upregulates BNIP3, a pro-apoptotic member of the Bcl-2 family, it was desired to further pharmacologically target this pathway for therapeutic potential, and utilised the BCL-2 inhibitor Venetoclax. Treatment of multiple human AML cell lines with a combination therapy of Venetoclax and either Daprodustat (2) or IOX5 (68, example 46) significantly reduced proliferation and increased the rate of apoptosis versus the single agent treatment (FIG. 12A-B). Furthermore, treatment of primary AML patient samples with the combination therapy of a PHD inhibitor and Venetoclax resulted in significant upregulation of early-and late stage apoptosis (FIG. 12C).

### Example 171—PHD Inhibition Results in Apoptosis of Multiple Myeloma (MM) Cell Lines

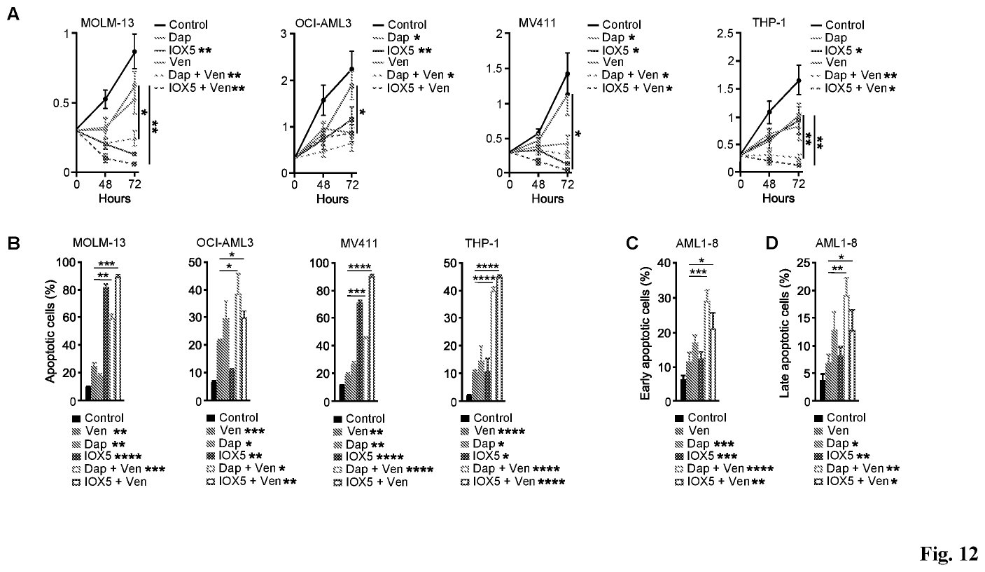

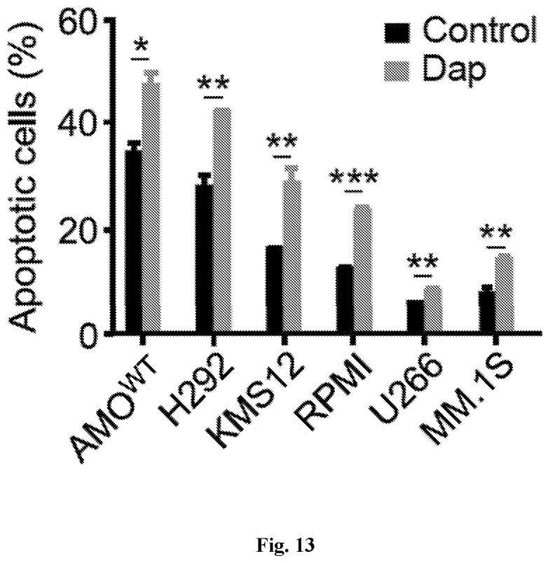

To test the impact of PHD inhibition on other blood cancers, a set of diverse human multiple myeloma cell lines derived from individual patients were treated with Daprodustat (2) and found significant apoptosis in these cells versus the control (FIG. 13).

### Example 172—Selectivity for PHD

Lack of selectivity of an enzyme inhibitor can lead to unpredictable and undesirable off-target effects. Currently available PHD inhibitors, including roxadustat, daprodustat, molidustat, desidustat and vadadustat, display only limited target selectivity for the PHDs, with, for example, inhibition by one or more of them being observed with collagen prolyl hydroxylases (CPHs); 2-oxoglutarate and iron dependent oxygenase domain containing 1 (OFGOD1); and jumonji domain containing 6 (JMJD6).

Compound 68 (Example 46) was assessed to observe its inhibitory activity of sites which are commonly inhibited by existing PHD inhibitors.

### Recombinant PHD2 Production, Purification and SPE-MS IC₅₀ Determination

Recombinant PHD2 was produced, purified and IC₅₀ determination as reported by Yeh et al^{a}.

### Recombinant FIH Production, Purification and SPE-MS IC₅₀ Determination

Recombinant FIH was produced, purified and IC₅₀ determination as reported by Yeh et al^{a}.

### Recombinant JMJD6 Production and Purification

Recombinant JMJD6 was produced as the full-length protein in E. coli and purified as reported by Cockman et al and Islam et al^{b,c}.

### JMJD6 IC₅₀ Determination

All reagents were from Sigma Aldrich and of the highest grade available. Ferrous ammonium sulphate (FAS) was prepared freshly by dissolving to 400 mM in 20 mM HCl and subsequently diluted to 1 mM in deionized water. 2-Oxoglutarate (2OG, 10 mM) and L-ascorbic acid (LAA, 50 mM) were prepared fresh by dissolving in deionized water.

Inhibition of the catalytic activity of recombinant human JMJD6 was assessed using an N-terminal peptide (RSKKRKKSKSRS) of RNA Binding Motif Protein 39 (RBM39 residues 31-42) and monitoring the appearance of the hydroxylated peptide product in 50 mM Tris.Cl pH 7.5. Titrations of 68 for IC₅₀ determinations (3-fold and 11-point IC₅₀) curves) were performed using an ECHO 550 acoustic dispenser (Labcyte) and dry dispensed into 384-well polypropylene assay plates. The final assay concentration of DMSO was kept constant at 0.5% (v/v). Full length JMJD6 was prepared at a concentration of 1.0 mM in 50 mM Tris.Cl pH 7.5 and 25 μl dispensed across the 384-well plates, JMJD6 was preincubated with compound dilutions for 15 minutes. The reaction was initiated by dispensing 25 μl of substrate (20 μM ferrous iron sulfate, 200 μM L-ascorbic acid, 10 μM RBM3931-42 and 20 μM 2-oxoglutarate) across each 384-well assay plate. The reaction was allowed to progress for 30 minutes, then quenched by dispensing 10% formic acid (5 μl). Peptide analysis was performed by Liquid Chromatography Mass Spectrometry (LCMS) using an Agilent 1290 infinity II LC system equipped with an Agilent 1290 multisampler and an Agilent 1290 high speed pump and connected to an Agilent 6550 accurate mass iFunnel quadrupole time of flight (QTOF) mass spectrometer. 10 ml of the assay mixture was injected onto a ZORBAX RRHD Eclipse Plus C₁₈ column (Agilent). Solvent A consisted of LCMS grade water containing 0.1% (v/v) formic acid and solvent B consisted of acetonitrile containing 0.1% (v/v) formic acid. Peptides were separated using a step wise gradient (0 min-95% solvent A, 1.0 min-80% solvent A, 3.0 min-45% solvent A, 4.0 min-45% solvent A, 5.0 min-0% solvent A, 6.0 min-0% solvent A, 7.0 min-95% solvent A). This was followed by a 1 min post run with 95% (v/v/) solvent A to re-equilibrate the column, all flow rates were 0.2 ml/min. The mass spectrometer was operated in the positive ion mode with a drying gas temperature (280° C.), drying gas flow rate (13 L/min), nebulizer pressure (40 psig), sheath gas temperature (350° C.), sheath gas flow rate (12 L/min), capillary voltage (4000 V), nozzle voltage (1000 V). All acquired data were analysed using Agilent MassHunter Qualitative Analysis (Version B.07.00) software.

### JMJD6 Solid Phase Extraction Mass Spectrometry (SPE-MS) Assays

The JMJD6 inhibitory activities of Daprodustat, Roxadustat and Molidustat were assessed using a 40-mer peptide substrate of bromodomain-containing protein 4 (BRD₄₅₁₁₋₅₅₀)^{b} by monitoring the hydroxylation of the peptide product in 50 mM Tris.HCl pH 7.5 buffer. Titrations of compounds were prepared using an ECHO 550 acoustic dispenser (Labcyte). An 11-point and 3-fold dilution for each compound was prepared and dry dispensed into 384-well polypropylene plates. A solution of full length JMJD6 was prepared at a concentration of 1.0 mM and 25 μl dispensed across the plate using a multidrop dispenser equipped with a low volume dispensing cassette (Thermo). Compound dilutions were pre-incubated with JMJD6 for 15 minutes. The enzyme reaction was initiated by 25 μl dispensing of the substrate mixture in 50 mM Tris. HCl pH 7.5 (200 mM L-ascorbate, 20 mM ferrous ammonium sulfate, 20 mM 2-oxoglutarate and 10 mM of the JMJD6 substrate BRD₄₅₁₁₋₅₅₀^{b}. Reactions were allowed to progressed for 15 minutes at room temperature and stopped by dispensing 10% (v/v) formic acid (5 μl). The final concentration of DMSO was 0.5% (v/v). Assay plates were transferred to a RapidFire RF365 high throughput sampling robot (Agilent) connected to an Agilent 6550 quadrupole-time-of-flight (Q-TOF) mass spectrometer. Samples were aspirated under vacuum and loaded onto a C₄ solid phase extraction (SPE) cartridge. The C4 SPE cartridge was washed with 0.1% (v/v) formic acid in water for 5.5 s at a flow rate of 1.5 ml/min to remove non-volatile buffer salts. The peptide was then eluted from the SPE with 80% (v/v) acetonitrile, 20% (v/v) water containing 0.1% (v/v) formic acid for 5.5 s at a flow rate of 1.6 ml/min into the mass spectrometer. The mass spectrometer was operated in the positive ion mode with a drying gas temperature of 280° C., drying gas flow rate of 13 L/min, nebulizer pressure of 40 psig, sheath gas temperature of 350° C., sheath gas flow rate of 12 L/min, capillary voltage of 4000 V, and nozzle voltage of 1000 V. Peak area data for the +8 charge state was integrated using RapidFire Integrator software (Agilent). The % conversion of the BRD4511-550 to the hydroxylated product was calculated using:
% conversion=100×hydroxylated/(hydroxylated+non-hydroxylated peptide): IC₅₀ data were determined from non-linear regression plots using GraphPad prism 6.0.

### KDM4A, KDM5B and KDM6B Liquid Chromatography Mass Spectrometry (LCMS) Assay

The inhibitory activity of 68 was assessed by monitoring demethylation of the respective peptide substrates for KDM4A, KDM5B and KDM6B. The peptide substrate for KDM4A was a 15-mer histone-H₃ derivative (ARTAQTARK(me3)STGGI) as reported by Hutchinson et al^{d} and synthesized by GL Biochem (Shanghai) Ltd (Shanghai, China).

The peptide substrate for KDM5B was a 21-mer histone-H₃ peptide (ARTK(me3)QTARKSTGGKAPRKQLA), as synthesized by Peptide Protein Research (Hampshire, UK). The KDM6B peptide substrate was a 17-mer histone-H₃ peptide (LATKAARK(me3)SAPATGGVK), as synthesized by GL Biochem (Shanghai) Ltd (Shanghai, China). Recombinant KDM4A, residues M1-L359, was produced in E. coli and purified as reported by Ng et al^{e}. Recombinant KDM5B, residues, M1-R822, was expressed in a baculoviral expression system and purified as previously described by Johansson et al^{f}. KDM6B, residues D1141-E1590, was expressed in E. coli and purified as previously described by Rose et al^{g}.

KDM4A reactions were performed under optimized buffer conditions (50 mM MES pH 7.0. KDM4A (0.15 mM) was pre-incubated for 15 minutes in the presence of 68 (100 mM) and the enzyme reaction initiated by addition of substrate (100 mM L-ascorbate, 10 mM ferrous ammonium sulphate, 10 mM 2-oxoglutarate and 10 mM peptide substrate). The enzyme reaction was progressed for 50 minutes, and the reaction stopped by addition of formic acid to a final concentration of 1% (v/v). Control reactions in the presence of 0.5% (v/v) DMSO and a control in the presence of a known inhibitor of KDM4A (50 mM 2, 4-pyridine dicarboxylic acid, ^{h}were also set up.

KDM5B enzyme reactions were performed under optimized buffer conditions (50 mM MES pH 7.0, 50 mM NaCl, 1 mM TCEP). KDM5A (0.15 mM) was pre-incubated for 15 minutes in the presence of 68 (100 mM) and the enzyme reaction was initiated by addition of substrate (100 mM L-ascorbate, 10 mM ferrous ammonium sulphate, 10 mM 2-oxoglutarate and 5 mM peptide). The enzyme reaction was progressed for 30 minutes, and the reaction stopped by addition of formic acid to a final concentration of 1% (v/v). Control reactions included a 0.5% DMSO control and a reaction with a known inhibitor of KDM5B (10 mM KDOAM25, ⁱ).

KDM6B reactions were performed under optimized buffer conditions (50 mM MES pH 7.0). KDM6B (0.15 mM) was pre-incubated for 15 minutes in the presence of 68 (100 mM) and the enzyme reaction initiated by addition of substrate (100 mM L-ascorbate, 10 mM ferrous ammonium sulphate, 10 mM 2-oxoglutarate and 5 mM peptide). The enzyme reaction was progressed for 30 minutes and the reaction stopped by addition of formic acid to a final concentration of 1% (v/v). Control reactions included a 0.5% DMSO control and a reaction with a known inhibitor of KDM6B (10 mM GSKJ1,^{j}).

Enzyme reactions were transferred to a 96-well polypropylene plate and peptide analysis was performed by LCMS using an Agilent 1290 infinity II LC system equipped with an Agilent 1290 multisampler and an Agilent 1290 high speed pump and connected to an Agilent 6550 accurate mass iFunnel quadrupole time of flight (QTOF) mass spectrometer. 4 ml of enzyme reaction were injected and loaded onto a ZORBAX RRHD Eclipse Plus C₁₈ column (Agilent Technologies, CA, US). Solvent A consisted of LCMS grade water containing 0.1% (v/v) formic acid and solvent B consisted of acetonitrile containing 0.1% (v/v) formic acid. Peptides were separated using a step wise gradient (0 min-95% solvent A, 1.0 min-80% solvent A, 3.0 min-45% solvent A, 4.0 min-45% solvent A, 5.0 min-0% solvent A, 6.0 min-0% solvent A, 7.0 min-95% solvent A). This was followed by a 3 min post run with 95% solvent A to re-equilibrate the column, all flow rates were 0.2 ml/min. The mass spectrometer was operated in the positive ion mode with a drying gas temperature of 280° C., drying gas flow rate of 13 L/min, nebulizer pressure of 40 psig, sheath gas temperature of 350° C., sheath gas flow rate of 12 L/min, capillary voltage of 4000 V, nozzle voltage of 1000 V. All acquired data were analyzed using Agilent MassHunter Qualitative Analysis (Version B.07.00) software.

### OGFOD1 Solid Phase Extraction Mass Spectrometry (SPE-MS) Assay

OGFOD1 gene was cloned into the pET-28a vector and expressed and purified as the full-length enzyme (Met1-Glu542) with an N-terminal 6-His tag in E. coli strain BL21 (DE3). The synthetic peptide substrate (Ala47 RPS23-Lys76 AKGIVLEKVGVEAKQPNSAIRKAVRVQLIK-NH2) was synthesized by GL Biochem (Shanghai, China) to >95% purity. Ferrous ammonium sulphate (FAS), 2-oxoglutarate (2-OG) and L-ascorbic acid (LAA) were from Sigma Aldrich. Ferrous ammonium sulphate was prepared fresh by dissolving 50-100 mg in 20 mM HCl to 400 mM concentration which was further diluted to 1 mM in deionized water. 2-OG (10 mM) and L-AA (50 mM) were both prepared fresh in deionized water.

IC₅₀ determinations were performed in 384-well plate format using polypropylene plates (Greiner Bio One, Cat Number 781096). Compounds were prepared as 20 mM DMSO stock solutions and all compound dispenses were performed using an ECHO 550 acoustic dispenser (Labcyte, Sunnyvale, CA). A positive control compound (2, 4-PDCA, 100 mM) was dispensed into column 1 (250 nl) and DMSO was dispensed into column 13 (250 nl). All test compounds were serially diluted (an approximately 3-fold dilution series across an 11-point IC₅₀) and 250 nl of each dilution dispensed in duplicate into the polypropylene plate. OGFOD1 was diluted to 0.3 mM in assay buffer (50 mM Tris.Cl pH 7.5) and was dispensed (25 ml) into the 384-well compound plates using a multidrop combi reagent dispenser (Thermo Scientific, Code 5840300) with a small tube plastic tip dispensing cassette (Thermo Scientific, Code 24073290). The compounds were pre-incubated with OGFOD1 for 15 minutes and the enzyme reaction initiated by dispense of 25 ml of substrate solution (200 mM LAA, 20 mM FAS, 20 mM 2-OG, 10 mM RPS23 (47-76) peptide) in assay buffer. The final concentration of DMSO in the assay was 0.5%. The reactions were allowed to progress for 20 min, then stopped by addition of 10% (v/v) formic acid (5 ml) and the assay plate transferred to a RapidFire RF 365 coupled to a 6550 Accurate-Mass Quadrupole Time-of-Flight (QTOF) mass spectrometer (Agilent). Samples were aspirated under vacuum and loaded onto a C₄ solid phase extraction (SPE) cartridge and the SPE cartridge washed with 0.1% (v/v) formic acid in LCMS grade water for 5.5 seconds at a flow rate of 1.5 ml/min to remove non-volatile buffer components. After the aqueous wash, peptides were eluted from the C4 SPE cartridge in an organic elution step (80% (v/v) acetonitrile, 20% (v/v) LCMS grade water containing 0.1% formic acid) at a flow rate of 1.6 ml/min for 5.5 seconds. The mass spectrometer was operated in positive ion mode with a drying gas temperature (280° C.), drying gas flow rate (13 L/min), nebulizer pressure (40 psig), sheath gas temperature (350° C.), sheath gas flow rate (12 L/min), capillary voltage (4000 V), nozzle voltage (1000 V). Ion data for the substrate and hydroxylated (+16) peptide product was extracted and peak area data integrated using RapidFire integrator software (version 4.3.017235, Agilent). The % conversion of peptide substrate to hydroxylated product was calculated in excel and IC₅₀ curves generated using graphpad prism version 7.0.

### CONCLUSION

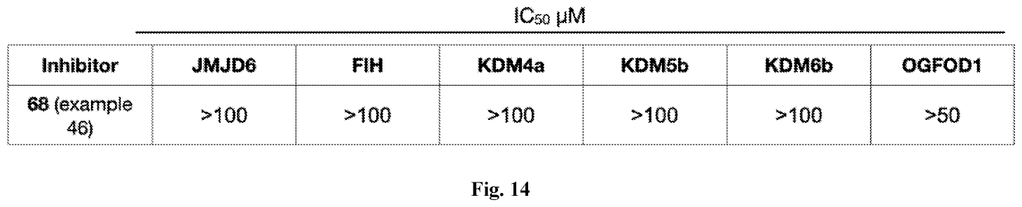

As shown in FIG. 14 of the present application, compound 68 (Example 46), had a very high IC₅₀ for each of the off-target sites tested. This demonstrates that the compound has good selectivity and inhibition of PHD specifically. Therefore compounds of the invention would be expected to have reduced side-effects compared to existing PHD inhibitors.

### REFERENCES

a Yeh, T. L. et al. Molecular and cellular mechanisms of HIF prolyl hydroxylase inhibitors in clinical trials. Chem Sci 8, 7651-7668 (2017). https://doi.org/10.1039/c7sc02103h
b Cockman, M. E. et al. Widespread hydroxylation of unstructured lysine-rich protein domains by JMJD6. Proc Natl Acad Sci USA119, e2201483119 (2022). https://doi.org/10.1073/pnas.2201483119
c Islam, M. S. et al. Biochemical and structural investigations clarify the substrate selectivity of the 2-oxoglutarate oxygenase JMJD6. J Biol Chem 294, 11637-11652 (2019). https://doi.org/10.1074/jbc.RA119.008693
d Hutchinson, S. E. et al. Enabling Lead Discovery for Histone Lysine Demethylases by High-Throughput RapidFire Mass Spectrometry. Journal of Biomolecular Screening 17, 39-48 (2011). https://doi.org/10.1177/1087057111416660
e Ng, S. S. et al. Crystal structures of histone demethylase JMJD2A reveal basis for substrate specificity. Nature 448, 87-91 (2007). https://doi.org/10.1038/nature05971
f Johansson, C. et al. Structural analysis of human KDM5B guides histone demethylase inhibitor development. Nature Chemical Biology 12, 539-545 (2016). https://doi.org/10.1038/nchembio.2087
g Rose, N. R. et al. Plant Growth Regulator Daminozide Is a Selective Inhibitor of Human KDM 2/7 Histone Demethylases. Journal of Medicinal Chemistry 55, 6639-6643 (2012). https://doi.org/10.1021/jm300677i
h Rose, N. R. et al. Inhibitor scaffolds for 2-oxoglutarate-dependent histone lysine demethylases. J Med Chem 51, 7053-7056 (2008). https://doi.org/10.1021/jm800936s
i Tumber, A. et al. Potent and Selective KDM5 Inhibitor Stops Cellular Demethylation of H3K4me3 at Transcription Start Sites and Proliferation of MMIS Myeloma Cells. Cell Chemical Biology 24, 371-380 (2017). https://doi.org/https://doi.org/10.1016/j.chembiol.2017.02.006
j Kruidenier, L. et al. A selective jumonji H₃K₂₇ demethylase inhibitor modulates the proinflammatory macrophage response. Nature 488, 404-408 (2012). https://doi.org/10.1038/nature11262

### Example 173—Targeting PHD1 Compromises AML but not Normal Haematopoiesis

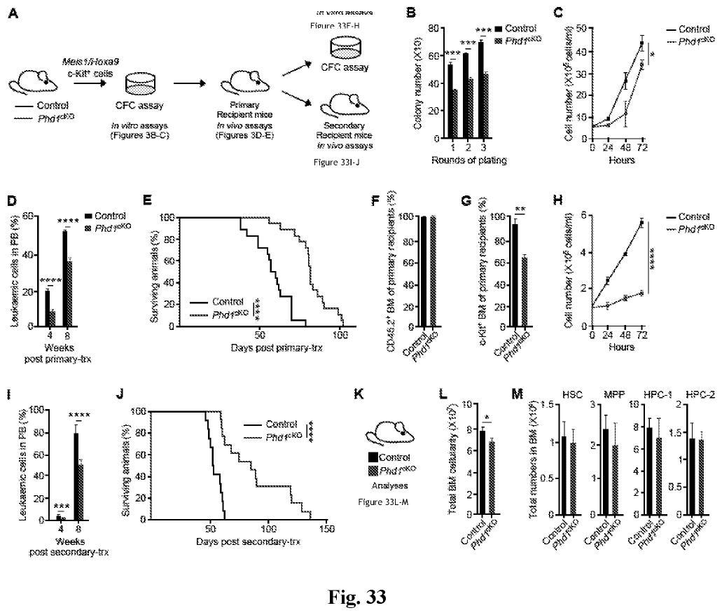

While amongst the PHD isoforms, PHD2 is thought to often make the most important contribution to setting the steady-state levels of HIF-1α under normoxia, both PHD2 and PHD1 isoforms have similar ability to hydroxylate HIF-α subunits^{18,20,21,24,25,60,61} Thus, the functional significance of PHD1 in initiation and propagation of AML driven by Meis1 and Hoxa9 was examined (FIG. 33A). Loss of Phd1 compromised serial replating and proliferative potential of Meis1/Hoxa9-transduced cells (FIGS. 33B-C), and impeded AML engraftment in vivo (FIGS. 33D-E). It was then investigated whether the loss of Phd1 impacts disease propagation (FIG. 33A). Notably, Phd1^{cKO} cells harvested from primary recipients showed reduced c-Kit expression, a marker that enriches for leukaemia propagating cells 62, and decreased growth when compared to control AML cells (FIGS. 33F-H). To test the ability of control and Phd1-deficient AML cells from primary recipients to propagate AML in secondary recipients, secondary transplantation experiments were performed. While transplantation of control cells resulted in aggressive AML in secondary recipients, mice transplanted with Phd1^{cKO} AML cells had a reduced leukaemic burden, with significantly increased survival (FIGS. 331-J), indicating that PHD1 is required for AML maintenance and propagation.

To investigate the role of Phd1 in steady-state haematopoiesis, Phd1^{cKO} and control mice were analysed under steady-state conditions (FIG. 33K). There were no adverse phenotypes in differentiated cells (data not shown), and despite displaying a reduced cellularity in the BM, loss of Phd1 had no impact on the HSPC compartment (FIGS. 33L-M). Thus, as described above for PHD2, PHD1 inactivation impedes AML initiation and propagation without significantly affecting steady-state haematopoiesis.

### Example 174—Pharmacological PHD Targeting Compromises LSCs and Extends Survival of Leukaemic Mice without any Toxic Effects on Normal Haematopoiesis

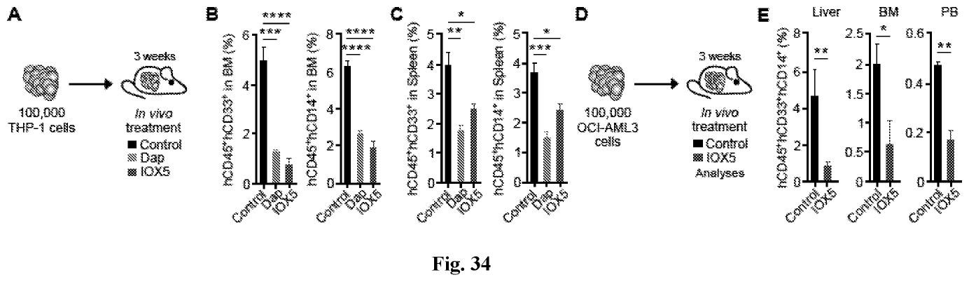

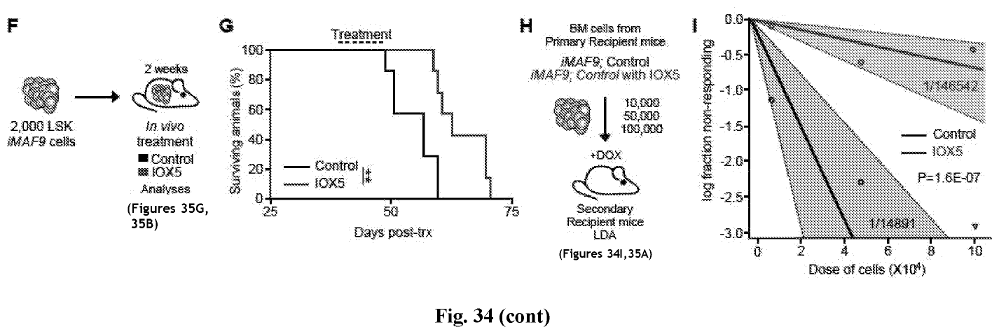

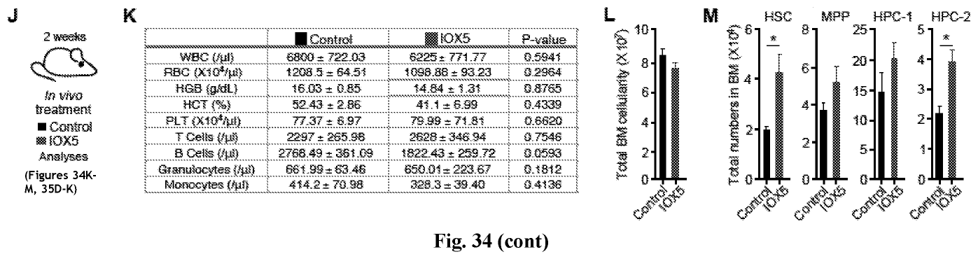

To test the anti-leukaemic potential of Dap and IOX5 (68, example 46) in vivo, Dap or IOX5 (68, example 46) was administered to mice engrafted with human THP-1 cells (harbouring an MLL-AF9 translocation) and it was found that the treatment was both well tolerated and significantly reduced leukaemic burden in recipient mice (FIGS. 34A-C and 35A). Next, it was examined whether IOX (68, example 46)₅ has a therapeutic impact on MLL-AF9-independent AML. Following engraftment of recipients with OCI-AML3 cells (harbouring DNMT3A, NRAS and NPM1 mutations) IOX5 (68, example 46) were administered and it was found that this treatment significantly reduced leukaemic burden (FIGS. 34D-E). Therefore, IOX5 (68, example 46) compromises both MLL-rearranged and non-MLL AML in vivo.

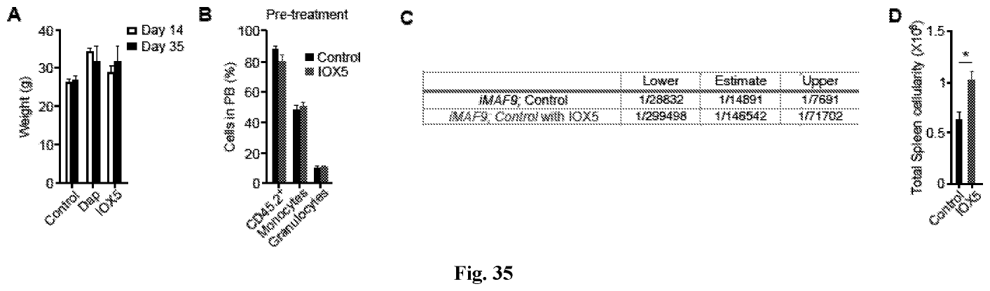

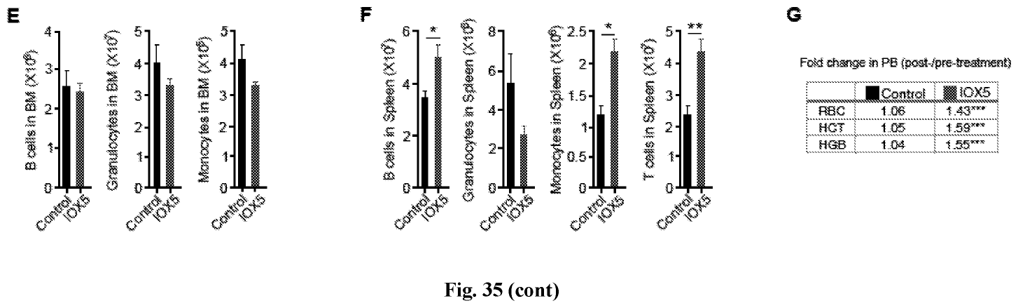

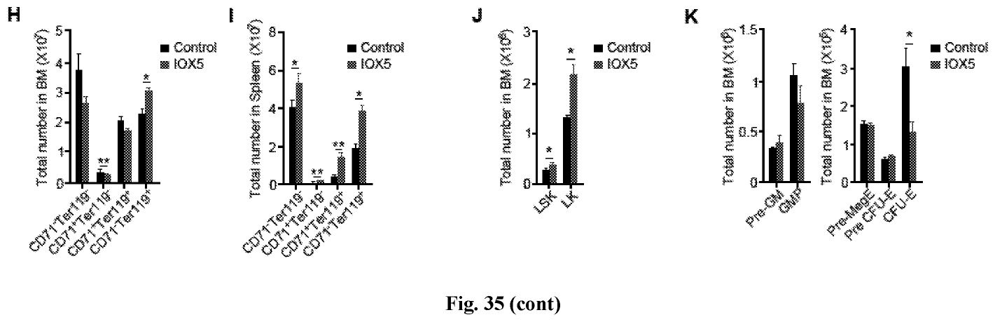

To further examine the translational utility of IOX5 (68, example 46) in AML, murine iMLL-AF9 LSK cells were transplanted into DOX-treated recipient mice (to activate iMLL-AF9 expression immediately after transplantation), and following confirmation of equal engraftment, mice were treated with IOX5 (68, example 46) for 14 days (FIGS. 34F and 35B). It was found that IOX5 (68, example 46) significantly increased survival of leukaemic mice compared to those treated with vehicle (FIG. 34G). Furthermore, a LDA revealed that IOX5 (68, example 46) treated recipients had significantly decreased LSC frequency compared to that of vehicle-treated recipients (FIGS. 34H-I and 35C), indicating that PHD inactivation compromises LSC maintenance and AML progression.

Given that Dap has been deemed safe in murine and human studies^{37,72,78}, the impact of IOX5 (68, example 46) on normal hematopoiesis was then examined. It was found that acute PHD inhibition with IOX5 (68, example 46) had no detrimental impact on the differentiated PB, BM or spleen compartments (FIGS. 34J-L and 35D-F), and, as expected, enhanced erythropoiesis (FIGS. 35G-I). Moreover, treatment with IOX5 (68, example 46) had no significant defects in the HSPC compartment (FIGS. 34M, 35J-K). Therefore, reflecting the genetic studies, chemically distinct small molecule PHD inhibitors, with related but distinct PHD binding modes, display strong anti-leukaemic activity but do not affect normal haematopoiesis.

### Example 175—IOX5 (Compound 68, Example 46) Targets AML in a HIF-α Dependent Manner

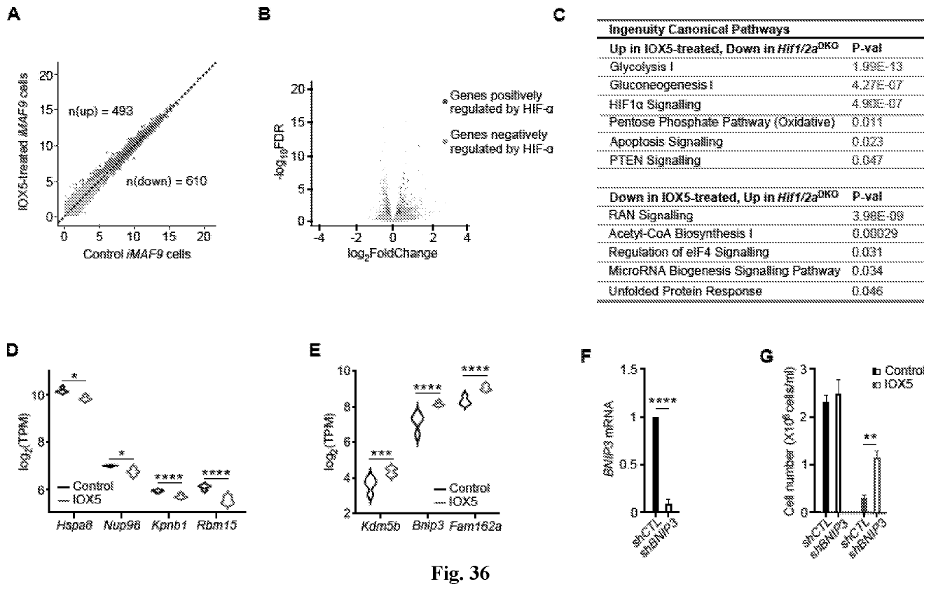

It was investigated the mechanism by which PHD inhibition compromises AML. RNA-seq analyses of iMLL-AF9 cells treated with IOX5 revealed dysregulated gene expression (FIG. 10A). Gene set enrichment analysis (GSEA) and Ingenuity Pathway Analysis (IPA) of affected genes implied significant upregulation of hypoxia and HIF-α signalling, as well as downstream HIF-mediated pathways including glycolysis, pentose phosphate pathway, fructose and mannose metabolism, and apoptosis^{79,80} (FIG. 10B, left panel and 36C, top panel). Consistent with the anti-leukaemic effect of IOX5 (68, example 46), downregulation of pathways which normally promote, or are required for, oncogenic transformation was observed (including MYC and E2F targets, TCA cycle, ribosome biogenesis, MTORC1 and RAN signalling)^{53,81-90} (FIGS. 10B, left panel and 36C, bottom panel). Given that IOX5 (68, example 46) functions to inhibit PHD-mediated HIF-α degradation, the dysregulated pathways in AML cells treated with IOX5 were compared to those lacking HIF-α, i.e. Hif½αDKO AML cells exposed to hypoxia¹⁶. It was observed that multiple pathways upregulated in IOX5 (68, example 46)-treated cells were downregulated in Hif½αDKO cells, and vice versa (FIGS. 10B, 36C). To explore the HIF-α dependency of the anti-leukaemic effect of IOX5 (68, example 46), control and Hif½αDKO cells were treated with IOX5 (68, example 46) and it was found that control AML cells manifest substantially reduced proliferation, while Hif½αDKO cells are refractory to IOX5 (68, example 46) and proliferate normally (FIG. 11A), indicating that IOX5 (68, example 46) requires the intact HIF system to exhibit its anti-leukaemic activity.

### Example 176—PHD Inhibition Promotes HIF-Dependent Anti-Leukaemic Programme

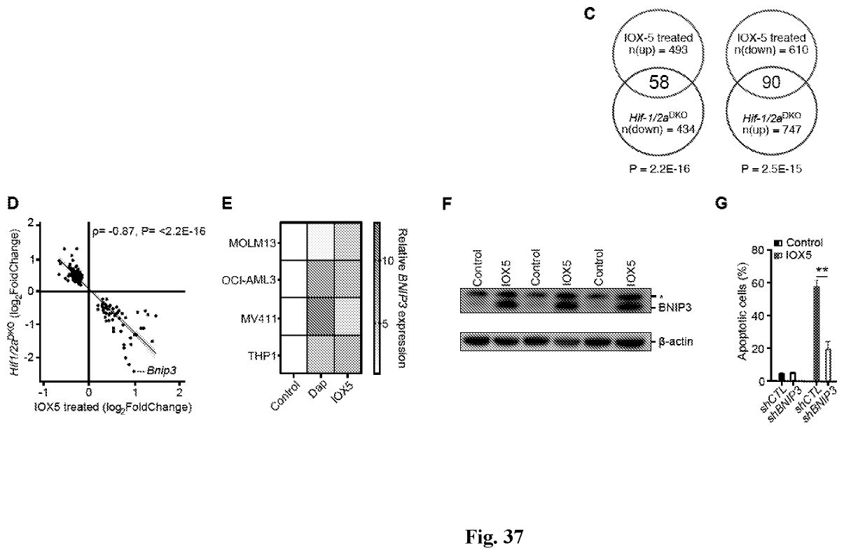

Considering the HIF-α dependency of the anti-leukaemic role of IOX5 (68, example 46), transcriptomic analyses of IOX5 (68, example 46)-treated AML cells was compared with available datasets of HIF-α dependent transcription^{30,32} and, as expected, multiple genes upregulated by IOX5 (68, example 46) are positively regulated by HIF-α, and vice versa (FIG. 36B). Furthermore, the intersection of up-and down-regulated transcripts in IOX5 (68, example 46)-treated cells with the down-and up-regulated transcripts in Hif½αDKO cells, respectively, was examined revealing HIF-dependent transcripts that are dysregulated by PHD inhibition (FIG. 37C). Examining transcripts that were downregulated upon IOX5 (68, example 46) treatment (and upregulated upon loss of Hif-α) revealed a number of genes known to be overexpressed or to have oncogenic roles in AML and CML, including Hspa8, Nup98, Kpnb1, Rbm15⁹¹⁻⁹⁴ (FIG. 36D). Inspecting transcripts which are upregulated upon IOX5 (68, example 46) treatment (and downregulated upon loss of Hif-α) showed increased expression of known HIF-target genes, including the 2OG-dependent JmjC histone demethylase Kdm5b, a known tumour suppressor in AML^{65,95}. Interestingly, analyses also revealed significant upregulation of the pro-apoptotic factor Bnip3, along with its mitochondrial partner protein Fam 162a⁹⁶⁻⁹⁸ (FIG. 36E). Spearman rank analyses of transcripts upregulated upon IOX5 treatment and downregulated upon loss of Hif-α revealed a significant correlation and identified Bnip3 as being inversely correlated with loss of Hif-α (FIG. 37D). Consistent with this observation, following PHD inhibition both Dap and IOX5 (68, example 46) strongly induced BNIP3 expression in diverse established human AML cells (FIG. 37E). Furthermore, IOX5 (68, example 46) also strongly upregulated BNIP3 protein in THP-1 cells (FIG. 37F) and it was observed that BNIP3 knockdown decreased the pro-apoptotic effect of IOX5 (68, example 46) (FIGS. 37G and 36F-G). The combined results show that the anti-leukaemic effect of PHD inhibition is HIF-dependent. Further, although the effects of HIF upregulation can be pleiotropic and context dependent²³, the present examples imply that the anti-leukaemic effect of PHD inhibition is mediated, at least in part, through increased expression of Bnip3.

### Example 177—PHD Inhibition Combined with Current AML Therapies Efficiently Ablates AML Cells

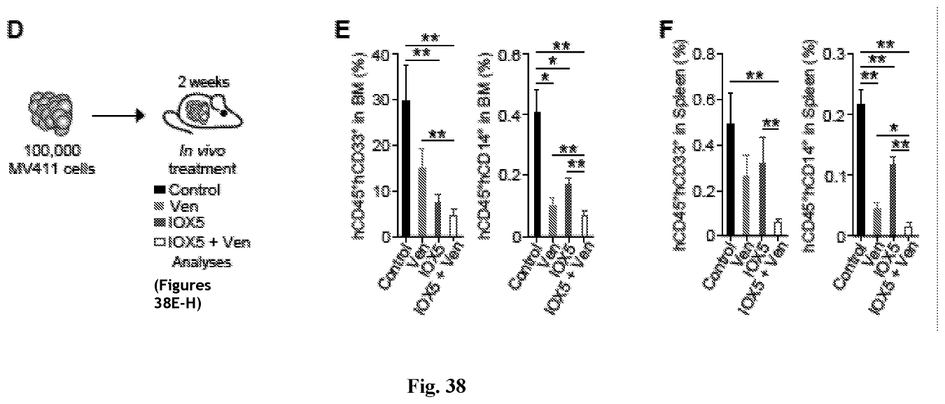

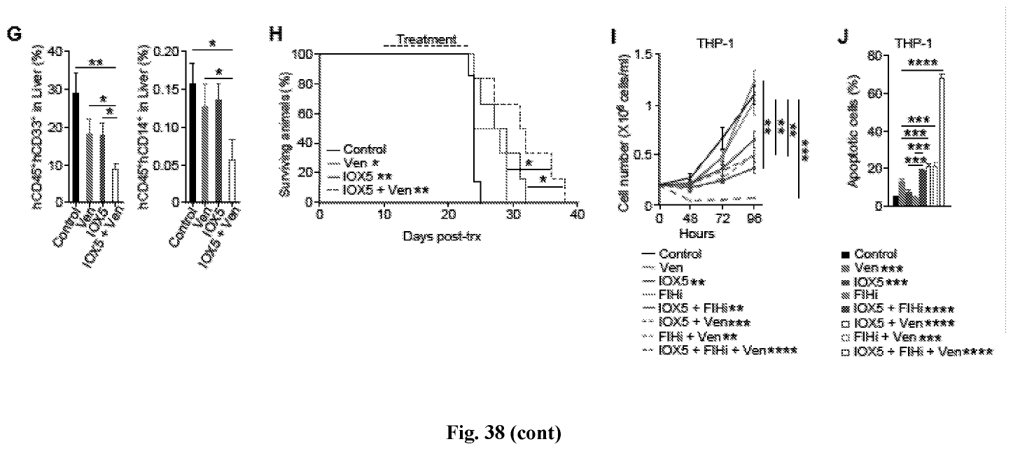

Further to findings in Example 170, the efficacy of IOX5 (68, example 46)+Venetoclax combination in vivo was investigated (FIG. 38D). MV411 AML cells were engrafted (with MLL-AF4 and FLT3-ITD mutation) and the recipient mice were treated with a vehicle, IOX5 (68, example 46), Venetoclax, and a combination of IOX5 (68, example 46)+Venetoclax. It was found that IOX5 (68, example 46) or Venetoclax alone decreased leukaemic burden and extended survival of the recipient mice (FIGS. 38E-H). Moreover, the combination therapy further compromised leukaemic burden and further extended mouse survival, compared to IOX5 (68, example 46) or Venetoclax alone (FIGS. 38E-H). Thus, combined PHD and BCL-2 inhibition acts in concert to efficiently promote AML cell apoptosis, decrease AML burden and prolong mouse survival, revealing a promising therapeutic strategy for AML treatment.

### Example 178—Combined PHD and FIH Inhibition to Eliminate AML Cells

The results presented so far indicate that PHD inhibition and consequent HIF-α stabilisation is sufficient to activate HIF-dependent programme to compromise AML cells. In addition to the post-transcriptional regulation of protein degradation by the PHD-VHL system^{25,100-103}, HIF-α activity is regulated by factor inhibiting HIF (FIH), which hydroxylates asparaginyl HIF-α residues to restrain HIF's transcriptional activity¹⁰⁴⁻¹⁰⁶. FIH inhibition increases the affinity of HIF-α to transcriptional co-activators p300/CBP, thus promoting HIF-mediated transcription. It has been found that FIH is comparably expressed in both human AML cells and primitive and mature blood cells from healthy donors (FIG. 12B). Given that both PHD and FIH inactivation may be needed to achieve optimal HIF activity²³, the impact of dual PHD/FIH inhibition on AML cells was investigated. It was found that a prodrug form of an FIH inhibitor (DM-NOFD, Dimethyl N-oxalyl-D-phenylalanine)^{23,107} alone had no impact on proliferation and survival of AML cells (FIGS. 38I-J). Strikingly, however, the combined treatment of AML cells with IOX5 (68, example 46) and DM-NOFD had a significantly more potent anti-leukaemic effect, compared to IOX5 (68, example 46) treatment alone (FIGS. 38I-J). Given that Venetoclax potentiates the anti-leukaemic activity of IOX5 (68, example 46), the effect of the triple combination of PHD and FIH inhibitors and Venetoclax in AML was investigated. Indeed, this combination very severely compromised AML cells, compared to single or double combinations used (FIGS. 38I-J). Together, these data suggest that both increased HIF-α stability (by PHD inhibition) as well as enhanced HIF-α transcriptional activity (by FIH inhibition) is desirable to elicit a strong therapeutic effect against AML, which can be further potentiated by Venetoclax.

### DISCUSSION

The identification of AML-specific and non-toxic therapies is a major challenge in haemato-oncology. The functional significance of the activation of the HIF system on AML has not previously been explored. It has been found that genetic inactivation of two suppressors of the HIF pathway, PHD1 and PHD2, attenuates AML initiation and propagation. Concordantly, pharmacological PHD inhibition compromises AML cells in a HIF-dependent manner, induces AML cell apoptosis, reduces AML burden and impairs disease progression. Notably, inhibition of PHDs has no detrimental consequences on normal haematopoiesis, implying a broad therapeutic window for deploying PHD inhibitors in AML. Indeed, PHD inhibition promotes HSC quiescence, facilitates haematopoietic regeneration following injury, enhances erythropoiesis and protects organs from chemotherapy-mediated damage in a HIF-dependent manner¹⁰⁸⁻¹¹¹. These results highlight the therapeutic potential of PHD targeting in AML and imply that PHD inhibition may compromise AML while counteracting AML-induced suppression of normal haematopoiesis (including anaemia) and the negative impact of chemotherapy. On the other hand, PHD inhibition by Dap impairs murine B-cell development⁷⁸, while DMOG, an inhibitor of 2OG-dependent oxygenases, compromises human monocyte Survival⁴⁶.

Given the availability of relatively non-toxic clinically used PHD inhibitors, including Daprodustat (Dap) and Molidustat, the findings of the present inventors are of considerable translational potential for AML treatment. Clinical trials testing Dap and Molidustat in renal anaemia have shown that chronic administration of these compounds is generally well-tolerated, does not cause severe adverse effects, and efficiently promotes erythropoiesis in patients with chronic kidney disease^{18,37}. While these compounds are relatively selective for PHD inhibition and may be suitable for AML treatment, they manifest varying inhibition of other 2OG-dependent oxygenases, including JMJD6^{17,34}, which is required for normal haematopoiesis and HSC maintenance¹¹². Given that normal haematopoiesis is suppressed by AML, it is undesirable to inhibit key regulators of haematopoiesis whilst targeting the disease. Thus, a structure guided approach was employed to generate a potent and selective PHD inhibitor, with particular emphasis on selectivity with respect to other 2OG-dependent oxygenase family members. A PHD-specific pocket located where HIF-α binds, and which is potentially involved in substrate binding has been targetted herein¹¹³. Indeed, knowing that the PHDs are highly selective for HIF-α²⁴, it has been hypothesised that designing a compound that both binds to the PHD-specific pocket, as well as active site Fe(II), might improve inhibition selectivity. The present inventors have found this to be the case for IOX5 (68, example 46).

IOX5 (68, example 46) displays potent in vitro and in vivo anti-leukaemic activity comparable to Dap, with both compounds increasing HIF-α levels and activating HIF-dependent transcriptional networks. In addition to suppressing numerous genes which promote oncogenic transformation, IOX5 (68, example 46) promotes the expression of a HIF-1 target Bnip3, a pro-apoptotic member of the BH3 family¹¹⁴⁻¹¹⁶. One of the multifaceted roles of BNIP3 is to activate apoptosis through localisation to the mitochondria and activation of BAX/BAK, which permeabilise the outer mitochondrial membrane and destabilise mitochondrial membrane potential, leading to subsequent cell death^{97,98,117-120}. BAX/BAK are restrained by the anti-apoptotic BCL-2, and BCL-2 inhibition by Venetoclax, which consequently promotes BAX/BAK-mediated apoptosis⁴⁷, has already significantly advanced AML treatments. With this in mind, IOX5 (68, example 46) and Venetoclax were combined with the aim of enabling BAX/BAK-dependent apoptosis, and discovered that BCL-2 inhibition potentiated the pro-apoptotic effect of PHD inactivation. Therefore, it is suggested herein that IOX5 (68, example 46) and Venetoclax converge to activate AML cell death, thus highlighting an attractive therapeutic strategy against this disease.

The present results indicate that PHD inhibition compromises AML driven by diverse genetic alterations. It has been found that PHD inactivation has a significant anti-leukaemic effect in AML driven by expression of Meis1 and Hoxa9, both of which are frequently overexpressed in several subsets of human AML⁴⁸⁻⁵². It was also demonstrated that PHD inhibition activates apoptosis in AML cells harbouring diverse genetic alterations, including MLL-AF9, which function upstream of Meis1 and Hoxa9, as well as AML-ETO, PML-RARα and FLT3-ITD (with and without NPM1 mutations). Dap and IOX5 (68, example 46) promote death in cells from primary AML samples from patients with poor risk AML harbouring complex karyotype with or without TP53 mutations.

Taken together, the presently presented preclinical investigations provide proof-of-concept evidence that selective PHD inhibition is highly anti-leukaemogenic, setting the stage for a novel therapeutic approach to AML treatment, without any toxic effects to normal haematopoiesis. HIF upregulation may be achieved by employing either the existing clinically used PHD inhibitors or highly selective PHD inhibitors developed specifically for AML treatment, such as IOX5 (68, example 46).

### REFERENCES

1 Horton, S. J. & Huntly, B. J. Recent advances in acute myeloid leukemia stem cell biology. Haematologica 97, 966-974 (2012). https://doi.org: 10.3324/haematol.2011.054734
2 Shlush, L. I. et al. Identification of pre-leukaemic haematopoietic stem cells in acute leukaemia. Nature 506, 328-333 (2014). https://doi.org: 10.1038/nature13038
3 Boddu, P. & Borthakur, G. Therapeutic targeting of isocitrate dehydrogenase mutant AML. Expert Opinion on Investigational Drugs 26, 525-530 (2017). https://doi.org: 10.1080/13543784.2017.1317745
4 Dawson, M. A., Kouzarides, T. & Huntly, B. J. P. Targeting Epigenetic Readers in Cancer. New England Journal of Medicine 367, 647-657 (2012). https://doi.org: 10.1056/nejmra1112635
5 DiNardo, C. D. Which novel agents hold the greatest promise in AML? Best Pract Res Clin Haematol 32, 101106 (2019). https://doi.org: 10.1016/j.beha.2019.101106
6 DiNardo, C. D. et al. Azacitidine and Venetoclax in Previously Untreated Acute Myeloid Leukemia. New England Journal of Medicine 383, 617-629 (2020). https://doi.org: 10.1056/NEJMoa2012971
7 Gallipoli, P., Giotopoulos, G. & Huntly, B. J. P. Epigenetic regulators as promising therapeutic targets in acute myeloid leukemia. Therapeutic Advances in Hematology 6, 103-119 (2015). https://doi.org: 10.1177/2040620715577614
8 Konopleva, M. Y. Mechanisms for resistance in AML insights into molecular pathways mediating resistance to venetoclax. Best Pract Res Clin Haematol 34, 101251 (2021). https://doi.org: 10.1016/j.beha.2021.101251
9 Estey, E. H. Acute myeloid leukemia: 2021 update on risk-stratification and management. Am J Hematol 95, 1368-1398 (2020). https://doi.org: 10.1002/ajh.25975
10 Papaemmanuil, E. et al. Genomic Classification and Prognosis in Acute Myeloid Leukemia. New England Journal of Medicine 374, 2209-2221 (2016). https://doi.org: 10.1056/nejmoa1516192
16 Vukovic, M. et al. Hif-1α and Hif-2α synergize to suppress AML development but are dispensable for disease maintenance. J Exp Med 212, 2223-2234 (2015). https://doi.org: 10.1084/jem.20150452
17 Islam, M. S., Leissing, T. M., Chowdhury, R., Hopkinson, R. J. & Schofield, C. J. 2-Oxoglutarate-Dependent Oxygenases. Annual Review of Biochemistry 87, 585-620 (2018). https://doi.org: 10.1146/annurev-biochem-061516-044724
18 Semenza, G. L. Regulation of Erythropoiesis by the Hypoxia-Inducible Factor Pathway: Effects of Genetic and Pharmacological Perturbations. Annual Review of Medicine 74, 307-319 (2023). https://doi.org: 10.1146/annurev-med-042921-102602
19 Semenza, G. L. HIF-1, O(2), and the 3 PHDs: how animal cells signal hypoxia to the nucleus. Cell 107, 1-3 (2001). https://doi.org: 10.1016/s0092-8674 (01)00518-9
20 Schofield, C. J. & Ratcliffe, P. J. Oxygen sensing by HIF hydroxylases. Nature Reviews Molecular Cell Biology 5, 343-354 (2004). https://doi.org: 10.1038/nrm1366
21 Appelhoff, R. J. et al. Differential function of the prolyl hydroxylases PHD1, PHD2, and PHD3 in the regulation of hypoxia-inducible factor. J Biol Chem 279, 38458-38465 (2004). https://doi.org: 10.1074/jbc.M406026200
22 Berra, E. et al. HIF prolyl-hydroxylase 2 is the key oxygen sensor setting low steady-state levels of HIF-1alpha in normoxia. Embo j 22, 4082-4090 (2003). https://doi.org: 10.1093/emboj/cdg392
23 Chan, M. C. et al. Tuning the Transcriptional Response to Hypoxia by Inhibiting Hypoxia-inducible Factor (HIF) Prolyl and Asparaginyl Hydroxylases. J Biol Chem 291,20661-20673 (2016). https://doi.org: 10.1074/jbc.M116.749291
24 Cockman, M. E. et al. Lack of activity of recombinant HIF prolyl hydroxylases (PHDs) on reported non-HIF substrates. eLife 8 (2019). https://doi.org: 10.7554/elife.46490
25 Kaelin, W. G., Jr. & Ratcliffe, P. J. Oxygen sensing by metazoans: the central role of the HIF hydroxylase pathway. Mol Cell 30, 393-402 (2008). https://doi.org: 10.1016/j.molcel.2008.04.009
26 Semenza, G. L. Hypoxia-inducible factor 1: master regulator of 02 homeostasis. Curr Opin Genet Dev 8, 588-594 (1998). https://doi.org: 10.1016/s0959-437x(98)80016-6
27 Semenza, G. L. HIF-1 mediates metabolic responses to intratumoral hypoxia and oncogenic mutations. Journal of Clinical Investigation 123, 3664-3671 (2013). https://doi.org: 10.1172/jci67230
28 Yang, M., Su, H., Soga, T., Kranc, K. R. & Pollard, P. J. Prolyl hydroxylase domain enzymes: important regulators of cancer metabolism. Hypoxia (Auckl)2, 127-142 (2014). https://doi.org: 10.2147/hp.S47968
29 Semenza, G. L. Oxygen sensing, hypoxia-inducible factors, and disease pathophysiology. Annu Rev Pathol 9, 47-71 (2014). https://doi.org: 10.1146/annurev-pathol-012513-104720
30 Mole, D. R. et al. Genome-wide Association of Hypoxia-inducible Factor (HIF)-1a and HIF-2α DNA Binding with Expression Profiling of Hypoxia-inducible Transcripts. Journal of Biological Chemistry 284, 16767-16775 (2009). https://doi.org: 10.1074/jbc.m901790200
31 Schödel, J., Mole, D. R. & Ratcliffe, P. J. Pan-genomic binding of hypoxia-inducible transcription factors. Biol Chem 394, 507-517 (2013). https://doi.org: 10.1515/hsz-2012-0351
32 Schödel, J. et al. High-resolution genome-wide mapping of HIF-binding sites by ChIP-seq. Blood 117, e207-217 (2011). https://doi.org: 10.1182/blood-2010-10-314427
33 Ariazi, J. L. et al. Discovery and Preclinical Characterization of GSK1278863 (Daprodustat), a Small Molecule Hypoxia Inducible Factor-Prolyl Hydroxylase Inhibitor for Anemia. Journal of Pharmacology and Experimental Therapeutics 363, 336-347 (2017). https://doi.org: 10.1124/jpet. 117.242503
34 Yeh, T.-L. et al. Molecular and cellular mechanisms of HIF prolyl hydroxylase inhibitors in clinical trials. Chem. Sci. 8, 7651-7668 (2017). https://doi.org: 10.1039/c7sc02103h
35 McDonough, M. A. et al. Cellular oxygen sensing: Crystal structure of hypoxia-inducible factor prolyl hydroxylase (PHD2). Proceedings of the National Academy of Sciences 103, 9814-9819 (2006). https://doi.org: 10.1073/pnas.0601283103
36 Figg, W. D. et al. Structural Basis of Prolyl Hydroxylase Domain Inhibition by Molidustat. ChemMedChem 16, 2082-2088 (2021). https://doi.org: 10.1002/cmdc.202100133
37 Singh, A. K. et al. Daprodustat for the Treatment of Anemia in Patients Undergoing Dialysis. New England Journal of Medicine 385, 2325-2335 (2021). https://doi.org: 10.1056/nejmoa2113379
38 Su, K., Li, Z., Yu, Y. & Zhang, X. The prolyl hydroxylase inhibitor roxadustat: Paradigm in drug discovery and prospects for clinical application beyond anemia. Drug Discov Today 25, 1262-1269 (2020). https://doi.org: 10.1016/j.drudis.2020.04.017
39 Nagashima, R., Ishikawa, H., Kuno, Y., Kohda, C. & Iyoda, M. HIF-PHD inhibitor regulates the function of group2 innate lymphoid cells and polarization of M2 macrophages. Scientific Reports 13, 1867 (2023). https://doi.org: 10.1038/s41598-023-29161-3
40 Nishide, S. et al. Controlling the Phenotype of Tumor-Infiltrating Macrophages via the PHD-HIF Axis Inhibits Tumor Growth in a Mouse Model. iScience 19, 940-954 (2019). https://doi.org: https://doi.org/10.1016/j.isci.2019.08.033
41 Okumura, C. Y. M. et al. A new pharmacological agent (AKB-4924) stabilizes hypoxia inducible factor-1 (HIF-1) and increases skin innate defenses against bacterial infection. Journal of Molecular Medicine 90, 1079-1089 (2012). https://doi.org: 10.1007/s00109-012-0882-3
42 Marks, E. et al. Oral Delivery of Prolyl Hydroxylase Inhibitor: AKB-4924 Promotes Localized Mucosal Healing in a Mouse Model of Colitis. Inflammatory Bowel Diseases 21, 267-275 (2014). https://doi.org: 10.1097/mib.0000000000000277
43 Robinson, A. et al. Mucosal Protection by Hypoxia-Inducible Factor Prolyl Hydroxylase Inhibition. Gastroenterology 134, 145-155 (2008). https://doi.org: https://doi.org/10.1053/j.gastro.2007.09.033
44 Keely, S. et al. Contribution of epithelial innate immunity to systemic protection afforded by prolyl hydroxylase inhibition in murine colitis. Mucosal Immunology 7, 114-123 (2014). https://doi.org: https://doi.org/10.1038/mi.2013.29
45 Rose, N. R., McDonough, M. A., King, O. N. F., Kawamura, A. & Schofield, C. J. Inhibition of 2-oxoglutarate dependent oxygenases. Chemical Society Reviews 40, 4364 (2011). https://doi.org: 10.1039/c0cs00203h
46 Crifo, B. et al. Hydroxylase Inhibition Selectively Induces Cell Death in Monocytes. The Journal of Immunology 202, 1521-1530 (2019). https://doi.org: 10.4049/jimmunol.1800912
47 Samra, B., Konopleva, M., Isidori, A., Daver, N. & DiNardo, C. Venetoclax-Based Combinations in Acute Myeloid Leukemia: Current Evidence and Future Directions. Front Oncol 10, 562558 (2020). https://doi.org: 10.3389/fonc.2020.562558
48 Drabkin, H. et al. Quantitative HOX expression in chromosomally defined subsets of acute myelogenous leukemia. Leukemia 16, 186-195 (2002). https://doi.org: 10.1038/sj.leu.2402354
49 Collins, C. T. & Hess, J. L. Deregulation of the HOXA9/MEIS1 axis in acute leukemia. Current Opinion in Hematology 23, 354-361 (2016). https://doi.org: 10.1097/moh.0000000000000245
50 Kroon, E. et al. Hoxa9 transforms primary bone marrow cells through specific collaboration with Meis1a but not Pbx1b. Embo j 17, 3714-3725 (1998). https://doi.org: 10.1093/emboj/17.13.3714
51 Lawrence, H. et al. Frequent co-expression of the HOXA9 and MEIS1 homeobox genes in human myeloid leukemias. Leukemia 13, 1993-1999 (1999). https://doi.org: 10.1038/sj.leu.2401578
52 Uckelmann, H. J. et al. Therapeutic targeting of preleukemia cells in a mouse model of NPM1 mutant acute myeloid leukemia. Science 367, 586-590 (2020). https://doi.org: 10.1126/science.aax5863
60 Chan, M. C., Holt-Martyn, J. P., Schofield, C. J. & Ratcliffe, P. J. Pharmacological targeting of the HIF hydroxylases-A new field in medicine development. Molecular Aspects of Medicine 47-48, 54-75 (2016). https://doi.org: https://doi.org/10.1016/j.mam.2016.01.001
61 Berra, E. HIF prolyl-hydroxylase 2 is the key oxygen sensor setting low steady-state levels of HIF-1 in normoxia. The EMBO Journal 22, 4082-4090 (2003). https://doi.org: 10.1093/emboj/cdg392
62 Somervaille, T. C. P. & Cleary, M. L. Identification and characterization of leukemia stem cells in murine MLL-AF9 acute myeloid leukemia. Cancer Cell 10, 257-268 (2006). https://doi.org: 10.1016/j.ccr.2006.08.020
71 Holt-Martyn, J. P. et al. Structure-Activity Relationship and Crystallographic Studies on 4-Hydroxypyrimidine HIF Prolyl Hydroxylase Domain Inhibitors.
ChemMedChem 15, 270-273 (2020). https://doi.org: 10.1002/cmdc.201900557
72 GSK. (GSK.com, 2023)
73 Döhner, H., Weisdorf, D. J. & Bloomfield, C. D. Acute Myeloid Leukemia. N Engl J Med 373, 1136-1152 (2015). https://doi.org: 10.1056/NEJMra1406184
74 Ginouves, A., Ilc, K., Mací as, N., Pouysségur, J. & Berra, E. PHDs overactivation during chronic hypoxia “desensitizes” HIFa and protects cells from necrosis. Proceedings of the National Academy of Sciences 105, 4745-4750 (2008). https://doi.org: doi: 10.1073/pnas.0705680105
75 Wiesener, M. S. et al. Induction of Endothelial PAS Domain Protein-1 by Hypoxia: Characterization and Comparison With Hypoxia-Inducible Factor-1a. Blood 92, 2260-2268 (1998). https://doi.org: 10.1182/blood.V92.7.2260
76 Jeknić, S., Kudo, T., Song, J. J. & Covert, M. W. An optimized reporter of the transcription factor hypoxia-inducible factor 1a reveals complex HIF-1α activation dynamics in single cells. Journal of Biological Chemistry 299, 104599 (2023). https://doi.org: https://doi.org/10.1016/j.jbc.2023.104599
77 Kshitiz et al. Lactate-dependent chaperone-mediated autophagy induces oscillatory HIF-1α activity promoting proliferation of hypoxic cells. Cell Systems 13, 1048-1064.e1047 (2022). https://doi.org: https://doi.org/10.1016/j.cels.2022.11.003
78 Burrows, N. et al. Dynamic regulation of hypoxia-inducible factor-1a activity is essential for normal B cell development. Nat Immunol 21, 1408-1420 (2020). https://doi.org: 10.1038/s41590-020-0772-8
79 Masoud, G. N. & Li, W. HIF-1α pathway: role, regulation and intervention for cancer therapy. Acta Pharm Sin B 5, 378-389 (2015). https://doi.org: 10.1016/j.apsb.2015.05.007
80 Kierans, S. J. & Taylor, C. T. Regulation of glycolysis by the hypoxia-inducible factor (HIF): implications for cellular physiology. The Journal of Physiology 599, 23-37 (2021).https://doi.org: 10.1113/jp280572
81 Akef, A., McGraw, K., Cappell, S. D. & Larson, D. R. Ribosome biogenesis is a downstream effector of the oncogenic U2AF1-S34F mutation. PLOS Biology 18, e3000920 (2020). https://doi.org: 10.1371/journal.pbio.3000920
82 Boudhraa, Z., Carmona, E., Provencher, D. & Mes-Masson, A. M. Ran GTPase: A Key Player in Tumor Progression and Metastasis. Front Cell Dev Biol 8, 345 (2020). https://doi.org: 10.3389/fcell.2020.00345
83 Fruman, D. A. et al. The PI3K Pathway in Human Disease. Cell 170, 605-635 (2017). https://doi.org: 10.1016/j.cell.2017.07.029
84 Kuntz, E. M. et al. Targeting mitochondrial oxidative phosphorylation eradicates therapy-resistant chronic myeloid leukemia stem cells. Nature Medicine 23, 1234-1240 (2017). https://doi.org: 10.1038/nm.4399
85 Laplante, M. & David. mTOR Signaling in Growth Control and Disease. Cell 149, 274-293 (2012). https://doi.org: 10.1016/j.cell.2012.03.017
86 Larramendy, M. L. et al. Overexpression of translocation-associated fusion genes of FGFRI, MYC, NPMI, and DEK, but absence of the translocations in acute myeloid leukemia. A microarray analysis. Haematologica 87, 569-577 (2002).
87 Mughal, M. K. et al. Acute myeloid leukaemia: expression of MYC protein and its association with cytogenetic risk profile and overall survival. Hematological Oncology 35, 350-356 (2017). https://doi.org: 10.1002/hon.2279
88 Segeren, H. A. et al. Excessive E2F Transcription in Single Cancer Cells Precludes Transient Cell-Cycle Exit after DNA Damage. Cell Reports 33, 108449 (2020). https://doi.org: 10.1016/j.celrep.2020.108449
89 Yuen, H.-F. et al. Ran GTPase promotes cancer progression via Met receptor-mediated downstream signaling. Oncotarget 7, 75854-75864 (2016). https://doi.org: 10.18632/oncotarget. 12420
90 Zheng, J. Energy metabolism of cancer: Glycolysis versus oxidative phosphorylation (Review). Oncol Lett 4, 1151-1157 (2012). https://doi.org: 10.3892/ol.2012.928
91 Moore, M. A. S. et al. NUP98 Dysregulation in Myeloid Leukemogenesis. Annals of the New York Academy of Sciences 1106, 114-142 (2007). https://doi.org: 10.1196/annals. 1392.019
92 Wang, T. et al. Inhibition of KPNB1 Inhibits Proliferation and Promotes Apoptosis of Chronic Myeloid Leukemia Cells Through Regulation of E2F₁. Onco Targets Ther 12, 10455-10467 (2019). https://doi.org: 10.2147/ott.S210048
93 Li, J. & Ge, Z. High HSPA8 expression predicts adverse outcomes of acute myeloid leukemia. BMC Cancer 21, 475 (2021). https://doi.org: 10.1186/s12885-021-08193-w
94 Yang, Y., Wang, S., Zhang, Y. & Zhu, X. Biological effects of decreasing RBM15 on chronic myelogenous leukemia cells. Leukemia & Lymphoma 53, 2237-2244 (2012). https://doi.org: 10.3109/10428194.2012.684350
95 Horton, J. R. et al. Characterization of a Linked Jumonji Domain of the KDM5/JARID1 Family of Histone H3 Lysine 4 Demethylases. J Biol Chem 291, 2631-2646 (2016). https://doi.org: 10.1074/jbc.M115.698449
96 Burton, T. R. & Gibson, S. B. The role of Bcl-2 family member BNIP3 in cell death and disease: NIPping at the heels of cell death. Cell Death Differ 16, 515-523 (2009). https://doi.org: 10.1038/cdd.2008.185
97 Loftus, S. K., Baxter, L. L., Cronin, J. C., Fufa, T. D. & Pavan, W. J. Hypoxia-induced HIF1α targets in melanocytes reveal a molecular profile associated with poor melanoma prognosis. Pigment Cell Melanoma Res 30, 339-352 (2017). https://doi.org: 10.1111/pcmr. 12579
98 Xia, J. H., Li, H. L., Li, B. J., Gu, X. H. & Lin, H. R. Acute hypoxia stress induced abundant differential expression genes and alternative splicing events in heart of tilapia. Gene 639, 52-61 (2018). https://doi.org: https://doi.org/10.1016/j.gene.2017.10.002
99 Juliusson, G. Venetoclax with intensive chemotherapy in younger patients with acute myeloid leukaemia. The Lancet Haematology 9, e317-e318 (2022). https://doi.org: 10.1016/s2352-3026 (22)00100-4
100 Maxwell, P. H. et al. The tumour suppressor protein VHL targets hypoxia-inducible factors for oxygen-dependent proteolysis. Nature 399, 271-275 (1999). https://doi.org: 10.1038/20459
101 Cockman, M. E. et al. Lack of activity of recombinant HIF prolyl hydroxylases (PHDs) on reported non-HIF substrates. Elife 8 (2019). https://doi.org: 10.7554/eLife.46490
102 Schofield, C. J. & Ratcliffe, P. J. Oxygen sensing by HIF hydroxylases. Nat Rev Mol Cell Biol 5, 343-354 (2004). https://doi.org: 10.1038/nrm1366
103 Jaakkola, P. et al. Targeting of HIF-alpha to the von Hippel-Lindau ubiquitylation complex by 02-regulated prolyl hydroxylation. Science 292, 468-472 (2001).
104 McNeill, L. A. et al. Hypoxia-inducible factor asparaginyl hydroxylase (FIH-1) catalyses hydroxylation at the beta-carbon of asparagine-803. Biochem J 367, 571-575 (2002). https://doi.org: 10.1042/BJ20021162
105 Lando, D. et al. FIH-1 is an asparaginyl hydroxylase enzyme that regulates the transcriptional activity of hypoxia-inducible factor. Genes Dev 16, 1466-1471 (2002). https://doi.org: 10.1101/gad.991402
106 Hewitson, K. S. et al. Hypoxia-inducible factor (HIF) asparagine hydroxylase is identical to factor inhibiting HIF (FIH) and is related to the cupin structural family. J Biol Chem 277, 26351-26355 (2002). https://doi.org: 10.1074/jbc.C₂₀₀₂₇₃₂₀₀
107 McDonough, M. A. et al. Selective Inhibition of Factor Inhibiting Hypoxia-Inducible Factor. Journal of the American Chemical Society 127, 7680-7681 (2005). https://doi.org: 10.1021/ja050841b
108 Forristal, C. E. et al. HIF-1α is required for hematopoietic stem cell mobilization and 4-prolyl hydroxylase inhibitors enhance mobilization by stabilizing HIF-1α. Leukemia 29, 1366-1378 (2015). https://doi.org: 10.1038/leu.2015.8
109 Speth, J. M., Hoggatt, J., Singh, P. & Pelus, L. M. Pharmacologic increase in HIF1α enhances hematopoietic stem and progenitor homing and engraftment. Blood 123, 203-207 (2014). https://doi.org: 10.1182/blood-2013-07-516336
110 Forristal, C. E. et al. Pharmacologic stabilization of HIF-1α increases hematopoietic stem cell quiescence in vivo and accelerates blood recovery after severe irradiation. Blood 121, 759-769 (2013). https://doi.org: 10.1182/blood-2012-02-408419
111 Haase, V. H. Therapeutic targeting of the HIF oxygen-sensing pathway: Lessons learned from clinical studies. Exp Cell Res 356, 160-165 (2017). https://doi.org: 10.1016/j.yexcr.2017.05.004
112 Lawson, H. et al. JMJD6 promotes self-renewal and regenerative capacity of hematopoietic stem cells. Blood Adv 5, 889-899 (2021). https://doi.org: 10.1182/bloodadvances.2020002702
113 Chowdhury, R. et al. Structural basis for oxygen degradation domain selectivity of the HIF prolyl hydroxylases. Nature Communications 7, 12673 (2016). https://doi.org: 10.1038/ncomms12673
114 Bruick, R. K. Expression of the gene encoding the proapoptotic Nip3 protein is induced by hypoxia. Proceedings of the National Academy of Sciences 97, 9082-9087 (2000). https://doi.org: 10.1073/pnas.97.16.9082
115 Raval, R. R. et al. Contrasting Properties of Hypoxia-Inducible Factor 1 (HIF-1) and HIF-2 in von Hippel-Lindau-Associated Renal Cell Carcinoma. Molecular and Cellular Biology 25, 5675-5686 (2005). https://doi.org: doi: 10.1128/MCB.25.13.5675-5686.2005
116 Sowter, H. M., Ratcliffe, P. J., Watson, P., Greenberg, A. H. & Harris, A. L. HIF-1-dependent regulation of hypoxic induction of the cell death factors BNIP3 and NIX in human tumors. Cancer Res 61, 6669-6673 (2001).
117 Kanzawa, T. et al. Arsenic trioxide induces autophagic cell death in malignant glioma cells by upregulation of mitochondrial cell death protein BNIP3. Oncogene 24, 980-991 (2005). https://doi.org: 10.1038/sj.onc. 1208095
118 Lee, Y. S. et al. BAX-dependent mitochondrial pathway mediates the crosstalk between ferroptosis and apoptosis. Apoptosis 25, 625-631 (2020). https://doi.org: 10.1007/s10495-020-01627-z
119 Kubli, D. A., Ycaza, J. E. & Gustafsson, A. B. Bnip3 mediates mitochondrial dysfunction and cell death through Bax and Bak. Biochem J 405, 407-415 (2007). https://doi.org: 10.1042/bj20070319
120 Yook, Y. H. et al. Nitric oxide induces BNIP3 expression that causes cell death in macrophages. Biochem Biophys Res Commun 321, 298-305 (2004). https://doi.org: 10.1016/j.bbrc.2004.06.144
121 Hu, Y. & Smyth, G. K. ELDA: Extreme limiting dilution analysis for comparing depleted and enriched populations in stem cell and other assays. Journal of Immunological Methods 347, 70-78 (2009). https://doi.org: https://doi.org/10.1016/j.jim.2009.06.008
123 Islam, M. S. et al. Biochemical and structural investigations clarify the substrate selectivity of the 2-oxoglutarate oxygenase JMJD6. J Biol Chem 294, 11637-11652 (2019). https://doi.org: 10.1074/jbc.RA119.008693
126 Rose, N. R. et al. Plant Growth Regulator Daminozide Is a Selective Inhibitor of Human KDM 2/7 Histone Demethylases. Journal of Medicinal Chemistry 55, 6639-6643 (2012). https://doi.org: 10.1021/jm300677j
127 Kwok, C., Zeisig, B. B., Qiu, J., Dong, S. & So, C. W. Transforming activity of AML1-ETO is independent of CBFbeta and ETO interaction but requires formation of homo-oligomeric complexes. Proc Natl Acad Sci USA 106, 2853-2858 (2009). https://doi.org: 10.1073/pnas.0810558106
128 Bottomly, D. et al. Integrative analysis of drug response and clinical outcome in acute myeloid leukemia. Cancer Cell 40, 850-864.e859 (2022). https://doi.org: https://doi.org/10.1016/j.ccell.2022.07.002
129 Tyner, J. W. et al. Functional genomic landscape of acute myeloid leukaemia. Nature 562, 526-531 (2018). https://doi.org: 10.1038/s41586-018-0623-z
130 Hewitson, K. S. et al. Hypoxia-inducible Factor (HIF) Asparagine Hydroxylase Is Identical to Factor Inhibiting HIF (FIH) and Is Related to the Cupin Structural Family. Journal of Biological Chemistry 277, 26351-26355 (2002). https://doi.org: 10.1074/jbc.c200273200
140 Ng, S. S. et al. Crystal structures of histone demethylase JMJD2A reveal basis for substrate specificity. Nature 448, 87-91 (2007). https://doi.org: 10.1038/nature05971
141 Johansson, C. et al. Structural analysis of human KDM5B guides histone demethylase inhibitor development. Nature Chemical Biology 12, 539-545 (2016). https://doi.org: 10.1038/nchembio.2087

Further embodiments of the invention are defined in the following numbered clauses.

1. A hypoxia inducible factor prolyl hydroxylase inhibitor (PHD inhibitor) for use in the treatment of blood cancer.

2. A PHD inhibitor according to clause 1, for use as defined in said clause, wherein the blood cancer is acute myeloid leukaemia (AML), chronic myeloid leukaemia (CML) or multiple myeloma (MM).

3. A PHD inhibitor according to clause 1 or clause 2, for use as defined in said clause, wherein said treatment comprises binding of the PHD inhibitor to the active site of prolyl hydroxlase domain (PHD), wherein the PHD inhibitor competes with 2-oxoglutarate for binding to said active site, and optionally wherein the PHD inhibitor competes with HIF-alpha for binding to said active site.

4. A PHD inhibitor according to any one of clauses 1 to 3, for use as defined in said clause, wherein the PHD inhibitor is a compound as defined in any one of clauses 12 to 35.

5. A PHD inhibitor according to any one of clauses 1 to 3, for use as defined in said clause, wherein the PHD inhibitor is a cobalt compound, for instance a cobalt salt (e.g. cobalt dichloride); a copper compound, for instance a copper salt; a nickel compound, for instance a nickel salt; an iron chelator, such as deferoxamine, 3,4-dihydroxybenzoic acid, 1,10-phenanthrolines, or quercetin; a 2-OG derivative mimic, or competitor (with respect to PHD binding), such as dimethyloxalylglycine (DMOG) which is a prodrug form of N-oxalylglycine (NOG); FG-2216; roxadustat; a quinolone, such as JNJ-42905343; a quinoxaline; a benzamidazole derivative, such as JNJ-42041935; an isoquinolone derivative; a 5-hydroxy-1,7 naphthyridine derivative, such as ISM5411; a monocyclic pyridine compound, such as vadadustat, and AKB6899; a pyrazolopyrimidine derivative; a pyrimidine-trione, such as daprodustat; an N-alkoxyquinolone, such as desidustat; a tetrahydropyran derivative; a dihydrothienopyridone derivative; a dihydrofuropyridoene derivative; a quinazoline-2,4-dione; a 4-oxo-2-thioxo-7-quinasoline; a 5-aminocarbonyl-4-hydroxypyrimidine derivative, such as MK8617; a spiroindolone; a 2,8-diazaspori[4,5]-decan-lone; a pyrazolone derivative, such as molidustat; a triazole substituted heteroaryl amide; a phenolic compound, such as ((S)-{2 [2-(5-cyano-3-hydroxy-pyridin-2-yl)-thiazol-4-yl]-acetylamino}-phenyl-acetic acid); a bicyclic heteroaryl derivative, such as (1,2,4-triazolo-[1,5-a]pyridine); a diacylhydrazine; pyrathione Zn, or (5-(3-(4-chlorophenoxyl) prop-1-yn-1-yl)-3-hydroxypicolinoyl)glycine.

6. A PHD inhibitor according to any one of clauses 1 to 3, for use as defined in said clause, wherein said PHD inhibitor is a compound of formula (II) or a pharmaceutically acceptable salt thereof

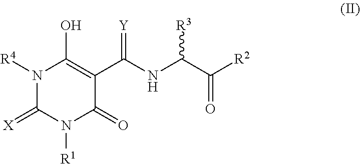

wherein
R¹ and R⁴ are each independently selected from the group consisting of H, —NR⁵R⁶, unsubstituted or substituted C₁₋₁₀ alkyl, unsubstituted or substituted C₂₋₁₀ alkenyl, unsubstituted or substituted C₂₋₁₀ alkynyl, unsubstituted or substituted C₃₋₈ cycloalkyl, unsubstituted or substituted —C₃₋₈ cycloalkylene-C₁₋₁₀ alkyl, unsubstituted or substituted C₅₋₈ cycloalkenyl, unsubstituted or substituted —C₅₋₈ cycloalkenylene-C₁₋₁₀ alkyl, unsubstituted or substituted C₃₋₈ heterocyclyl, unsubstituted or substituted —C₃₋₈ heterocyclylene-C₁₋₁₀ alkyl, unsubstituted or substituted aryl, unsubstituted or substituted-arylene-C₁₋₁₀ alkyl, unsubstituted or substituted-heteroaryl and unsubstituted or substituted-heteroarylene-C₁₋₁₀ alkyl;
R² is-NR⁷R⁸ or —OR⁹;
R³ is H or unsubstituted or substituted C₁₋₄ alkyl;
where R⁵ and R⁶ are each independently selected from the group consisting of H, unsubstituted or substituted C₁₋₁₀ alkyl, unsubstituted or substituted-C₃₋₈ cycloalkyl, unsubstituted or substituted —C₃₋₈ cycloalkylene-C₁₋₁₀ alkyl, —C₃₋₈ heterocyclyl, unsubstituted or substituted —C₃₋₈ heterocyclylene-C₁₋₁₀ alkyl, unsubstituted or substituted aryl, unsubstituted or substituted-arylene-C₁₋₁₀ alkyl, unsubstituted or substituted heteroaryl, unsubstituted or substituted-heteroarylene-C₁₋₁₀ alkyl, unsubstituted or substituted —C(O)C₁₋₄ alkyl, unsubstituted or substituted —C(O)C₃₋₆ cycloalkyl, —C(O)C₃₋₆ heterocyclyl, unsubstituted or substituted —C(O) aryl, unsubstituted or substituted —C(O) heteroaryl and unsubstituted or substituted —S(O)₂C₁₋₄ alkyl, or, when R⁵ and R⁶ are attached to the same nitrogen, R⁵ and R⁶ taken together with the nitrogen to which they are attached form a 5-or 6-or 7-membered saturated heterocyclic ring which is unsubstituted or substituted and which optionally contains one other heteroatom selected from oxygen, nitrogen and sulphur,
R⁷ and R⁸ are each independently selected from the group consisting of H, unsubstituted or substituted C₁₋₁₀ alkyl, unsubstituted or substituted C₂₋₁₀ alkenyl, unsubstituted or substituted C₂₋₁₀ alkynyl, unsubstituted or substituted C₃₋₈ cycloalkyl, unsubstituted or substituted C₃₋₈ heterocyclyl, unsubstituted or substituted aryl, and unsubstituted or substituted heteroaryl, and
R⁹ is H or C₁₋₁₀ alkyl which is unsubstituted or substituted with one or more substituents independently selected from the group consisting of unsubstituted or substituted C₃₋₆ cycloalkyl, unsubstituted or substituted C₃₋₈ heterocyclyl, unsubstituted or substituted aryl, and unsubstituted or substituted heteroaryl;
X is O or S; and
Y is O or S.

7. A PHD inhibitor according to clause 6, for use as defined in said clause, wherein
R¹ is unsubstituted or substituted C₃₋₈ cycloalkyl;
R² is OH;
R³ is H;
R⁴ is unsubstituted or substituted C₃₋₈ cycloalkyl;
X is O; and
Y is O.

8. A PHD inhibitor according to clause 5 or clause 6, for use as defined in said clause, wherein the PHD inhibitor is a compound of formula (IIa) or a pharmaceutically acceptable salt thereof

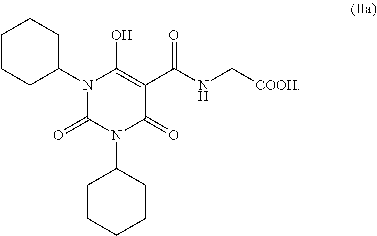 [SMILES: O=C(O)CNC(=O)c1c(O)n(C2CCCCC2)c(=O)n(C2CCCCC2)c1=O]

9. A PHD inhibitor according to any one of clauses 1 to 3, for use as defined in said clause, wherein said PHD inhibitor is a compound of formula (III) or a pharmaceutically acceptable salt thereof

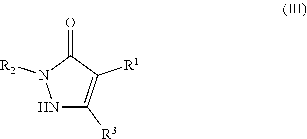

in which
R¹ represents a heteroaryl group of the formula

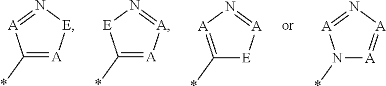

wherein * denotes the linkage point with the dihydropyrazolone ring,
A in each individual occurrence denotes C—R⁴ or N, wherein at most two ring members A represent N at the same time, and
E denotes O, S or N—R⁵,
R² represents a heteroaryl group of the formula

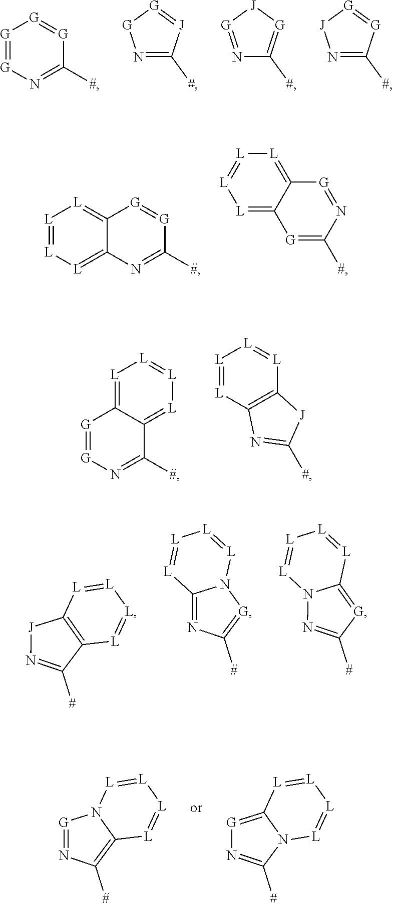

wherein #denotes the linkage point with the dihydropyrazolone ring, G in each individual occurrence denotes C—R⁶ or N, wherein at most two ring members G represent N at the same time,
   J denotes O, S or N—R⁷, and
   L in each individual occurrence denotes C—R⁸ or N, wherein at most two ring members L represent N at the same time,
   wherein R⁴, R⁶ and R⁸ are the same or different and are each independently selected from H or a substituent chosen from the series consisting of halogen, —CN, nitro, C₁₋₆ alkyl, —C₃₋₇-cycloalkyl, 4-to 10-membered heterocyclyl, phenyl, 5 or 6-membered heteroaryl, —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹, wherein
   (i) C₁₋₆ alkyl is unsubstituted or substituted one to three times by the same or different groups independently selected from halogen, CN, oxo, —C₃₋₇-cycloalkyl, 4-to 10-membered heterocyclyl, phenyl, 5-or 6-membered heteroaryl-C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹, wherein the cycloalkyl, heterocyclyl, phenyl and heteroaryl groups may unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl,
   (ii) C₃₋₇ cycloalkyl, 4-to 10-membered heterocyclyl, phenyl and 5-or 6-membered heteroaryl may be unsubstituted or substituted one to three times by the same or different groups independently selected from halogen, —CN, nitro, C₁₋₆ alkyl, —C₃₋₇-cycloalkyl, 4-to 10 membered heterocyclyl, phenyl, 5 or 6-membered heteroaryl, —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹,
   wherein the alkyl group is unsubstituted or substituted one to three times by the same or different groups independently selected from halogen, CN, hydroxyl, trifluoromethoxy, (C₁₋₄)-alkoxy, amino, mono-(C₁₋₄)-alkylamino, di-(C₁₋₄)-alkylamino, hydroxycarbonyl, (C₁₋₄)-oxycarbonyl, (C₃₋₇)-cycloalkyl, 4-to 7-membered heterocyclyl, phenyl and/or 5-or 6-membered heteroaryl,
   (iii) R⁹, R¹⁰, R¹¹, R¹³, R¹⁴, R¹⁷, R¹⁹, R²¹, R²⁴, R²⁵, R²⁶, R²⁸, R²⁹ and R³⁰ independently of one another for each individual occurrence represent groups selected from H, C₁₋₆ alkyl, C₃₋₇ cycloalkyl, 4-to 10-membered heterocyclyl, phenyl and 5-or 6-membered heteroaryl, wherein
   C₃₋₇ cycloalkyl, 4-to 10-membered heterocyclyl, phenyl and 5-or 6-membered heteroaryl may be unsubstituted or substituted one to three times by the same or different groups independently selected from halogen, CN, C₁₋₄-alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl and
   C₁₋₆ alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl, (C₁₋₄)-alkoxycarbonyl, C₃₋₇ cycloalkyl, C₄₋₇ heterocyclyl, phenyl and/or 5-or 6-membered heteroaryl,
   (iv) R¹², R¹⁵, R¹⁶, R¹⁸, R²⁰, R²², R²³, R²⁷ and R³¹ independently of one another for each individual occurrence represent groups selected from H and C₁₋₆ alkyl,
   wherein C₁₋₆ alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl and/or (C₁₋₄)-oxycarbonyl, and/or wherein
   (v) R¹¹ and R¹², R¹⁴ and R¹⁵, R¹⁶ and R¹⁷, R¹⁸ and R¹⁹, R²⁰ and R²¹, R²¹ and R²², R²³ and R²⁴, R²⁶ and R²⁷ and R³⁰ and R³¹ in each case paired together with the atoms to which they are bonded can form a 5-or 6-membered heterocyclyl ring, which may be unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl,
and
   R⁵ and R⁷ are the same or different and independently and are each selected from H, C₁₋₆ alkyl, C₃₋₇ cycloalkyl, C₄₋₇ heterocyclyl, phenyl and 5-or 6-membered heteroaryl, wherein
   (i) C₁₋₆ alkyl is unsubstituted or substituted one to three times by the same or different groups independently selected from halogen, —CN, nitro, —C₃₋₇-cycloalkyl, 4-to 10-membered heterocyclyl, phenyl, 5 or 6-membered heteroaryl, —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹, wherein the cycloalkyl, heterocyclyl, phenyl and heteroaryl groups may be unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-oxycarbonyl, and
   (ii) C₃₋₇ cycloalkyl, 4 to 7 membered heterocyclyl, phenyl and 5-or 6-membered heteroaryl are unsubstituted or substituted one to three times by the same or different groups independently selected from C₁₋₆ alkyl, halogen, —CN, nitro, —C₃₋₇-cycloalkyl, 4-to 10-membered heterocyclyl, phenyl, 5 or 6-membered heteroaryl, —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, SR²⁹ and —NR³⁰R³¹
   wherein the alkyl group is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl, C₁₋₄ oxycarbonyl, C₃₋₇ cycloalkyl, 4-to 7-membered heterocyclyl, phenyl and/or 5-or 6-membered heteroaryl,
   wherein
   (a) R⁹, R¹⁰, R¹¹, R¹³, R¹⁴, R¹⁷, R¹⁹, R²¹, R²⁴, R²⁵, R²⁶, R²⁸, R²⁹ and R³⁰ independently of one another for each individual occurrence represent a group selected from H, C₁₋₆ alkyl, C₃₋₇ cycloalkyl, 4-to 7-membered heterocyclyl, phenyl and 5-or 6-membered heteroaryl, wherein
   C₃₋₇ cycloalkyl, 4-to 7-membered heterocycloalkyl, phenyl and 5-or 6-membered heteroaryl are unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-oxycarbonyl, and
   (C₁₋₆)-alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl, C₁₋₄ oxycarbonyl, C₃₋₇ cycloalkyl, 4-to 7-membered heterocyclyl, phenyl and/or 5-or 6-membered heteroaryl
   (b) R¹², R¹⁵, R¹⁶, R¹⁸, R²⁰, R²², R²³, R²⁷ and R³¹ independently of one another for each individual occurrence represent a group selected from H and C₁₋₆ alkyl,
   wherein C₁₋₆ alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl and/or (C₁₋₄)-oxycarbonyl, and/or
   (c) R¹¹ and R¹², R¹⁴ and R¹⁵, R¹⁶ and R¹⁷, R¹⁸ and R¹⁹, R²⁰ and R²¹, R²¹ and R²², R²³ and R²⁴, R²⁶ and R²⁷ and R³⁰ and R³¹ in each case paired together with the atoms to which they are bonded can form a 5-or 6-membered heterocyclyl ring, which can be unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-oxycarbonyl, and
   R³ represents H, C₁₋₆ alkyl or C₃₋₇ cycloalkyl.

10. A PHD inhibitor according to clause 9, for use as defined in said clause, wherein
R¹ denotes a group of the formula

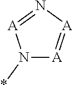

wherein * denotes the linkage point with the dihydropyrazolone ring,
A in each individual occurrence denotes CH or N, wherein at most two ring members A represent N at the same time, and
wherein R² represent a heteroaryl group of the formula

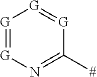

wherein #denotes the linkage point with the dihydropyrazolone ring, G in each individual occurrence denotes C—R⁶ or N, wherein at most two ring members G represent N at the same time,
wherein R⁶ represent H or 5 or 6-membered heterocyclyl, and
wherein R³ is H.

11. A PHD inhibitor according to clause 9 or clause 10, for use as defined in said clause, wherein the PHD inhibitor is a compound of formula (IIIa) or a pharmaceutically acceptable salt thereof

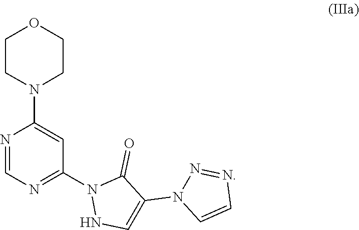 [SMILES: O=c1c(-n2ccnn2)c[nH]n1-c1cc(N2CCOCC2)ncn1]

12. A compound which is a substituted azine of formula (I) or a pharmaceutically acceptable salt thereof

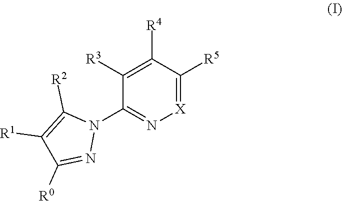

wherein
X is CR⁶ or N;
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —N(R^{t})C(O)N(R^{u})(R^{v}), —CN, —C(O)OR^{w} or —C(O)N(R^{x})R⁷;
R² is H, —OR^{q} or unsubstituted or substituted C₁₋₆ alkyl; and R³ is H, —OR⁸ or unsubstituted or substituted C₁₋₆ alkyl; or R² is —N═ and R³ is ═C(R^{y})— and R² and
R³ together form a group of formula —N═C(R^{y})—;
R⁴ is H, unsubstituted or substituted C₁₋₆ alkyl, —OR⁹ or —C(O)OR¹⁰;
R⁵ is H, unsubstituted or substituted C₁₋₆ alkyl, unsubstituted or substituted aryl, unsubstituted or substituted heteroaryl, —N(R^{t})C(O)N(R^{u})(R^{v}), —CN, —C(O)OR^{w}, or —C(O)N(R^{x})R⁷;
R⁶ is H or unsubstituted or substituted C₁₋₆ alkyl;
R⁷ is —CH(R¹¹)—Ar, —CH(R¹¹)-Ary-Ar, -Ary-Ar, CH(R¹¹)-Cyc, —CH₂C≡CCH₃, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR² or unsubstituted or substituted C₁₋₄ alkyl;
R⁸, R⁰ and R¹⁰ are each independently selected from H and unsubstituted or substituted C₁₋₆ alkyl; and
R^{t}, R^{u}, R^{v}, R^{w}, R^{x}, R^{y}, and R^{z} are each independently selected from H, unsubstituted or substituted C₁₋₆ alkyl, and unsubstituted or substituted phenyl; and
R^{q} is H, unsubstituted or substituted C₁₋₆ alkyl, or unsubstituted or substituted phenyl;
provided that one of R¹ and R⁵ is —C(O)N(R^{x})R⁷ and the other of R¹ and R⁵ is H, unsubstituted or substituted C₁₋₆ alkyl, unsubstituted or substituted aryl, unsubstituted or substituted heteroaryl, —N(R^{t})C(O)N(R^{u})(R^{v}), —CN or —C(O)OR^{w}.

13. A compound according to clause 12 wherein:
R⁰ is H or unsubstituted C₁₋₆ alkyl;
R¹ is H, —CN, —C(O)OR^{w} or —C(O)N(R^{x})R⁷;
R² is H, OH or unsubstituted C₁₋₆ alkyl; and R³ is H or —OR⁸; or R² is —N═ and R³ is ═C(R^{y}) and R² and R³ together form a group of formula —N═C(R^{y});
R⁴ is H, —OR⁹ or —C(O)OR¹⁰;
R⁵ is H, —CN, —C(O)OR^{w}, or —C(O)N(R^{x})R⁷;
R⁶ is H;
R⁷ is —CH(R¹¹)—Ar, —CH(R¹¹)-Ary-Ar, -Ary-Ar or —CH(R¹¹) Cyc, wherein Ar is unsubstituted or substituted aryl or unsubstituted or substituted heteroaryl, Ary is unsubstituted arylene or unsubstituted heteroarylene, Cyc is unsubstituted or substituted C₃₋₁₀ cycloalkyl, and R¹¹ is H, —C(O)OR² or unsubstituted C₁₋₄ alkyl;
R⁸, R⁹ and R¹⁰ are each independently selected from H and unsubstituted or substituted C₁₋₆ alkyl; and
R^{x} is H, R^{z} is H, R^{y} is H or unsubstituted C₁₋₆ alkyl, and R^{w} is H, unsubstituted C₁₋₆ alkyl, or C₁₋₆ alkyl which is substituted with phenyl or —OC(O)R^{ww} wherein R^{ww} is phenyl, unsubstituted C₁₋₆ alkyl, —N(R^{a})(R^{b}), C(O)Re, —OR^{d} or an amino acid, wherein R^{a}, R^{b}, R^{c} and R^{d} are each independently selected from H, unsubstituted or substituted C₁₋₆ alkyl and an amino acid;
provided that one of R¹ and R⁵ is —C(O)N(R^{x})R⁷ and the other of R¹ and R⁵ is H, —CN or —C(O)OR^{w}.

14. A compound according to clause 12 or clause 13 wherein:
R⁰ is H or methyl;
R¹ is H, —CN, —C(O)OR^{w}, or —C(O)N(R^{x})R⁷;
R² is H or methyl; and R³ is H or —OR⁸; or R² is —N═ and R³ is ═C(R^{y})— and R² and R³ together form a group of formula —N═C(R^{y})—;
R⁴ is H, —OR⁹ or —C(O)OR¹⁰;
R⁵ is H, —CN, —C(O)OR^{w}, or —C(O)N(R^{x})R⁷;
R⁶ is H;
R⁷ is —CH(R¹¹)—Ar, —CH(R¹¹)-Ary-Ar, -Ary-Ar or —CH(R¹¹)-Cyc; wherein Ar is unsubstituted phenyl, unsubstituted pyrimidyl, unsubstituted benzothiazole or phenyl substituted with —C(O)OH, —C(O)OMe, —C(O)OEt, —C(O)NH₂, —C(O)N(H)Me, —OMe or N-morpholino; Ary is unsubstituted phenylene or unsubstituted pyridylene; Cyc is unsubstituted cyclohexyl or cyclohexyl substituted with —CF₃ or —OCF₃; and R¹¹ is H, —C(O)OR^{z} or methyl;
R⁸, R⁹ and R¹⁰ are each independently selected from H, unsubstituted C₁₋₆ alkyl, and C₁₋₆ alkyl which is substituted with phenyl or —OC(O)R⁹⁹ wherein R⁹⁹ is phenyl, unsubstituted C₁₋₆ alkyl, —N(R^{a})(R^{b}), —C(O)R^{c}, —OR^{d} or an amino acid, wherein R^{a}, R^{b}, R^{c} and R^{d} are each independently selected from H, unsubstituted or substituted C₁₋₆ alkyl and an amino acid; and
R^{x} is H;
R^{z} is H;
R^{w} is H, unsubstituted C₁₋₆ alkyl, or C₁₋₆ alkyl which is substituted with phenyl or —OC(O)R^{ww} wherein R^{ww} is phenyl or unsubstituted C₁₋₆ alkyl; and
R^{y} is H or methyl;
provided that one of R¹ and R⁵ is —C(O)N(R^{x})R⁷ and the other of R¹ and R⁵ is H, —CN or —C(O)OR^{w}.

15. A compound according to any one of clauses 12 to 14 wherein either:
(1) (a) R⁵ is —C(O)N(R^{x})R⁷ and (b) R³ is —OR⁸ or R⁴ is —OR⁹; or
(2) (a) R¹ is —C(O)N(R^{x})R⁷ and (b) R⁴ is —OR⁹ or —C(O)OR¹⁰, or R⁵ is —C(O)OR^{w}.

16. A compound according to any one clauses 12 to 15 wherein the substituted azine has the formula (Ia)

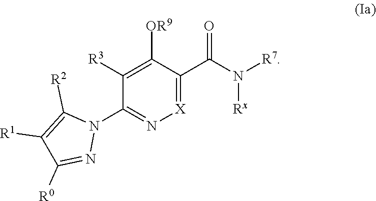

wherein
X is CR⁶ or N;
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —CN or —C(O)OR^{w};
R² is H, —OR^{q} or unsubstituted or substituted C₁₋₆ alkyl;
R³ is H or unsubstituted or substituted C₁₋₆ alkyl;
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, —CH₂C≡CCH₃, Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR² or unsubstituted or substituted C₁₋₄ alkyl;
R⁹ is H or unsubstituted or substituted C₁₋₆ alkyl;
R^{w}, R^{x} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.

17. A compound according to clause 16 wherein R⁹ is H.

18. A compound according to any one of clauses 12 to 17 wherein the substituted azine has any one of the following structures:

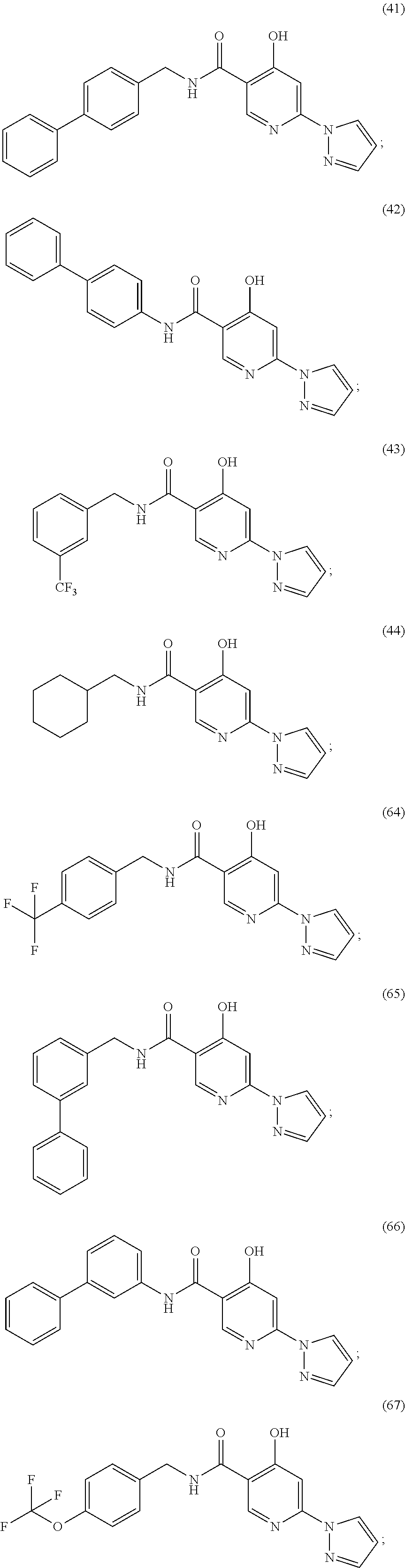

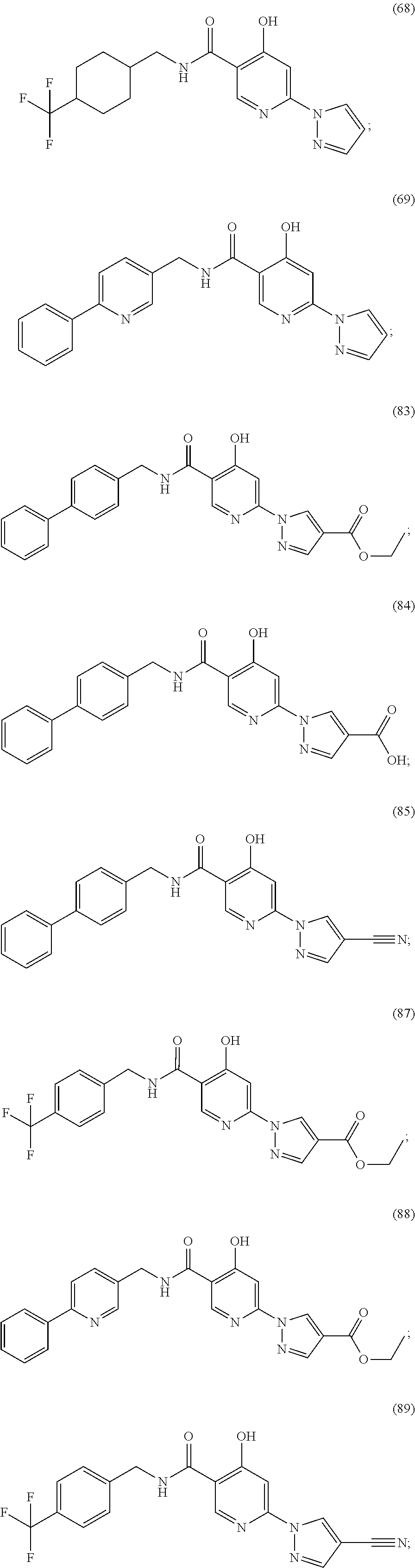

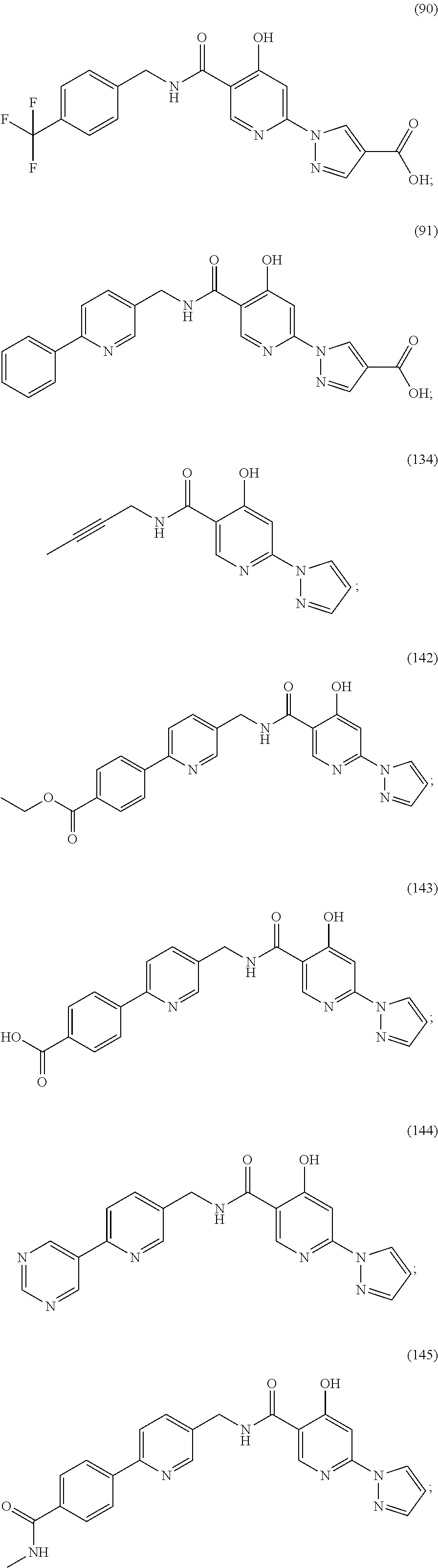

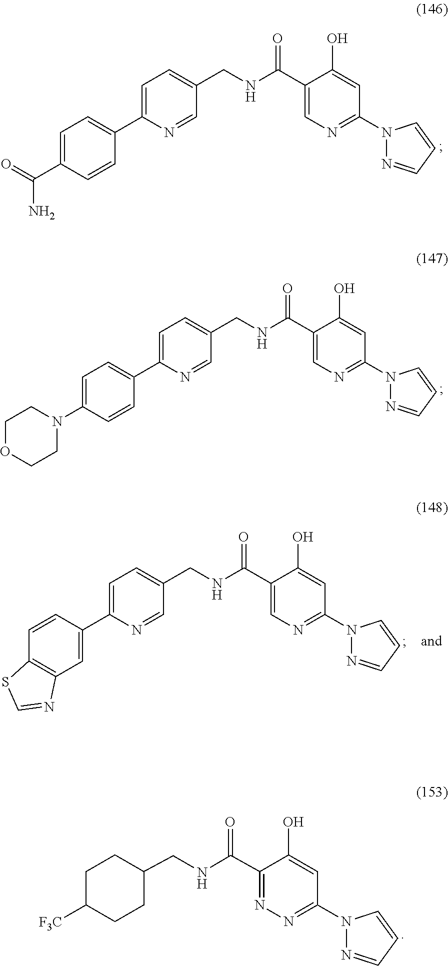 [SMILES: NC(=O)c1ccc(-c2ccc(CNC(=O)c3cnc(-n4cccn4)cc3O)cn2)cc1 ; O=C(NCc1ccc(-c2ccc(N3CCOCC3)cc2)nc1)c1cnc(-n2cccn2)cc1O ; O=C(NCc1ccc(-c2ccc3scnc3c2)nc1)c1cnc(-n2cccn2)cc1O ; O=C(NCC1CCC(C(F)(F)F)CC1)c1nnc(-n2cccn2)cc1O]

19. A compound according to any one of clauses 12 to 15 wherein the substituted azine has the formula (Ib)

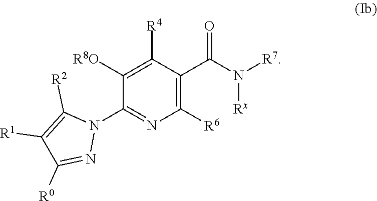

wherein
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —CN or —C(O)OR^{w};
R² is H, —OR^{q} or unsubstituted or substituted C₁₋₆ alkyl;
R⁴ is H or unsubstituted or substituted C₁₋₆ alkyl;
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and
R¹¹ is H, —C(O)OR^{z} or unsubstituted or substituted C₁₋₄ alkyl;
R⁸ is H or unsubstituted or substituted C₁₋₄ alkyl;
R^{w}, R^{x} and R^{z} are each independently selected from H, unsubstituted or substituted C₁₋₄ alkyl, and unsubstituted or substituted phenyl; and
R^{q} is H, unsubstituted or substituted C₁₋₄ alkyl, or unsubstituted or substituted phenyl.

20. A compound according to clause 19 wherein R⁸ is H.

21. A compound according to clause 12 or clause 20 wherein the substituted azine has any one of the following structures

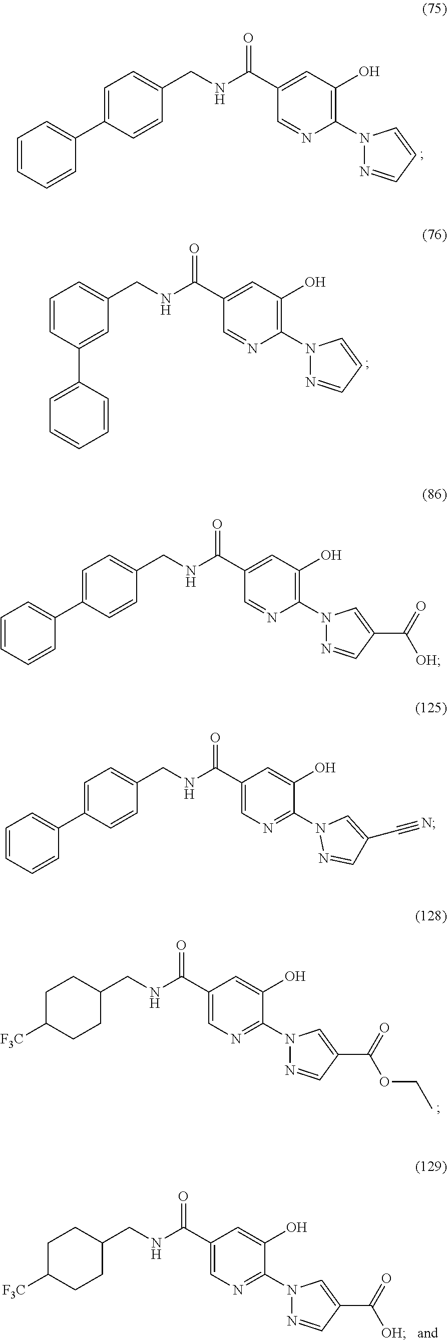

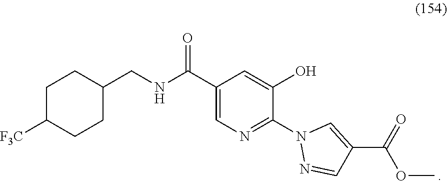 [SMILES: COC(=O)c1cnn(-c2ncc(C(=O)NCC3CCC(C(F)(F)F)CC3)cc2O)c1 ; "."]

22. A compound according to any one of clauses 12 to 15 wherein the substituted azine has the formula (Ic)

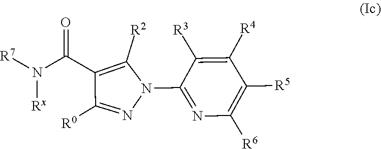

wherein
R⁰ is H or unsubstituted or substituted C₁₋₆ alkyl;
R² is H, —OR^{q} or unsubstituted or substituted C₁₋₆ alkyl;
R³ is H, —OR⁸ or unsubstituted or substituted C₁₋₆ alkyl;
R⁴ is H, unsubstituted or substituted C₁₋₆ alkyl, —OR⁹ or —C(O)OR¹⁰;
R⁵ is H, unsubstituted or substituted C₁₋₆ alkyl, unsubstituted or substituted aryl, unsubstituted or substituted heteroaryl, —CN or —C(O)OR^{w};
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and
R¹¹ is H, —C(O)OR² or unsubstituted or substituted C₁₋₄ alkyl;
R⁸, R⁹ and R¹⁰ are each independently selected from H and unsubstituted or substituted C₁₋₄ alkyl;
R^{w}, R^{x} and R^{z} are each independently selected from H, unsubstituted or substituted C₁₋₄ alkyl, and unsubstituted or substituted phenyl;
R^{q} is H, unsubstituted or substituted C₁₋₄ alkyl, or unsubstituted or substituted phenyl.

23. A compound according to clause 22 wherein (i) R⁴ is OH or C(O)OH, and/or (ii) R⁵ is C(O)OH.

24. A compound according to clause 22 or clause 23 wherein the substituted azine has any one of the following structures:

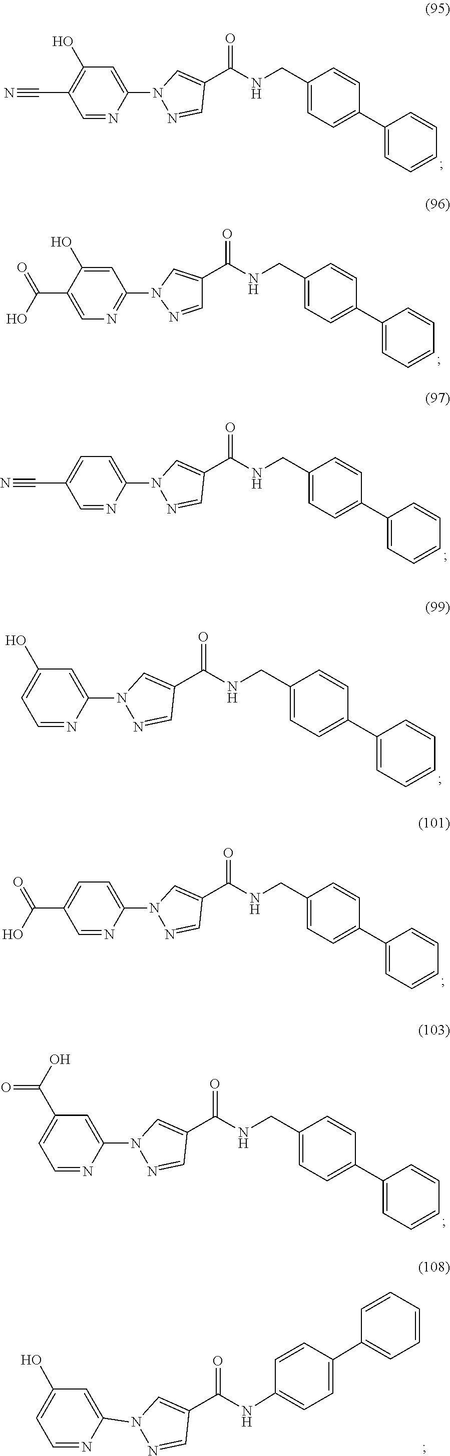

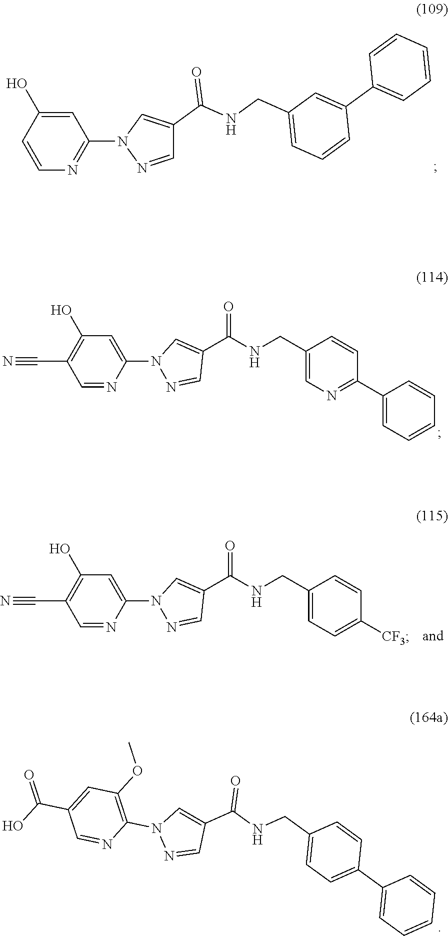

25. A compound according to any one of clauses 12 to 15 wherein the substituted azine has the formula (Id)

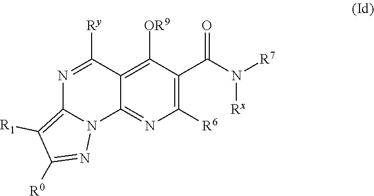

wherein
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —CN or —C(O)OR^{w};
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and
R¹¹ is H, —C(O)OR^{z} or unsubstituted or substituted C₁₋₄ alkyl;
R⁹ is H or unsubstituted or substituted C₁₋₆ alkyl; and
R^{w}, R^{x}, R^{y}, and R^{z} are each independently selected from H, unsubstituted or substituted C₁₋₄ alkyl, and unsubstituted or substituted phenyl.

26. A compound according to clause 25 wherein R⁹ is H.

27. A compound according to clause 25 or clause 26 wherein the substituted azine has any one of the following structures:

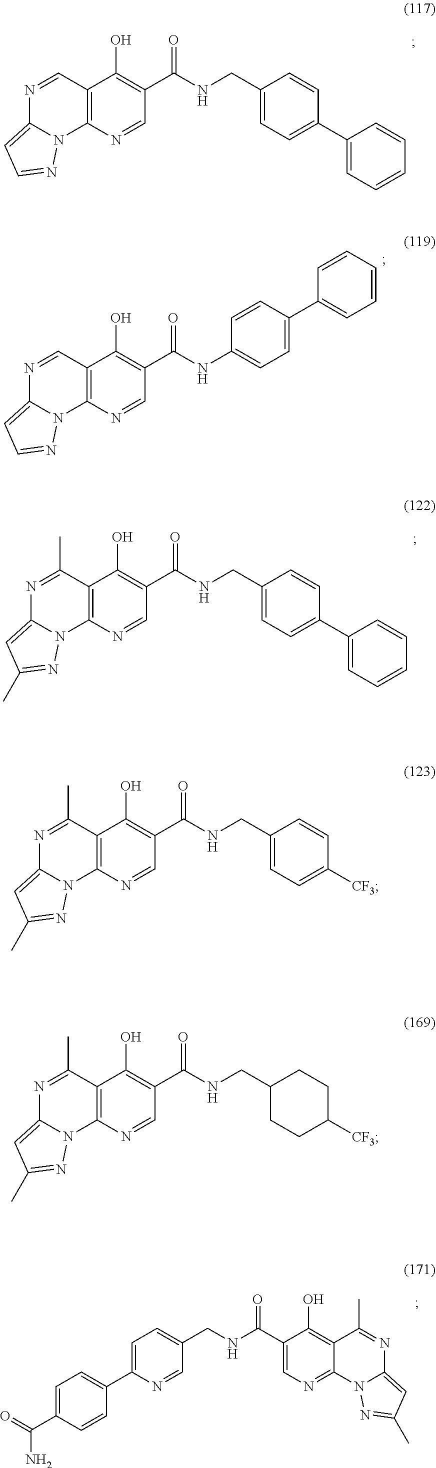 [SMILES: O=C(NCc1ccc(-c2ccccc2)cc1)c1cnc2c(cnc3ccnn32)c1O ; O=C(Nc1ccc(-c2ccccc2)cc1)c1cnc2c(cnc3ccnn32)c1O ; Cc1cc2nc(C)c3c(O)c(C(=O)NCc4ccc(-c5ccccc5)cc4)cnc3n2n1 ; Cc1cc2nc(C)c3c(O)c(C(=O)NCc4ccc(C(F)(F)F)cc4)cnc3n2n1 ; Cc1cc2nc(C)c3c(O)c(C(=O)NCC4CCC(C(F)(F)F)CC4)cnc3n2n1 ; Cc1cc2nc(C)c3c(O)c(C(=O)NCc4ccc(-c5ccc(C(N)=O)cc5)nc4)cnc3n2n1]

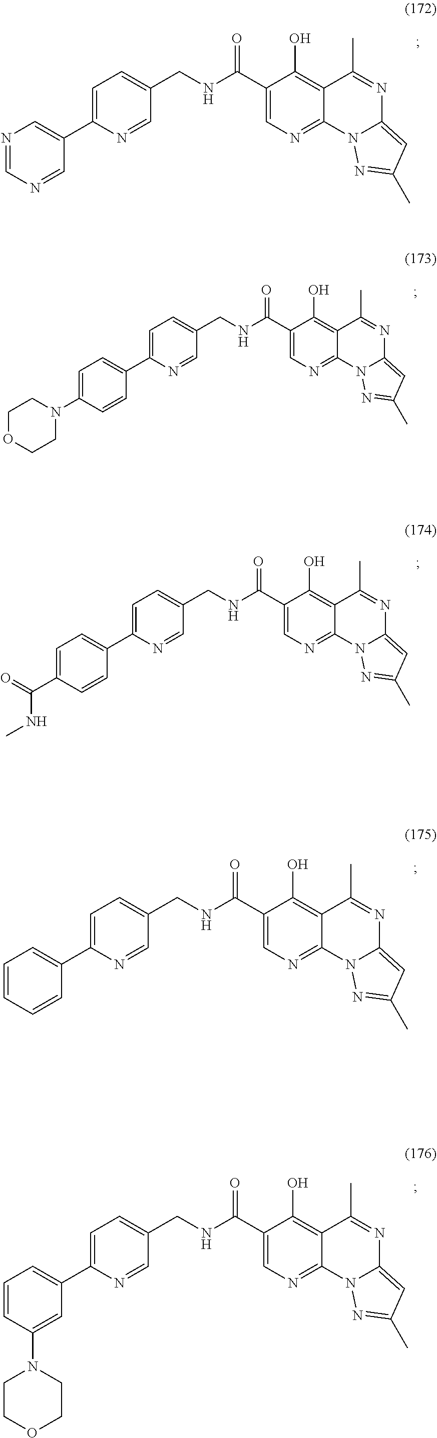 [SMILES: Cc1cc2nc(C)c3c(O)c(C(=O)NCc4ccc(-c5cncnc5)nc4)cnc3n2n1 ; Cc1cc2nc(C)c3c(O)c(C(=O)NCc4ccc(-c5ccc(N6CCOCC6)cc5)nc4)cnc3n2n1 ; CNC(=O)c1ccc(-c2ccc(CNC(=O)c3cnc4c(c(C)nc5cc(C)nn54)c3O)cn2)cc1 ; Cc1cc2nc(C)c3c(O)c(C(=O)NCc4ccc(-c5ccccc5)nc4)cnc3n2n1 ; Cc1cc2nc(C)c3c(O)c(C(=O)NCc4ccc(-c5cccc(N6CCOCC6)c5)nc4)cnc3n2n1]

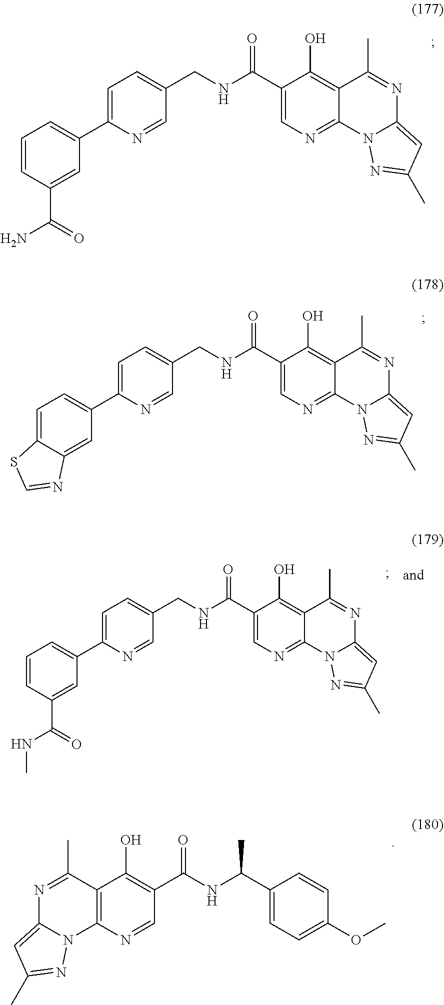

28. A compound according to any one of clauses 12 to 14 wherein the substituted azine has any one of the following structures:

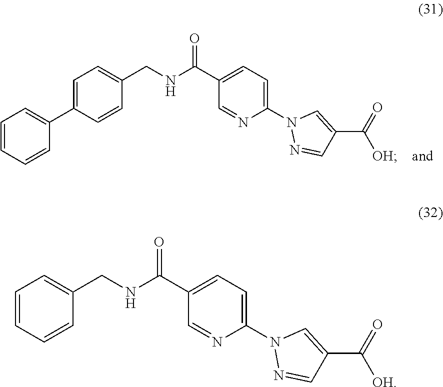 [SMILES: O=C(O)c1cnn(-c2ccc(C(=O)NCc3ccc(-c4ccccc4)cc3)cn2)c1 ; "and" ; O=C(O)c1cnn(-c2ccc(C(=O)NCc3ccccc3)cn2)c1]

29. A compound according to clause 16 wherein R⁹ is unsubstituted or substituted C₁₋₄alkyl, optionally wherein the substituted azine of formula (Ia) has any one of the following structures

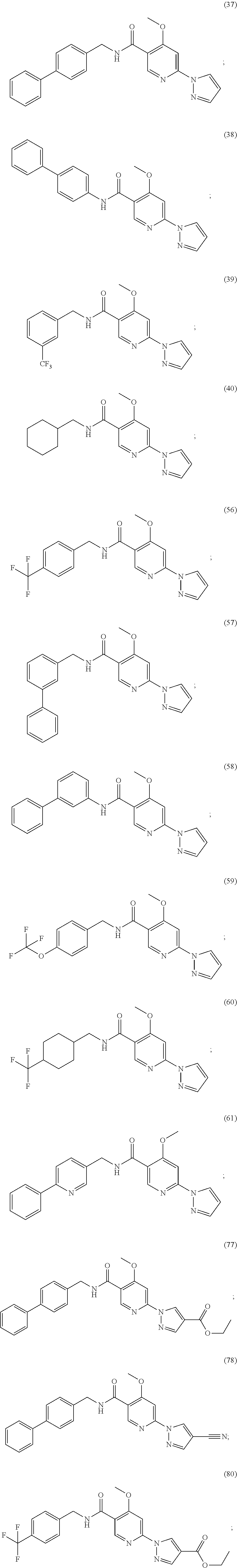

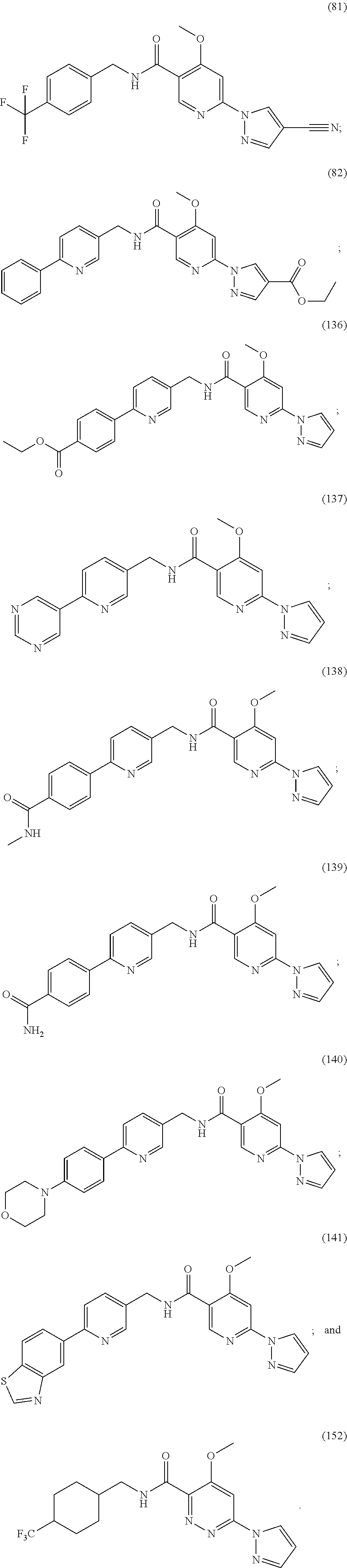

30. A compound according to clause 19 wherein R⁸ is unsubstituted or substituted C₁₋₆ alkyl, optionally wherein the substituted azine of formula (Ib) has any one of the following structures

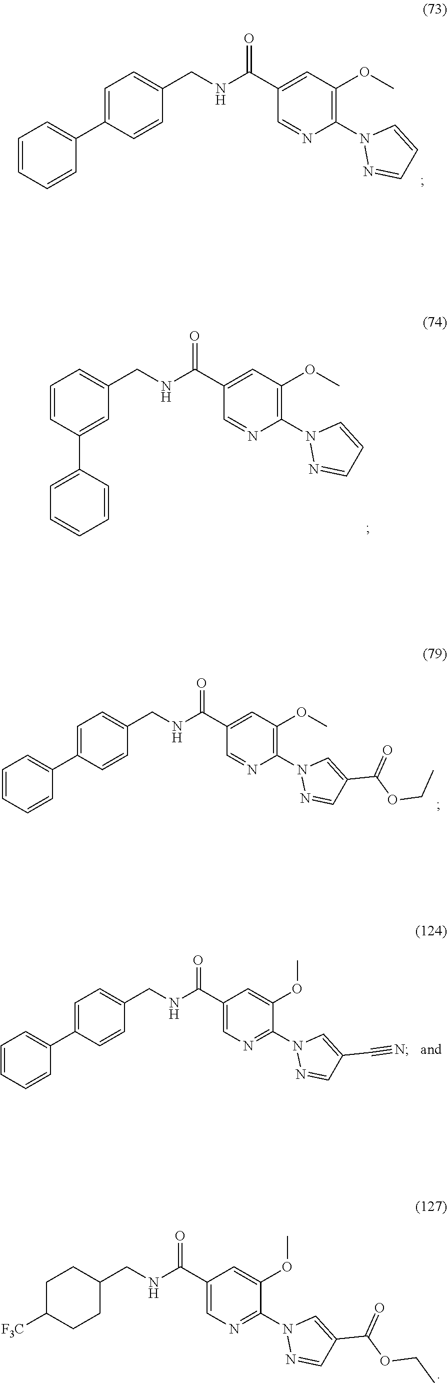 [SMILES: COc1cc(C(=O)NCc2ccc(-c3ccccc3)cc2)cnc1-n1cccn1 ; COc1cc(C(=O)NCc2cccc(-c3ccccc3)c2)cnc1-n1cccn1 ; CCOC(=O)c1cnn(-c2ncc(C(=O)NCc3ccc(-c4ccccc4)cc3)cc2OC)c1 ; COc1cc(C(=O)NCc2ccc(-c3ccccc3)cc2)cnc1-n1cc(C#N)cn1 ; CCOC(=O)c1cnn(-c2ncc(C(=O)NCC3CCC(C(F)(F)F)CC3)cc2OC)c1]

31. A compound according to clause 22 wherein R⁴ is —OR⁹ or —C(O)OR¹⁰, and/or R⁵ is —C(O)OR^{w}, wherein each of R⁹, R¹⁰ and R¹¹ is independently unsubstituted or substituted C₁₋₆ alkyl; optionally wherein the substituted azine of formula (Ic) has any one of the following structures

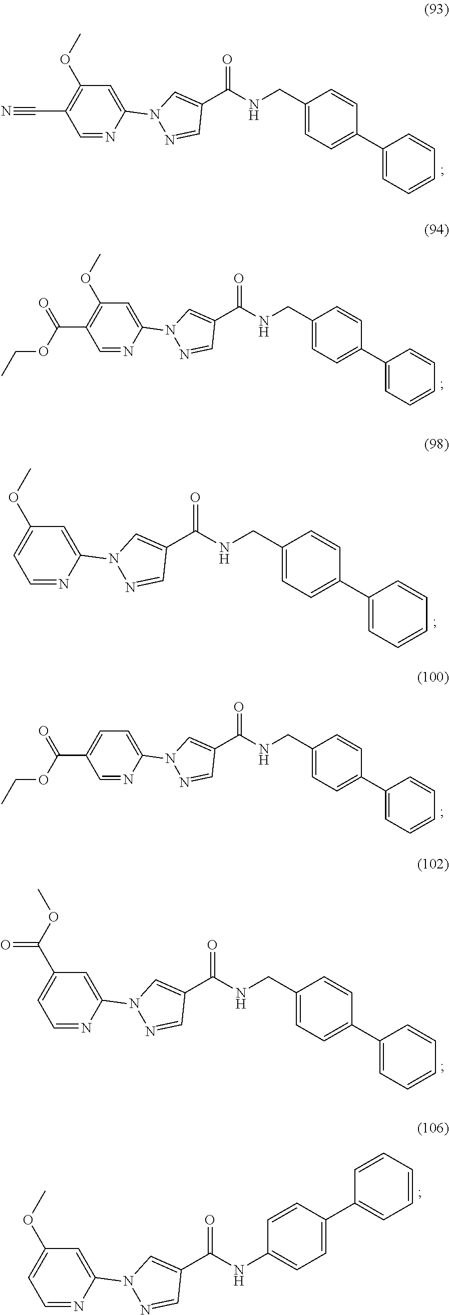 [SMILES: COc1cc(-n2cc(C(=O)NCc3ccc(-c4ccccc4)cc3)cn2)ncc1C#N ; CCOC(=O)c1cnc(-n2cc(C(=O)NCc3ccc(-c4ccccc4)cc3)cn2)cc1OC ; COc1ccnc(-n2cc(C(=O)NCc3ccc(-c4ccccc4)cc3)cn2)c1 ; CCOC(=O)c1ccc(-n2cc(C(=O)NCc3ccc(-c4ccccc4)cc3)cn2)nc1 ; COC(=O)c1ccnc(-n2cc(C(=O)NCc3ccc(-c4ccccc4)cc3)cn2)c1 ; COc1ccnc(-n2cc(C(=O)Nc3ccc(-c4ccccc4)cc3)cn2)c1]

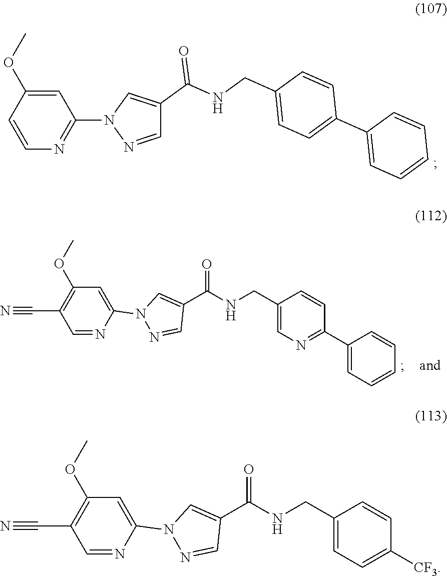 [SMILES: COc1ccnc(-n2cc(C(=O)NCc3ccc(-c4ccccc4)cc3)cn2)c1 ; COc1cc(-n2cc(C(=O)NCc3ccc(-c4ccccc4)nc3)cn2)ncc1C#N ; COc1cc(-n2cc(C(=O)NCc3ccc(C(F)(F)F)cc3)cn2)ncc1C#N]

32. A compound according to clause 25 wherein R⁹ is unsubstituted or substituted C₁₋₆ alkyl; optionally wherein the substituted azine of formula (Id) has the following structure

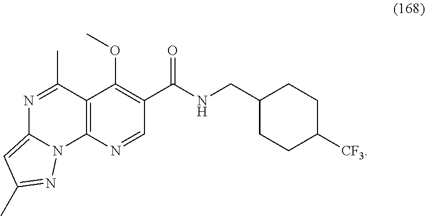 [SMILES: COc1c(C(=O)NCC2CCC(C(F)(F)F)CC2)cnc2c1c(C)nc1cc(C)nn12]

33. A compound which is a substituted pyrimidine of formula (IV) or a pharmaceutically acceptable salt thereof

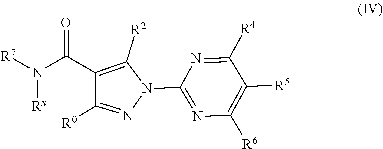

wherein
   R⁰ is H or unsubstituted or substituted C₁₋₆ alkyl;
   R² is H, —OR^{q} or unsubstituted or substituted C₁₋₆ alkyl;
      R⁴ is —OR⁹
      R⁵ is H, unsubstituted or substituted C₁₋₆ alkyl, unsubstituted or substituted aryl, unsubstituted or substituted heteroaryl, —CN or —C(O)OR^{w};
         R⁶ is H or unsubstituted or substituted C₁₋₆ alkyl;
      R⁷ is —CH(R¹¹)—Ar, —CH(R¹¹)-Ary-Ar, -Ary-Ar, —CH(R¹¹) Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR² or unsubstituted or substituted C₁₋₄ alkyl;
         R⁹ is selected from H and unsubstituted or substituted C₁₋₆ alkyl;
         R^{x} is H, unsubstituted or substituted C₁₋₄ alkyl, or unsubstituted or substituted phenyl;
         R^{w} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.

34. A compound according to clause 33 wherein R⁴ is OH, and preferably wherein R⁴ is OH and R⁵ is CN.

35. A compound according to clause 33 or clause 34 wherein the substituted pyrimidine has any one of the following structures

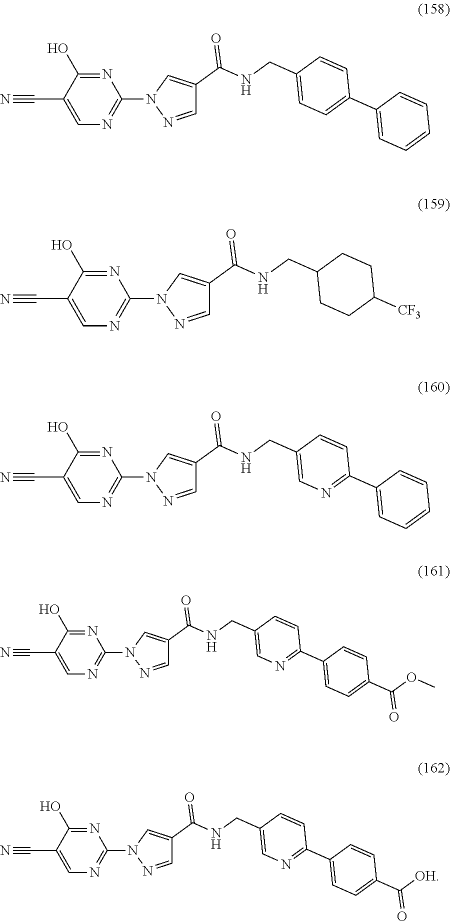 [SMILES: N#Cc1cnc(-n2cc(C(=O)NCc3ccc(-c4ccccc4)cc3)cn2)nc1O ; N#Cc1cnc(-n2cc(C(=O)NCC3CCC(C(F)(F)F)CC3)cn2)nc1O ; N#Cc1cnc(-n2cc(C(=O)NCc3ccc(-c4ccccc4)nc3)cn2)nc1O ; COC(=O)c1ccc(-c2ccc(CNC(=O)c3cnn(-c4ncc(C#N)c(O)n4)c3)cn2)cc1 ; N#Cc1cnc(-n2cc(C(=O)NCc3ccc(-c4ccc(C(=O)O)cc4)nc3)cn2)nc1O]

36. A pharmaceutical composition comprising a compound as defined in any one of clauses 12 to 35 and a pharmaceutically acceptable carrier or diluent.

37. A compound as defined in any one of clauses 12 to 35, or a pharmaceutical composition as defined in clause 36, for use in treating the human or animal body by therapy.

38. A compound as defined in any one of clauses 12 to 35, or a pharmaceutical composition as defined in clause 36, for use in treating blood cancer.

39. A PHD inhibitor as defined in any one of clauses 5 to 11, or a compound as defined in any one of clauses 12 to 35, or a pharmaceutical composition as defined in clause 36, for use in a method of treating blood cancer, wherein said method comprises (i) administering said PHD inhibitor, compound or pharmaceutical composition and (ii) subsequently, sequentially, or simultaneously administering one or more biologically active agents for use in the treatment of blood cancer.

40. A HIF-alpha increasing agent for use in treating blood cancer.

41. A HIF-alpha increasing agent according to clause 40, for use as defined in said clause, wherein the HIF-alpha increasing agent is: a PHD inhibitor as defined in any of clauses 5 to 11, a compound as defined in any one of clauses 12 to 35, a double stranded RNA, a small interfering RNA, CRISPR and guide RNA, a zinc finger protein, a transcription activator-like effector nuclease, a designer receptor exclusively activated by designer drugs, an amino acid, a signalling molecule, a peptide, a protein, an antibody, a nucleic acid, an oligonucleotide, or a cell.

42. A hypoxia inducible factor prolyl hydroxylase inhibitor (PHD inhibitor) for use in the treatment of blood cancer by simultaneous, separate or sequential co-administration with a factor inhibiting HIF inhibitor (FIH inhibitor).

43. A PHD inhibitor for use according to clause 42 wherein the PHD inhibitor is a PHD inhibitor as defined in any of clauses 5 to 11 or a compound as defined in any one of clauses 12 to 35.

44. A PHD inhibitor for use according to clause 42 or clause 43 wherein the PHD inhibitor is a compound of the following formula or a pharmaceutically acceptable salt thereof

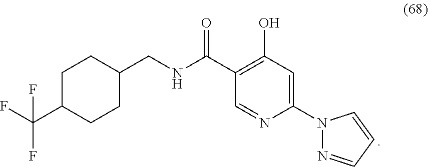 [SMILES: O=C(NCC1CCC(C(F)(F)F)CC1)c1cnc(-n2cccn2)cc1O]

45. A PHD inhibitor for use according to any one of clauses 42 to 44 wherein the FIH inhibitor is dimethyl N-oxalyl-D-phenylalanine (DM-NOFD) or a pharmaceutically acceptable salt thereof.

46. A PHD inhibitor for use according to any one of clauses 42 to 45 wherein the treatment of blood cancer further comprises administration of a B-cell lymphoma 2 (BCL2) inhibitor, optionally wherein the BCL2 inhibitor is venetoclax or a pharmaceutically acceptable salt thereof.

47. A factor inhibiting HIF inhibitor (FIH inhibitor) for use in the treatment of blood cancer by simultaneous, separate or sequential co-administration with a hypoxia inducible factor prolyl hydroxylase inhibitor (PHD inhibitor).

48. An FIH inhibitor for use according to clause 47 wherein the FIH inhibitor is dimethyl N-oxalyl-D-phenylalanine (DM-NOFD) or a pharmaceutically acceptable salt thereof.

49. An FIH inhibitor for use according to clause 47 or clause 48 wherein the PHD inhibitor is a PHD inhibitor as defined in any of clauses 5 to 11 or a compound as defined in any one of clauses 12 to 35
50. An FIH inhibitor for use according to any one of clauses 47 to 49 wherein the PHD inhibitor is a compound of the following formula or a pharmaceutically acceptable salt thereof

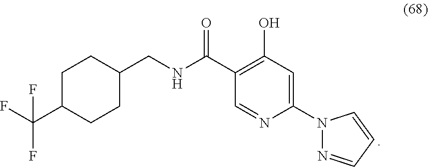 [SMILES: O=C(NCC1CCC(C(F)(F)F)CC1)c1cnc(-n2cccn2)cc1O]

51. An FIH inhibitor for use according to any one of clauses 47 to 50 wherein the treatment of blood cancer further comprises administration of a BCL2 inhibitor, optionally wherein the BCL2 inhibitor is venetoclax or a pharmaceutically acceptable salt thereof.

52. A combination comprising a hypoxia inducible factor prolyl hydroxylase inhibitor (PHD inhibitor) and a factor inhibiting HIF inhibitor (FIH inhibitor).

53. A combination according to clause 52 wherein the PHD inhibitor is as further defined in clause 43 or 44, and/or wherein the FIH inhibitor is as further defined in clause 45.

54. A combination according to clause 52 or clause 53 which further comprises a BCL2 inhibitor, optionally wherein the BCL2 inhibitor is venetoclax or a pharmaceutically acceptable salt thereof.

55. A combination according to any one of clauses 52 to 54, for use in the treatment of blood cancer.

56. A pharmaceutical composition comprising a hypoxia inducible factor prolyl hydroxylase inhibitor (PHD inhibitor), a factor inhibiting HIF inhibitor (FIH inhibitor), and a pharmaceutically acceptable carrier or diluent.

57. A pharmaceutical composition according to clause 56 wherein the PHD inhibitor is as further defined in clause 43 or 44, and/or wherein the FIH inhibitor is as further defined in clause 45.

58. A pharmaceutical composition according to clause 56 or clause 57 which further comprises a BCL2 inhibitor, optionally wherein the BCL2 inhibitor is venetoclax or a pharmaceutically acceptable salt thereof.

59. A pharmaceutical composition according to any one of clauses 56 to 58, for use in the treatment of blood cancer.

60. A hypoxia inducible factor prolyl hydroxylase inhibitor (PHD inhibitor) for use in the treatment of blood cancer by simultaneous, separate or sequential co-administration with a BCL2 inhibitor.

61. A PHD inhibitor for use according to clause 60 wherein the PHD inhibitor is as further defined in clause 43 or 44.

62. A PHD inhibitor for use according to clause 60 or clause 61 wherein the BCL2 inhibitor is venetoclax or a pharmaceutically acceptable salt thereof.

63. A BCL2 inhibitor for use in the treatment of blood cancer by simultaneous, separate or sequential co-administration with a hypoxia inducible factor prolyl hydroxylase inhibitor (PHD inhibitor).

64. A BCL2 inhibitor for use according to clause 63 wherein the PHD inhibitor is as further defined in clause 43 or 44.

65. A BCL2 inhibitor for use according to clause 63 or clause 64 wherein the BCL2 inhibitor is venetoclax or a pharmaceutically acceptable salt thereof.

66. A combination comprising a hypoxia inducible factor prolyl hydroxylase inhibitor (PHD inhibitor) and a BCL2 inhibitor.

67. A combination according to clause 66 wherein the PHD inhibitor is as further defined in clause 43 or 44, and/or wherein the BCL2 inhibitor is venetoclax or a pharmaceutically acceptable salt thereof.

68. A combination according to clause 66 or clause 67, for use in the treatment of blood cancer.

69. A pharmaceutical composition comprising a hypoxia inducible factor prolyl hydroxylase inhibitor (PHD inhibitor), a BCL2 inhibitor, and a pharmaceutically acceptable carrier or diluent.

70. A pharmaceutical composition according to clause 69 wherein the PHD inhibitor is as further defined in clause 43 or 44, and/or wherein the BCL2 inhibitor is venetoclax or a pharmaceutically acceptable salt thereof.

71. A pharmaceutical composition according to clause 69 or clause 70, for use in the treatment of blood cancer.

72. A PHD inhibitor, FIH inhibitor, BCL2 inhibitor, combination or pharmaceutical composition as defined in any one of clauses 42 to 51, 55, 59 to 65, 68 and 71 optionally wherein the blood cancer is is acute myeloid leukaemia (AML), chronic myeloid leukaemia (CML) or multiple myeloma (MM).

1. A hypoxia inducible factor prolyl hydroxylase inhibitor (PHD inhibitor) for use in the treatment of blood cancer.

2. A PHD inhibitor according to claim 1, for use as defined in said claim, wherein the blood cancer is acute myeloid leukaemia (AML), chronic myeloid leukaemia (CML) or multiple myeloma (MM).

3. A PHD inhibitor according to claim 1 or claim 2, for use as defined in said claim, wherein said treatment comprises binding of the PHD inhibitor to the active site of prolyl hydroxlase domain (PHD), wherein the PHD inhibitor competes with 2-oxoglutarate for binding to said active site, and optionally wherein the PHD inhibitor competes with HIF-alpha for binding to said active site.

4. A PHD inhibitor according to any one of claims 1 to 3, for use as defined in said claim, wherein the PHD inhibitor is a compound as defined in any one of claims 10 to 22.

5. A PHD inhibitor according to any one of claims 1 to 3, for use as defined in said claim, wherein the PHD inhibitor is a cobalt compound, for instance a cobalt salt (e.g. cobalt dichloride); a copper compound, for instance a copper salt; a nickel compound, for instance a nickel salt; an iron chelator, such as deferoxamine, 3,4-dihydroxybenzoic acid, 1,10-phenanthrolines, or quercetin; a 2-OG derivative mimic, or competitor (with respect to PHD binding), such as dimethyloxalylglycine (DMOG) which is a prodrug form of N-oxalylglycine (NOG); FG-2216; roxadustat; a quinolone, such as JNJ-42905343; a quinoxaline; a benzamidazole derivative, such as JNJ-42041935; an isoquinolone derivative; a 5-hydroxy-1,7 naphthyridine derivative, such as ISM5411; a monocyclic pyridine compound, such as vadadustat, and AKB6899; a pyrazolopyrimidine derivative; a pyrimidine-trione, such as daprodustat; an N-alkoxyquinolone, such as desidustat; a tetrahydropyran derivative; a dihydrothienopyridone derivative; a dihydrofuropyridoene derivative; a quinazoline-2,4-dione; a 4-oxo-2-thioxo-7-quinasoline; a 5-aminocarbonyl-4-hydroxypyrimidine derivative, such as MK8617; a spiroindolone; a 2,8-diazaspori[4,5]-decan-lone; a pyrazolone derivative, such as molidustat; a triazole substituted heteroaryl amide; a phenolic compound, such as ((S)-{2 [2-(5-cyano-3-hydroxy-pyridin-2-yl)-thiazol-4-yl]-acetylamino}-phenyl-acetic acid); a bicyclic heteroaryl derivative, such as (1,2,4-triazolo-[1,5-a]pyridine); a diacylhydrazine; pyrathione Zn, or (5-(3-(4-chlorophenoxyl) prop-1-yn-1-yl)-3-hydroxypicolinoyl)glycine.

6. A PHD inhibitor according to any one of claims 1 to 3, for use as defined in said claim, wherein said PHD inhibitor is a compound of formula (II) or a pharmaceutically acceptable salt thereof 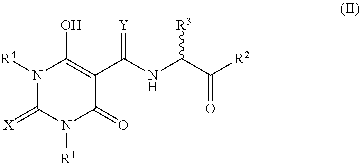
wherein
R¹ and R⁴ are each independently selected from the group consisting of H, —NR⁵R⁶, unsubstituted or substituted C₁₋₁₀ alkyl, unsubstituted or substituted C₂₋₁₀ alkenyl, unsubstituted or substituted C₂₋₁₀ alkynyl, unsubstituted or substituted C₃₋₈ cycloalkyl, unsubstituted or substituted-C₃₋₈ cycloalkylene-C₁₋₁₀ alkyl, unsubstituted or substituted C₅₋₈ cycloalkenyl, unsubstituted or substituted —C₅₋₈ cycloalkenylene-C₁₋₁₀ alkyl, unsubstituted or substituted C₃₋₈ heterocyclyl, unsubstituted or substituted —C₃₋₈ heterocyclylene-C₁₋₁₀ alkyl, unsubstituted or substituted aryl, unsubstituted or substituted-arylene-C₁₋₁₀ alkyl, unsubstituted or substituted-heteroaryl and unsubstituted or substituted-heteroarylene-C₁₋₁₀ alkyl;
R² is —NR⁷R⁸ or —OR⁹;
R³ is H or unsubstituted or substituted C₁₋₄ alkyl;
where R⁵ and R⁶ are each independently selected from the group consisting of H, unsubstituted or substituted C₁₋₁₀ alkyl, unsubstituted or substituted-C₃₋₈ cycloalkyl, unsubstituted or substituted —C₃₋₈ cycloalkylene-C₁₋₁₀ alkyl, C₃₋₈ heterocyclyl, unsubstituted or substituted —C₃₋₈ heterocyclylene-C₁₋₁₀ alkyl, unsubstituted or substituted aryl, unsubstituted or substituted-arylene-C₁₋₁₀ alkyl, unsubstituted or substituted heteroaryl, unsubstituted or substituted-heteroarylene-C₁₋₁₀ alkyl, unsubstituted or substituted —C(O)C₁₋₄ alkyl, unsubstituted or substituted —C(O)C₃₋₆ cycloalkyl, —C(O)C₃₋₆ heterocyclyl, unsubstituted or substituted —C(O) aryl, unsubstituted or substituted —C(O)heteroaryl and unsubstituted or substituted —S(O)₂C₁₋₄ alkyl, or, when R⁵ and R⁶ are attached to the same nitrogen, R⁵ and R⁶ taken together with the nitrogen to which they are attached form a 5-or 6-or 7-membered saturated heterocyclic ring which is unsubstituted or substituted and which optionally contains one other heteroatom selected from oxygen, nitrogen and sulphur,
R⁷ and R⁸ are each independently selected from the group consisting of H, unsubstituted or substituted C₁₋₁₀ alkyl, unsubstituted or substituted C₂₋₁₀ alkenyl, unsubstituted or substituted C₂₋₁₀ alkynyl, unsubstituted or substituted C₃₋₈ cycloalkyl, unsubstituted or substituted C₃₋₈ heterocyclyl, unsubstituted or substituted aryl, and unsubstituted or substituted heteroaryl, and
R⁹ is H or C₁₋₁₀ alkyl which is unsubstituted or substituted with one or more substituents independently selected from the group consisting of unsubstituted or substituted C₃₋₆ cycloalkyl, unsubstituted or substituted C₃₋₈ heterocyclyl, unsubstituted or substituted aryl, and unsubstituted or substituted heteroaryl;
X is O or S; and
Y is O or S.

7. A PHD inhibitor according to claim 6, for use as defined in said claim, wherein the PHD inhibitor is a compound of formula (IIa) or a pharmaceutically acceptable salt thereof

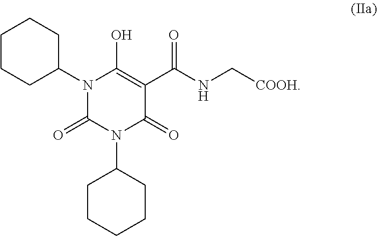 [SMILES: O=C(O)CNC(=O)c1c(O)n(C2CCCCC2)c(=O)n(C2CCCCC2)c1=O]

8. A PHD inhibitor according to any one of claims 1 to 3, for use as defined in said claim, wherein said PHD inhibitor is a compound of formula (III) or a pharmaceutically acceptable salt thereof 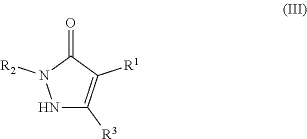
in which
R¹ represents a heteroaryl group of the formula 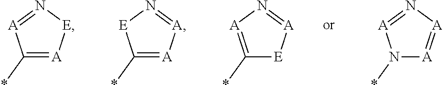
wherein * denotes the linkage point with the dihydropyrazolone ring,
A in each individual occurrence denotes C—R⁴ or N, wherein at most two ring members A represent N at the same time, and
E denotes O, S or N—R⁵,
R² represents a heteroaryl group of the formula 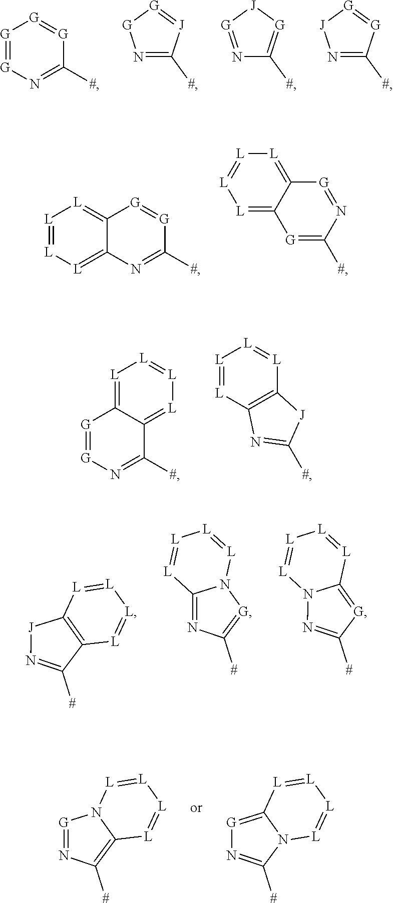
wherein #denotes the linkage point with the dihydropyrazolone ring, G in each individual occurrence denotes C—R⁶ or N, wherein at most two ring members G represent N at the same time,
J denotes O, S or N—R⁷, and
L in each individual occurrence denotes C—R⁸ or N, wherein at most two ring members L represent N at the same time,
wherein R⁴, R⁶ and R⁸ are the same or different and are each independently selected from H or a substituent chosen from the series consisting of halogen, —CN, nitro, C₁₋₆ alkyl, —C₃₋₇-cycloalkyl, 4-to 10-membered heterocyclyl, phenyl, 5 or 6-membered heteroaryl, —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹, wherein
(i) C₁₋₆ alkyl is unsubstituted or substituted one to three times by the same or different groups independently selected from halogen, CN, oxo, —C₃₋₇-cycloalkyl, 4-to 10-membered heterocyclyl, phenyl, 5-or 6-membered heteroaryl —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹,
wherein the cycloalkyl, heterocyclyl, phenyl and heteroaryl groups may unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl,
(ii) C₃₋₇ cycloalkyl, 4-to 10-membered heterocyclyl, phenyl and 5-or 6-membered heteroaryl may be unsubstituted or substituted one to three times by the same or different groups independently selected from halogen, —CN, nitro, C₁₋₆ alkyl, —C₃₋₇-cycloalkyl, 4-to 10 membered heterocyclyl, phenyl, 5 or 6-membered heteroaryl, —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹,
wherein the alkyl group is unsubstituted or substituted one to three times by the same or different groups independently selected from halogen, —CN, hydroxyl, trifluoromethoxy, (C₁₋₄)-alkoxy, amino, mono-(C₁₋₄)-alkylamino, di-(C₁₋₄)-alkylamino, hydroxycarbonyl, (C₁₋₄)-oxycarbonyl, (C₃₋₇)-cycloalkyl, 4-to 7-membered heterocyclyl, phenyl and/or 5-or 6-membered heteroaryl,
(iii) R⁹, R¹⁰, R¹¹, R¹³, R¹⁴, R¹⁷, R¹⁹, R²¹, R²⁴, R²⁵, R²⁶, R²⁸, R²⁹ and R³⁰ independently of one another for each individual occurrence represent groups selected from H, C₁₋₆ alkyl, C₃₋₇ cycloalkyl, 4-to 10-membered heterocyclyl, phenyl and 5-or 6-membered heteroaryl, wherein
C₃₋₇ cycloalkyl, 4-to 10-membered heterocyclyl, phenyl and 5-or 6-membered heteroaryl may be unsubstituted or substituted one to three times by the same or different groups independently selected from halogen, CN, C₁₋₄-alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl and
C₁₋₆ alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl, (C₁₋₄)-alkoxycarbonyl, C₃₋₇ cycloalkyl, C₄₋₇ heterocyclyl, phenyl and/or 5-or 6-membered heteroaryl,
(iv) R¹², R¹⁵, R¹⁶, R¹⁸, R²⁰, R²², R²³, R²⁷ and R³¹ independently of one another for each individual occurrence represent groups selected from H and C₁₋₆ alkyl,
wherein C₁₋₆ alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl and/or (C₁₋₄)-oxycarbonyl,
and/or wherein
(v) R¹¹ and R¹², R¹⁴ and R¹⁵, R¹⁶ and R¹⁷, R¹⁸ and R¹⁹, R²⁰ and R²¹, R²¹ and R²², R²³ and R²⁴, R²⁶ and R²⁷ and R³⁰ and R³¹ in each case paired together with the atoms to which they are bonded can form a 5-or 6-membered heterocyclyl ring, which may be unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-alkoxycarbonyl,
and
R⁵ and R⁷ are the same or different and independently and are each selected from H, C₁₋₆ alkyl, C₃₋₇ cycloalkyl, C₄₋₇ heterocyclyl, phenyl and 5-or 6-membered heteroaryl, wherein
(i) C₁₋₆ alkyl is unsubstituted or substituted one to three times by the same or different groups independently selected from halogen, —CN, nitro, —C₃₋₇-cycloalkyl, 4-to 10-membered heterocyclyl, phenyl, 5 or 6-membered heteroaryl, —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, —SR²⁹ and —NR³⁰R³¹,
wherein the cycloalkyl, heterocyclyl, phenyl and heteroaryl groups may be unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-oxycarbonyl, and
(ii) C₃₋₇ cycloalkyl, 4 to 7 membered heterocyclyl, phenyl and 5-or 6-membered heteroaryl are unsubstituted or substituted one to three times by the same or different groups independently selected from C₁₋₆ alkyl, halogen, —CN, nitro, —C₃₋₇-cycloalkyl, 4-to 10-membered heterocyclyl, phenyl, 5 or 6-membered heteroaryl, —C(O)R⁹, —C(O)OR¹⁰, —C(O)NR¹¹R¹², —OC(O)R¹³, —OC(O)NR¹⁴R¹⁵, —NR¹⁶C(O)R¹⁷, —NR¹⁸C(O)OR¹⁹, —NR²⁰C(O)NR²¹R²², —NR²³SO₂R²⁴, —SO₂R²⁵, —SO₂NR²⁶R²⁷, —OR²⁸, SR²⁹ and —NR³⁰R³¹,
wherein the alkyl group is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl, C₁₋₄ oxycarbonyl, C₃₋₇ cycloalkyl, 4-to 7-membered heterocyclyl, phenyl and/or 5-or 6-membered heteroaryl,
wherein
(a) R⁹, R¹⁰, R¹¹, R¹³, R¹⁴, R¹⁷, R¹⁹, R²¹, R²⁴, R²⁵, R²⁶, R²⁸, R²⁹ and R³⁰ independently of one another for each individual occurrence represent a group selected from H, C₁₋₆ alkyl, C₃₋₇ cycloalkyl, 4-to 7-membered heterocyclyl, phenyl and 5-or 6-membered heteroaryl, wherein
C₃₋₇ cycloalkyl, 4-to 7-membered heterocycloalkyl, phenyl and 5-or 6-membered heteroaryl are unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-oxycarbonyl, and
(C₁₋₆)-alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl, C₁₋₄ oxycarbonyl, C₃₋₇ cycloalkyl, 4-to 7-membered heterocyclyl, phenyl and/or 5-or 6-membered heteroaryl
(b) R¹², R¹⁵, R¹⁶, R¹⁸, R²⁰, R²², R²³, R²⁷ and R³¹ independently of one another for each individual occurrence represent a group selected from H and C₁₋₆ alkyl,
wherein C₁₋₆ alkyl is unsubstituted or substituted one to three times by the same or different groups independently selected from halogen, CN, hydroxyl, trifluoromethoxy, C₁₋₄ alkoxy, amino, mono-(C₁₋₄)-alkylamino, di-(C₁₋₄)-alkylamino, hydroxycarbonyl and/or (C₁₋₄)-oxycarbonyl, and/or
(c) R¹¹ and R¹², R¹⁴ and R¹⁵, R¹⁶ and R¹⁷, R¹⁸ and R¹⁹, R²⁰ and R²¹, R²¹ and R²², R²³ and R²⁴, R²⁶ and R²⁷ and R³⁰ and R³¹ in each case paired together with the atoms to which they are bonded can form a 5-or 6-membered heterocyclyl ring, which can be unsubstituted or substituted one to three times by the same or different groups independently selected from halogen, CN, C₁₋₄ alkyl, trifluoromethyl, hydroxyl, C₁₋₄ alkoxy, trifluoromethoxy, oxo, amino, mono-(C₁₋₄)-alkylamino, di-(C₁₋₄)-alkylamino, hydroxycarbonyl and/or (C₁₋₄)-oxycarbonyl, and
R³ represents H, C₁₋₆ alkyl or C₃₋₇ cycloalkyl.

9. A PHD inhibitor according to claim 8, for use as defined in said claim, wherein the PHD inhibitor is a compound of formula (IIIa) or a pharmaceutically acceptable salt thereof

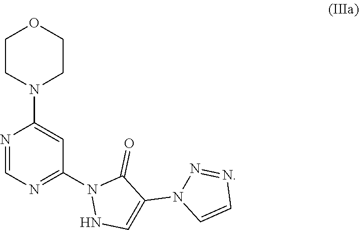 [SMILES: O=c1c(-n2ccnn2)c[nH]n1-c1cc(N2CCOCC2)ncn1]

10. A compound which is a substituted azine of formula (I) or a pharmaceutically acceptable salt thereof 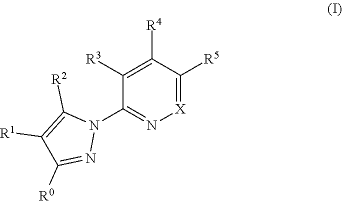
wherein
X is CR⁶ or N;
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —N(R^{t})C(O)N(R^{u})(R^{v}), —CN, —C(O)OR^{w} or —C(O)N(R^{x})R⁷;
R² is H, —OR^{q} or unsubstituted or substituted C₁₋₆ alkyl; and R³ is H, —OR⁸ or unsubstituted or substituted C₁₋₆ alkyl; or R² is —N═ and R³ is ═C(R^{y})— and R² and R³ together form a group of formula —N═C(R^{y})—;
R⁴ is H, unsubstituted or substituted C₁₋₆ alkyl, —OR⁹ or —C(O)OR¹⁰;
R⁵ is H, unsubstituted or substituted C₁₋₆ alkyl, unsubstituted or substituted aryl, unsubstituted or substituted heteroaryl, —N(R^{t})C(O)N(R^{u})(R^{v}), —CN, —C(O)OR^{w}, or —C(O)N(R^{x})R⁷;
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, —CH₂C≡CCH₃, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR² or unsubstituted or substituted C₁₋₄ alkyl;
R⁸, R⁹ and R¹⁰ are each independently selected from H and unsubstituted or substituted C₁₋₆ alkyl; and
R^{t}, R^{u}, R^{v}, R^{w}, R^{x}, R^{y}, and R^{z} are each independently selected from H, unsubstituted or substituted C₁₋₆ alkyl, and unsubstituted or substituted phenyl; and
R^{q} is H, unsubstituted or substituted C₁₋₆ alkyl, or unsubstituted or substituted phenyl;
provided that one of R¹ and R⁵ is —C(O)N(R^{x})R⁷ and the other of R¹ and R⁵ is H, unsubstituted or substituted C₁₋₆ alkyl, unsubstituted or substituted aryl, unsubstituted or substituted heteroaryl, —N(R^{t})C(O)N(R^{u})(R^{v}), —CN or —C(O)OR^{w}.

11. A compound according to claim 10 wherein:
R⁰ is H or unsubstituted C₁₋₆ alkyl;
R¹ is H, —CN, —C(O)OR^{w} or —C(O)N(R^{x})R⁷;
R² is H, OH or unsubstituted C₁₋₆ alkyl; and R³ is H or —OR⁸; or R² is —N═ and R³ is ═C(R^{y}) and R² and R³ together form a group of formula —N═C(R^{y})—;
R⁴ is H, —OR⁹ or —C(O)OR¹⁰;
R⁵ is H, —CN, —C(O)OR^{w}, or —C(O)N(R^{x})R⁷;
R⁶ is H;
R⁷ is —CH(R¹¹)—Ar, —CH(R¹¹)-Ary-Ar, -Ary-Ar or —CH(R¹¹)-Cyc, wherein Ar is unsubstituted or substituted aryl or unsubstituted or substituted heteroaryl, Ary is unsubstituted arylene or unsubstituted heteroarylene, Cyc is unsubstituted or substituted C₃₋₁₀ cycloalkyl, and R¹¹ is H, —C(O)OR² or unsubstituted C₁₋₄ alkyl;
R⁸, R⁹ and R¹⁰ are each independently selected from H and unsubstituted or substituted C₁₋₆ alkyl; and
R^{x} is H, R^{z} is H, R^{y} is H or unsubstituted C₁₋₆ alkyl, and R^{w} is H, unsubstituted C₁₋₆ alkyl, or C₁₋₆ alkyl which is substituted with phenyl or —OC(O)R^{ww} wherein R^{ww} is phenyl, unsubstituted C₁₋₆ alkyl, —N(R^{a})(R^{b}), —C(O)R^{c}, —OR^{d} or an amino acid, wherein R^{a}, R^{b}, R^{c} and R^{d} are each independently selected from H, unsubstituted or substituted C₁₋₆ alkyl and an amino acid;
provided that one of R¹ and R⁵ is —C(O)N(R^{x})R⁷ and the other of R¹ and R⁵ is H, —CN or —C(O)OR^{w}.

12. A compound according to claim 10 or claim 11 wherein:
R⁰ is H or methyl;
R¹ is H, —CN, —C(O)OR^{w}, or —C(O)N(R^{x})R⁷;
R² is H or methyl; and R³ is H or —OR⁸; or R² is —N═ and R³ is ═C(R^{y})— and R² and R³ together form a group of formula —N═C(R^{y});
R⁴ is H, —OR⁹ or —C(O)OR¹⁰;
R⁵ is H, —CN, —C(O)OR^{w}, or —C(O)N(R^{x})R⁷;
R⁶ is H;
R⁷ is —CH(R¹¹)—Ar, —CH(R¹¹)-Ary-Ar, -Ary-Ar or —CH(R¹¹)-Cyc; wherein Ar is unsubstituted phenyl, unsubstituted pyrimidyl, unsubstituted benzothiazole or phenyl substituted with —C(O)OH, —C(O)OMe, —C(O)OEt, —C(O)NH₂, —C(O)N(H)Me, —OMe or N-morpholino; Ary is unsubstituted phenylene or unsubstituted pyridylene; Cyc is unsubstituted cyclohexyl or cyclohexyl substituted with —CF₃ or —OCF₃; and R¹¹ is H, —C(O)OR^{z} or methyl;
R⁸, R⁰ and R¹⁰ are each independently selected from H, unsubstituted C₁₋₆ alkyl, and C₁₋₆ alkyl which is substituted with phenyl or —OC(O)R⁹⁹ wherein R⁹⁹ is phenyl, unsubstituted C₁₋₆ alkyl, —N(R^{a})(R^{b}), —C(O)R^{c}, —OR^{d} or an amino acid, wherein R^{a}, R^{b},
R^{c} and R^{d} are each independently selected from H, unsubstituted or substituted C₁₋₆ alkyl and an amino acid; and
R^{x} is H;
R^{z} is H;
R^{w} is H, unsubstituted C₁₋₆ alkyl, or C₁₋₆ alkyl which is substituted with phenyl or —OC(O)R^{ww} wherein R^{ww} is phenyl or unsubstituted C₁₋₆ alkyl; and
R^{y} is H or methyl;
provided that one of R¹ and R⁵ is —C(O)N(R^{x})R⁷ and the other of R¹ and R⁵ is H, —CN or —C(O)OR^{w}.

13. A compound according to any one claims 10 to 12 wherein the substituted azine has the formula (Ia) 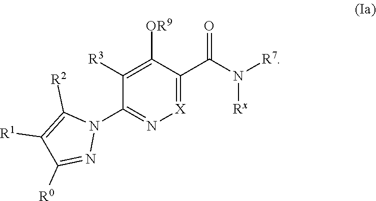
wherein
X is CR⁶ or N;
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —CN or —C(O)OR^{w};
R² is H, —OR^{q} or unsubstituted or substituted C₁₋₆ alkyl;
R³ is H or unsubstituted or substituted C₁₋₆ alkyl;
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, —CH₂C≡CCH₃, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR² or unsubstituted or substituted C₁₋₄ alkyl;
R⁹ is H or unsubstituted or substituted C₁₋₆ alkyl;
R^{w}, R^{x} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.

14. A compound according to any one of claims 10 to 13 wherein the substituted azine has any one of the following structures

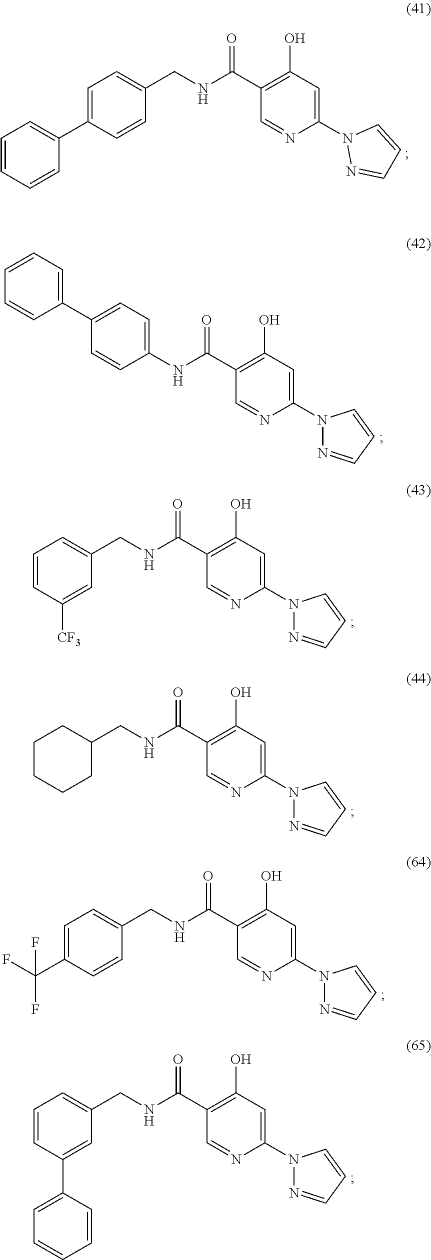 [SMILES: O=C(NCc1ccc(-c2ccccc2)cc1)c1cnc(-n2cccn2)cc1O ; O=C(Nc1ccc(-c2ccccc2)cc1)c1cnc(-n2cccn2)cc1O ; O=C(NCc1cccc(C(F)(F)F)c1)c1cnc(-n2cccn2)cc1O ; O=C(NCC1CCCCC1)c1cnc(-n2cccn2)cc1O ; O=C(NCc1ccc(C(F)(F)F)cc1)c1cnc(-n2cccn2)cc1O ; O=C(NCc1cccc(-c2ccccc2)c1)c1cnc(-n2cccn2)cc1O]

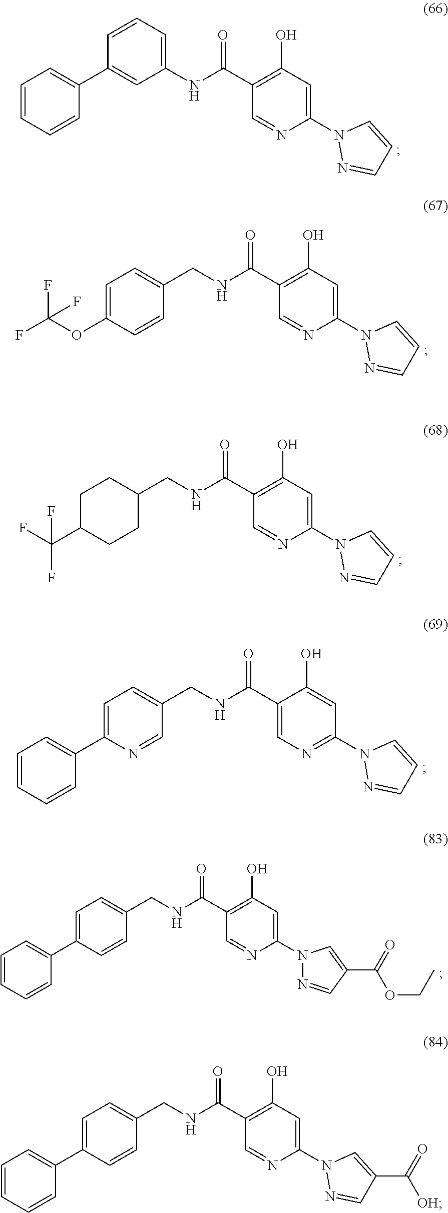 [SMILES: O=C(Nc1cccc(-c2ccccc2)c1)c1cnc(-n2cccn2)cc1O ; O=C(NCc1ccc(OC(F)(F)F)cc1)c1cnc(-n2cccn2)cc1O ; O=C(NCC1CCC(C(F)(F)F)CC1)c1cnc(-n2cccn2)cc1O ; O=C(NCc1ccc(-c2ccccc2)nc1)c1cnc(-n2cccn2)cc1O ; CCOC(=O)c1cnn(-c2cc(O)c(C(=O)NCc3ccc(-c4ccccc4)cc3)cn2)c1 ; O=C(O)c1cnn(-c2cc(O)c(C(=O)NCc3ccc(-c4ccccc4)cc3)cn2)c1]

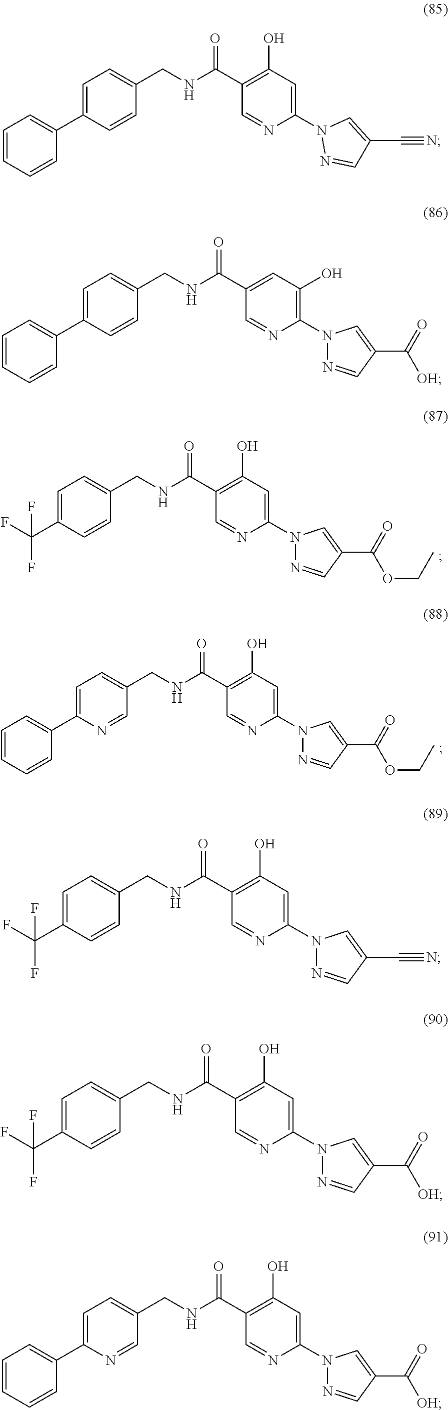

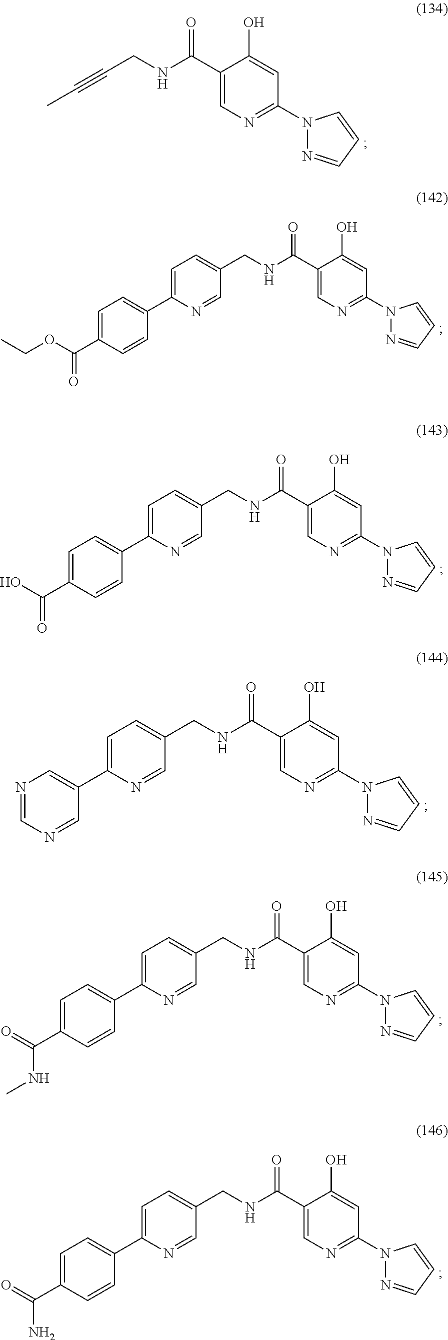 [SMILES: CC#CCNC(=O)c1cnc(-n2cccn2)cc1O ; CCOC(=O)c1ccc(-c2ccc(CNC(=O)c3cnc(-n4cccn4)cc3O)cn2)cc1 ; O=C(O)c1ccc(-c2ccc(CNC(=O)c3cnc(-n4cccn4)cc3O)cn2)cc1 ; O=C(NCc1ccc(-c2cncnc2)nc1)c1cnc(-n2cccn2)cc1O ; CNC(=O)c1ccc(-c2ccc(CNC(=O)c3cnc(-n4cccn4)cc3O)cn2)cc1 ; NC(=O)c1ccc(-c2ccc(CNC(=O)c3cnc(-n4cccn4)cc3O)cn2)cc1]

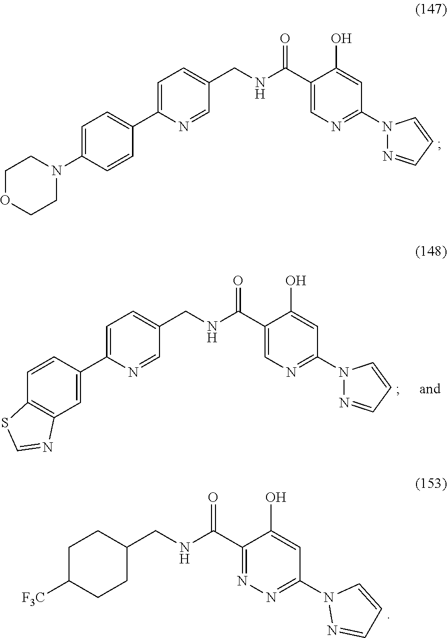 [SMILES: O=C(NCc1ccc(-c2ccc(N3CCOCC3)cc2)nc1)c1cnc(-n2cccn2)cc1O ; O=C(NCc1ccc(-c2ccc3scnc3c2)nc1)c1cnc(-n2cccn2)cc1O ; O=C(NCC1CCC(C(F)(F)F)CC1)c1nnc(-n2cccn2)cc1O]

15. A compound according to any one of claims 10 to 12 wherein the substituted azine has the formula (Ib) 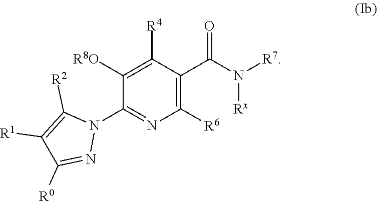
wherein
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —CN or —C(O)OR^{w};
R² is H, —OR^{q} or unsubstituted or substituted C₁₋₆ alkyl;
R⁴ is H or unsubstituted or substituted C₁₋₆ alkyl;
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and
R¹¹ is H, —C(O)OR^{z} or unsubstituted or substituted C₁₋₄ alkyl;
R⁸ is H or unsubstituted or substituted C₁₋₄ alkyl;
R^{w}, R^{x} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.

16. A compound according to claim 10 or claim 15 wherein the substituted azine has any one of the following structures

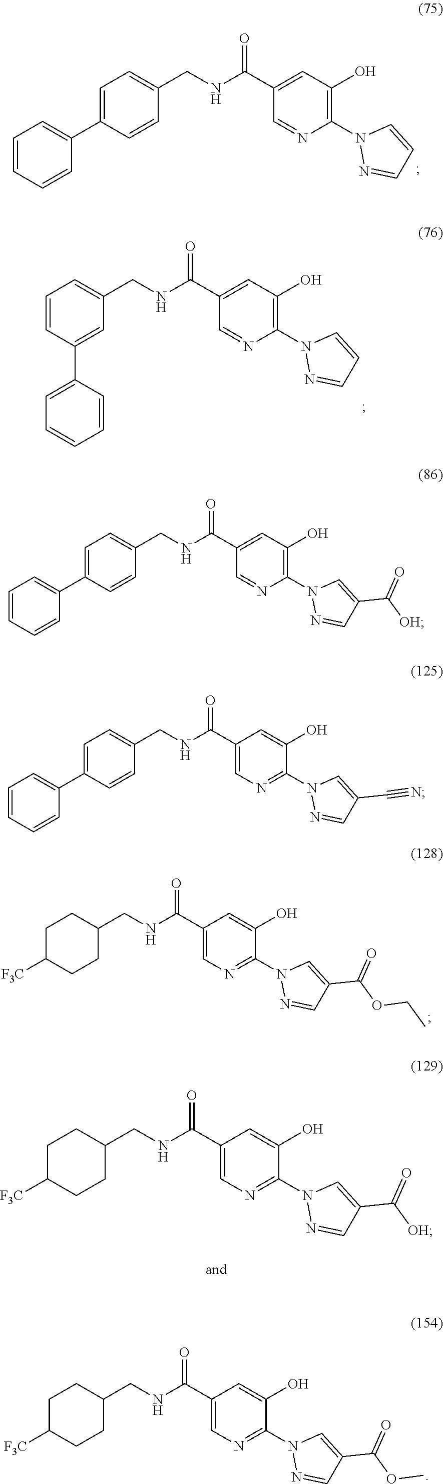

17. A compound according to any one of claims 10 to 12 wherein the substituted azine has the formula (Ic) 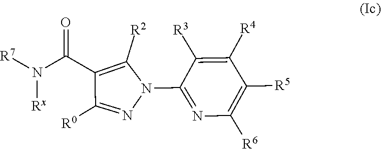
wherein
R⁰ is H or unsubstituted or substituted C₁₋₆ alkyl;
R² is H, —OR^{q} or unsubstituted or substituted C₁₋₆ alkyl;
R³ is H, —OR⁸ or unsubstituted or substituted C₁₋₆ alkyl;
R⁴ is H, unsubstituted or substituted C₁₋₆ alkyl, —OR⁹ or —C(O)OR¹⁰;
R⁵ is H, unsubstituted or substituted C₁₋₆ alkyl, unsubstituted or substituted aryl, unsubstituted or substituted heteroaryl, —CN or —C(O)OR^{w};
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR² or unsubstituted or substituted C₁₋₄ alkyl;
R⁸, R⁹ and R¹⁰ are each independently selected from H and unsubstituted or substituted C₁₋₆ alkyl;
R^{w}, R^{x} and R^{z} are each independently selected from H, unsubstituted or substituted C₁₋₄ alkyl, and unsubstituted or substituted phenyl;
R^{q} is H, unsubstituted or substituted C₁₋₄ alkyl, or unsubstituted or substituted phenyl.

18. A compound according to claim 10 or claim 17 wherein the substituted azine has any one of the following structures:

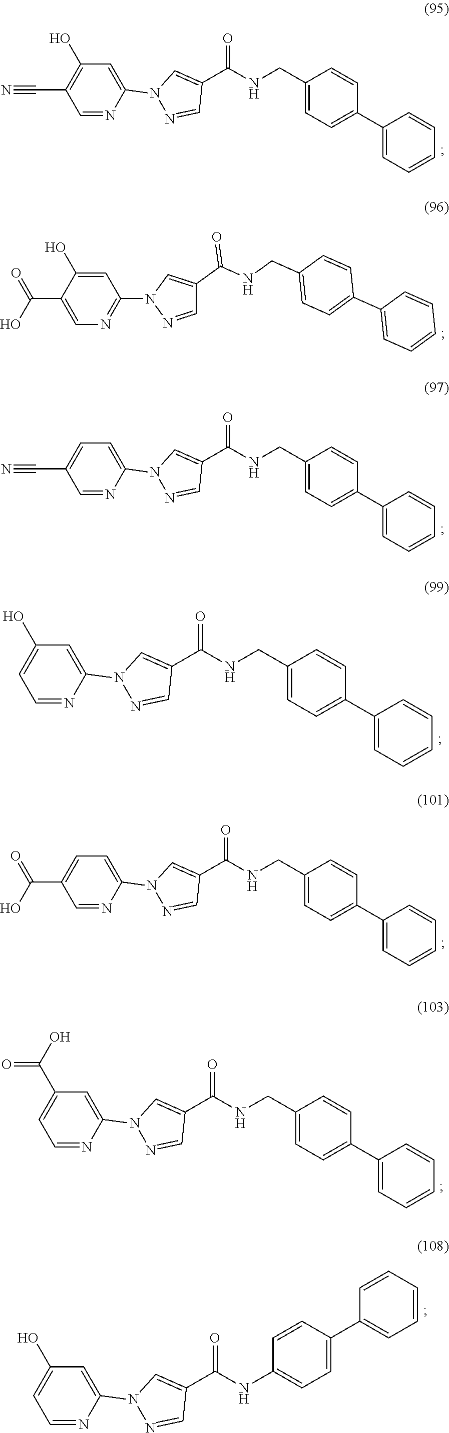

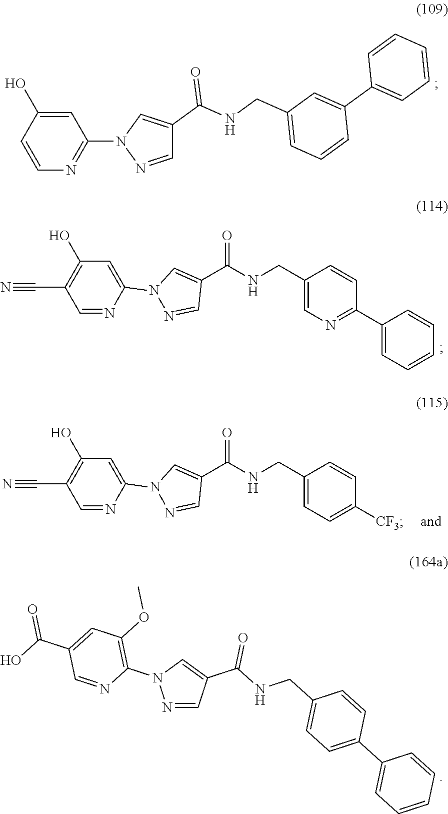 [SMILES: O=C(NCc1cccc(-c2ccccc2)c1)c1cnn(-c2cc(O)ccn2)c1 ; N#Cc1cnc(-n2cc(C(=O)NCc3ccc(-c4ccccc4)nc3)cn2)cc1O ; N#Cc1cnc(-n2cc(C(=O)NCc3ccc(C(F)(F)F)cc3)cn2)cc1O ; COc1cc(C(=O)O)cnc1-n1cc(C(=O)NCc2ccc(-c3ccccc3)cc2)cn1]

19. A compound according to any one of claims 10 to 12 wherein the substituted azine has the formula (Id) 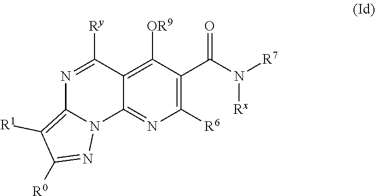
wherein
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —CN or —C(O)OR^{w};
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR² or unsubstituted or substituted C₁₋₄ alkyl;
R⁹ is H or unsubstituted or substituted C₁₋₆ alkyl; and
R^{w}, R^{x}, R^{y}, and R^{z} are each independently selected from H, unsubstituted or substituted C₁₋₄ alkyl, and unsubstituted or substituted phenyl.

20. A compound according to claim 10 or claim 19 wherein the substituted azine has any one of the following structures

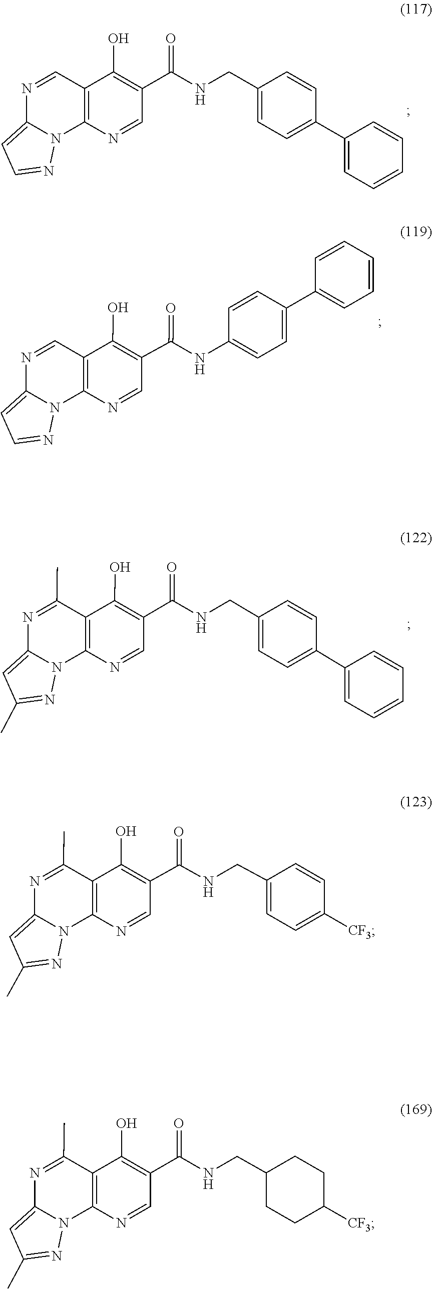 [SMILES: O=C(NCc1ccc(-c2ccccc2)cc1)c1cnc2c(cnc3ccnn32)c1O ; O=C(Nc1ccc(-c2ccccc2)cc1)c1cnc2c(cnc3ccnn32)c1O ; Cc1cc2nc(C)c3c(O)c(C(=O)NCc4ccc(-c5ccccc5)cc4)cnc3n2n1 ; Cc1cc2nc(C)c3c(O)c(C(=O)NCc4ccc(C(F)(F)F)cc4)cnc3n2n1 ; Cc1cc2nc(C)c3c(O)c(C(=O)NCC4CCC(C(F)(F)F)CC4)cnc3n2n1]

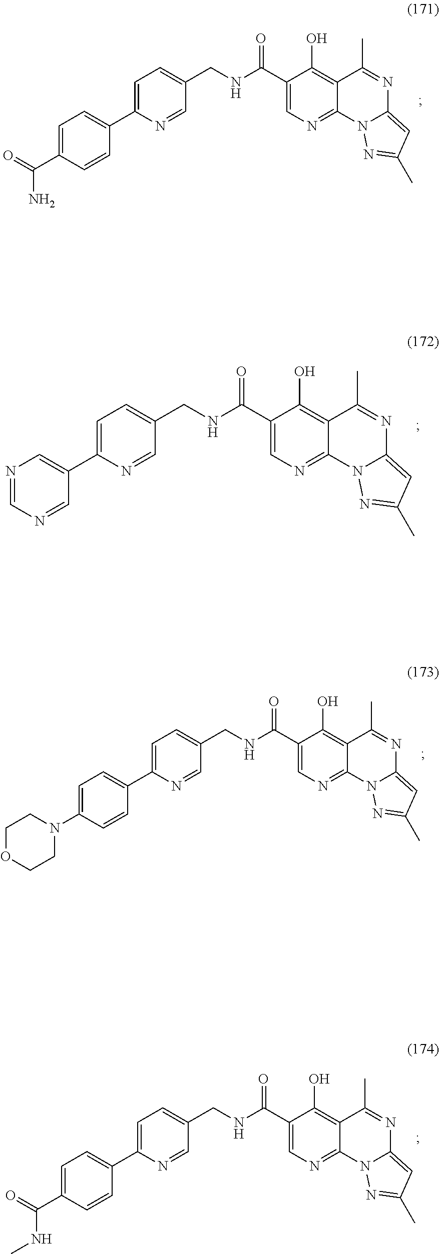 [SMILES: Cc1cc2nc(C)c3c(O)c(C(=O)NCc4ccc(-c5ccc(C(N)=O)cc5)nc4)cnc3n2n1 ; Cc1cc2nc(C)c3c(O)c(C(=O)NCc4ccc(-c5cncnc5)nc4)cnc3n2n1 ; Cc1cc2nc(C)c3c(O)c(C(=O)NCc4ccc(-c5ccc(N6CCOCC6)cc5)nc4)cnc3n2n1 ; CNC(=O)c1ccc(-c2ccc(CNC(=O)c3cnc4c(c(C)nc5cc(C)nn54)c3O)cn2)cc1]

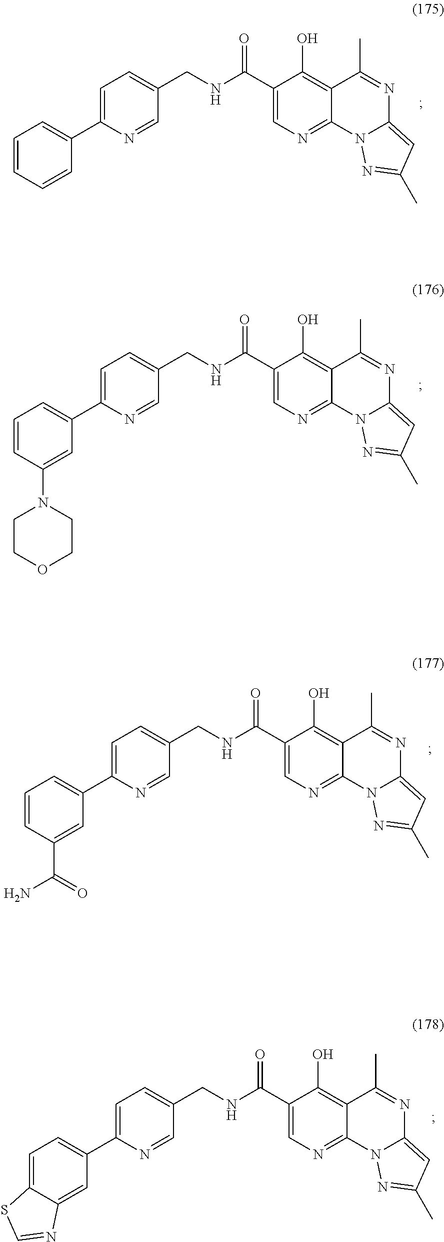 [SMILES: Cc1cc2nc(C)c3c(O)c(C(=O)NCc4ccc(-c5ccccc5)nc4)cnc3n2n1 ; Cc1cc2nc(C)c3c(O)c(C(=O)NCc4ccc(-c5cccc(N6CCOCC6)c5)nc4)cnc3n2n1 ; Cc1cc2nc(C)c3c(O)c(C(=O)NCc4ccc(-c5cccc(C(N)=O)c5)nc4)cnc3n2n1 ; Cc1cc2nc(C)c3c(O)c(C(=O)NCc4ccc(-c5ccc6scnc6c5)nc4)cnc3n2n1]

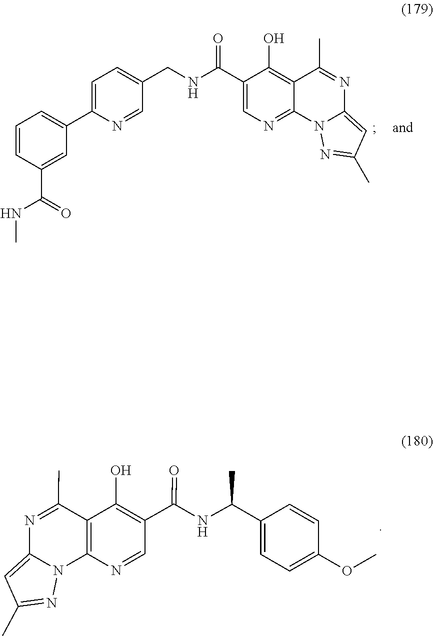

21. A compound which is a substituted pyrimidine of formula (IV) or a pharmaceutically acceptable salt thereof 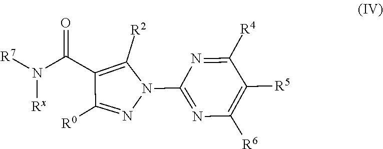
wherein
R⁰ is H or unsubstituted or substituted C₁₋₆ alkyl;
R² is H, —OR^{q} or unsubstituted or substituted C₁₋₆ alkyl;
R⁴ is —OR⁹
R⁵ is H, unsubstituted or substituted C₁₋₆ alkyl, unsubstituted or substituted aryl, unsubstituted or substituted heteroaryl, —CN or —C(O)OR^{w};
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹) Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, C(O)OR² or unsubstituted or substituted C₁₋₄ alkyl;
R⁹ is selected from H and unsubstituted or substituted C₁₋₆ alkyl;
R^{x} is H, unsubstituted or substituted C₁₋₄ alkyl, or unsubstituted or substituted phenyl;
R^{w} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.

22. A compound according to claim 21 wherein the substituted pyrimidine has any one of the following structures

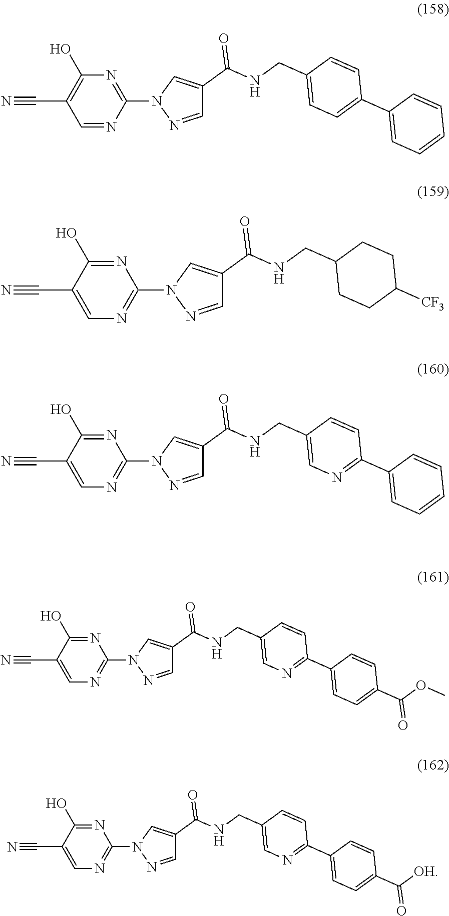 [SMILES: N#Cc1cnc(-n2cc(C(=O)NCc3ccc(-c4ccccc4)cc3)cn2)nc1O ; N#Cc1cnc(-n2cc(C(=O)NCC3CCC(C(F)(F)F)CC3)cn2)nc1O ; N#Cc1cnc(-n2cc(C(=O)NCc3ccc(-c4ccccc4)nc3)cn2)nc1O ; COC(=O)c1ccc(-c2ccc(CNC(=O)c3cnn(-c4ncc(C#N)c(O)n4)c3)cn2)cc1 ; N#Cc1cnc(-n2cc(C(=O)NCc3ccc(-c4ccc(C(=O)O)cc4)nc3)cn2)nc1O]

23. A pharmaceutical composition comprising a compound as defined in any one of claims 10 to 22 and a pharmaceutically acceptable carrier or diluent.

24. A compound as defined in any one of claims 10 to 22, or a pharmaceutical composition as defined in claim 23, for use in treating the human or animal body by therapy.

25. A HIF-alpha increasing agent for use in treating blood cancer.

26. A hypoxia inducible factor prolyl hydroxylase inhibitor (PHD inhibitor) for use in the treatment of blood cancer by simultaneous, separate or sequential co-administration with a factor inhibiting HIF inhibitor (FIH inhibitor).

27. A PHD inhibitor for use according to claim 26 wherein the PHD inhibitor is a compound of the following formula or a pharmaceutically acceptable salt thereof 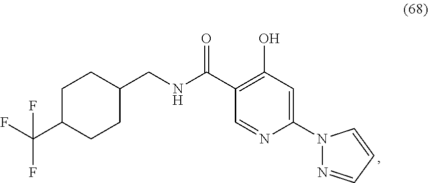 [SMILES: O=C(NCC1CCC(C(F)(F)F)CC1)c1cnc(-n2cccn2)cc1O]
and wherein the FIH inhibitor is dimethyl N-oxalyl-D-phenylalanine (DM-NOFD) or a pharmaceutically acceptable salt thereof.

28. A PHD inhibitor for use according to claim 26 or claim 27 wherein the treatment of blood cancer further comprises administration of a B-cell lymphoma 2 (BCL2) inhibitor, optionally wherein the BCL2 inhibitor is venetoclax or a pharmaceutically acceptable salt thereof.

29. A factor inhibiting HIF inhibitor (FIH inhibitor) for use in the treatment of blood cancer by simultaneous, separate or sequential co-administration with a hypoxia inducible factor prolyl hydroxylase inhibitor (PHD inhibitor).

30. A combination comprising a hypoxia inducible factor prolyl hydroxylase inhibitor (PHD inhibitor) and a factor inhibiting HIF inhibitor (FIH inhibitor).

31. A pharmaceutical composition comprising a hypoxia inducible factor prolyl hydroxylase inhibitor (PHD inhibitor), a factor inhibiting HIF inhibitor (FIH inhibitor), and a pharmaceutically acceptable carrier or diluent.

32. A combination according to claim 30 or a pharmaceutical composition according to claim 31 which further comprises a BCL2 inhibitor, optionally wherein the BCL2 inhibitor is venetoclax or a pharmaceutically acceptable salt thereof.

33. A combination according to claim 30 or 32 or a pharmaceutical composition according to claim 31 or 32, for use in the treatment of blood cancer.

34. A hypoxia inducible factor prolyl hydroxylase inhibitor (PHD inhibitor) for use in the treatment of blood cancer by simultaneous, separate or sequential co-administration with a BCL2 inhibitor.

35. A PHD inhibitor for use according to claim 34 wherein the PHD inhibitor is a compound of the following formula or a pharmaceutically acceptable salt thereof 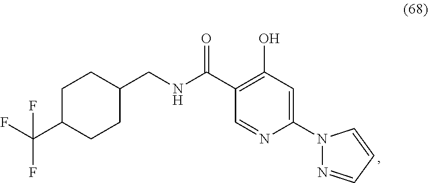 [SMILES: O=C(NCC1CCC(C(F)(F)F)CC1)c1cnc(-n2cccn2)cc1O]
and wherein the wherein the BCL2 inhibitor is venetoclax or a pharmaceutically acceptable salt thereof.

36. A BCL2 inhibitor for use in the treatment of blood cancer by simultaneous, separate or sequential co-administration with a hypoxia inducible factor prolyl hydroxylase inhibitor (PHD inhibitor).

37. A combination comprising a hypoxia inducible factor prolyl hydroxylase inhibitor (PHD inhibitor) and a BCL2 inhibitor.

38. A pharmaceutical composition comprising a hypoxia inducible factor prolyl hydroxylase inhibitor (PHD inhibitor), a BCL2 inhibitor, and a pharmaceutically acceptable carrier or diluent.

39. A combination according to claim 37 or a pharmaceutical composition according to claim 38, for use in the treatment of blood cancer.


SEQUENCE LISTING (9 sequences)

SEQ ID NO: 1 - protein, 19 aa, synthetic construct
  MOD_RES 19: note = amidated C-terminus
DLDLEMLAPYIPMDDDFQL

SEQ ID NO: 2 - RNA, 21 nt, synthetic construct
  source 1..21: mol_type = other RNA
gcctcggtttctatttataat

SEQ ID NO: 3 - RNA, 20 nt, synthetic construct
  source 1..20: mol_type = other RNA
ttctccgaacgtgtcacgtt

SEQ ID NO: 4 - DNA, 13 bp, synthetic construct
  source 1..13: mol_type = other DNA
agatcggaagagc

SEQ ID NO: 5 - protein, 12 aa, synthetic construct
RSKKRKKSKSRS

SEQ ID NO: 6 - protein, 14 aa, synthetic construct
  MOD_RES 9: note = lysine tri-methylation
ARTAQTARKSTGGI

SEQ ID NO: 7 - protein, 21 aa, synthetic construct
  MOD_RES 4: note = lysine tri-methylation
ARTKQTARKSTGGKAPRKQLA

SEQ ID NO: 8 - protein, 17 aa, synthetic construct
  MOD_RES 8: note = lysine tri-methylation
LATKAARKSAPATGGVK

SEQ ID NO: 9 - protein, 30 aa, synthetic construct
  MOD_RES 30: note = amidated C-terminus
AKGIVLEKVGVEAKQPNSAIRKAVRVQLIK